(19) United States
(12) Patent Application Publication
(10) Pub. No.: US 2021/0353652 A1
(43) Pub. Date: Nov. 18, 2021

(54) TUMOR MICROENVIRONMENT-ACTIVATED DRUG-BINDER CONJUGATES, AND USES RELATED THERETO

(71) Applicants: Trustees of Tufts College, Medford, MA (US); Avacta Life Sciences, Limited, Wetherby (GB)

(72) Inventors: Matthew Vincent, Amesbury, MA (US); Hung-sen Lai, Andover, MA (US); Amrik Basran, Cambridge (GB); William W. Bachovchin, Cambridge, MA (US)

(21) Appl. No.: 15/734,445

(22) PCT Filed: Jun. 4, 2019
(86) PCT No.: PCT/US19/35374

§ 371 Date: Dec. 2, 2020

Related U.S. Application Data
(60) Provisional application No. 62/680,300, filed on Jun. 4, 2018.

(51) Int. Cl. A61K 31/69; A61K 47/68; A61K 47/65; A61K 47/54; A61P 35/00

(52) U.S. Cl. CPC A61K 31/69; A61K 47/6849; A61P 35/00; A61K 47/545; A61K 47/65

(57) ABSTRACT

Disclosed are binder-drug conjugates that are activated extracellular, with both the binder and the free drug moiety have pharmacological activity.

### RELATED APPLICATIONS

This application claims the benefit of priority to U.S. Provisional Patent Application No. 62/680,300, filed Jun. 4, 2018.

### BACKGROUND

PD-1-PD-L1 interaction is known to drive T cell dysfunction, which can be blocked by anti-PD-1/PD-L1 antibodies. However, studies have also shown that the function of the PD-1-PD-L1 axis is affected by the complex immunologic regulation network. In most advanced cancers, except Hodgkin lymphoma (which has high PD-L1/L2 expression) and melanoma (which has high tumor mutational burden), the objective response rate with anti-PD-1/PD-L1 monotherapy is only about 20%, and immune-related toxicities and hyperprogression can occur in a small subset of patients during PD-1/PD-L1 blockade therapy. The lack of efficacy in up to 80% of patients was not necessarily associated with negative PD-1 and PD-L1 expression, suggesting that the roles of PD-1/PD-L1 in immune suppression and the mechanisms of action of antibodies remain to be better defined. Likewise, similar limitations have been observed with CTLA-4 and other checkpoint pathways. Accordingly, important synergizing immune regulatory mechanisms within or outside of the PD-1/PD-L1, CTLA-4 and other checkpoint networks need to be targeted to increase the response rate, and in some cases to reduce the toxicities, of immune checkpoint blockade therapies.

In this regard, drug agents that induce innate immune responses, such as STING, RIG-I and TLR agonists, are believed to have the potential to increase the effectiveness of immuno-oncology checkpoint inhibitors. However, these types of agents are often too toxic for systemic use, as the dose limiting toxicities are the product of innate immune activation throughout the body, and the maximum tolerated doses do not achieve therapeutic doses in many patients.

The present invention is based on a new system for, inter alia, co-delivery of these two classes of therapeutic agents—inducers of innate immunity that cause a localized inflammatory event in the tumor that invokes a potent immune response with one or more checkpoint inhibitors or costimulatory agonists that promote or maintain an adaptive immune response—in a format that addresses the systemic toxicity issues of either component, particularly the innate immunity inducer, but holding it in an pharmacologically inactive form until released by a protease in the tumor microenvironment. Put more simply, one agent induces an antitumor immune response and the other makes sure that it works when it gets to the tumor. The checkpoint inhibitor or co-stimulatory agonist along with the TME enzyme release help to locate the drugs in the tumor and improve the therapeutic index relative to that of the components drugs individually.

### SUMMARY

One aspect of the present invention relates to a binder-drug conjugate comprising:
(i) a cell binding moiety that binds to a cell surface feature on a target cell in a disease state of a tissue, which cell surface feature undergoes slow internalization when bound by the binder-drug conjugate;
(ii) a drug moiety that has a pharmacological effect on bystander cells proximate to the target cell, which drug moiety has an EC50 for the pharmacological effect which is attenuated by at least 2-fold when part of the binder-drug conjugate relative to a free drug moiety released from the binder-drug conjugate; and
(iii) a linker moiety covalently linking the polypeptide binder moiety to the drug moiety, which linker moiety includes a substrate recognition sequence that is cleavable by an enzyme present extracellularly in the disease tissue, wherein in the presence of the enzyme the linker moiety can be cleaved and releases the free drug moiety.

In certain embodiments, the drug moiety has an EC50 for the pharmacological effect which is attenuated by at least 5-fold when part of the binder-drug conjugate relative to a free drug moiety released from the binder-drug conjugate, and more preferably attenuated at least 10, 20, 30, 40, 50, 75, 100, 250, 500 or even 1000-fold.

In certain embodiments, the disease tissue is a tumor. In certain embodiments, the target cell is a tumor cell. In certain embodiments, the target cell is a macrophage, monocyte derived suppressor cells (MDSC), dendritic cells, fiboblasts, T-cells, NK cell, Mast Cells, Granulocytes, Eiosinophils and B-cells.

In certain embodiments, the binder-drug conjugate, when bound with the surface feature on the target cell has an internalization half-time of at least 6 hours, more preferably at least 10, 12, 14, 16, 18, 20, 24, 36, 48, 60, 75 or even 100 hours.

In certain embodiments, the cell surface feature is a protein selectively expressed or upregulated by the target cell in the disease tissue relative to normal cells from a healthy state of the tissue. For instance, the protein is detectable on the surface of the target cells at levels 2 fold higher than normal cells from the tissue, even more preferably levels at least 5, 10, 20, 30, 40, 50, 75, 100, 250, 500 or even 1000-fold higher than normal cells from the tissue.

In certain embodiments, the cell surface feature is a protein selectively expressed or upregulated by the target cell in the disease tissue relative to cells from other tissues, particularly cells from critical organs. For instance, the protein is detectable on the surface of the target cells at levels 2 fold higher than cells from other tissues, even more preferably levels at least 5, 10, 20, 30, 40, 50, 75, 100, 250, 500 or even 1000-fold higher than cells from other tissues.

In certain embodiments, the cell surface feature is a checkpoint protein and the binder moiety is an antagonist of that checkpoint. Examples of checkpoint proteins include those selected from the group consisting of CTLA-4, PD-1, LAG-3, BTLA, KIR, TIM-3, PD-L1, PD-L2, B7-H3, B7-H4, HVEM, GAL9, CD160, VISTA, BTNL2, TIGIT, PVR, BTN1A1, BTN2A2, BTN3A2 and CSF-1R, more preferably CTLA-4, PD-1, LAG-3, TIM-3, BTLA, VISTA, HVEM, TIGIT, PVR, PD-L1 and CD160.

In certain embodiments, the cell surface feature is a co-stimulatory receptor and the binder moiety is a costimulatory agonist of the receptor. Examples include the surface feature being a cotimulatory receptor or ligand selected from the group consisting of 4-1BB, 4-1BB-L, OX40, OX40-L, GITR, CD28, CD40, CD40-L, ICOS, ICOS-L, LIGHT, and CD27, more preferably 4-1BB, OX40, GITR, CD40 and ICOS.

In certain embodiments, the cell binding moiety is an antibody, such as a humanized antibody, a human antibody, or a chimeric antibody, or comprises an antigen-binding portion thereof that binds the cell surface feature, such as Fab, F(ab)2, F(ab′), F(ab′)2, F(ab′)3, Fd, Fv, disulfide linked Fv, dAb or sdAb (or nanobody), CDR, scFv, (scFv)2, di-scFv, bi-scFv, tascFv (tandem scFv), AVIBODY (e.g., diabody, triabody, tetrabody), T-cell engager (BiTE), scFv-Fc, Fcab, mAb2, small modular immunopharmaceutical (SMIP), Genmab/unibody or duobody, V-NAR domain, IgNAR, minibody, IgGACH2, DVD-Ig, probody, intrabody, or a multispecificity antibody.

In other embodiments, the binder moiety is non-antibody scaffold, such as selected from the group consisting of Affibodies, Affimers, Affilins, Anticalins, Atrimers, Avimer, DARPins, FN3 scaffolds (e.g. Adnectins and Centyrins), Fynomers, Kunitz domains, Nanofitin, Pronectins, OBodies, tribodies, Avimers, bicyclic peptides and Cys-knots.

In certain embodiments, the linker moiety includes two, three or even four substrate recognition sequences that are cleavable by the same or different enzymes present in the disease tissue (at least one of which is present extracellularly), wherein in the simultaneous or serial presence of the various enzymes the linker moiety can be cleaved completely so as to release the free drug moiety. For instance, a linker with two different substrate recognition sequence can be created to require cleavage by both an MMP and FAPα. In preferred embodiments, the cleavage by one of the two enzymes requires the cleavage by the other enzyme to have happened first—i.e., MMP cleavage can be required before FAPα cleavage by creating a linker which is a poor substrate for FAPα when intact, and is improved as a substrate for cleavage by FAPα after MMP cleavage has occurred.

To further illustrate, the binder-drug conjugate can be represented by one of the formula

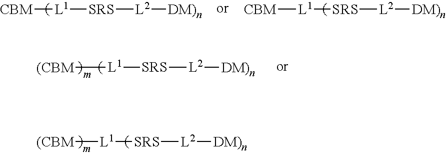

wherein
   CBM represents a cell binding moiety which may be the same or different for each occurrence;
   L¹ represents a spacer or a bond;
   SRS represents a substrate recognition sequence;
   L² represents a self immolative linker or a bond;
   DM represents a drug moiety;
   m represents an integer from 1 to 6; and
   n represents an integer from 1 to 500, more preferably 1 to 100, 1 to 10 or 1 to 5.

In certain embodiments, L¹ is a hydrocarbon (straight chain or cyclic) such as 6-maleimidocaproyl, maleimidopropanoyl and maleimidomethyl cyclohexane-1-carboxylate, or L¹ is N-Succinimidyl 4-(2-pyridylthio) pentanoate, N-Succinimidyl 4-(N-maleimidomethyl) cyclohexane-1 carboxylate, N-Succinimidyl (4-iodo-acetyl) aminobenzoate

In certain embodiments, L¹ is a polyether such as a poly(ethylene glycol) or other hydrophilic linker. For instance, where the CBM includes a thiol (such as a cysteine residue), L¹ can be a poly(ethylene glycol) coupled to the thiol group through a maleimide moiety, such as represented in the formula

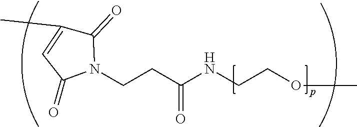

wherein p represents an integer from 1 to 100, preferably 6 to 50, more preferably 6 to 12.

In other embodiments, where the CBM includes a thiol and L¹ is a hydrocarbon moiety coupled to the thiol group through a maleimide moiety, L¹ can be represented in the formula

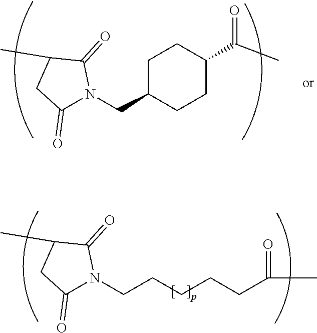

wherein p represent an integer from 1 to 20, preferably 1 to 4.

In certain embodiments of the binder-drug conjugates of the present invention the substrate recognition sequence is cleaved by an extracellular protease, preferably a serine protease, a metalloprotease or cysteine protease with a protease activity located in the extracellular domain of the target tissue—i.e., as a cell-surface protease or a secreted/released protease.

In certain embodiments, the protease is present extracellularly in the disease state of the tissue in a patient at levels at least 5, 10, 20, 30, 40, 50, 75, 100, 250, 500 or even 1000-fold higher than it is present extracellularally in the healthy state of the tissue in a patient.

In certain embodiments, the protease is present extracellularly in the disease state of the tissue in a patient at levels at least 5, 10, 20, 30, 40, 50, 75, 100, 250, 500 or even 1000-fold higher than other tissue of the patient.

In certain embodiments, the protease is a matrix metalloproteinase. The matrix metalloproteinase can be a membrane bound matrix metalloproteinases (such as MMP14-17 and MMP24-25) or a secreted matrix metalloproteinase (such as MMP1-13 and MMP18-23 and MMP26-28). In certain embodiments, the metalloproteinase is MMP1, MMP2, MMP3, MMP4, MMP9, MMP11, MMP13, MMP14, MMP17 or MMP19, and more preferably is MMP2, MMP9 or MMP14.

In certain embodiments, the protease is an A Disintegrin and Metalloproteinase (ADAM), or an A Disintegrin or Metalloproteinase with Thrombospondin Motifs (ADAMTS).

In certain embodiments, the protease is a legumain, a matriptase (MT-SP1), a neutrophil elastase, a TMPRSS, a thrombin, a u-type plasminogen activator (uPA, also referred to as urokinase), PSMA or CD10 (CALLA).

In certain embodiments, the protease is a post-proline cleaving protease, such as fiboblast activating protein alpha (FAPα).

In certain embodiments of the subject binder-drug conjugate, the substrate recognition sequence is cleaved by fiboblast activating protein alpha (FAPα) and is represented by

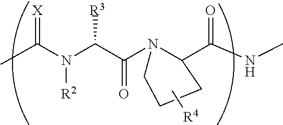

wherein
R² represents H or a (C₁-C₆) alkyl, and preferably is H,
R³ represents H or a (C₁-C₆) alkyl, preferably is methyl, ethyl, propyl, or isopropyl, and more preferably methyl;
R⁴ is absent or represents a (C₁-C₆) alkyl, —OH, —NH₂, or halogen;
X represents O or S; and
—NH— represents an amine that is part of L² if L² is a self immolative linker or part of DM if L² is a bond.

In certain embodiments, R² is H, R³ is methyl, R⁴ is absent and X is O.

In certain embodiments, L² is a self immolative linker selected from the group consisting of —NH—(CH₂)₄—C(═O)—, —NH—(CH₂)₃—C(═O)—, p-aminobenzyloxycarbonyl (PABC) and 2,4-bis(hydroxymethyl)aniline. In certain embodiments, L2 is p-aminobenzyloxycarbonyl (PABC), particularly in the case of the subject recognition sequence being cleaved by FAP□ as p-aminobenzyloxycarbonyl (PABC) tills the P′1 specificity requirements for FAP□.

In certain embodiments, free drug moiety interacts with an intracellular target and the pharmacological effect of the drug moiety is dependent on the free drug moiety being cell permeable, i.e., and able to interact with its intracellular target, whereas when part of the binder-drug conjugate the drug moiety is substantially cell impermeable. For instance, the rate of accumulation of the binder-drug conjugate intracellularly is less than 50% of the rate for the free drug moiety, more preferably less than 25%, 10%, 5%, 1% or even less than 0.1% of the rate for the free drug moiety. For instance, the EC50 for the pharmacological effect of the free drug moiety is at least 2 fold less than (more potent than) the binder-drug conjugate, more preferably at least 5, 10, 20, 30, 40, 50, 100, 250, 500 or even 1000 less than the binder-drug conjugate.

In certain embodiments, the free drug moiety interacts with an extracellular target and the pharmacological effect of the drug moiety is substantially attenuated when covalently linked to L¹. For instance, the EC50 for the pharmacological effect of the free drug moiety is at least 2 fold less than (more potent than) the binder-drug conjugate, more preferably at least 5, 10, 20, 30, 40, 50, 100, 250, 500 or even 1000 less than the binder-drug conjugate.

In certain embodiments, the binder-drug conjugate has a therapeutic index when delivered systemically that is at least 2-fold greater than the systemic delivery of the free drug moiety, and even more preferably at least 5, 10, 20, 30, 40, 50, 100, 250, 500 or even 1000 greater than the systemic delivery of the free drug moiety.

In certain embodiments, the free drug moiety is an immunomodulator—which includes drug moieties acting as immune activating agents and/or inducers of an innate immunity pathway response. In certain embodiments, the free drug moiety induces the production of IFN-α. In certain embodiments, the free drug moiety induces the production of proinflammatory cytokines. In certain embodiments, the free drug moiety induces the production of IL-1β. In certain embodiments, the free drug moiety induces the production of IL-18.

In certain embodiments, the free drug moiety promotes the expansion and survival of effector cells including NK, γδ T, and CD8+ T cells.

In certain embodiments, the free drug moiety is an immuno-DASH inhibitor that inhibits the enzymatic activity of DPP8 and DPP9, and induces macrophage pyroptosis in vitro and/or in vivo.

In certain embodiments, the free drug moiety is a damage-associated molecular pattern molecule. In certain embodiments, the free drug moiety is a pathogen-associated molecular pattern molecule.

In certain embodiments, the free drug moiety is a STING agonist.

In certain embodiments, the free drug moiety is a RIG-1 agonist.

In certain embodiments, the free drug moiety is a Toll-like receptor (TLR) agonist, such as a selected from the group consisting of a TLR1/2 agonist, a TLR2 agonist, a TLR3 agonist, a TLR4 agonist, a TLR5 agonist, a TLR6/2 agonist, a TLR7 agonist, a TLR7/8 agonist, a TLR7/9 agonist, a TLR8 agonist, a TLR9 agonist, and a TLR11 agonist, preferably selected from the group consisting of a TLR3 agonist, a TLR7 agonist, a TLR7/8 agonist, and a TLR9 agonist.

In certain embodiments, the free drug moiety is a cyclic dinucleotide.

In certain embodiments, the free drug moiety is ADU-S100.

In certain embodiments, the free drug moiety is a RIG-1 agonist, wherein the RIG-1 agonist is KIN700, KIN1148, KIN600, KIN500, KIN100, KIN101, KIN400, KIN2000, or SB-9200.

In certain embodiments, the free drug moiety is selected from a group consisting of: S-27609, CL307, UC-IV150, imiquimod, gardiquimod, resiquimod, motolimod, VTS-1463GS-9620, GSK2245035, TMX-101, TMX-201, TMX-202, isatoribine, AZD8848, MEDI9197, 3M-051, 3M-852, 3M-052, 3M-854A, S-34240, KU34B, or CL663.

In certain embodiments, the free drug moiety is cytotoxic to cancer associated fibroblasts (CAFs).

In certain embodiments, the free drug moiety polarizes tumor associated macrophage populations towards M1 macrophage and/or inhibits M2 macrophage immunosuppressive activity.

In certain embodiments, the free drug moiety accelerates T-cell priming and/or dendritic cell trafficking.

In certain embodiments, the free drug moiety inhibits or depletes Treg cells, such as by blocking immunosuppressive function or migration to lymph nodes and/or the tumor microenvironment.

In certain embodiments, wherein the therapeutic index (TI) of the binder-drug conjugate is at least 5 times greater than the therapeutic index for the free drug moiety when given systemically, more preferably at least 10, 20, 30, 40, 50, 75 or even 100 times greater.

In certain embodiments, the free drug moiety is a low-molecular inhibitor, i.e., having a molecular weight less than 5000 amu, preferably less than 2500 amu and even more preferably less than 1500 amu.

Another aspect of the invention provides a binder-drug conjugate comprising a polypeptide including one or more small domain binding polypeptide sequences (such as an antibody fragment or non-antibody scaffold), preferably one more affimer sequences, that bind to a cell surface protein on cells in a tumor, and having one or more drug-conjugate moieties appended thereto, which drug-conjugate moieties are represented in the formulas

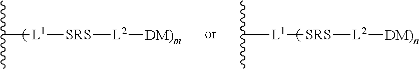

wherein
   L¹ represents a spacer or a bond;
   SRS represents a substrate recognition sequence for an extracellular protease which is expressed in the extracellular space of a tumor;
   L² represents a self immolative linker or a bond;
   DM represents a drug moiety;
   m represents an integer from 1 to 6, preferably 1, 2 or 3; and
   n represents an integer from 1 to 500, more preferably 1 to 100, 1 to 10 or 1 to 5.

The binder-drug conjugate, when bound with the surface feature on the target cell has an internalization half-time of at least 6 hours, more preferably at least 10, 12, 14, 16, 18, 20, 24, 36, 48, 60, 75 or even 100 hours.

In certain embodiments, at least one of the small domain binding polypeptide sequences is a PD-L1 binding moiety.

In certain embodiments, the polypeptide of the binder-drug conjugate binds to PD-L1 with a Kd of 1×10⁻⁶M or less (and more preferably with a Kd of 1× 10⁻⁷M, 1× 10⁻⁸M, 1× 10⁻⁹M, 1×10⁻¹M, or even 1× 10⁻¹¹M or less, particularly in embodiments wherein the polypeptide is bivalent or higher order multivalent for PD-L1 binding) and which inhibits interaction of the PD-L1 to which it is bound with PD-1.

Another aspect of the invention provides a multispecific binder-drug conjugate comprising
(i) a polypeptide including two or more different binding domain polypeptide sequences that selectively bind to two different cell surface proteins on two different cell types in a tumor, and
(ii) one or more drug-conjugate moieties appended to the polypeptide, which drug-conjugate moieties are represented in the formulas

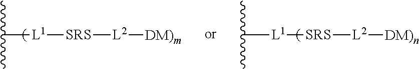

wherein
   L¹ represents a spacer or a bond;
   SRS represents a substrate recognition sequence for an extracellular protease which is expressed in the extracellular space of a tumor;
   L² represents a self immolative linker or a bond;
   DM represents a drug moiety;
   m represents an integer from 1 to 6, preferably 1, 2 or 3; and
   n represents an integer from 1 to 500, more preferably 1 to 100, 1 to 10 or 1 to 5.

The multispecific binder-drug conjugate, when bound with either of the surface proteins has an internalization half-time of at least 6 hours, more preferably at least 10, 12, 14, 16, 18, 20, 24, 36, 48, 60, 75 or even 100 hours.

In certain embodiments of the multispecific binder-drug conjugate, the polypeptide includes a first binding domain polypeptide sequence that selectively bind to a tumor cell antigen and a second binding domain polypeptide sequence that selectively binds to a cell selected from the group consisting of macrophage, monocyte derived suppressor cells (MDSC), dendritic cells, fiboblasts, NK cell, Mast Cells, Granulocytes, Eiosinophils and B-cells.

In certain embodiments of the multispecific binder-drug conjugate, the polypeptide includes a first binding domain polypeptide sequence that is checkpoint inhibitor or costimulatory agonist and binds to a checkpoint protein or costimulatory receptor protein expressed on tumor infiltrating lymphocytes (such as LAG-3, TIM-3, TIGIT, PD-1, BTLA or CTLA-4 in the case of checkpoints, and CD28, ICOS, OX40, GITR, CD137 or CD27 in the case of co-stimulatory proteins), and the second binding domain polypeptide sequence that is a checkpoint inhibitor that binds to checkpoint expressed on tumor cells (such as PD-L1, PD-L2, CD80, CD86, B7-H3, B7-H4, CD155, HVEM or galectin-9).

Yet another aspect of the present invention relates to a combination PD-L1 inhibitor/innate immune stimulator comprising a PD-L1 binding polypeptide and a drug moiety conjugated thereto which is a sterile inducer of a innate immune response (such as an immuno-DASH inhibitor, STING agonist, TRL7/8 agonist or RIG-1 agonist), wherein the PD-L1 binding polypeptide causes accumulation of the PD-L1 inhibitor/innate stimulator in tumors relative to other tissue of a patient, and wherein the drug moiety is selectively released from the PD-L1 binding polypeptide in the tumor microenvironment relative to other tissue of a patient.

In certain embodiments of the drug-conjugates of the invention, the molecule includes a PD-L1 binding moiety which is an affimer polypeptide sequence which binds to PD-L1 with a Kd of 1× 10⁻⁶M or less (and more preferably with a Kd of 1× 10⁻⁷M, 1× 10⁻⁸ M 1× 10⁻⁹M 1×10⁻¹⁰M or less) and inhibits interaction of the PD-L1 to which it is bound with PD-1.

In certain embodiments, the PD-L1 binding affimer polypeptide binds human PD-L1 and blocks interactions with human PD-1. In certain embodiments, the PD-L1 binding affimer polypeptide bind PD-L1 with a Kd of 1×10⁻⁷M or less, Kd of 1×10⁻⁸M or less, Kd of 1× 10⁻⁹M or less, or even a Kd of 1× 10⁻¹⁰M or less. In certain embodiments, the PD-L1 binding affimer polypeptide bind PD-L1 with a K_{off} of 10⁻³ s⁻¹ or slower, 10⁻⁴ s⁻¹ or slower, or even 10⁻⁵ s⁻¹ or slower. In certain embodiments, the PD-L1 binding affimer polypeptide bind PD-L1 with a Kₒₙ of 10³ M⁻¹ s⁻¹ or faster, 10⁴ M⁻¹ s⁻¹ or faster, 10⁵ M⁻¹ s⁻¹ or faster, or even 10⁶ M⁻¹ s⁻¹ or faster. In certain embodiments, the PD-L1 binding affimer polypeptide bind PD-L1 with an IC50 in a competitive binding assay with human PD-1 of 1 μM or less, 100 nM or less, 40 nM or less, 20 nM or less, 10 nM or less, 1 nM or less, or even 0.1 nM or less.

In certain embodiments, the PD-L1 binding affimer polypeptide has Tm of 65° C. or higher, and 70° C. or higher, 75° C. or higher, 80° C. or higher or 85° C. or higher. In certain embodiments, the protein has Tm of 65° C. or higher, and 70° C. or higher, 75° C. or higher, 80° C. or higher or 85° C. or higher.

In certain embodiments, the PD-L1 binding affimer polypeptide has an amino acid sequence represented in general formula (I)

FR1-(Xaa)ₙ-FR2-(Xaa)ₘ-FR3 (I)

wherein
FR1 is a polypeptide sequence represented by MIPGGLSEAK PATPEIQEIV DKVKPQLEEK TNETYGKLEA VQYKTQVLA (SEQ ID No. 1) or a polypeptide sequence having at least 70% homology thereto;
FR2 is a polypeptide sequence represented by GTNYYIKVRA GDNKYMHLKV FKSL (SEQ ID No. 2) or a polypeptide sequence having at least 70% homology thereto;
FR3 is a polypeptide sequence represented by EDLVLTGYQV DKNKDDELTG F (SEQ ID No. 3) or a polypeptide sequence having at least 70% homology thereto; and
Xaa, individually for each occurrence, is an amino acid residue; and
n and m are each, independently, an integer from 3 to 20.

For certain embodiments, the FR1 may a polypeptide sequence having at least 80%, 85%, 90%, 95% or even 98% homology with SEQ ID No. 1. For certain embodiments, FR2 is a polypeptide sequence having at least 80%, 85%, 90%, 95% or even 98% homology with SEQ ID No. 2. For certain embodiments, FR3 is a polypeptide sequence having at least 80%, 85%, 90%, 95% or even 98% homology with SEQ ID No. 2.

For those embodiments in which at least one drug-conjugate moiety is appended to the affimer sequence through a thiol side chain of a cysteine introduced into the affimer sequence, the cysteine will preferably be provided in a portion of the affimer sequence regions corresponding to FR1, FR2 and/or FR3, and more preferably with a replacement to an amino acid residue in the affimer the side chain of which is solvent accessible and is not involved in hydrogen bonding with other portions of the affimer. In general, cysteines will not be introduced into the loops (Xaa)ₙ or (Xaa)ₘ.

In certain embodiments, the PD-L1 binding affimer polypeptide has an amino acid sequence represented in the general formula:

(SEQ ID No. 4)
MIP-Xaa1-GLSEAKPATPEIQEIVDKVKPQLEEKTNETYGKLEAVQYKT
QVLA-(Xaa)ₙ-Xaa2-TNYYIKVRAGDNKYMHLKVF-Xaa3-Xaa4-
Xaa5-(Xaa)ₘ-Xaa6-D-Xaa7-VLTGYQVDKNKDDELTGF

wherein
Xaa, individually for each occurrence, is an amino acid residue;
n and m are each, independently, an integer from 3 to 20;
Xaa1 is Gly, Ala, Val, Arg, Lys, Asp, or Glu;
Xaa2 is Gly, Ala, Val, Ser or Thr;
Xaa3 is Arg, Lys, Asn, Gln, Ser, or Thr;
Xaa4 is Gly, Ala, Val, Ser or Thr;
Xaa5 is Ala, Val, Ile, Leu, Gly or Pro;
Xaa6 is Gly, Ala, Val, Asp or Glu; and
Xaa7 is Ala, Val, Ile, Leu, Arg or Lys.

For certain embodiments, Xaa1 is Gly, Ala, Arg or Lys, more even more preferably Gly or Arg. For certain embodiments, Xaa2 is Gly or Ser. For certain embodiments, Xaa3 is Arg Arg, Lys, Asn or Gln, more preferably Lys or Asn. For certain embodiments, Xaa4 is Gly or Ser. For certain embodiments, Xaa5 is Ala, Val, Ile, Leu, Gly or Pro, more preferably Ile, Leu or Pro, and even more preferably Leu or Pro. For certain embodiments, Xaa6 is Ala, Val, Asp or Glu, even more preferably Ala or Glu. For certain embodiments, Xaa7 is Ile, Leu or Arg, more preferably Leu or Arg.

For those embodiments in which at least one drug-conjugate moiety is appended to the affimer sequence through a thiol side chain of a cysteine introduced into the affimer sequence, the cysteine will preferably be provided in a portion of the affimer sequence other than with the loop sequences (Xaa)ₙ or (Xaa)ₘ. Accordingly, the SEQ ID No. 4 may include from 1 to cysteines in place of amino acid residues at varying positions of that sequence.

In certain embodiments, the PD-L1 binding affimer polypeptide has an amino acid sequence represented in the general formula:

(SEQ ID No. 5)
MIPRGLSEAKPATPEIQEIVDKVKPQLEEKTNETYGKLEAVQYKTQVLA-
(Xaa)ₙ-STNYYIKVRAGDNKYMHLKVFNGP-(Xaa)ₘ-ADRVLTGYQVD
KNKDDELTGF

wherein Xaa, individually for each occurrence, is an amino acid residue; and n and m are each, independently, an integer from 3 to 20.

For those embodiments in which at least one drug-conjugate moiety is appended to the affimer sequence through a thiol side chain of a cysteine introduced into the affimer sequence, the cysteine will preferably be provided in a portion of the affimer sequence other than with the loop sequences (Xaa)ₙ or (Xaa)ₘ. Accordingly, the SEQ ID No. 5 may include from 1 to cysteines in place of amino acid residues at varying positions of that sequence.

In certain embodiments of the above sequences, (Xaa)ₙ (“loop 2”) is an amino acid sequence represented in the general formula (II)

-aa1-aa2-aa3-Gly-Pro-aa4-aa5-Trp-aa6- (II)

wherein
aa1 represents an amino acid residue with a basic sidechain;
aa2 represents an amino acid residue, preferably an amino acid residue with a neutral polar or non-polar sidechain or a charged (acidic or basic) sidechain, more preferably a small aliphatic sidechain, a neutral polar side chain or a basic or acid side chain;
aa3 represents an amino acid residue with an aromatic or basic sidechain;
aa4 represents an amino acid residue with a neutral polar or non-polar sidechain or a charged (acidic or basic) sidechain; preferably a neutral polar sidechain or a charged (acidic or basic) sidechain;
aa5 represents an amino acid residue with a neutral polar or a charged (acidic or basic) or a small aliphatic or an aromatic sidechain; preferably a neutral polar sidechain or a charged sidechain; and
aa6 represents an amino acid residue with an aromatic or acid sidechain.

For certain embodiments, aa1 represents Lys, Arg or His, more preferably Lys or Arg. For certain embodiments, aa2 represents Ala, Pro, Ile, Gln, Thr, Asp, Glu, Lys, Arg or His, more preferably Ala, Gln, Asp or Glu. For certain embodiments, aa3 represents Phe, Tyr, Trp, Lys, Arg or His, preferably Phe, Tyr, Trp, more preferably His or Tyr, Trp or His. For certain embodiments, aa4 represents Ala, Pro, Ile, Gln, Thr, Asp, Glu, Lys, Arg or His, more preferably Gln, Lys, Arg, His, Asp or Glu. For certain embodiments, aa5 represents Ser, Thr, Asn, Gln, Asp, Glu, Arg or His, more preferably Ser, Asn, Gln, Asp, Glu or Arg. For certain embodiments, aa6 represents Phe, Tyr, Trp, Asp or Glu; preferably Trp or Asp; more preferably Trp.

In certain other embodiments of the above sequences, (Xaa)ₙ (“loop 2”) is an amino acid sequence represented in the general formula (III)

-aa1-aa2-aa3-Phe-Pro-aa4-aa5-Phe-Trp- (III)
wherein
aa1 represents an amino acid residue with a basic sidechain or aromatic sidechain;
aa2 represents an amino acid residue, preferably an amino acid residue with a neutral polar or non-polar sidechain or a charged (acidic or basic) sidechain, more preferably a small aliphatic sidechain, a neutral polar side chain or a basic or acid side chain;
aa3 represents an amino acid residue with an aromatic or basic sidechain, preferably Phe, Tyr, Trp, Lys, Arg or His, more preferably Phe, Tyr, Trp or His, and even more preferably Tyr, Trp or His;
aa4 represents an amino acid residue with a neutral polar or non-polar sidechain or a charged (acidic or basic) sidechain; preferably a neutral polar sidechain or a charged (acidic or basic) sidechain; more preferably Ala, Pro, Ile, Gln, Thr, Asp, Glu, Lys, Arg or His, and even more preferably Gln, Lys, Arg, His, Asp or Glu; and
aa5 represents an amino acid residue with a neutral polar or a charged (acidic or basic) or a small aliphatic or an aromatic sidechain; preferably a neutral polar sidechain or a charged sidechain; more preferably Ser, Thr, Asn, Gln, Asp, Glu, Arg or His, and even more preferably Ser, Asn, Gln, Asp, Glu or Arg.

For certain embodiments, aa1 represents Lys, Arg, His, Ser, Thr, Asn or Gln, more preferably Lys, Arg, His, Asn or Gln, and even more preferably Lys or Asn. For certain embodiments, aa2 represents Ala, Pro, Ile, Gln, Thr, Asp, Glu, Lys, Arg or His, more preferably Ala, Gln, Asp or Glu. For certain embodiments, aa3 represents Phe, Tyr, Trp, Lys, Arg or His, more preferably Phe, Tyr, Trp or His, and even more preferably Tyr, Trp or His. For certain embodiments, aa4 represents Ala, Pro, Ile, Gln, Thr, Asp, Glu, Lys, Arg or His, and even more preferably Gln, Lys, Arg, His, Asp or Glu. For certain embodiments, aa5 represents Ser, Thr, Asn, Gln, Asp, Glu, Arg or His, and even more preferably Ser, Asn, Gln, Asp, Glu or Arg.

In certain embodiments of the above sequences, (Xaa)ₙ (“loop 2”) is an amino acid sequence selected from SEQ ID Nos. 6 to 40, or an amino acid sequence having at least 80% homology thereto, and more preferably an amino acid sequence having at least 85%, 90%, 95% or even 98% homology thereto.

In certain embodiments of the above sequences, (Xaa)ₙ (“loop 2”) is an amino acid sequence selected from SEQ ID Nos. 6 to 40, or an amino acid sequence having at least 80% identity thereto, and more preferably an amino acid sequence having at least 85%, 90%, 95% or even 98% identity thereto.

In certain embodiments of the above sequences, (Xaa)ₘ (“loop 4”) is an amino acid sequence represented in the general formula (IV)

-aa7-aa8-aa9-aa10-aa11-aa12-aa13-aa14-aa15- (IV)

wherein
aa7 represents an amino acid residue with neutral polar or non-polar sidechain or an acidic sidechain;
aa8 represents an amino acid residue, preferably an amino acid residue with a neutral polar or non-polar sidechain or a charged (acidic or basic) sidechain or aromatic sidechain, more preferably a charged (acidic or basic) sidechain;
aa9 represents an amino acid residue, preferably an amino acid residue with a neutral polar or non-polar sidechain or a charged (acidic or basic) sidechain or aromatic sidechain, more preferably a neutral polar side chain or an acid side chain;
aa10 represents an amino acid residue, preferably an amino acid residue with a neutral polar or non-polar sidechain or a charged (acidic or basic) sidechain or aromatic sidechain, more preferably a neutral polar side chain or a basic or acid side chain;
aa11 represents an amino acid residue, preferably an amino acid residue with a neutral polar sidechain or a charged (acidic or basic) sidechain or a nonpolar aliphatic sidechain or an aromatic sidechain, more preferably a neutral polar side chain or a basic or acid side chain;
aa12 represents an amino acid residue, preferably an amino acid residue with a neutral polar sidechain or a charged (acidic or basic) sidechain or a nonpolar aliphatic sidechain or an aromatic sidechain, more preferably an acid side chain;
aa13 represents an amino acid residue, preferably an amino acid residue with a neutral polar sidechain or a charged (acidic or basic) sidechain or a nonpolar aliphatic sidechain or an aromatic sidechain, more preferably an acid side chain;
aa14 represents an amino acid residue, preferably an amino acid residue with a neutral polar sidechain or a charged (acidic or basic) sidechain; and
aa15 represents an amino acid residue, preferably an amino acid residue with a neutral polar or neutral non-polar sidechain or a charged (acidic or basic) sidechain.

For certain embodiments, aa7 represents Gly, Ala, Val, Pro, Trp, Gln, Ser, Asp or Glu, and even more preferably Gly, Ala, Trp, Gln, Ser, Asp or Glu. For certain embodiments, aa8 represents Asp, Glu, Lys, Arg, His, Gln, Ser, Thr, Asn, Ala, Val, Pro, Gly, Tyr or Phe, and even more preferably Asp, Glu, Lys, Arg, His or Gln. For certain embodiments, aa9 represents Gln, Ser, Thr, Asn, Asp, Glu, Arg, Lys, Gly, Leu, Pro or Tyr, and even more preferably Gln, Thr or Asp. For certain embodiments, aa10 represents Asp, Glu, Arg, His, Lys, Ser, Gln, Asn, Ala, Leu, Tyr, Trp, Pro or Gly, and even more preferably Asp, Glu, His, Gln, Asn, Leu, Trp or Gly. For certain embodiments, aa11 represents Asp, Glu, Ser, Thr, Gln, Arg, Lys, His, Val, Ile, Tyr or Gly and even more preferably Asp, Glu, Ser, Thr, Gln, Lys or His. For certain embodiments, aa12 represents Asp, Glu, Ser, Thr, Gln, Asn, Lys, Arg, Val, Leu, Ile, Trp, Tyr, Phe or Gly and even more preferably Asp, Glu, Ser, Tyr, Trp, Arg or Lys. For certain embodiments, aa13 represents Ser, Thr, Gln, Asn, Val, Ile, Leu, Gly, Pro, Asp, Glu, His, Arg, Trp, Tyr or Phe and even more preferably Ser, Thr, Gln, Asn, Val, Ile, Leu, Gly, Asp or Glu. For certain embodiments, aa14 represents Ala, Ile, Trp, Pro, Asp, Glu, Arg, Lys, His, Ser, Thr, Gln or Asn and even more preferably Ala, Pro, Asp, Glu, Arg, Lys, Ser, Gln or Asn. For certain embodiments, aa15 represents His, Arg, Lys, Asp, Ser, Thr, Gln, Asn, Ala, Val, Leu, Gly or Phe and even more preferably His, Arg, Lys, Asp, Ser, Thr, Gln or Asn.

In certain embodiments of the above sequences, (Xaa)ₙ (“loop 4”) is an amino acid sequence selected from SEQ ID Nos. 41 to 75, or an amino acid sequence having at least 80% homology thereto, and more preferably an amino acid sequence having at least 85%, 90%, 95% or even 98% homology thereto.

In certain embodiments of the above sequences, (Xaa)ₙ (“loop 4”) is an amino acid sequence selected from SEQ ID Nos. 41 to 75, or an amino acid sequence having at least 80% identity thereto, and more preferably an amino acid sequence having at least 85%, 90%, 95% or even 98% identity thereto.

In certain embodiments, the PD-L1 binding affimer polypeptide has an amino acid sequence selected from SEQ ID Nos. 76 to 84, or an amino acid sequence having at least 70% homology thereto, and even more preferably at least 75%, 80%, 85%, 90%, 95% or even 98% homology thereto.

In certain embodiments, the PD-L1 binding affimer polypeptide has an amino acid sequence selected from SEQ ID Nos. 76 to 84, or an amino acid sequence having at least 70% identity thereto, and even more preferably at least 75%, 80%, 85%, 90%, 95% or even 98% identity thereto.

In certain embodiments, the PD-L1 binding affimer polypeptide has an amino acid sequence can be encoded by a nucleic acid having a coding sequence corresponding to nucleotides 1-336 of one of SEQ ID Nos. 85 to 92, or a coding sequence at least 70% identical thereto, and even more preferably at least 75%, 80%, 85%, 90%, 95% or even 98% identity thereto.

In certain embodiments, the PD-L1 binding affimer polypeptide has an amino acid sequence can be encoded by a nucleic acid having a coding sequence that hybridizes to any one of SEQ ID Nos. 85 to 92 under stringent conditions of 6× sodium chloride/sodium citrate (SSC) at 45° C. followed by a wash in 0.2×SSC at 65° C.

In certain embodiments, the PD-L1 binding affimer described herein bind PD-L1 in a manner competitive with PD-L1 binding by anti-PD-L1 antibodies Atezolizumab, Avelumab and/or Durvalumab.

In certain embodiments, the PD-L1 binding affimer polypeptide forms a crystal structure with PD-L1 comprising an interface involving at least 10 residues of PD-L1 selected from Ile-54, Tyr-56, Glu-58, Glu-60, Asp-61, Lys-62, Asn-63, Gln 66, Val-68, Val-76, Val-111, Arg-113, Met-115, Ile-116, Ser-117, Gly-120, Ala-121, Asp-122, Tyr-123, and Arg-125.

In certain embodiments, the PD-L1 binding affimer polypeptide binding to PD-L1 (a) increases T-cell proliferation in a mixed lymphocyte reaction (MLR) assay; (b) increases interferon-γ production in an MLR assay; and/or (c) increases interleukin-2 (TL-2) secretion in an MLR assay.

In certain embodiments, the binder-drug conjugates of the present invention are fusion protein which may include, in addition to the PD-L1 binding affimer polypeptide or other target binding moieties, to illustrate, one or more additional amino acid sequences selected from the group consisting of: secretion signal sequences, peptide linker sequences, affinity tags, transmembrane domains, cell surface retention sequence, substrate recognition sequences for post-translational modifications, multimerization domains to create multimeric structures of the protein aggregating through protein-protein interactions, half-life extending polypeptide moieties, polypeptide sequences for altering tissue localization and antigen binding site of an antibody, and one or more additional affimer polypeptide sequences binding to other and different targets.

In certain embodiments, the fusion protein includes a half-life extending polypeptide moiety such as selected from the group consisting of an Fc domain or portion thereof, an albumin protein or portion thereof, an albumin-binding polypeptide moiety, transferrin or portion thereof, a transferrin-binding polypeptide moiety, fibronectin or portion thereof, or a fibronectin-binding polypeptide moiety.

Where the fusion protein includes an Fe domain or a portion thereof, in certain embodiments it is an Fe domain that retains FcN binding.

Where the fusion protein includes an Fe domain or a portion thereof, in certain embodiments the Fe domain or a portion thereof is from IgA, IgD, IgE, IgG, and IgM or a subclass (isotype) thereof such as IgG1, IgG2, IgG3, IgG4, IgA1 or IgA2.

In certain embodiments, the fusion protein has an amino acid sequence of SEQ ID No. 108 or SEQ ID No. 109 or a sequence having at least 70% homology thereto, and even more preferably at least 75%, 80%, 85%, 90%, 95% or even 98% identity thereto.

Where the fusion protein includes an Fe domain or a portion thereof, in certain embodiments the Fe domain or a portion thereof retains effector function selected from C1q binding, complement dependent cytotoxicity (CDC), antibody-dependent cell-mediated cytotoxicity (ADCC); phagocytosis; down regulation of B cell receptor, or a combination thereof.

In certain embodiments, where the fusion protein includes a half-life extending polypeptide moiety, that moiety increases the serum half-life of the protein by at least 5-fold relative to its absence from the protein, more preferably 10-fold, 20-fold, 30-fold, 40-fold, 50-fold, 60-fold, 70-fold, 80-fold, 90-fold, 100-fold, 200-fold, 500-fold or even 1000-fold.

In certain embodiments, the fusion protein of the invention are provided as a pharmaceutical preparation suitable for therapeutic use in a human patient, further comprising one ore more pharmaceutically acceptable excipients, buffers, salts or the like.

Still another aspect of the present invention relates pharmaceutical preparations suitable for therapeutic use in a human patient, comprising (i) a binder-drug conjugate or a combination PD-L1 inhibitor/innate immunity stimulator described herein, and (ii) one ore more pharmaceutically acceptable excipients, buffers, salts or the like.

In certain embodiments of the drug-conjugates of the invention, the free drug moiety is an immuno-DASH inhibitor. In certain embodiments, the immuno-DASH inhibitor has an in vitro intracellular IC50 in human macrophage for DPP8 and DPP9 inhibition less than 200 nM. In certain embodiments, the in vitro cell-free IC50 for DPP8 and/or DPP9 (and preferably for both DPP8 and DPP9) inhibition is less than 100 nM, 10 nM, 1.0 nM, 0.1 nM, 0.01 nM or even 0.001 nM. In certain embodiments, the EnPlex IC50 for DPP8 and/or DPP9 (and preferably for both DPP8 and DPP) inhibition is less than 100 nM, 10 nM, 1.0 nM, 0.1 nM, 0.01 nM, 0.001 nM (1 picomolar) or even 0.0001 nM (100 femtomolar). In certain embodiments, the Ki for DPP8 and/or DPP9 (and preferably for both DPP8 and DPP) inhibition is less than 100 nM, 10 nM, 1.0 nM, 0.1 nM, 0.01 nM, 0.001 nM (1 picomolar) or even 0.0001 nM (100 femtomolar).

In certain embodiments, the subject immuno-DASH inhibitors also inhibit Fibroblast Activating Protein (FAP) within the concentration range of the drug being an effective antitumor agent. For instance, the immuno-DASH inhibitor can have a Ki for inhibition FAP less than 100 nM, 10 nM, 1.0 nM, 0.1 nM, 0.01 nM, 0.001 nM (1 picomolar) or even 0.0001 nM (100 femtomolar).

In certain embodiments, the subject immuno-DASH inhibitors inhibit human Fibroblast Activating Protein (FAP) with an IC50 at least 2 fold higher than the IC50 for induction of pyroptosis of human macrophage, more preferably at least 3, 4, 5, 10, 20, 30, 40, 50 or even at least 100 fold higher—i.e., the immuno-DASH is a potent inducer of pyroptosis than FAP inhibition.

In certain embodiments, the immuno-DASH inhibitor exhibits slow binding inhibition kinetics. In certain embodiments, the immuno-DASH inhibitor has a koff rate for interaction with DPP4 less than 1×10−4/sec, and preferably less than 5×10−5/sec, 3×10−5/sec or even less than 1×10-5/sec.

In certain embodiments, the immuno-DASH inhibitor is administered to the patient as a binder-drug conjugate in a sufficient amount to cause a decrease in the number of tumor-associated macrophages.

In certain embodiments, the immuno-DASH inhibitor is administered to the patient as a binder-drug conjugate in a sufficient amount to reduce monocytic myeloid-derived suppressor cells in the tumor.

In certain embodiments, the immuno-DASH inhibitor is administered to the patient as a binder-drug conjugate in a sufficient amount to reduce T-cell suppressive activity of granulocytic myeloid-derived suppressor cells in the tumor.

In certain embodiments, the immuno-DASH inhibitor is administered to the patient as a binder-drug conjugate in an amount that produces full tumor regression at the therapeutically effective amount and the therapeutically effective amount is less than the binder-drug conjugate's maximum tolerated dose.

In certain embodiments, the immuno-DASH inhibitor is administered to the patient as a binder-drug conjugate alone or in combination with a PGE2 inhibitor, such as a cPLA-2 inhibitor.

In certain embodiments, the immuno-DASH inhibitor is administered to the patient as a binder-drug conjugate alone or in combination with a DPP4 inhibitor, such as sitagliptin, vildagliptin, saxagliptin, linagliptin, and alogliptin.

### BRIEF DESCRIPTION OF THE DRAWINGS

FIGS. 1A, 1B and 1C: Structure and characterisation of AVA04-182 Fc fusion protein

FIG. 2: Binding kinetics of AVA04-182 Fc to mouse PD-L1 evaluated by Biacore

FIG. 3: Competition with mouse PD-L1/mouse PD-1 of AVA04-182 Fc by ELISA

FIG. 4: Mouse mixed lymphocyte reaction of AVA04-182 Fc by ELISA

FIGS. 5A and 5B: Structure and characterisation of AVA04-251 Fc fusion protein

FIG. 6: Binding kinetics of AVA04-251 Fc to human PD-L1 evaluated by Biacore.

FIG. 7: Inhibition of PD-1/PD-L1 interaction by AVA04-251 Fc evaluated by NFAT gene reported assay (Promega)

FIGS. 8A, 8B and 8C: Structure and characterisation of AVA04-251 BH cys in-line fusion protein.

FIG. 9: Chemical structure of Compound 6323.

FIG. 10: Synthesis scheme of Compound 6323.

FIG. 11: Chemical structure of Compound 6325.

FIG. 12: Synthesis scheme of Compound 6325.

FIG. 13: Synthesis scheme of AVA04-251 BH cys-6323 using maleimide chemistry.

FIG. 14: Synthesis scheme of AVA04-183 Fc-6325 using NHS chemistry.

FIG. 15: Effect of combination treatment (AVA04-182 Fc+VbP) on tumour growth in a syngeneic murine bladder cancer (MB49) model.

FIG. 16: Effect on tumour growth after tumour challenge in a syngeneic murine bladder cancer (MB49) model.

FIG. 17: Effect of combination treatment (AVA04-251 Fc+VbP) on tumour growth in a humanized syngeneic model of colorectal cancer (MC38 HuPD-L1).

FIG. 18: Effect of combination treatment (AVA04-251 Fc+VbP) on tumour growth in a humanized syngeneic model of colorectal cancer (MC38 HuPD-L1).

FIG. 19: Effect on tumour growth after tumour challenge in a humanized syngeneic model of colorectal cancer (MC38 HuPD-L1).

FIG. 20: Comparison of AVA04-251 BH cys binding to human PD-L1 before and after conjugation to IR Dye 800CW using maleimide chemistry.

FIG. 21: Comparison of AVA04-251 Fc binding to human PD-L1 before and after conjugation to IR Dye 800CW using NHS chemistry.

FIG. 22: Biodistribution of AVA04-251 Fc-800 in a A375 mouse xenograft model.

FIG. 23: Tumor penetration of AVA04-251 Fc-800 in a A375 mouse xenograft model.

FIG. 24: In vitro rhFAPα cleavage of Affimer-linker-VbP pro-drugs.

FIG. 25: In vitro rhFAPα cleavage kinetics of Affimer-linker-VbP pro-drugs.

FIG. 26: Evaluation of a linker-VbP pro-drug compared to VbP in an acute toxicity study in Sprague Dawley rats.

FIG. 27: In vitro Affimer-linker-VbP pro-drug induced pyroptosis in the J774 mouse macrophage cell line.

FIG. 28: In vivo Cys-modified linker-VbP pro-drug induced G-CSF stimulation in BALB/c mice.

FIG. 29: Ipilimumab (biosimilar)/AVA04-141 transiently expressed in Expi293 cells, purified yield of −160 mg/L post Protein A purification.

FIG. 30: Bevacizumab (biosimilar)/AVA04-251 transiently expressed in Expi293 cells could be purified to greater than 97% yield, and Biacore demonstrates that the bi-specific antibody-Affimer fusions are able to engage both targets whether the constructed included a flexible linker [(G4S)3] or rigid linker [A(EAAAK)3].

FIG. 31: Illustrative examples of anti-PD-L1 affimer formatting that can be used to generate anti-PD-L1-Drug Conjugates of the present invention, including Fc fusions (showing a divalent PD-L1 binder format and a bispecific, divalent PD-L1 binder and Target X binder format), various formats of inline antibody fusions, a BiTE format and an inline fusion of the anti-PD-L1 affimer with a receptor trap domain. Each of these formats can be derivatized with one or more drug-conjugates.

FIG. 32: Affimers can be formatted at various sites on an Fe, and so should translate to IgG-Affimer fusions. Typical (unoptimised) expression yields in the range 400-800 mg/l. Analytical SEC-HPLC used to assess purity.

FIG. 33: Illustrates the selectivity of cleavage of the FAPα substrate recognition sequence, even between closely enzymes such as FAPα and PREP. Only FAPα is able to cleave and release the free drug moiety.

FIG. 34: Illustrates an FAPα cleavable linker designed to increase DAR and retain enzyme release of each drug moiety. With this linker design, DARs greater than 25, 50 or even 100 are feasible.

FIGS. 35A and 35B: Shows that FAPα is selectively overexpressed in the tumor microenvironment of most solid tumors. FAPα is up-regulated in malignant human epithelial tissues relative to normal epithelial tissues as demonstrated by mRNA analysis (FIG. 35A), histochemistry (FIG. 35A) and detection of enzymatic activity (FIG. 35B).

FIG. 36: FAPα-activated linkers are only activated selectively by FAPα.

FIGS. 37A and 37B: Free drug moiety Val-boroPro induces pyroptosis in AML cell lines in vitro. Human PDX model demonstrates efficacy of Val-boroPro itself against MV4-11 AML cells (human acute monocytic leukemia model) in vivo. One million MV4-11 cells injected into the tail vein of 10-week-old female NOD-SCID Il2rg−/−mice. Val-boroPro was administered intraperitoneally at 20 mg/mouse once a day—cycle schedule was 5 days on drug, 2 days off.

FIG. 38: From the crystal-derived structure of anti-PD-L1 affimer ACA04-261 bound to human PD-L1 derived, FIG. 16 provides a list of amino acid residues involved in the interface of contact between the two proteins.

### DETAILED DESCRIPTION

### I. Overview

One aspect of the present invention relates to a binder-drug conjugate comprising:
(i) a cell binding moiety that binds to a cell surface feature on a target cell in a disease state of a tissue, which cell surface feature undergoes slow internalization when bound by the binder-drug conjugate;
(ii) a drug moiety that has a pharmacological effect on bystander cells proximate to the target cell, which drug moiety has an EC50 for the pharmacological effect which is attenuated by at least 2-fold when part of the binder-drug conjugate relative to a free drug moiety released from the binder-drug conjugate; and
(iii) a linker moiety covalently linking the polypeptide binder moiety to the drug moiety, which linker moiety includes a substrate recognition sequence that is cleavable by an enzyme present extracellularly in the disease tissue, wherein in the presence of the enzyme the linker moiety can be cleaved and releases the free drug moiety.

### II. Definitions

To facilitate an understanding of the present invention, a number of terms and phrases are defined below.

a. Affimer

The term “Stefin Polypeptide” refers to a sub-group of proteins in the cystatin superfamily, a family which encompasses proteins that contain multiple cystatin-like sequences.

The stefin sub-group of the cystatin family is relatively small (around 100 amino acids) single domain proteins. They receive no known post-translational modification, and lack disulphide bonds, suggesting that they will be able to fold identically in a wide range of extra- and intracellular environments. Stefin A itself is a monomeric, single chain, single domain protein of 98 amino acids. The structure of Stefin A has been solved, facilitating the rational mutation of Stefin A into the Affimer Scaffold. The only known biological activity of cystatins is the inhibition of cathepsin activity, which allowed us to exhaustively test for residual biological activity of our engineered proteins.

The term “Affimer” (or “Affimer Scaffold” or “Affimer Polypeptide”) refers to small, highly stable proteins that are a recombinantly engineered variants of Stefin Polypeptides. Affimer proteins display two peptide loops and an N-terminal sequence that can all be randomised to bind to desired target proteins with high affinity and specificity, in a similar manner to monoclonal antibodies. Stabilisation of the two peptides by the Steffin protein scaffold constrains the possible conformations that the peptides can take, increasing the binding affinity and specificity compared to libraries of free peptides. These engineered non-antibody binding proteins are designed to mimic the molecular recognition characteristics of monoclonal antibodies in different applications. Variations to other parts of the Stefin polypeptide sequence can be carried out, with such variations improving the properties of these affinity reagents, such as increase stability, make them robust across a range of temperatures and pH and the like. Preferably the Affimer includes a sequence derived from stefin A, sharing substantial identify with a stefin A wild type sequence, such as human Stefin A. It will be apparent to a person skilled in the art that modifications may be made to the scaffold sequence without departing from the invention. In particular, an Affimer Scaffold can have an amino acid sequences that is at least 25%, 35%, 45%, 55% or 60% identity to the corresponding sequences to human Stefin A, preferably at least 70%, preferably at least 80%, preferably at least 85%, preferably at least 90%, preferably at least 92%, preferably at least 94%, preferably at least 95% identical, e.g., where the sequence variations do not adversely affect the ability of the scaffold to bind to the desired target (such as PD-L1), and e.g., which do not restore or generate biological functions such as those which are possessed by wild type stefin A but which are abolished in mutational changes described herein.

An “Binder-drug conjugate” refers to a polypeptide including an Affimer Polypeptide sequence and having any other modifications (e.g., conjugation, post-translational modifications, etc) so as to represent the therapeutically active protein intended for delivery to a patient.

“Programmed death-ligand 1”, also known as “PD-L1”, “cluster of differentiation 274”, “CD274”, “B7 homolog 1” or “B7-H1”, refers a protein that, in the case of humans, is encoded by the CD274 gene. The human PD-L1 is a 40 kDa type 1 transmembrane protein that plays a major role in suppressing the immune system under different circumstances. A representative human PD-L1 sequence is provided by UniProtKB Primary accession number Q9NZQ7, and will include other human isoforms thereof. PD-L1 binds to its receptor, PD-1, found on activated T cells, B cells, and myeloid cells, to modulate activation or inhibition. PD-L1 also has an appreciable affinity for the costimulatory molecule CD80 (B7-1). Engagement of PD-L1 with its receptor PD-1 (“Programmed cell death protein i” or “CD279”) on T cells delivers a signal that inhibits TCR-mediated activation of IL-2 production and T cell proliferation. In this regard, PD-L1 is considered a checkpoint, and its upregulated expression in tumors contributes to inhibition of T-cell mediated antitumor responses. While PD-L1 will be used generally with reference to PD-L1 from various mammalian species, it will be understood throughout the application that any reference to PD-L1 includes human PD-L1 and is, preferably, referring to human PD-L1 per se.

A “PD-L1 Binder-drug conjugate” refers to a binder-drug conjugate having at least one Affimer Polypeptide that binds to PD-L1, particularly human PD-L1, with a dissociation constant (Kd) of at least 10⁻⁶M.

b. Polypeptides

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art.

The terms “amino acid residue” and “amino acid” are used interchangeably and means, in the context of a polypeptide, an amino acid that is participating in one more peptide bonds of the polypeptide. In general, the abbreviations used herein for designating the amino acids are based on recommendations of the IUPAC-IUB Commission on Biochemical Nomenclature (see Biochemistry (1972) 11:1726-1732). For instance, Met, Ile, Leu, Ala and Gly represent “residues” of methionine, isoleucine, leucine, alanine and glycine, respectively. By the residue is meant a radical derived from the corresponding □-amino acid by eliminating the OH portion of the carboxyl group and the H portion of the □-amino group. The term “amino acid side chain” is that part of an amino acid exclusive of the —CH(NH2)COOH portion, as defined by K. D. Kopple, “Peptides and Amino Acids”, W. A. Benjamin Inc., New York and Amsterdam, 1966, pages 2 and 33.

For the most part, the amino acids used in the application of this invention are those naturally occurring amino acids found in proteins, or the naturally occurring anabolic or catabolic products of such amino acids which contain amino and carboxyl groups. Particularly suitable amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan, and those amino acids and amino acid analogs which have been identified as constituents of peptidylglycan bacterial cell walls.

Amino acid residues having “basic sidechains” include Arg, Lys and His. Amino acid residues having “acidic sidechains” include Glu and Asp. Amino acid residues having “neutral polar sidechains” include Ser, Thr, Asn, Gln, Cys and Tyr. Amino acid residues having “neutral non-polar sidechains” include Gly, Ala, Val, Ile, Leu, Met, Pro, Trp and Phe. Amino acid residues having “non-polar aliphatic sidechains” include Gly, Ala, Val, Ile and Leu. Amino acid residues having “hydrophobic sidechains” include Ala, Val, Ile, Leu, Met, Phe, Tyr and Trp. Amino acid residues having “small hydrophobic sidechains” include Ala and Val. Amino acid residues having “aromatic sidechains” include Tyr, Trp and Phe.

The term amino acid residue further includes analogs, derivatives and congeners of any specific amino acid referred to herein, as for instance, the subject affimers (particularly if generated by chemical synthesis) can include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminiopimelic acid, ornithine, or diaminobutyric acid. Other naturally occurring amino acid metabolites or precursors having side chains which are suitable herein will be recognized by those skilled in the art and are included in the scope of the present invention.

Also included are the (D) and (L) stereoisomers of such amino acids when the structure of the amino acid admits of stereoisomeric forms. The configuration of the amino acids and amino acid residues herein are designated by the appropriate symbols (D), (L) or (DL), furthermore when the configuration is not designated the amino acid or residue can have the configuration (D), (L) or (DL). It will be noted that the structure of some of the compounds of this invention includes asymmetric carbon atoms. It is to be understood accordingly that the isomers arising from such asymmetry are included within the scope of this invention. Such isomers can be obtained in substantially pure form by classical separation techniques and by sterically controlled synthesis. For the purposes of this application, unless expressly noted to the contrary, a named amino acid shall be construed to include both the (D) or (L) stereoisomers.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a protein of interest.

A “conservative amino acid substitution” is one in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies of the invention do not abrogate the binding of the polypeptide, soluble protein, or antibody containing the amino acid sequence, to the target binding site. Methods of identifying amino acid conservative substitutions which do not eliminate binding are well-known in the art.

A polypeptide, soluble protein, antibody, polynucleotide, vector, cell, or composition which is “isolated” is a polypeptide, soluble protein, antibody, polynucleotide, vector, cell, or composition which is in a form not found in nature. Isolated polypeptides, soluble proteins, antibodies, polynucleotides, vectors, cells, or compositions include those which have been purified to a degree that they are no longer in a form in which they are found in nature. In some embodiments, a polypeptide, soluble protein, antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The term “fusion protein” or “fusion polypeptide” as used herein refers to a hybrid protein expressed by a nucleic acid molecule comprising nucleotide sequences of at least two genes.

The term “linker” or “linker region” as used herein refers to a linker inserted between a first polypeptide (e.g., copies of an affimer) and a second polypeptide (e.g., another affimer, an Fc domain, a ligand binding domain, etc). In some embodiments, the linker is a peptide linker. Linkers should not adversely affect the expression, secretion, or bioactivity of the polypeptides. Preferably, linkers are not antigenic and do not elicit an immune response.

An “Affimer-Antibody fusion” refers to a fusion protein including an affimer polypeptide portion and a variable region of an antibody. Affimer-Antibody fusions include full length antibodies having, for example, one or more affimer polypeptide sequences appended to the C-terminus or N-terminus of one or more of its VH and/or VL chains, i.e., at least one chain of the assembled antibody is a fusion protein with an affimer polypeptide. Affimer-Antibody fusions also include embodiments wherein one or more affimer polypeptide sequences are provided as part of a fusion protein with an antigen binding site or variable region of an antibody fragment.

The term “antibody” as used herein refers to an immunoglobulin molecule that recognizes and specifically binds a target, such as a protein, polypeptide, peptide, carbohydrate, polynucleotide, lipid, or a combination of any of the foregoing, through at least one antigen-binding site wherein the antigen-binding site is usually within the variable region of the immunoglobulin molecule. As used herein, the term encompasses intact polyclonal antibodies, intact monoclonal antibodies, antibody fragments (such as Fab, Fab′, F(ab′)2, and Fv fragments), single chain Fv (scFv) antibodies provided those fragments have been formatted to include an Fc or other FcγRIII binding domain, multispecific antibodies, bispecific antibodies, monospecific antibodies, monovalent antibodies, chimeric antibodies, humanized antibodies, human antibodies, fusion proteins comprising an antigen-binding site of an antibody (formatted to include an Fc or other FcγRIII binding domain), and any other modified immunoglobulin molecule comprising an antigen-binding site as long as the antibodies exhibit the desired biological activity.

While the antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu.

The term “variable region” of an antibody refers to the variable region of an antibody light chain, or the variable region of an antibody heavy chain, either alone or in combination. Generally, the variable region of heavy and light chains each consist of four framework regions (FR) and three complementarity determining regions (CDRs), also known as “hypervariable regions”. The CDRs in each chain are held together in close proximity by the framework regions and, with the CDRs from the other chain, contribute to the formation of the antigen-binding sites of the antibody. There are at least two techniques for determining CDRs: (1) an approach based on cross-species sequence variability (i.e., Kabat et al., 1991, Sequences of Proteins of Immunological Interest, 5th Edition, National Institutes of Health, Bethesda Md.), and (2) an approach based on crystallographic studies of antigen-antibody complexes (Al Lazikani et al., 1997, J. Mol. Biol., 273:927-948). In addition, combinations of these two approaches are sometimes used in the art to determine CDRs.

The term “humanized antibody” as used herein refers to forms of non-human (e.g., murine) antibodies that are specific immunoglobulin chains, chimeric immunoglobulins, or fragments thereof that contain minimal non-human sequences. Typically, humanized antibodies are human immunoglobulins in which residues of the CDRs are replaced by residues from the CDRs of a non-human species (e.g., mouse, rat, rabbit, or hamster) that have the desired specificity, affinity, and/or binding capability. In some instances, the Fv framework region residues of a human immunoglobulin are replaced with the corresponding residues in an antibody from a non-human species. The humanized antibody can be further modified by the substitution of additional residues either in the Fv framework region and/or within the replaced non-human residues to refine and optimize antibody specificity, affinity, and/or binding capability. The humanized antibody may comprise variable domains containing all or substantially all of the CDRs that correspond to the non-human immunoglobulin whereas all or substantially all of the framework regions are those of a human immunoglobulin sequence. In some embodiments, the variable domains comprise the framework regions of a human immunoglobulin sequence. In some embodiments, the variable domains comprise the framework regions of a human immunoglobulin consensus sequence. The humanized antibody can also comprise at least a portion of an immunoglobulin constant region or domain (Fc), typically that of a human immunoglobulin. A humanized antibody is usually considered distinct from a chimeric antibody.

The terms “epitope” and “antigenic determinant” are used interchangeably herein and refer to that portion of an antigen capable of being recognized and specifically bound by a particular antibody, a particular affimer or other particular binding domain. When the antigen is a polypeptide, epitopes can be formed both from contiguous amino acids and noncontiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation.

As use herein, the term “specifically binds to” or is “specific for” refers to measurable and reproducible interactions such as binding between a target and an affimer, antibody or other binding partner, which is determinative of the presence of the target in the presence of a heterogeneous population of molecules including biological molecules. For example, an affimer that specifically binds to a target is an affimer that binds this target with greater affinity, avidity (if multimeric formatted), more readily, and/or with greater duration than it binds to other targets.

c. Checkpoint Inhibitors, Co-Stimulatory Agonists and Chemotherapeutics

A “checkpoint molecule” refers to proteins that are expressed by tissues and/or immune cells and reduce the efficacy of an immune response in a manner dependent on the level of expression of the checkpoint molecule. When these proteins are blocked, the “brakes” on the immune system are released and, for example, T cells are able to kill cancer cells more effectively. Examples of checkpoint proteins found on T cells or cancer cells include PD-1/PD-L1 and CTLA-4/B7-1/B7-2, PD-L2, NKG2A, KIR, LAG-3, TIM-3, CD96, VISTA and TIGIT.

A “checkpoint inhibitor” refers to a drug entity that reverses the immunosuppressive signaling from a checkpoint molecule.

A “costimulatory molecule” refers to an immune cell such as a T cell cognate binding partner which specifically binds to costimulatory ligands thereby mediating co-stimulation, such as, but not limited to proliferation. Costimulatory molecules are cell surface molecules other than the antigen receptor or ligand which facilitate an effective immune response. Co-stimulatory molecules include, but are not limited to MHCI molecules, BTLA receptor and Toll ligands, and OX40, CD27, CD28, CDS, ICAM-1, LFA-1 (CD11a/CD18), ICOS (CD278) and 4-1BB (CD137). Examples of costimulatory molecules include but are not limited to: CDS, ICAM-1, GITR, BAFFR, HVEM (LIGHTR), SLAMF7, NKp80 (KLRF1), NKp44, NKp30, NKp46, CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CD11 d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244,2B4), CD84, CD96 (Tactile), CEACAMI, CRTAM, Ly9 (CD229), CD160 (BY55), PSGL1, CD100 (SEMA4D), CD69, SLAMF6 (NTB-A, Ly108), SLAM (SLAMFI, CD150, IPO-3), BLAME (SLAMF8), SELPLG (CD162), LTBR, LAT, GADS, SLP-76, PAG/Cbp, CD19a, and CD83 ligand.

A “costimulatory agonists” refers to a drug entity that activates (agonizes) the costimulatory molecule, such as costimulatory ligand would do, and produces an immunostimulatory signal or otherwise increases the potency or efficacy of an immune response.

A “chemotherapeutic agent” is a chemical compound useful in the treatment of cancer. Examples of chemotherapeutic agents include alkylating agents such as thiotepa and cyclophosphamide (CYTOXAN); alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL); beta-lapachone; lapachol; colchicines; betulinic acid; a camptothecin (including the synthetic analogue topotecan (HYCAMTIN), CPT-11 (irinotecan, CAMPTOSAR), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omegaI1 (see, e.g., Nicolaou et al., Angew. Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN, morpholino-doxorubicin, cyanomorpholino-doxorubicin, 2-pyrrolino-doxorubicin, doxorubicin HCl liposome injection (DOXIL) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR), tegafur (UFTORAL), capecitabine (XELODA), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c-Kit inhibitors; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE, FILDESIN); dacarbazine; mannomustine; mitobronitol; mitolactol; pipobroman; gacytosine; arabinoside (“Ara-C”); thiotepa; taxoids, e.g., paclitaxel (TAXOL), albumin-engineered nanoparticle formulation of paclitaxel (ABRAXANE), and doxetaxel (TAXOTERE); chloranbucil; 6-thioguanine; mercaptopurine; methotrexate; platinum analogs such as cisplatin and carboplatin; vinblastine (VELBAN); platinum; etoposide (VP-16); ifosfamide; mitoxantrone; vincristine (ONCOVIN); oxaliplatin; leucovovin; vinorelbine (NAVELBINE); novantrone; edatrexate; daunomycin; aminopterin; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN) combined with 5-FU and leucovovin.

Also included in this definition are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX tamoxifen), raloxifene (EVISTA), droloxifene, 4-hydroxytamoxifen, trioxifene, keoxifene, LY117018, onapristone, and toremifene (FARESTON); anti-progesterones; estrogen receptor down-regulators (ERDs); estrogen receptor antagonists such as fulvestrant (FASLODEX); agents that function to suppress or shut down the ovaries, for example, leutinizing hormone-releasing hormone (LHRH) agonists such as leuprolide acetate (LUPRON and ELIGARD), goserelin acetate, buserelin acetate and tripterelin; anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE), exemestane (AROMASIN), formestanie, fadrozole, vorozole (RIVISOR), letrozole (FEMARA), and anastrozole (ARIMIDEX). In addition, such definition of chemotherapeutic agents includes bisphosphonates such as clodronate (for example, BONEFOS or OSTAC), etidronate (DIDROCAL), NE-58095, zoledronic acid/zoledronate (ZOMETA), alendronate (FOSAMAX), pamidronate (AREDIA), tiludronate (SKELID), or risedronate (ACTONEL); as well as troxacitabine (a 1,3-dioxolane nucleoside cytosine analog); anti-sense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE vaccine and gene therapy vaccines, for example, ALLOVECTIN vaccine, LEUVECTIN vaccine, and VAXID vaccine; topoisomerase 1 inhibitor (e.g., LURTOTECAN); an anti-estrogen such as fulvestrant; a Kit inhibitor such as imatinib or EXEL-0862 (a tyrosine kinase inhibitor); EGFR inhibitor such as erlotinib or cetuximab; an anti-VEGF inhibitor such as bevacizumab; arinotecan; rmRH (e.g., ABARELIX); lapatinib and lapatinib ditosylate (an ErbB-2 and EGFR dual tyrosine kinase small-molecule inhibitor also known as GW572016); 17AAG (geldanamycin derivative that is a heat shock protein (Hsp) 90 poison), and pharmaceutically acceptable salts, acids or derivatives of any of the above.

As used herein, the term “cytokine” refers generically to proteins released by one cell population that act on another cell as intercellular mediators or have an autocrine effect on the cells producing the proteins. Examples of such cytokines include lymphokines, monokines; interleukins (“ILs”) such as IL-1, IL-1α, IL-2, IL-3, IL-4, IL-5, IL-6, IL-7, IL-8, IL-9, IL10, IL-11, IL-12, IL-13, IL-15, IL-17A-F, IL-18 to IL-29 (such as IL-23), IL-31, including PROLEUKIN rIL-2; a tumor-necrosis factor such as TNF-α or TNF-β, TGF-β1-3; and other polypeptide factors including leukemia inhibitory factor (“LIF”), ciliary neurotrophic factor (“CNTF”), CNTF-like cytokine (“CLC”), cardiotrophin (“CT”), and kit ligand (“KL”).

As used herein, the term “chemokine” refers to soluble factors (e.g., cytokines) that have the ability to selectively induce chemotaxis and activation of leukocytes. They also trigger processes of angiogenesis, inflammation, wound healing, and tumorigenesis. Example chemokines include IL-8, a human homolog of murine keratinocyte chemoattractant (KC).

d. Treatments

The term “dysfunctional”, as used herein, also includes refractory or unresponsive to antigen recognition, specifically, impaired capacity to translate antigen recognition into down-stream T-cell effector functions, such as proliferation, cytokine production (e.g., IL-2) and/or target cell killing.

The term “anergy” refers to the state of unresponsiveness to antigen stimulation resulting from incomplete or insufficient signals delivered through the T-cell receptor (e.g. increase in intracellular Ca⁺² in the absence of ras-activation). T cell anergy can also result upon stimulation with antigen in the absence of co-stimulation, resulting in the cell becoming refractory to subsequent activation by the antigen even in the context of costimulation. The unresponsive state can often be overridden by the presence of Interleukin-2. Anergic T-cells do not undergo clonal expansion and/or acquire effector functions.

The term “exhaustion” refers to T cell exhaustion as a state of T cell dysfunction that arises from sustained TCR signaling that occurs during many chronic infections and cancer. It is distinguished from anergy in that it arises not through incomplete or deficient signaling, but from sustained signaling. It is defined by poor effector function, sustained expression of inhibitory receptors and a transcriptional state distinct from that of functional effector or memory T cells. Exhaustion prevents optimal control of infection and tumors.

“Enhancing T-cell function” means to induce, cause or stimulate a T-cell to have a sustained or amplified biological function, or renew or reactivate exhausted or inactive T-cells. Examples of enhancing T-cell function include: increased secretion of γ-interferon from CD8+ T-cells, increased proliferation, increased antigen responsiveness (e.g., viral, pathogen, or tumor clearance) relative to such levels before the intervention. In one embodiment, the level of enhancement is as least 50%, alternatively 60%, 70%, 80%, 90%, 100%, 120%, 150%, 200%. The manner of measuring this enhancement is known to one of ordinary skill in the art.

A “T cell dysfunctional disorder” is a disorder or condition of T-cells characterized by decreased responsiveness to antigenic stimulation. In a particular embodiment, a T-cell dysfunctional disorder is a disorder that is specifically associated with inappropriate increased levels of PD-1. A T-cell dysfunctional disorder can also be associated with inappropriate increased levels of PD-L1 in the tumor which gives rise to suppression of T-cell antitumor function(s). In another embodiment, a T-cell dysfunctional disorder is one in which T-cells are anergic or have decreased ability to secrete cytokines, proliferate, or execute cytolytic activity. In a specific aspect, the decreased responsiveness results in ineffective control of a pathogen or tumor expressing an immunogen. Examples of T cell dysfunctional disorders characterized by T-cell dysfunction include unresolved acute infection, chronic infection and tumor immunity.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0× length of the treatment duration.

The terms “cancer” and “cancerous” as used herein refer to or describe the physiological condition in mammals in which a population of cells are characterized by unregulated cell growth. Examples of cancer include, but are not limited to, carcinoma, blastoma, sarcoma, and hematologic cancers such as lymphoma and leukemia.

The terms “tumor” and “neoplasm” as used herein refer to any mass of tissue that results from excessive cell growth or proliferation, either benign (noncancerous) or malignant (cancerous) including pre-cancerous lesions. Tumor growth is generally uncontrolled and progressive, does not induce or inhibit the proliferation of normal cells. Tumor can affect a variety of cells, tissues or organs, including but not limited to selected from bladder, bone, brain, breast, cartilage, glial cells, esophagus, fallopian tube, gall bladder, heart, intestine, kidney, liver, lung, lymph node, neural tissue, ovary, pancreas, prostate, skeletal muscle, skin, spinal cord, spleen, stomach, testis, thymus, thyroid, trachea, urethra, ureter, urethra, uterus, vagina organ or tissue or the corresponding cells. Tumors include cancers, such as sarcoma, carcinoma, plasmacytoma or (malignant plasma cells). Tumors of the present invention, may include, but are not limited to leukemias (e.g., acute leukemia, acute lymphoblastic leukemia, acute myeloid leukemia, acute myeloid leukemia, acute promyelocytic leukemia, acute myeloid-monocytic leukemia, acute monocytic leukemia, acute leukemia, chronic leukemia, chronic myeloid leukemia, chronic lymphocytic leukemia, polycythemia vera), lymphomas (Hodgkin's disease, non-Hodgkin's disease), primary macroglobulinemia disease, heavy chain disease, and solid tumors such as sarcomas cancer (e.g., fibrosarcoma, myxosarcoma, liposarcoma, chondrosarcoma, osteosarcoma, chordoma, endothelium sarcoma, lymphangiosarcoma, angiosarcoma, lymphangioendothelio sarcoma, synovioma vioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinoma, carcinoma, bronchogenic carcinoma, medullary carcinoma, renal cell carcinoma, hepatoma, Nile duct carcinoma, choriocarcinoma, spermatogonia Tumor, embryonal carcinoma, Wilms' tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, retinoblastoma), esophageal cancer, gallbladder, kidney cancer, multiple myeloma. Preferably, a “tumor” includes, but is not limited to: pancreatic cancer, liver cancer, lung cancer, stomach cancer, esophageal cancer, head and neck squamous cell carcinoma, prostate cancer, colon cancer, breast cancer, lymphoma, gallbladder cancer, renal cancer, leukemia, multiple myeloma, ovarian cancer, cervical cancer and glioma.

The term “metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

The terms “cancer cell” and “tumor cell” refer to the total population of cells derived from a cancer or tumor or pre-cancerous lesion, including both non-tumorigenic cells, which comprise the bulk of the cancer cell population, and tumorigenic stem cells (cancer stem cells). As used herein, the terms “cancer cell” or “tumor cell” will be modified by the term “non-tumorigenic” when referring solely to those cells lacking the capacity to renew and differentiate to distinguish those tumor cells from cancer stem cells.

The term “effective amount” as used herein refers to an amount to provide therapeutic or prophylactic benefit.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progression free survival” (PFS) refers to the length of time during and after treatment during which the disease being treated (e.g., cancer) does not get worse.

Progression-free survival may include the amount of time patients have experienced a complete response or a partial response, as well as the amount of time patients have experienced stable disease.

As used herein, “overall response rate” (ORR) refers to the sum of complete response (CR) rate and partial response (PR) rate.

As used herein, “overall survival” refers to the percentage of individuals in a group who are likely to be alive after a particular duration of time.

The term “treatment” as used herein refers to the individual trying to change the process or treatment of a clinical disease caused by intervention of a cell, may be either preventive intervention course of clinical pathology. Including but not limited to treatment to prevent the occurrence or recurrence of disease, alleviation of symptoms, reducing the direct or indirect pathological consequences of any disease, preventing metastasis, slow the rate of disease progression, amelioration or remission of disease remission or improved prognosis.

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The terms “agonist” and “agonistic” as used herein refer agents that are capable of, directly or indirectly, substantially inducing, activating, promoting, increasing, or enhancing the biological activity of a target or target pathway. The term “agonist” is used herein to include any agent that partially or fully induces, activates, promotes, increases, or enhances the activity of a protein or other target of interest.

The terms “antagonist” and “antagonistic” as used herein refer to or describe an agent that is capable of, directly or indirectly, partially or fully blocking, inhibiting, reducing, or neutralizing a biological activity of a target and/or pathway. The term “antagonist” is used herein to include any agent that partially or fully blocks, inhibits, reduces, or neutralizes the activity of a protein or other target of interest.

The terms “modulation” and “modulate” as used herein refer to a change or an alteration in a biological activity. Modulation includes, but is not limited to, stimulating an activity or inhibiting an activity. Modulation may be an increase in activity or a decrease in activity, a change in binding characteristics, or any other change in the biological, functional, or immunological properties associated with the activity of a protein, a pathway, a system, or other biological targets of interest.

The term “immune response” as used herein includes responses from both the innate immune system and the adaptive immune system. It includes both cell-mediated and/or humoral immune responses. It includes both T-cell and B-cell responses, as well as responses from other cells of the immune system such as natural killer (NK) cells, monocytes, macrophages, etc.

The term “pharmaceutically acceptable” refers to a substance approved or approvable by a regulatory agency of the Federal government or a state government or listed in the U.S. Pharmacopeia or other generally recognized pharmacopeia for use in animals, including humans.

The terms “pharmaceutically acceptable excipient, carrier or adjuvant” or “acceptable pharmaceutical carrier” refer to an excipient, carrier or adjuvant that can be administered to a subject, together with at least one agent of the present disclosure, and which does not destroy the pharmacological activity thereof and is nontoxic when administered in doses sufficient to deliver a therapeutic effect. In general, those of skill in the art and the U.S. FDA consider a pharmaceutically acceptable excipient, carrier, or adjuvant to be an inactive ingredient of any formulation.

The terms “effective amount” or “therapeutically effective amount” or “therapeutic effect” refer to an amount of a binder-drug conjugate described herein effective to “treat” a disease or disorder in a subject such as, a mammal. In the case of cancer or a tumor, the therapeutically effective amount of an PD-L1 binding Binder-drug conjugate has a therapeutic effect and as such can boost the immune response, boost the anti-tumor response, increase cytolytic activity of immune cells, increase killing of tumor cells by immune cells, reduce the number of tumor cells; decrease tumorigenicity, tumorigenic frequency or tumorigenic capacity; reduce the number or frequency of cancer stem cells; reduce the tumor size; reduce the cancer cell population; inhibit or stop cancer cell infiltration into peripheral organs including, for example, the spread of cancer into soft tissue and bone; inhibit and stop tumor or cancer cell metastasis; inhibit and stop tumor or cancer cell growth; relieve to some extent one or more of the symptoms associated with the cancer; reduce morbidity and mortality; improve quality of life; or a combination of such effects.

The terms “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus those in need of treatment include those already with the disorder; those prone to have the disorder; and those in whom the disorder is to be prevented. In the case of cancer or a tumor, a subject is successfully “treated” according to the methods of the present invention if the patient shows one or more of the following: an increased immune response, an increased anti-tumor response, increased cytolytic activity of immune cells, increased killing of tumor cells by immune cells, a reduction in the number of or complete absence of cancer cells; a reduction in the tumor size; inhibition of or an absence of cancer cell infiltration into peripheral organs including the spread of cancer cells into soft tissue and bone; inhibition of or an absence of tumor or cancer cell metastasis; inhibition or an absence of cancer growth; relief of one or more symptoms associated with the specific cancer; reduced morbidity and mortality; improvement in quality of life; reduction in tumorigenicity; reduction in the number or frequency of cancer stem cells; or some combination of effects.

e. Miscellaneous

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has 30 or fewer carbon atoms in its backbone (e.g., C₁-C₃₀ for straight chain, C₃-C₃₀ for branched chain), for example, 20 or fewer. Likewise, certain cycloalkyls have from 3-10 carbon atoms in their ring structure, for example, 5, 6 or 7 carbons in the ring structure. “Alkyl” (or “lower alkyl”) as used throughout the specification and claims is intended to include both “unsubstituted alkyls” and “substituted alkyls”.

The term “aralkyl”, as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, for example, from one to four or one to six carbon atoms in its backbone structure. Likewise, “lower alkenyl” and “lower alkynyl” have similar chain lengths. In some embodiments, alkyl groups are lower alkyls. In some embodiments, a substituent designated herein as alkyl is a lower alkyl.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with such substituents as described above, for example, halogen, azide, alkyl, aralkyl, alkenyl, alkynyl, cycloalkyl, hydroxyl, amino, nitro, sulfhydryl, imino, amido, phosphionate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The terms “heterocyclyl” or “heterocyclic group” refer to 3- to 10-membered ring structures, for example, 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “heteroaryl” refers to a monovalent aromatic monocyclic ring system wherein at least one ring atoms is a heteroatom independently selected from the group consisting of O, N and S. The term 5-membered heteroaryl refers to a heteroaryl wherein the number of ring atoms is 5. Examples of 5-membered heteroaryl groups include pyrrolyl, pyrazolyl, oxazolyl, isoxazolyl, thiazolyl, isothiazolyl, oxadiazolyl, thiadiazolyl, furazanyl, imidazolinyl, and triazolyl.

The term “heterocycloalkyl” refers to a monocyclic or bicyclic monovalent saturated or non-aromatic unsaturated ring system wherein from 1 to 4 ring atoms are heteroatoms independently selected from the group consisting of O, N and S. The term “3 to 10-membered heterocycloalkyl” refers to a heterocycloalkyl wherein the number of ring atoms is from 3 to 10. Examples of 3 to 10-membered heterocycloalkyl include 3 to 6-membered heterocycloalkyl. Bicyclic ring systems include fused, bridged, and spirocyclic ring systems. More particular examples of heterocycloalkyl groups include azepanyl, azetidinyl, aziridinyl, imidazolidinyl, morpholinyl, oxazolidinyl, oxazolidinyl, piperazinyl, piperidinyl, pyrazolidinyl, pyrrolidinyl, quinuclidinyl, and thiomorpholinyl.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle”, as used herein, refers to an aromatic or non-aromatic ring in which each atom of the ring is carbon.

The term “heteroatom” as used herein means an atom of any element other than carbon or hydrogen. Exemplary heteroatoms are nitrogen, oxygen, sulfur and phosphorous.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

“Halogen” or “halo” by themselves or as part of another substituent refers to fluorine, chlorine, bromine and iodine, or fluoro, chloro, bromo and iodo.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described hereinabove. The permissible substituents can be one or more and the same or different for appropriate organic compounds. Substituents can include, for example, a halogen, a hydroxyl, a carbonyl (such as a carboxyl, an ester, a formyl, or a ketone), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like. For purposes of this invention, the heteroatoms such as nitrogen may have hydrogen substituents and/or any permissible substituents of organic compounds described herein which satisfy the valencies of the heteroatoms. This invention is not intended to be limited in any manner by the permissible substituents of organic compounds.

By the terms “amino acid residue” and “peptide residue” is meant an amino acid or peptide molecule without the —OH of its carboxyl group. In general the abbreviations used herein for designating the amino acids and the protective groups are based on recommendations of the IUPAC-IUB Commission on Biochemical Nomenclature (see Biochemistry (1972) 11:1726-1732). For instance Met, Ile, Leu, Ala and Gly represent “residues” of methionine, isoleucine, leucine, alanine and glycine, respectively. By the residue is meant a radical derived from the corresponding.alpha.-amino acid by eliminating the OH portion of the carboxyl group and the H portion of the .alpha.-amino group. The term “amino acid side chain” is that part of an amino acid exclusive of the —CH(NH₂)COOH portion, as defined by K. D. Kopple, “Peptides and Amino Acids”, W. A. Benjamin Inc., New York and Amsterdam, 1966, pages 2 and 33.

For the most part, the amino acids used in the application of this invention are those naturally occurring amino acids found in proteins, or the naturally occurring anabolic or catabolic products of such amino acids which contain amino and carboxyl groups. Particularly suitable amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan, and those amino acids and amino acid analogs which have been identified as constituents of peptidylglycan bacterial cell walls.

The term amino acid residue further includes analogs, derivatives and congeners of any specific amino acid referred to herein, as for instance, the subject compound can include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminiopimelic acid, ornithine, or diaminobutyric acid. Other naturally occurring amino acid metabolites or precursors having side chains which are suitable herein will be recognized by those skilled in the art and are included in the scope of the present invention.

Also included are the (D) and (L) stereoisomers of such amino acids when the structure of the amino acid admits of stereoisomeric forms. The configuration of the amino acids and amino acid residues herein are designated by the appropriate symbols (D), (L) or (DL), furthermore when the configuration is not designated the amino acid or residue can have the configuration (D), (L) or (DL). It will be noted that the structure of some of the compounds of this invention includes asymmetric carbon atoms. It is to be understood accordingly that the isomers arising from such asymmetry are included within the scope of this invention. Such isomers can be obtained in substantially pure form by classical separation techniques and by sterically controlled synthesis. For the purposes of this application, unless expressly noted to the contrary, a named amino acid shall be construed to include both the (D) or (L) stereoisomers.

As noted above, certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as, falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

The term “IC₅₀” refers to the concentration of an inhibitor where the response (or binding) is reduced by half, and can be measured in whole cell, animals or in vitro cell-free (purified enzyme) systems. Inhibition of cell-free enzyme may also be reported as Ki values with some formal kinetics measurements.

The term “ICIC₅₀” or “IIC₅₀” is the measure of DPP8 and DPP9 inhibition in the context of a whole cell such that cell permeability becomes a factor (DPP8 and DPP9, which are cell permeable, the purified enzymes miss the cell permeable requirements for measuring IC₅₀)

The term “DPP8” refers to the protein dipeptidyl peptidase 8.

The term “DPP9” refers to the protein dipeptidyl peptidase 9.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

The terms “P1 position” and “P2 position”, in the case of a dipeptide (or dipeptide ananlog), refer to the carboxy and amino terminal residues, respectively. In the case of the subject I-DASH inhibitors, the P1 position is the amino acid (or amino acid analog) in which the boronic acid replaces the carboxy terminus.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

As used herein, reference to “about” or “approximately” a value or parameter includes (and describes) embodiments that are directed to that value or parameter. For example, description referring to “about X” includes description of “X”.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### III. Exemplary Embodiments

One aspect of the invention provides binder-drug conjugate comprising (i) a cell binding moiety, such as an antibody, antibody fragment, non-antibody scaffold or other polypeptide entity) that bind to a cell surface feature, such as protein, upregulated or otherwise selectively displayed on cells in a tumor, and (ii) one or more drug-conjugate moieties appended thereto, which drug-conjugate moieties are represented in the formulas

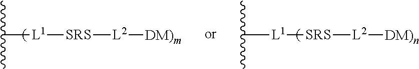

wherein
   L¹ represents a spacer or a bond;
   SRS represents a substrate recognition sequence for an extracellular protease which is expressed in the extracellular space of a tumor;
   L² represents a self immolative linker or a bond;
   DM represents a drug moiety;
   m represents an integer from 1 to 6, preferably 1, 2 or 3; and
   n represents an integer from 1 to 500, more preferably 1 to 100, 1 to 10 or 1 to 5.

The binder-drug conjugate, when bound with the surface feature on the target cell has an internalization half-time of at least 6 hours, more preferably at least 10, 12, 14, 16, 18, 20, 24, 36, 48, 60, 75 or even 100 hours.

a. Substrate Recognition Sequence

In certain embodiments, the Substrate Recognition Sequence is a moiety (typically a peptide or peptidyl moiety) that is cleaved by an enzyme expressed in the tissue in which the cell to which the binding moiety is directed. By “cleavage site that is cleavable selectively in the vicinity of the targeted cells” we include the meaning of a site that can only be cleaved by an agent which resides selectively in the vicinity of the targeted cells, so as to reduce the release of free drug moiety away from the disease tissue. Preferably, the enzyme that cleaves the Substrate Recognition Sequence resides in the vicinity of the target cells at a concentration at least five times or ten times higher than the concentration of the enzyme outside the vicinity of the target cells, and more preferably at a concentration at least 100 or 500 or 1000 times higher. Most preferably, the enzyme that cleaves the Substrate Recognition Sequence is found only in the vicinity of the target cells. For example, when the target cells are particular tumor cells (e.g. breast tumour cells), the Substrate Recognition Sequence may be one that is cleaved by an enzyme which resides selectively in the particular tumor (e.g. breast tumor) but which enzyme does not reside outside the vicinity of the particular tumor (e.g. breast tumor).

By ‘in the vicinity of cells’, we include the meaning of either on the surface of the cells or in the interstial fluid in that tissue, or both, or in the environment that immediately surrounds the cells e.g. blood, lymph, and other body fluids.

The Substrate Recognition Sequence is selectively cleaved in the vicinity of the target cells so that the free drug moiety is preferentially released from the conjugate in the vicinity of the target cells so as to exert its pharmacological activities preferentially on the cells/tissue nearby to the target cells, rather than on wanted (healthy) cells. Thus, it is preferred that the Substrate Recognition Sequence is selectively cleaved such that the drug moiety is released as the free drug moiety in the vicinity of the target cells at least five times or ten times more than the extent to which the free drug moiety it is released in the vicinity of healthy cells/tissues, and more preferably at least 100 or 500 or 1000 times more.

For a given target cell, the skilled person will be able to identify appropriate Substrate Recognition Sequences that are selectively cleavable in the vicinity of the target cell, using established methods in the art. For example, which proteases cleave which peptides can be assessed by consulting peptide libraries and studying an MS analysis of the fragmentation profile following cleavage. Also, published literature of protease cleavage motifs and peptide cleavage data can be searched as described further below.

Generally, the Substrate Recognition Sequence is a protease cleavage site. Thus, when the target cells are tumour cells, the Substrate Recognition Sequence may be cleavable selectively by proteases that reside in the vicinity of the tumour cells. In other words, the Substrate Recognition Sequence may be one that is cleavable by a tumour associated protease. It is well known that during tumour development, tumours aberrantly express proteases which allow them to invade local tissues and eventually metastasise.

The protease may be a metalloproteinase (MMP1-28) including both membrane bound (MMP14-17 and MMP24-25) and secreted forms (MMP1-13 and MMP18-23 and MMP26-28). The protease may belong to the A Disintegrin and Metalloproteinase (ADAM) and A Disintegrin, or Metalloproteinase with Thrombospondin Motifs (ADAMTS) families of proteases. Other examples include CD10 (CALLA) and prostate specific antigen (PSA). In certain preferred embodiments, the protease is Fibroblast Activation Protein (FAPD). It is appreciated that the proteases may or may not be membrane bound.

Protease cleavage sites are well known in the scientific literature, and can readily serve as the basis for a given Substrate Recognition Sequence being included in the drug-conjugate moieties using established synthetic techniques known in the art.

To the extent representing a protease whose extracellular concention is upregulated/increased in the target tissue by changes in expression, cellular trafficking or, in the case of intracellular enzymes that may become extracellular, by cell lysis caused by the disease state, Substrate Recognition Sequence may utilized which are designed to be selectively cleavable by one or a select sub-group of human proteases selected from the group consisting of (MEROPS peptidase database number provided in parentheses; Rawlings N. D., Morton F. R., Kok, C. Y., Kong, J. & Barrett A. J. (2008) MEROPS: the peptidase database. Nucleic Acids Res. 36 Database issue, D320-325): pepsin A (MER000885), gastricsin (MER000894), memapsin-2 (MER005870), renin (MER000917), cathepsin D (MER000911), cathepsin E (MER000944), memapsin-1 (MER005534), napsin A (MER004981), Mername-AA034 peptidase (MER014038), pepsin A4 (MER037290), pepsin A5 (Homo sapiens) (MER037291), hCG1733572 (Homo sapiens)-type putative peptidase (MER107386), napsin B pseudogene (MER004982), CYMP g.p. (Homo sapiens) (MER002929), subfamily A1A unassigned peptidases (MER181559), mouse mammary tumor virus retropepsin (MER048030), rabbit endogenous retrovirus endopeptidase (MER043650), S71-related human endogenous retropepsin (MER001812), RTVL-H-type putative peptidase (MER047117), RTVL-H-type putative peptidase (MER047133), RTVL-H-type putative peptidase (MER047160), RTVL-H-type putative peptidase (MER047206), RTVL-H-type putative peptidase (MER047253), RTVL-H-type putative peptidase (MER047260), RTVL-H-type putative peptidase (MER047291), RTVL-H-type putative peptidase (MER047418), RTVL-H-type putative peptidase (MER047440), RTVL-H-type putative peptidase (MER047479), RTVL-H-type putative peptidase (MER047559), RTVL-H-type putative peptidase (MER047583), RTVL-H-type putative peptidase (MER015446), human endogenous retrovirus retropepsin homologue 1 (MER015479), human endogenous retrovirus retropepsin homologue 2 (MER015481), endogenous retrovirus retropepsin pseudogene 1 (Homo sapiens chromosome 14) (MER029977), endogenous retrovirus retropepsin pseudogene 2 (Homo sapiens chromosome 8) (MER029665), endogenous retrovirus retropepsin pseudogene 3 (Homo sapiens chromosome 17) (MER002660), endogenous retrovirus retropepsin pseudogene 3 (Homo sapiens chromosome 17) (MER030286), endogenous retrovirus retropepsin pseudogene 3 (Homo sapiens chromosome 17) (MER047144), endogenous retrovirus retropepsin pseudogene 5 (Homo sapiens chromosome 12) (MER029664), endogenous retrovirus retropepsin pseudogene 6 (Homo sapiens chromosome 7) (MER002094), endogenous retrovirus retropepsin pseudogene 7 (Homo sapiens chromosome 6) (MER029776), endogenous retrovirus retropepsin pseudogene 8 (Homo sapiens chromosome Y) (MER030291), endogenous retrovirus retropepsin pseudogene 9 (Homo sapiens chromosome 19) (MER029680), endogenous retrovirus retropepsin pseudogene 10 (Homo sapiens chromosome 12) (MER002848), endogenous retrovirus retropepsin pseudogene 11 (Homo sapiens chromosome 17) (MER004378), endogenous retrovirus retropepsin pseudogene 12 (Homo sapiens chromosome 11) (MER003344), endogenous retrovirus retropepsin pseudogene 13 (Homo sapiens chromosome 2 and similar) (MER029779), endogenous retrovirus retropepsin pseudogene 14 (Homo sapiens chromosome 2) (MER029778), endogenous retrovirus retropepsin pseudogene 15 (Homo sapiens chromosome 4) (MER047158), endogenous retrovirus retropepsin pseudogene 15 (Homo sapiens chromosome 4) (MER047332), endogenous retrovirus retropepsin pseudogene 15 (Homo sapiens chromosome 4) (MER003182), endogenous retrovirus retropepsin pseudogene 16 (MER047165), endogenous retrovirus retropepsin pseudogene 16 (MER047178), endogenous retrovirus retropepsin pseudogene 16 (MER047200), endogenous retrovirus retropepsin pseudogene 16 (MER047315), endogenous retrovirus retropepsin pseudogene 16 (MER047405), endogenous retrovirus retropepsin pseudogene 16 (MER030292), endogenous retrovirus retropepsin pseudogene 17 (Homo sapiens chromosome 8) (MER005305), endogenous retrovirus retropepsin pseudogene 18 (Homo sapiens chromosome 4) (MER030288), endogenous retrovirus retropepsin pseudogene 19 (Homo sapiens chromosome 16) (MER001740), endogenous retrovirus retropepsin pseudogene 21 (Homo sapiens) (MER047222), endogenous retrovirus retropepsin pseudogene 21 (Homo sapiens) (MER047454), endogenous retrovirus retropepsin pseudogene 21 (Homo sapiens) (MER047477), endogenous retrovirus retropepsin pseudogene 21 (Homo sapiens) (MER004403), endogenous retrovirus retropepsin pseudogene 22 (Homo sapiens chromosome X) (MER030287), subfamily A2A non-peptidase homologues (MER047046), subfamily A2A non-peptidase homologues (MER047052), subfamily A2A non-peptidase homologues (MER047076), subfamily A2A non-peptidase homologues (MER047080), subfamily A2A non-peptidase homologues (MER047088), subfamily A2A non-peptidase homologues (MER047089), subfamily A2A non-peptidase homologues (MER047091), subfamily A2A non-peptidase homologues (MER047092), subfamily A2A non-peptidase homologues (MER047093), subfamily A2A non-peptidase homologues (MER047094), subfamily A2A non-peptidase homologues (MER047097), subfamily A2A non-peptidase homologues (MER047099), subfamily A2A non-peptidase homologues (MER047101), subfamily A2A non-peptidase homologues (MER047102), subfamily A2A non-peptidase homologues (MER047107), subfamily A2A non-peptidase homologues (MER047108), subfamily A2A non-peptidase homologues (MER047109), subfamily A2A non-peptidase homologues (MER047110), subfamily A2A non-peptidase homologues (MER047111), subfamily A2A non-peptidase homologues (MER047114), subfamily A2A non-peptidase homologues (MER047118), subfamily A2A non-peptidase homologues (MER047121), subfamily A2A non-peptidase homologues (MER047122), subfamily A2A non-peptidase homologues (MER047126), subfamily A2A non-peptidase homologues (MER047129), subfamily A2A non-peptidase homologues (MER047130), subfamily A2A non-peptidase homologues (MER047134), subfamily A2A non-peptidase homologues (MER047135), subfamily A2A non-peptidase homologues (MER047137), subfamily A2A non-peptidase homologues (MER047140), subfamily A2A non-peptidase homologues (MER047141), subfamily A2A non-peptidase homologues (MER047142), subfamily A2A non-peptidase homologues (MER047148), subfamily A2A non-peptidase homologues (MER047149), subfamily A2A non-peptidase homologues (MER047151), subfamily A2A non-peptidase homologues (MER047154), subfamily A2A non-peptidase homologues (MER047155), subfamily A2A non-peptidase homologues (MER047156), subfamily A2A non-peptidase homologues (MER047157), subfamily A2A non-peptidase homologues (MER047159), subfamily A2A non-peptidase homologues (MER047161), subfamily A2A non-peptidase homologues (MER047163), subfamily A2A non-peptidase homologues (MER047166), subfamily A2A non-peptidase homologues (MER047171), subfamily A2A non-peptidase homologues (MER047173), subfamily A2A non-peptidase homologues (MER047174), subfamily A2A non-peptidase homologues (MER047179), subfamily A2A non-peptidase homologues (MER047183), subfamily A2A non-peptidase homologues (MER047186), subfamily A2A non-peptidase homologues (MER047190), subfamily A2A non-peptidase homologues (MER047191), subfamily A2A non-peptidase homologues (MER047196), subfamily A2A non-peptidase homologues (MER047198), subfamily A2A non-peptidase homologues (MER047199), subfamily A2A non-peptidase homologues (MER047201), subfamily A2A non-peptidase homologues (MER047202), subfamily A2A non-peptidase homologues (MER047203), subfamily A2A non-peptidase homologues (MER047204), subfamily A2A non-peptidase homologues (MER047205), subfamily A2A non-peptidase homologues (MER047207), subfamily A2A non-peptidase homologues (MER047208), subfamily A2A non-peptidase homologues (MER047210), subfamily A2A non-peptidase homologues (MER047211), subfamily A2A non-peptidase homologues (MER047212), subfamily A2A non-peptidase homologues (MER047213), subfamily A2A non-peptidase homologues (MER047215), subfamily A2A non-peptidase homologues (MER047216), subfamily A2A non-peptidase homologues (MER047218), subfamily A2A non-peptidase homologues (MER047219), subfamily A2A non-peptidase homologues (MER047221), subfamily A2A non-peptidase homologues (MER047224), subfamily A2A non-peptidase homologues (MER047225), subfamily A2A non-peptidase homologues (MER047226), subfamily A2A non-peptidase homologues (MER047227), subfamily A2A non-peptidase homologues (MER047230), subfamily A2A non-peptidase homologues (MER047232), subfamily A2A non-peptidase homologues (MER047233), subfamily A2A non-peptidase homologues (MER047234), subfamily A2A non-peptidase homologues (MER047236), subfamily A2A non-peptidase homologues (MER047238), subfamily A2A non-peptidase homologues (MER047239), subfamily A2A non-peptidase homologues (MER047240), subfamily A2A non-peptidase homologues (MER047242), subfamily A2A non-peptidase homologues (MER047243), subfamily A2A non-peptidase homologues (MER047249), subfamily A2A non-peptidase homologues (MER047251), subfamily A2A non-peptidase homologues (MER047252), subfamily A2A non-peptidase homologues (MER047254), subfamily A2A non-peptidase homologues (MER047255), subfamily A2A non-peptidase homologues (MER047263), subfamily A2A non-peptidase homologues (MER047265), subfamily A2A non-peptidase homologues (MER047266), subfamily A2A non-peptidase homologues (MER047267), subfamily A2A non-peptidase homologues (MER047268), subfamily A2A non-peptidase homologues (MER047269), subfamily A2A non-peptidase homologues (MER047272), subfamily A2A non-peptidase homologues (MER047273), subfamily A2A non-peptidase homologues (MER047274), subfamily A2A non-peptidase homologues (MER047275), subfamily A2A non-peptidase homologues (MER047276), subfamily A2A non-peptidase homologues (MER047279), subfamily A2A non-peptidase homologues (MER047280), subfamily A2A non-peptidase homologues (MER047281), subfamily A2A non-peptidase homologues (MER047282), subfamily A2A non-peptidase homologues (MER047284), subfamily A2A non-peptidase homologues (MER047285), subfamily A2A non-peptidase homologues (MER047289), subfamily A2A non-peptidase homologues (MER047290), subfamily A2A non-peptidase homologues (MER047294), subfamily A2A non-peptidase homologues (MER047295), subfamily A2A non-peptidase homologues (MER047298), subfamily A2A non-peptidase homologues (MER047300), subfamily A2A non-peptidase homologues (MER047302), subfamily A2A non-peptidase homologues (MER047304), subfamily A2A non-peptidase homologues (MER047305), subfamily A2A non-peptidase homologues (MER047306), subfamily A2A non-peptidase homologues (MER047307), subfamily A2A non-peptidase homologues (MER047310), subfamily A2A non-peptidase homologues (MER047311), subfamily A2A non-peptidase homologues (MER047314), subfamily A2A non-peptidase homologues (MER047318), subfamily A2A non-peptidase homologues (MER047320), subfamily A2A non-peptidase homologues (MER047321), subfamily A2A non-peptidase homologues (MER047322), subfamily A2A non-peptidase homologues (MER047326), subfamily A2A non-peptidase homologues (MER047327), subfamily A2A non-peptidase homologues (MER047330), subfamily A2A non-peptidase homologues (MER047333), subfamily A2A non-peptidase homologues (MER047362), subfamily A2A non-peptidase homologues (MER047366), subfamily A2A non-peptidase homologues (MER047369), subfamily A2A non-peptidase homologues (MER047370), subfamily A2A non-peptidase homologues (MER047371), subfamily A2A non-peptidase homologues (MER047375), subfamily A2A non-peptidase homologues (MER047376), subfamily A2A non-peptidase homologues (MER047381), subfamily A2A non-peptidase homologues (MER047383), subfamily A2A non-peptidase homologues (MER047384), subfamily A2A non-peptidase homologues (MER047385), subfamily A2A non-peptidase homologues (MER047388), subfamily A2A non-peptidase homologues (MER047389), subfamily A2A non-peptidase homologues (MER047391), subfamily A2A non-peptidase homologues (MER047394), subfamily A2A non-peptidase homologues (MER047396), subfamily A2A non-peptidase homologues (MER047400), subfamily A2A non-peptidase homologues (MER047401), subfamily A2A non-peptidase homologues (MER047403), subfamily A2A non-peptidase homologues (MER047406), subfamily A2A non-peptidase homologues (MER047407), subfamily A2A non-peptidase homologues (MER047410), subfamily A2A non-peptidase homologues (MER047411), subfamily A2A non-peptidase homologues (MER047413), subfamily A2A non-peptidase homologues (MER047414), subfamily A2A non-peptidase homologues (MER047416), subfamily A2A non-peptidase homologues (MER047417), subfamily A2A non-peptidase homologues (MER047420), subfamily A2A non-peptidase homologues (MER047423), subfamily A2A non-peptidase homologues (MER047424), subfamily A2A non-peptidase homologues (MER047428), subfamily A2A non-peptidase homologues (MER047429), subfamily A2A non-peptidase homologues (MER047431), subfamily A2A non-peptidase homologues (MER047434), subfamily A2A non-peptidase homologues (MER047439), subfamily A2A non-peptidase homologues (MER047442), subfamily A2A non-peptidase homologues (MER047445), subfamily A2A non-peptidase homologues (MER047449), subfamily A2A non-peptidase homologues (MER047450), subfamily A2A non-peptidase homologues (MER047452), subfamily A2A non-peptidase homologues (MER047455), subfamily A2A non-peptidase homologues (MER047457), subfamily A2A non-peptidase homologues (MER047458), subfamily A2A non-peptidase homologues (MER047459), subfamily A2A non-peptidase homologues (MER047463), subfamily A2A non-peptidase homologues (MER047468), subfamily A2A non-peptidase homologues (MER047469), subfamily A2A non-peptidase homologues (MER047470), subfamily A2A non-peptidase homologues (MER047476), subfamily A2A non-peptidase homologues (MER047478), subfamily A2A non-peptidase homologues (MER047483), subfamily A2A non-peptidase homologues (MER047488), subfamily A2A non-peptidase homologues (MER047489), subfamily A2A non-peptidase homologues (MER047490), subfamily A2A non-peptidase homologues (MER047493), subfamily A2A non-peptidase homologues (MER047494), subfamily A2A non-peptidase homologues (MER047495), subfamily A2A non-peptidase homologues (MER047496), subfamily A2A non-peptidase homologues (MER047497), subfamily A2A non-peptidase homologues (MER047499), subfamily A2A non-peptidase homologues (MER047502), subfamily A2A non-peptidase homologues (MER047504), subfamily A2A non-peptidase homologues (MER047511), subfamily A2A non-peptidase homologues (MER047513), subfamily A2A non-peptidase homologues (MER047514), subfamily A2A non-peptidase homologues (MER047515), subfamily A2A non-peptidase homologues (MER047516), subfamily A2A non-peptidase homologues (MER047520), subfamily A2A non-peptidase homologues (MER047533), subfamily A2A non-peptidase homologues (MER047537), subfamily A2A non-peptidase homologues (MER047569), subfamily A2A non-peptidase homologues (MER047570), subfamily A2A non-peptidase homologues (MER047584), subfamily A2A non-peptidase homologues (MER047603), subfamily A2A non-peptidase homologues (MER047604), subfamily A2A non-peptidase homologues (MER047606), subfamily A2A non-peptidase homologues (MER047609), subfamily A2A non-peptidase homologues (MER047616), subfamily A2A non-peptidase homologues (MER047619), subfamily A2A non-peptidase homologues (MER047648), subfamily A2A non-peptidase homologues (MER047649), subfamily A2A non-peptidase homologues (MER047662), subfamily A2A non-peptidase homologues (MER048004), subfamily A2A non-peptidase homologues (MER048018), subfamily A2A non-peptidase homologues (MER048019), subfamily A2A non-peptidase homologues (MER048023), subfamily A2A non-peptidase homologues (MER048037), subfamily A2A unassigned peptidases (MER047164), subfamily A2A unassigned peptidases (MER047231), subfamily A2A unassigned peptidases (MER047386), skin aspartic protease (MER057097), presenilin 1 (MER005221), presenilin 2 (MER005223), impas 1 peptidase (MER019701), impas 1 peptidase (MER184722), impas 4 peptidase (MER019715), impas 2 peptidase (MER019708), impas 5 peptidase (MER019712), impas 3 peptidase (MER019711), possible family A22 pseudogene (Homo sapiens chromosome 18) (MER029974), possible family A22 pseudogene (Homo sapiens chromosome 11) (MER023159), cathepsin V (MER004437), cathepsin X (MER004508), cathepsin F (MER004980), cathepsin L (MER000622), cathepsin S (MER000633), cathepsin O (MER001690), cathepsin K (MER000644), cathepsin W (MER003756), cathepsin H (MER000629), cathepsin B (MER000686), dipeptidyl-peptidase I (MER001937), bleomycin hydrolase (animal) (MER002481), tubulointerstitial nephritis antigen (MER016137), tubulointerstitial nephritis antigen-related protein (MER021799), cathepsin L-like pseudogene 1 (Homo sapiens) (MER002789), cathepsin B-like pseudogene (chromosome 4, Homo sapiens) (MER029469), cathepsin B-like pseudogene (chromosome 1, Homo sapiens) (MER029457), CTSLL2 g.p. (Homo sapiens) (MER005210), CTSLL3 g.p. (Homo sapiens) (MER005209), calpain-1 (MER000770), calpain-2 (MER000964), calpain-3 (MER001446), calpain-9 (MER004042), calpain-8 (MER021474), calpain-15 (MER004745), calpain-5 (MER002939), calpain-11 (MER005844), calpain-12 (MER029889), calpain-10 (MER013510), calpain-13 (MER020139), calpain-14 (MER029744), Mername-AA253 peptidase (MER005537), calpamodulin (MER000718), hypothetical protein flj40251 (MER003201), ubiquitinyl hydrolase-L1 (MER000832), ubiquitinyl hydrolase-L3 (MER000836), ubiquitinyl hydrolase-BAP1 (MER003989), ubiquitinyl hydrolase-UCH37 (MER005539), ubiquitin-specific peptidase 5 (MER002066), ubiquitin-specific peptidase 6 (MER000863), ubiquitin-specific peptidase 4 (MER001795), ubiquitin-specific peptidase 8 (MER001884), ubiquitin-specific peptidase 13 (MER002627), ubiquitin-specific peptidase 2 (MER004834), ubiquitin-specific peptidase 11 (MER002693), ubiquitin-specific peptidase 14 (MER002667), ubiquitin-specific peptidase 7 (MER002896), ubiquitin-specific peptidase 9× (MER005877), ubiquitin-specific peptidase 10 (MER004439), ubiquitin-specific peptidase 1 (MER004978), ubiquitin-specific peptidase 12 (MER005454), ubiquitin-specific peptidase 16 (MER005493), ubiquitin-specific peptidase 15 (MER005427), ubiquitin-specific peptidase 17 (MER002900), ubiquitin-specific peptidase 19 (MER005428), ubiquitin-specific peptidase 20 (MER005494), ubiquitin-specific peptidase 3 (MER005513), ubiquitin-specific peptidase 9Y (MER004314), ubiquitin-specific peptidase 18 (MER005641), ubiquitin-specific peptidase 21 (MER006258), ubiquitin-specific peptidase 22 (MER012130), ubiquitin-specific peptidase 33 (MER014335), ubiquitin-specific peptidase 29 (MER012093), ubiquitin-specific peptidase 25 (MER011115), ubiquitin-specific peptidase 36 (MER014033), ubiquitin-specific peptidase 32 (MER014290), ubiquitin-specific peptidase 26 (Homo sapiens-type) (MER014292), ubiquitin-specific peptidase 24 (MER005706), ubiquitin-specific peptidase 42 (MER011852), ubiquitin-specific peptidase 46 (MER014629), ubiquitin-specific peptidase 37 (MER014633), ubiquitin-specific peptidase 28 (MER014634), ubiquitin-specific peptidase 47 (MER014636), ubiquitin-specific peptidase 38 (MER014637), ubiquitin-specific peptidase 44 (MER014638), ubiquitin-specific peptidase 50 (MER030315), ubiquitin-specific peptidase 35 (MER014646), ubiquitin-specific peptidase 30 (MER014649), Mername-AA091 peptidase (MER014743), ubiquitin-specific peptidase 45 (MER030314), ubiquitin-specific peptidase 51 (MER014769), ubiquitin-specific peptidase 34 (MER014780), ubiquitin-specific peptidase 48 (MER064620), ubiquitin-specific peptidase 40 (MER015483), ubiquitin-specific peptidase 41 (MER045268), ubiquitin-specific peptidase 31 (MER015493), Mername-AA129 peptidase (MER016485), ubiquitin-specific peptidase 49 (MER016486), Mername-AA187 peptidase (MER052579), USP17-like peptidase (MER030192), ubiquitin-specific peptidase 54 (MER028714), ubiquitin-specific peptidase 53 (MER027329), ubiquitin-specific endopeptidase 39 [misleading] (MER064621), Mername-AA090 non-peptidase homologue (MER014739), ubiquitin-specific peptidase [misleading] (MER030140), ubiquitin-specific peptidase 52 [misleading] (MER030317), NEK2 pseudogene (MER014736), C19 pseudogene (Homo sapiens: chromosome 5) (MER029972), Mername-AA088 peptidase (MER014750), autophagin-2 (MER013564), autophagin-1 (MER013561), autophagin-3 (MER014316), autophagin-4 (MER064622), Cezanne deubiquitinylating peptidase (MER029042), Cezanne-2 peptidase (MER029044), tumor necrosis factor alpha-induced protein 3 (MER029050), trabid peptidase (MER029052), VCIP135 deubiquitinating peptidase (MER152304), otubain-1 (MER029056), otubain-2 (MER029061), CyID protein (MER030104), UfSP1 peptidase (MER042724), UfSP2 peptidase (MER060306), DUBA deubiquitinylating enzyme (MER086098), KIAA0459 (Homo sapiens)-like protein (MER122467), Otud1 protein (MER125457), glycosyltransferase 28 domain containing 1, isoform CRA_c (Homo sapiens)-like (MER123606), hin1L g.p. (Homo sapiens) (MER139816), ataxin-3 (MER099998), ATXN3L putative peptidase (MER115261), Josephin domain containing 1 (Homo sapiens) (MER125334), Josephin domain containing 2 (Homo sapiens) (MER124068), YOD1 peptidase (MER116559), legumain (plant alpha form) (MER044591), legumain (MER001800), glycosylphosphatidylinositol:protein transamidase (MER002479), legumain pseudogene (Homo sapiens) (MER029741), family C13 unassigned peptidases (MER175813), caspase-1 (MER000850), caspase-3 (MER000853), caspase-7 (MER002705), caspase-6 (MER002708), caspase-2 (MER001644), caspase-4 (MER001938), caspase-5 (MER002240), caspase-8 (MER002849), caspase-9 (MER002707), caspase-10 (MER002579), caspase-14 (MER012083), paracaspase (MER019325), Mername-AA143 peptidase (MER021304), Mername-AA186 peptidase (MER020516), putative caspase (Homo sapiens) (MER021463), FLIP protein (MER003026), Mername-AA142 protein (MER021316), caspase-12 pseudogene (Homo sapiens) (MER019698), Mername-AA093 caspase pseudogene (MER014766), subfamily C14A non-peptidase homologues (MER185329), subfamily C14A non-peptidase homologues (MER179956), separase (Homo sapiens-type) (MER011775), separase-like pseudogene (MER014797), SENP1 peptidase (MER011012), SENP3 peptidase (MER011019), SENP6 peptidase (MER011109), SENP2 peptidase (MER012183), SENP5 peptidase (MER014032), SENP7 peptidase (MER014095), SENP8 peptidase (MER016161), SENP4 peptidase (MER005557), pyroglutamyl-peptidase I (chordate) (MER011032), Mername-AA073 peptidase (MER029978), Sonic hedgehog protein (MER002539), Indian hedgehog protein (MER002538), Desert hedgehog protein (MER012170), dipeptidyl-peptidase III (MER004252), Mername-AA164 protein (MER020410), LOC138971 g.p. (Homo sapiens) (MER020074), Atp23 peptidase (MER060642), prenyl peptidase 1 (MER004246), aminopeptidase N (MER000997), aminopeptidase A (MER001012), leukotriene A4 hydrolase (MER001013), pyroglutamyl-peptidase II (MER012221), cytosol alanyl aminopeptidase (MER002746), cystinyl aminopeptidase (MER002060), aminopeptidase B (MER001494), aminopeptidase PILS (MER005331), arginyl aminopeptidase-like 1 (MER012271), leukocyte-derived arginine aminopeptidase (MER002968), aminopeptidase Q (MER052595), aminopeptidase 0 (MER019730), Tata binding protein associated factor (MER026493), angiotensin-converting enzyme peptidase unit 1 (MER004967), angiotensin-converting enzyme peptidase unit 2 (MER001019), angiotensin-converting enzyme-2 (MER011061), Mername-AA153 protein (MER020514), thimet oligopeptidase (MER001737), neurolysin (MER010991), mitochondrial intermediate peptidase (MER003665), Mername-AA154 protein (MER021317), leishmanolysin-2 (MER014492), leishmanolysin-3 (MER180031), matrix metallopeptidase-1 (MER001063), matrix metallopeptidase-8 (MER001084), matrix metallopeptidase-2 (MER001080), matrix metallopeptidase-9 (MER001085), matrix metallopeptidase-3 (MER001068), matrix metallopeptidase-10 (Homo sapiens-type) (MER001072), matrix metallopeptidase-11 (MER001075), matrix metallopeptidase-7 (MER001092), matrix metallopeptidase-12 (MER001089), matrix metallopeptidase-13 (MER001411), membrane-type matrix metallopeptidase-1 (MER001077), membrane-type matrix metallopeptidase-2 (MER002383), membrane-type matrix metallopeptidase-3 (MER002384), membrane-type matrix metallopeptidase-4 (MER002595), matrix metallopeptidase-20 (MER003021), matrix metallopeptidase-19 (MER002076), matrix metallopeptidase-23B (MER004766), membrane-type matrix metallopeptidase-5 (MER005638), membrane-type matrix metallopeptidase-6 (MER012071), matrix metallopeptidase-21 (MER006101), matrix metallopeptidase-22 (MER014098), matrix metallopeptidase-26 (MER012072), matrix metallopeptidase-28 (MER013587), matrix metallopeptidase-23A (MER037217), macrophage elastase homologue (chromosome 8, Homo sapiens) (MER030035), Mername-AA156 protein (MER021309), matrix metallopeptidase-like 1 (MER045280), subfamily M10A non-peptidase homologues (MER175912), subfamily M10A non-peptidase homologues (MER187997), subfamily M10A non-peptidase homologues (MER187998), subfamily M10A non-peptidase homologues (MER180000), meprin alpha subunit (MER001111), meprin beta subunit (MER005213), procollagen C-peptidase (MER001113), mammalian tolloid-like 1 protein (MER005124), mammalian-type tolloid-like 2 protein (MER005866), ADAMTS9 peptidase (MER012092), ADAMTS14 peptidase (MER016700), ADAMTS15 peptidase (MER017029), ADAMTS16 peptidase (MER015689), ADAMTS17 peptidase (MER016302), ADAMTS18 peptidase (MER016090), ADAMTS19 peptidase (MER015663), ADAMS peptidase (MER003902), ADAM9 peptidase (MER001140), ADAM10 peptidase (MER002382), ADAM12 peptidase (MER005107), ADAM19 peptidase (MER012241), ADAM15 peptidase (MER002386), ADAM17 peptidase (MER003094), ADAM20 peptidase (MER004725), ADAMDEC1 peptidase (MER000743), ADAMTS3 peptidase (MER005100), ADAMTS4 peptidase (MER005101), ADAMTS1 peptidase (MER005546), ADAM28 peptidase (Homo sapiens-type) (MER005495), ADAMTS5 peptidase (MER005548), ADAMTS8 peptidase (MER005545), ADAMTS6 peptidase (MER005893),

ADAMTS7 peptidase (MER005894), ADAM30 peptidase (MER006268), ADAM21 peptidase (Homo sapiens-type) (MER004726), ADAMTS10 peptidase (MER014331), ADAMTS12 peptidase (MER014337), ADAMTS13 peptidase (MER015450), ADAM33 peptidase (MER015143), ovastacin (MER029996), ADAMTS20 peptidase (Homo sapiens-type) (MER026906), procollagen I N-peptidase (MER004985), ADAM2 protein (MER003090), ADAM6 protein (MER047044), ADAM7 protein (MER005109), ADAM18 protein (MER012230), ADAM32 protein (MER026938), non-peptidase homologue (Homo sapiens chromosome 4) (MER029973), family M12 non-peptidase homologue (Homo sapiens chromosome 16) (MER047654), family M12 non-peptidase homologue (Homo sapiens chromosome 15) (MER047250), ADAM3B protein (Homo sapiens-type) (MER005199), ADAM11 protein (MER001146), ADAM22 protein (MER005102), ADAM23 protein (MER005103), ADAM29 protein (MER006267), protein similar to ADAM21 peptidase preproprotein (Homo sapiens) (MER026944), Mername-AA225 peptidase homologue (Homo sapiens) (MER047474), putative ADAM pseudogene (chromosome 4, Homo sapiens) (MER029975), ADAM3A g.p. (Homo sapiens) (MER005200), ADAM1 g.p. (Homo sapiens) (MER003912), subfamily M12B non-peptidase homologues (MER188210), subfamily M12B non-peptidase homologues (MER188211), subfamily M12B non-peptidase homologues (MER188212), subfamily M12B non-peptidase homologues (MER188220), neprilysin (MER001050), endothelin-converting enzyme 1 (MER001057), endothelin-converting enzyme 2 (MER004776), DINE peptidase (MER005197), neprilysin-2 (MER013406), Kell blood-group protein (MER001054), PHEX peptidase (MER002062), i-AAA peptidase (MER001246), i-AAA peptidase (MER005755), paraplegin (MER004454), Afg3-like protein 2 (MER005496), Afg3-like protein 1A (MER014306), pappalysin-1 (MER002217), pappalysin-2 (MER014521), famesylated-protein converting enzyme 1 (MER002646), metalloprotease-related protein-1 (MER030873), aminopeptidase AMZ2 (MER011907), aminopeptidase AMZ1 (MER058242), carboxypeptidase A1 (MER001190), carboxypeptidase A2 (MER001608), carboxypeptidase B (MER001194), carboxypeptidase N (MER001198), carboxypeptidase E (MER001199), carboxypeptidase M (MER001205), carboxypeptidase U (MER001193), carboxypeptidase A3 (MER001187), metallocarboxypeptidase D peptidase unit 1 (MER003781), metallocarboxypeptidase Z (MER003428), metallocarboxypeptidase D peptidase unit 2 (MER004963), carboxypeptidase A4 (MER013421), carboxypeptidase A6 (MER013456), carboxypeptidase A5 (MER017121), metallocarboxypeptidase 0 (MER016044), cytosolic carboxypeptidase-like protein 5 (MER033174), cytosolic carboxypeptidase 3 (MER033176), cytosolic carboxypeptidase 6 (MER033178), cytosolic carboxypeptidase 1 (MER033179), cytosolic carboxypeptidase 2 (MER037713), metallocarboxypeptidase D non-peptidase unit (MER004964), adipocyte-enhancer binding protein 1 (MER003889), carboxypeptidase-like protein X1 (MER013404), carboxypeptidase-like protein X2 (MER078764), cytosolic carboxypeptidase (MER026952), family M14 non-peptidase homologues (MER199530), insulysin (MER001214), mitochondrial processing peptidase beta-subunit (MER004497), nardilysin (MER003883), eupitrilysin (MER004877), mitochondrial processing peptidase non-peptidase alpha subunit (MER001413), ubiquinol-cytochrome c reductase core protein I (MER003543), ubiquinol-cytochrome c reductase core protein II (MER003544), ubiquinol-cytochrome c reductase core protein domain 2 (MER043998), insulysin unit 2 (MER046821), nardilysin unit 2 (MER046874), insulysin unit 3 (MER078753), mitochondrial processing peptidase subunit alpha unit 2 (MER124489), nardilysin unit 3 (MER142856), LOC133083 g.p. (Homo sapiens) (MER021876), subfamily M16B non-peptidase homologues (MER188757), leucyl aminopeptidase (animal) (MER003100), Mername-AA040 peptidase (MER003919), leucyl aminopeptidase-1 (Caenorhabditis-type) (MER013416), methionyl aminopeptidase 1 (MER001342), methionyl aminopeptidase 2 (MER001728), aminopeptidase P2 (MER004498), Xaa-Pro dipeptidase (eukaryote) (MER001248), aminopeptidase P1 (MER004321), mitochondrial intermediate cleaving peptidase 55 kDa (MER013463), mitochondrial methionyl aminopeptidase (MER014055), Mername-AA020 peptidase homologue (MER010972), proliferation-association protein 1 (MER005497), chromatin-specific transcription elongation factor 140 kDa subunit (MER026495), proliferation-associated protein 1-like (Homo sapiens chromosome X) (MER029983), Mername-AA226 peptidase homologue (Homo sapiens) (MER056262), Mername-AA227 peptidase homologue (Homo sapiens) (MER047299), subfamily M24A non-peptidase homologues (MER179893), aspartyl aminopeptidase (MER003373), Gly-Xaa carboxypeptidase (MER033182), carnosine dipeptidase II (MER014551), carnosine dipeptidase I (MER015142), Mername-AA161 protein (MER021873), aminoacylase (MER001271), glutamate carboxypeptidase II (MER002104), NAALADASE L peptidase (MER005239), glutamate carboxypeptidase III (MER005238), plasma glutamate carboxypeptidase (MER005244), Mername-AA103 peptidase (MER015091), Fxna peptidase (MER029965), transferrin receptor protein (MER002105), transferrin receptor 2 protein (MER005152), glutaminyl cyclise (MER015095), glutamate carboxypeptidase II (Homo sapiens)-type non-peptidase homologue (MER026971), nicalin (MER044627), membrane dipeptidase (MER001260), membrane-bound dipeptidase-2 (MER013499), membrane-bound dipeptidase-3 (MER013496), dihydro-orotase (MER005767), dihydropyrimidinase (MER033266), dihydropyrimidinase related protein-1 (MER030143), dihydropyrimidinase related protein-2 (MER030155), dihydropyrimidinase related protein-3 (MER030151), dihydropyrimidinase related protein-4 (MER030149), dihydropyrimidinase related protein-5 (MER030136), hypothetical protein like 5730457F11RIK (MER033184), 1300019j08rik protein (MER033186)), guanine aminohydrolase (MER037714), Kael putative peptidase (MER001577), OSGEPL1-like protein (MER013498), S2P peptidase (MER004458), subfamily M23B non-peptidase homologues (MER199845), subfamily M23B non-peptidase homologues (MER199846), subfamily M23B non-peptidase homologues (MER199847), subfamily M23B non-peptidase homologues (MER137320), subfamily M23B non-peptidase homologues (MER201557), subfamily M23B non-peptidase homologues (MER199417), subfamily M23B non-peptidase homologues (MER199418), subfamily M23B non-peptidase homologues (MER199419), subfamily M23B non-peptidase homologues (MER199420), subfamily M23B non-peptidase homologues (MER175932), subfamily M23B non-peptidase homologues (MER199665), Pohl peptidase (MER020382), Jab1/MPN domain metalloenzyme (MER022057), Mername-AA165 peptidase (MER021865), Brcc36 isopeptidase (MER021890), histone H2A deubiquitinase MYSM1 (MER021887), AMSH deubiquitinating peptidase (MER030146), putative peptidase (Homo sapiens chromosome 2) (MER029970), Mername-AA168 protein (MER021886), COP9 signalosome subunit 6 (MER030137), 26S proteasome non-ATPase regulatory subunit 7 (MER030134), eukaryotic translation initiation factor 3 subunit 5 (MER030133), 1FP38 peptidase homologue (MER030132), subfamily M67A non-peptidase homologues (MER191181), subfamily M67A unassigned peptidases (MER191144), granzyme B (Homo sapiens-type) (MER000168), testisin (MER005212), tryptase beta (MER000136), kallikrein-related peptidase 5 (MER005544), corin (MER005881), kallikrein-related peptidase 12 (MER006038), DESC1 peptidase (MER006298), tryptase gamma 1 (MER011036), kallikrein-related peptidase 14 (MER011038), hyaluronan-binding peptidase (MER003612), transmembrane peptidase, serine 4 (MER011104), intestinal serine peptidase (rodent) (MER016130), adrenal secretory serine peptidase (MER003734), tryptase delta 1 (Homo sapiens) (MER005948), matriptase-3 (MER029902), marapsin (MER006119), tryptase-6 (MER006118), ovochymase-1 domain 1 (MER099182), transmembrane peptidase, serine 3 (MER005926), kallikrein-related peptidase 15 (MER000064), Mername-AA031 peptidase (MER014054), TMPRSS13 peptidase (MER014226), Mername-AA038 peptidase (MER062848), Mername-AA204 peptidase (MER029980), cationic trypsin (Homo sapiens-type) (MER000020), elastase-2 (MER000118), mannan-binding lectin-associated serine peptidase-3 (MER031968), cathepsin G (MER000082), myeloblastin (MER000170), granzyme A (MER001379), granzyme M (MER001541), chymase (Homo sapiens-type) (MER000123), tryptase alpha (MER000135), granzyme K (MER001936), granzyme H (MER000166), chymotrypsin B (MER000001), elastase-1 (MER003733), pancreatic endopeptidase E (MER000149), pancreatic elastase II (MER000146), enteropeptidase (MER002068), chymotrypsin C (MER000761), prostasin (MER002460), kallikrein 1 (MER000093), kallikrein-related peptidase 2 (MER000094), kallikrein-related peptidase 3 (MER000115), mesotrypsin (MER000022), complement component C1r-like peptidase (MER016352), complement factor D (MER000130), complement component activated C1r (MER000238), complement component activated C1s (MER000239), complement component C2a (MER000231), complement factor B (MER000229), mannan-binding lectin-associated serine peptidase 1 (MER000244), complement factor I (MER000228), pancreatic endopeptidase E form B (MER000150), pancreatic elastase IIB (MER000147), coagulation factor XIIa (MER000187), plasma kallikrein (MER000203) coagulation factor Xia (MER000210), coagulation factor IXa (MER000216), coagulation factor Vila (MER000215), coagulation factor Xa (MER000212), thrombin (MER000188), protein C (activated) (MER000222), acrosin (MER000078), hepsin (MER000156), hepatocyte growth factor activator (MER000186), mannan-binding lectin-associated serine peptidase 2 (MER002758), u-plasminogen activator (MER000195), t-plasminogen activator (MER000192), plasmin (MER000175), kallikrein-related peptidase 6 (MER002580), neurotrypsin (MER004171), kallikrein-related peptidase 8 (MER005400), kallikrein-related peptidase 10 (MER003645), epitheliasin (MER003736), kallikrein-related peptidase 4 (MER005266), prosemin (MER004214), chymopasin (MER001503), kallikrein-related peptidase 11 (MER004861), kallikrein-related peptidase 11 (MER216142), trypsin-2 type A (MER000021), HtrA1 peptidase (Homo sapiens-type) (MER002577), HtrA2 peptidase (MER208413), HtrA2 peptidase (MER004093), HtrA3 peptidase (MER014795), HtrA4 peptidase (MER016351), Tysnd1 peptidase (MER050461), TMPRSS12 peptidase (MER017085), HAT-like putative peptidase 2 (MER021884), trypsin C (MER021898), kallikrein-related peptidase 7 (MER002001), matriptase (MER003735), kallikrein-related peptidase 13 (MER005269), kallikrein-related peptidase 9 (MER005270), matriptase-2 (MER005278), umbelical vein peptidase (MER005421), LCLP peptidase (MER001900), spinesin (MER014385), marapsin-2 (MER021929), complement factor D-like putative peptidase (MER056164), ovochymase-2 (MER022410), HAT-like 4 peptidase (MER044589), ovochymase 1 domain 1 (MER022412), epidermis-specific SP-like putative peptidase (MER029900), testis serine peptidase 5 (MER029901), Mername-AA258 peptidase (MER000285), polyserase-IA unit 1 (MER030879), polyserase-IA unit 2 (MER030880), testis serine peptidase 2 (human-type) (MER033187), hypothetical acrosin-like peptidase (Homo sapiens) (MER033253), HAT-like 5 peptidase (MER028215), polyserase-3 unit 1 (MER061763), polyserase-3 unit 2 (MER061748), peptidase similar to tryptophan/serine protease (MER056263), polyserase-2 unit 1 (MER061777), Mername-AA123 peptidase (MER021930), HAT-like 2 peptidase (MER099184), hCG2041452-like protein (MER099172), hCG22067 (Homo sapiens) (MER099169), brain-rescue-factor-1 (Homo sapiens) (MER098873), hCG2041108 (Homo sapiens) (MER099173), polyserase-2 unit 2 (MER061760), polyserase-2 unit 3 (MER065694), Mername-AA201 (peptidase homologue) MER099175, secreted trypsin-like serine peptidase homologue (MER030000), polyserase-1A unit 3 (MER029880), azurocidin (MER000119), haptoglobin-1 (MER000233), haptoglobin-related protein (MER000235), macrophage-stimulating protein (MER001546), hepatocyte growth factor (MER000185), protein Z (MER000227), TESP1 protein (MER047214), LOC136242 protein (MER016132), plasma kallikrein-like protein 4 (MER016346), PRSS35 protein (MER016350), DKFZp586H2123-like protein (MER066474), apolipoprotein (MER000183), psi-KLK1 pseudogene (Homo sapiens) (MER033287), tryptase pseudogene I (MER015077), tryptase pseudogene II (MER015078), tryptase pseudogene III (MER015079), subfamily S1A unassigned peptidases (MER216982), subfamily S1A unassigned peptidases (MER216148), amidophosphoribosyltransferase precursor (MER003314), glutamine-fructose-6-phosphate transaminase 1 (MER003322), glutamine.fructose-6-phosphate amidotransferase (MER012158), Mername-AA144 protein (MER021319), asparagine synthetase (MER033254), family C44 non-peptidase homologues (MER159286), family C44 unassigned peptidases (MER185625) family C44 unassigned peptidases (MER185626), secernin 1 (MER045376), secernin 2 (MER064573), secernin 3 (MER064582), acid ceramidase precursor (MER100794), N-acylethanolamine acid amidase precursor (MER141667), proteasome catalytic subunit 1 (MER000556), proteasome catalytic subunit 2 (MER002625), proteasome catalytic subunit 3 (MER002149), proteasome catalytic subunit li (MER000552), proteasome catalytic subunit 2i (MER001515), proteasome catalytic subunit 3i (MER000555), proteasome catalytic subunit 5t (MER026203), protein serine kinase c17 (MER026497), proteasome subunit alpha 6 (MER000557), proteasome subunit alpha 2 (MER000550), proteasome subunit alpha 4 (MER000554), proteasome subunit alpha 7 (MER033250), proteasome subunit alpha 5 (MER000558), proteasome subunit alpha 1 (MER000549), proteasome subunit alpha 3 (MER000553), proteasome subunit XAPC7 (MER004372), proteasome subunit beta 3 (MER001710), proteasome subunit beta 2 (MER002676), proteasome subunit beta 1 (MER000551), proteasome subunit beta 4 (MER001711), Mername-AA230 peptidase homologue (Homo sapiens) (MER047329), Mername-AA231 pseudogene (Homo sapiens) (MER047172), Mername-AA232 pseudogene (Homo sapiens) (MER047316), glycosylasparaginase precursor (MER003299), isoaspartyl dipeptidase (threonine type) (MER031622), taspase-1 (MER016969), gamma-glutamyltransferase 5 (mammalian-type) (MER001977), gamma-glutamyltransferase 1 (mammalian-type) (MER001629), gamma-glutamyltransferase 2 (Homo sapiens) (MER001976), gamma-glutamyltransferase-like protein 4 (MER002721), gamma-glutamyltransferase-like protein 3 (MER016970), similar to gamma-glutamyltransferase 1 precursor (Homo sapiens) (MER026204), similar to gamma-glutamyltransferase 1 precursor (Homo sapiens) (MER026205), Mername-AA211 putative peptidase (MER026207), gamma-glutamyltransferase 6 (MER159283), gamma-glutamyl transpeptidase homologue (chromosome 2, Homo sapiens) (MER037241), polycystin-1 (MER126824), KIAA1879 protein (MER159329), polycystic kidney disease 1-like 3 (MER172554), gamma-glutamyl hydrolase (MER002963), guanine 5″-monophosphate synthetase (MER043387), carbamoyl-phosphate synthase (Homo sapiens-type) (MER078640), dihydro-orotase (N-terminal unit) (Homo sapiens-type) (MER060647) DJ-1 putative peptidase (MER003390), Mername-AA100 putative peptidase (MER014802), Mername-AA101 non-peptidase homologue (MER014803), KIAA0361 protein (Homo sapiens-type) (MER042827), F1134283 protein (Homo sapiens) (MER044553), non-peptidase homologue chromosome 21 open reading frame 33 (Homo sapiens) (MER160094), family C56 non-peptidase homologues (MER177016), family C56 non-peptidase homologues (MER176613), family C56 non-peptidase homologues (MER176918), EGF-like module containing mucin-like hormone receptor-like 2 (MER037230), CD97 antigen (human type) (MER037286), EGF-like module containing mucin-like hormone receptor-like 3 (MER037288), EGF-like module containing mucin-like hormone receptor-like 1 (MER037278), EGF-like module containing mucin-like hormone receptor-like 4 (MER037294), cadherin EGF LAG seven-pass G-type receptor 2 precursor (Homo sapiens) (MER045397), Gpr64 (Mus musculus)-type protein (MER123205), GPR56 (Homo sapiens)-type protein (MER122057), latrophilin 2 (MER122199), latrophilin-1 (MER126380), latrophilin 3 (MER124612), protocadherin Flamingo 2 (MER124239), ETL protein (MER126267), G protein-coupled receptor 112 (MER126114), seven transmembrane helix receptor (MER125448), Gpr114 protein (MER159320), GPR126 vascular inducible G protein-coupled receptor (MER140015), GPR125 (Homo sapiens)-type protein (MER159279), GPR116 (Homo sapiens)-type G-protein coupled receptor (MER159280), GPR128 (Homo sapiens)-type G-protein coupled receptor (MER162015), GPR133 (Homo sapiens)-type protein (MER159334), GPR110 G-protein coupled receptor (MER159277), GPR97 protein (MER159322), KPG 006 protein (MER161773), KPG_008 protein (MER161835), KPG_009 protein (MER159335), unassigned homologue (MER166269), GPR113 protein (MER159352), brain-specific angiogenesis inhibitor 2 (MER159746), PIDD auto-processing protein unit 1 (MER020001), PIDD auto-processing protein unit 2 (MER063690), MUC1 self-cleaving mucin (MER074260), dystroglycan (MER054741), proprotein convertase 9 (MER022416), site-1 peptidase (MER001948), furin (MER000375), proprotein convertase 1 (MER000376), proprotein convertase 2 (MER000377), proprotein convertase 4 (MER028255), PACE4 proprotein convertase (MER000383), proprotein convertase 5 (MER002578), proprotein convertase 7 (MER002984), tripeptidyl-peptidase II (MER000355), subfamily S8A non-peptidase homologues (MER201339), subfamily S8A non-peptidase homologues (MER191613), subfamily S8A unassigned peptidases (MER191611), subfamily S8A unassigned peptidases (MER191612), subfamily S8A unassigned peptidases (MER191614), tripeptidyl-peptidase I (MER003575), prolyl oligopeptidase (MER000393), dipeptidyl-peptidase IV (eukaryote) (MER000401), acylaminoacyl-peptidase (MER000408), fibroblast activation protein alpha subunit (MER000399), PREPL A protein (MER004227), dipeptidyl-peptidase 8 (MER013484), dipeptidyl-peptidase 9 (MER004923), FLJ1 putative peptidase (MER017240), Mername-AA194 putative peptidase (MER017353), Mername-AA195 putative peptidase (MER017367), Mername-AA196 putative peptidase (MER017368), Mername-AA197 putative peptidase (MER017371), C14orf29 protein (MER033244), hypothetical protein (MER033245), hypothetical esterase/lipase/thioesterase (MER047309), protein bat5 (MER037840), hypothetical protein flj40219 (MER033212), hypothetical protein flj37464 (MER033240), hypothetical protein flj33678 (MER033241), dipeptidylpeptidase homologue DPP6 (MER000403), dipeptidylpeptidase homologue DPP10 (MER005988), protein similar to Mus musculus chromosome 20 open reading frame 135 (MER037845), kynurenine formamidase (MER046020), thyroglobulin precursor (MER011604), acetylcholinesterase (MER033188), cholinesterase (MER033198), carboxylesterase D1 (MER033213), liver carboxylesterase (MER033220), carboxylesterase 3 (MER033224), carboxylesterase 2 (MER033226), bile salt-dependent lipase (MER033227), carboxylesterase-related protein (MER033231), neuroligin 3 (MER033232), neuroligin 4, X-linked (MER033235), neuroligin 4, Y-linked (MER033236), esterase D (MER043126), arylacetamide deacetylase (MER033237), KIAA1363-like protein (MER033242), hormone-sensitive lipase (MER033274), neuroligin 1 (MER033280), neuroligin 2 (MER033283), family S9 non-peptidase homologues (MER212939), family S9 non-peptidase homologues (MER211490), subfamily S9C unassigned peptidases (MER192341), family S9 unassigned peptidases (MER209181), family S9 unassigned peptidases (MER200434), family S9 unassigned peptidases (MER209507), family S9 unassigned peptidases (MER209142), serine carboxypeptidase A (MER000430), vitellogenic carboxypeptidase-like protein (MER005492), RISC peptidase (MER010960), family S15 unassigned peptidases (MER199442), family S15 unassigned peptidases (MER200437), family S15 unassigned peptidases (MER212825), lysosomal Pro-Xaa carboxypeptidase (MER000446), dipeptidyl-peptidase II (MER004952), thymus-specific serine peptidase (MER005538), epoxide hydrolase-like putative peptidase (MER031614), Loc328574-like protein (MER033246), abhydrolase domain-containing protein 4 (MER031616), epoxide hydrolase (MER000432), mesoderm specific transcript protein (MER199890), mesoderm specific transcript protein (MER017123), cytosolic epoxide hydrolase (MER029997), cytosolic epoxide hydrolase (MER213866), similar to hypothetical protein FLJ22408 (MER031608), CGI-58 putative peptidase (MER030163), Williams-Beuren syndrome critical region protein 21 epoxide hydrolase (MER031610), epoxide hydrolase (MER031612), hypothetical protein flj22408 (epoxide hydrolase) (MER031617), monoglyceride lipase (MER033247), hypothetical protein (MER033249), valacyclovir hydrolase (MER033259), Ccg1-interacting factor b (MER210738), glycosylasparaginase precursor (MER003299), isoaspartyl dipeptidase (threonine type) (MER031622). taspase-1 (MER016969), gamma-glutamyltransferase 5 (mammalian-type) (MER001977), gamma-glutamyltransferase 1 (mammalian-type) (MER001629), gamma-glutamyltransferase 2 (Homo sapiens) (MER001976), gamma-glutamyltransferase-like protein 4 (MER002721). gamma-glutamyltransferase-like protein 3 (MER016970). similar to gamma-glutamyltransferase 1 precursor (Homo sapiens) (MER026204). similar to gamma-glutamyltransferase 1 precursor (Homo sapiens) (MER026205). Mername-AA211 putative peptidase (MER026207). gamma-glutamyltransferase 6 (MER159283). gamma-glutamyl transpeptidase homologue (chromosome 2, Homo sapiens) (MER037241). polycystin-1 (MER126824), KIAA1879 protein (MER159329). polycystic kidney disease 1-like 3 (MER172554). gamma-glutamyl hydrolase (MER002963). guanine 5″-monophosphate synthetase (MER043387). carbamoyl-phosphate synthase (Homo sapiens-type) (MER078640). dihydro-orotase (N-terminal unit) (Homo sapiens-type) (MER060647). DJ-1 putative peptidase (MER003390). Mername-AA100 putative peptidase (MER014802). Mername-AA101 non-peptidase homologue (MER014803). KIAA0361 protein (Homo sapiens-type) (MER042827). FLJ34283 protein (Homo sapiens) (MER044553). non-peptidase homologue chromosome 21 open reading frame 33 (Homo sapiens) (MER160094). family C56 non-peptidase homologues (MER177016), family C56 non-peptidase homologues (MER176613). family C56 non-peptidase homologues (MER176918). EGF-like module containing mucin-like hormone receptor-like 2 (MER037230). CD97 antigen (human type) (MER037286). EGF-like module containing mucin-like hormone receptor-like 3 (MER037288). EGF-like module containing mucin-like hormone receptor-like 1 (MER037278). EGF-like module containing mucin-like hormone receptor-like 4 (MER037294). cadherin EGF LAG seven-pass G-type receptor 2 precursor (Homo sapiens) (MER045397), Gpr64 (Mus musculus)-type protein (MER123205). GPR56 (Homo sapiens)-type protein (MER122057). latrophilin 2 (MER122199). latrophilin-1 (MER126380). latrophilin 3 (MER124612). protocadherin Flamingo 2 (MER124239). ETL protein (MER126267). G protein-coupled receptor 112 (MER126114). seven transmembrane helix receptor (MER125448). Gpr114 protein (MER159320). GPR126 vascular inducible G protein-coupled receptor (MER140015). GPR125 (Homo sapiens)-type protein (MER159279). GPR116 (Homo sapiens)-type G-protein coupled receptor (MER159280). GPR128 (Homo sapiens)-type G-protein coupled receptor (MER162015). GPR133 (Homo sapiens)-type protein (MER159334) GPR110 G-protein coupled receptor (MER159277), GPR97 protein (MER159322), KPG_006 protein (MER161773) KPG_008 protein (MER161835), KPG_009 protein (MER159335), unassigned homologue (MER166269), GPR113 protein (MER159352), brain-specific angiogenesis inhibitor 2 (MER159746), PIDD auto-processing protein unit 1 (MER020001), PIDD auto-processing protein unit 2 (MER063690), MUC1 self-cleaving mucin (MER074260), dystroglycan (MER054741), proprotein convertase 9 (MER022416), site-1 peptidase (MER001948), furin (MER000375), proprotein convertase 1 (MER000376), proprotein convertase 2 (MER000377), proprotein convertase 4 (MER028255), PACE4 proprotein convertase (MER000383), proprotein convertase 5 (MER002578), proprotein convertase 7 (MER002984), tripeptidyl-peptidase II (MER000355), subfamily S8A non-peptidase homologues (MER201339), subfamily S8A non-peptidase homologues (MER191613), subfamily S8A unassigned peptidases (MER191611), subfamily S8A unassigned peptidases (MER191612), subfamily S8A unassigned peptidases (MER191614), tripeptidyl-peptidase I (MER003575), prolyl oligopeptidase (MER000393), dipeptidyl-peptidase IV (eukaryote) (MER000401), acylaminoacyl-peptidase (MER000408), fibroblast activation protein alpha subunit (MER000399), PREPL A protein (MER004227), dipeptidyl-peptidase 8 (MER013484), dipeptidyl-peptidase 9 (MER004923), FLJ1 putative peptidase (MER017240), Mername-AA194 putative peptidase (MER017353), Mername-AA195 putative peptidase (MER017367), Mername-AA196 putative peptidase (MER017368), Mername-AA197 putative peptidase (MER017371), C14orf29 protein (MER033244), hypothetical protein (MER033245), hypothetical esterase/lipase/thioesterase (MER047309), protein bat5 (MER037840), hypothetical protein flj40219 (MER033212), hypothetical protein flj37464 (MER033240), hypothetical protein flj33678 (MER033241), dipeptidylpeptidase homologue DPP6 (MER000403), dipeptidylpeptidase homologue DPP10 (MER005988), protein similar to Mus musculus chromosome 20 open reading frame 135 (MER037845), kynurenine formamidase (MER046020), thyroglobulin precursor (MER011604), acetylcholinesterase (MER033188), cholinesterase (MER033198), carboxylesterase D1 (MER033213), liver carboxylesterase (MER033220), carboxylesterase 3 (MER033224), carboxylesterase 2 (MER033226), bile salt-dependent lipase (MER033227), carboxylesterase-related protein (MER033231), neuroligin 3 (MER033232), neuroligin 4, X-linked (MER033235), neuroligin 4, Y-linked (MER033236), esterase D (MER043126), arylacetamide deacetylase (MER033237), KIAA1363-like protein (MER033242), hormone-sensitive lipase (MER033274), neuroligin 1 (MER033280), neuroligin 2 (MER033283), family S9 non-peptidase homologues (MER212939), family S9 non-peptidase homologues (MER211490), subfamily S9C unassigned peptidases (MER192341), family S9 unassigned peptidases (MER209181), family S9 unassigned peptidases (MER200434), family S9 unassigned peptidases (MER209507), family S9 unassigned peptidases (MER209142), serine carboxypeptidase A (MER000430), vitellogenic carboxypeptidase-like protein (MER005492), RISC peptidase (MER010960), family S15 unassigned peptidases (MER199442), family S15 unassigned peptidases (MER200437), family S15 unassigned peptidases (MER212825), lysosomal Pro-Xaa carboxypeptidase (MER000446), dipeptidyl-peptidase II (MER004952), thymus-specific serine peptidase (MER005538), epoxide hydrolase-like putative peptidase (MER031614), Loc328574-like protein (MER033246), abhydrolase domain-containing protein 4 (MER031616), epoxide hydrolase (MER000432), mesoderm specific transcript protein (MER199890), mesoderm specific transcript protein (MER017123), cytosolic epoxide hydrolase (MER029997), cytosolic epoxide hydrolase (MER213866), similar to hypothetical protein FLJ22408 (MER031608), CGI-58 putative peptidase (MER030163), Williams-Beuren syndrome critical region protein 21 epoxide hydrolase (MER031610), epoxide hydrolase (MER031612), hypothetical protein flj22408 (epoxide hydrolase) (MER031617), monoglyceride lipase (MER033247), hypothetical protein (MER033249), valacyclovir hydrolase (MER033259), Ccg1-interacting factor b (MER210738).

In some embodiments, the Substrate Recognition Sequence is a peptide moiety of up to 15 amino acids in length. The Substrate Recognition Sequence is cleaved by a protease. In some embodiments, the protease is co-localized with the target of the cell binding moiety in a tissue, and the protease cleaves the Substrate Recognition Sequence in the drug-conjugate moiety when the binder-drug conjugate is exposed to the protease. In some embodiments, the protease is not active or is significantly less active in tissues that do not significantly express the cell surface feature. In some embodiments, the protease is not active or is significantly less active in healthy, e.g., non-diseased tissues.

In certain embodiments, the Substrate Recognition Sequence is cleaved by a protease selected from the following:
ADAMS or ADAMTS, e.g. ADAM8, ADAM9, ADAM10, ADAM12, ADAM15, ADAM17/TACE, ADAMDEC1, ADAMTS1, ADAMTS4 or ADAMTS5.
Aspartate proteases, e.g., BACE or Renin.
Aspartic cathepsins (to the extent upregulated or released by cell lysis in the extracellular space), e.g., Cathepsin D or Cathepsin E.
Caspases (to the extent upregulated or released by cell lysis in the extracellular space), e.g., Caspase 1, Caspase 2, Caspase 3, Caspase 4, Caspase 5, Caspase 6, Caspase 7, Caspase 8, Caspase 9, Caspase 10 or Caspase 14.
Cysteine cathepsins, e.g., Cathepsin B, Cathepsin C, Cathepsin K, Cathepsin L, Cathepsin S, Cathepsin V/L2, Cathepsin X/Z/P.
Cysteine proteinases, e.g., Cruzipain, Legumain or Otubain-2.
KLKs, e.g., KLK4, KLK5, KLK6, KLK7, KLK8, KLK10, KLK11, KLK13 or KLK14.
Metallo proteinases, e.g., Meprin, Neprilysin, PSMA or BMP-1,
MMPs, e.g., MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, MMP27.
Serine proteases, e.g., activated protein C, Cathepsin A, Cathepsin G, Chymase, coagulation factor proteases (e.g., FVIIa, FIXa, FXa, FXIa, FXIIa), Elastase, Granzyme B, Guanidinobenzoatase, HtrA1, Human Neutrophil Elastase, Lactoferrin, Marapsin, NS3/4A, PACE4, Plasmin, PSA, tPA, Thrombin, Tryptase or uPA
Type II Transmembrane Serine Proteases (TTSPs), e.g., DESC1, DPP-4, FAP, Hepsin, Matriptase-2, MT-SP1/Matriptase, TMPRSS2, TMPRSS3, TMPRSS4

For example, suitable Substrate Recognition Sequences that can be included binder-drug conjugate, i.e., SRS is peptide moiety selected from the group consisting of: TGRGPSWV, SARGPSRW, TARGPSFK, LSGRSDNH, GGWHTGRN, HTGRSGAL, PLTGRSGG, AARGPAIH, RGPAFNPM, SSRGPAYL, RGPATPIM, RGPA, GGQPSGMWGW, FPRPLGITGL, VHMPLGFLGP, SPLTGRSG, SAGFSLPA, LAPLGLQRR, SGGPLGVR, PLGL, GPRSFGL, and GPRSFG.

In some embodiments, the Substrate Recognition Sequence is a substrate for an MMP, such as a sequence selected from the group consisting of ISSGLLSS, QNQALRMA, AQNLLGMV, STFPFGMF, PVGYTSSL, DWLYWPGI, MIAPVAYR, RPSPMWAY, WATPRPMR, FRLLDWQW, LKAAPRWA, GPSHLVLT, LPGGLSPW, MGLFSEAG, SPLPLRVP, RMHLRSLG, LAAPLGLL, AVGLLAPP, LLAPSHRA, PAGLWLDP, and ISSGLSS.

In some embodiments, the Substrate Recognition Sequence is a substrate for an MMP, such as a sequence selected from the group consisting of ISSGLSS, QNQALRMA, AQNLLGMV, STFPFGMF, PVGYTSSL, DWLYWPGI, ISSGLLSS, LKAAPRWA, GPSHLVLT, LPGGLSPW, MGLFSEAG, SPLPLRVP, RMHLRSLG, LAAPLGLL, AVGLLAPP, LLAPSHRA, and PAGLWLDP.

In some embodiments, the Substrate Recognition Sequence is a substrate for thrombin, such as GPRSFGL or GPRSFG.

In certain embodiments of the subject binder-drug conjugate, the substrate recognition sequence is cleaved by fiboblast activating protein alpha (FAPD) and is represented by

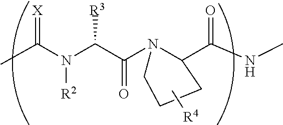

wherein
R² represents H or a (C₁-C₆) alkyl, and preferably is H;
R³ represents H or a (C₁-C₆) alkyl, preferably is methyl, ethyl, propyl, or isopropyl, and more preferably methyl;
R⁴ is absent or represents a (C₁-C₆) alkyl, —OH, —NH₂, or halogen; X represents O or S; and
—NH— represents an amine that is part of L² if L² is a self immolative linker or part of DM if L² is a bond.

In certain embodiments, R² is H, R³ is methyl, R⁴ is absent and X is O.

b. Self Immolative

The binder-drug conjugates of the invention can employ a heterocyclic self-immolative moiety covalently linked to the drug moiety and the cleavable Substrate Recognition Sequence moiety. A self-immolative moiety may be defined as a bifunctional chemical group which is capable of covalently linking together two spaced chemical moieties into a normally stable molecule, releasing one of said spaced chemical moieties from the molecule by means of enzymatic cleavage; and following said enzymatic cleavage, spontaneously cleaving from the remainder of the bifunctional chemical group to release the other of said spaced chemical moieties. In accordance with the present invention, the self-immolative moiety is covalently linked at one of its ends, directly or indirectly through a Spacer unit, to the ligand by an amide bond and covalently linked at its other end to a chemical reactive site (functional group) pending from the drug. The derivatization of the drug moiety with the self-immolative moiety may render the drug less pharmacologically active (e.g. less toxic) or not active at all until the drug is cleaved.

The binder-drug conjugate is generally stable in circulation, or at least that should be the case in the absence of an enzyme capable of cleaving the amide bond between the substrate recognition sequence and the self-immolative moiety. However, upon exposure of the binder-drug conjugate to a suitable enzyme, the amide bond is cleaved initiating a spontaneous self-immolative reaction resulting in the cleavage of the bond covalently linking the self-immolative moiety to the drug, to thereby effect release of the free drug moiety in its underivatized or pharmacologically active form.

The self-immolative moiety in conjugates of the invention either incorporate one or more heteroatoms and thereby provides improved solubility, improves the rate of cleavage and decreases propensity for aggregation of the conjugate. These improvements of the heterocyclic self-immolative linker constructs of the present invention over non-heterocyclic, PAB-type linkers may result in surprising and unexpected biological properties such as increased efficacy, decreased toxicity, and more desirable pharmacokinetics.

In certain embodiments, L² is a benzyloxycarbonyl group.

In certain embodiments, L² is

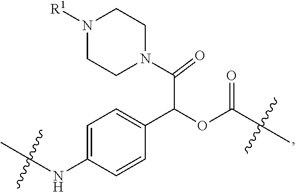

wherein R¹ is hydrogen, unsubstituted or substituted C₁₋₃ alkyl, or unsubstituted or substituted heterocyclyl. In certain embodiments, R¹ is hydrogen. In certain instances, R¹ is methyl.

In certain embodiments, L² is selected from

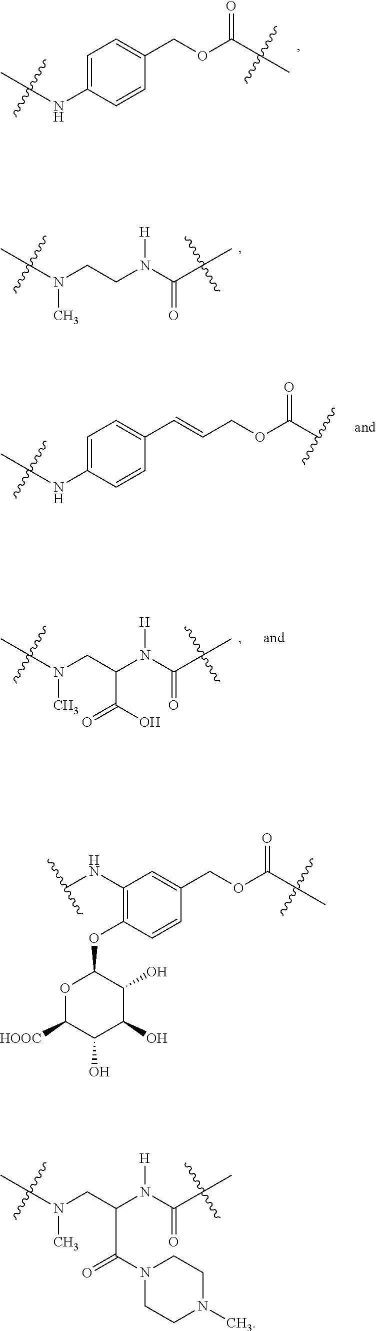

In certain embodiments, the self-immolative moiety L₂ is selected from

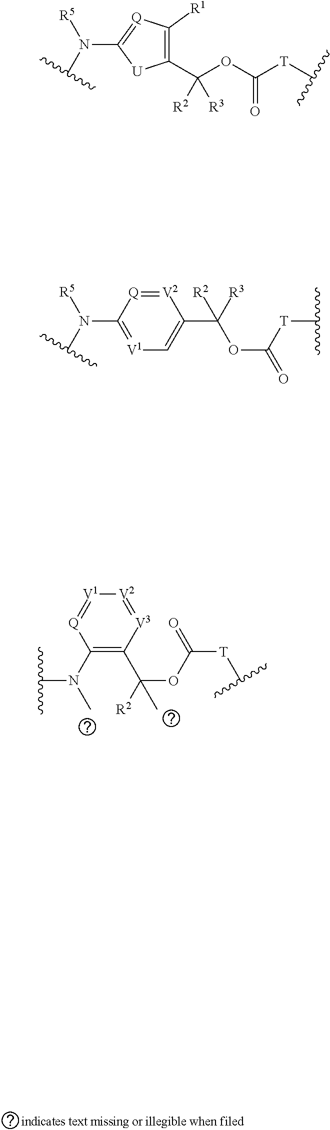

wherein
U is O, S or NR⁶;
Q is CR⁴ or N;
V¹, V² and V³ are independently CR⁴ or N provided that for formula II and III at least one of Q, V¹ and V² is N;
T is NH, NR⁶, O or S pending from said drug moiety;
R¹, R², R³ and R⁴ are independently selected from H, F, Cl, Br, I, OH, —N(R⁵)₂, —N(R⁵)₃⁻, C₁-C₈ alkyihalide, carboxylate, sulfate, sulfamate, sulfonate, —SO₂R⁵, —S(═O)R⁵, —SR⁵, —SO₂N(R⁵)₂, —C(═O)R⁵, —CO₂R⁵, —C(═O)N(R⁵)₂, —CN, —N₃, —NO₂, C₁-C₈alkoxy, C₁-C₈ halosubstituted alkyl, polyethyleneoxy, phosphonate, phosphate, C₁-C₈ alkyl, C₁-C₈ substituted alkyl, C₂-C₈ alkenyl, C₂—C₈ substituted alkenyl, C₂-C₈ alkynyl, C₂-C₈ substituted alkynyl, C₆-C₂₀ aryl, C₆-C₂₀ substituted aryl, C₁-C₂₀ heterocycle, and C₁-C₂₀ substituted heterocycle; or when taken together, R² and R³ form a carbonyl (═O), or spiro carbocyclic ring of 3 to 7 carbon atoms; and
R⁵ and R⁶ are independently selected from H. C₁-C₈ alkyl, C₁-C₈ substituted alkyl, C₂-C₈ alkenyl, C₂-C₈ substituted alkenyl, C₂-C₈alkynyl, C₂-C₈ substituted alkynyl, C₆-C₂₀ aryl, C₆-C₂₀ substituted aryl, C₁-C₂₀ heterocycle, and C₁-C₂₀ substituted heterocycle,
where C₁-C₈ substituted alkyl, C₂-C₈ substituted alkenyl, C₂-C₈ substituted alkynyl, C₆-C₂₀ substituted aryl, and C₂-C₂₀ substituted heterocycle are independently substituted with one or more substituents selected from F, Cl, Br, I, OH, —N(R⁵)₂, —N(R⁵)₃⁺. C₁-C₈alkylhalide, carboxylate, sulfate, sulfamate, sulfonate, C₁-C₈alkylsulfonate, C₁-C₈alkylamino, 4-dialkylaminopyridinium, C₁-C₈alkylhydroxyl, C₁-C₈alkylthiol, —SO₂R⁵, —S(═O)R⁵, —SR, —SO₂N(R⁵)₂, —C(═O)R⁵, —CO₂R⁵, —C(═O)N(R⁵)₂, —CN, —N₃, —NO₂. C₁-C₈ alkoxy, C₁-C₈ trifluoroalkyl, C₁-C₈ alkyl, C₃-C₁₂ carbocycle, C₆-C₂₀ aryl, C₂-C₂₀ heterocycle, polyethyleneoxy, phosphonate, and phosphate.

It will be understood that when T is NH, it is derived from a primary amine (—NH₂) pending from the drug moiety (prior to coupling to the self-immolative moiety) and when T is N, it is derived from a secondary amine (—NH—) from the drug moiety (prior to coupling to the self-immolative moiety). Similarly, when T is O or S, it is derived from a hydroxyl (—OH) or sulfhydryl (—SH) group respectively pending from the drug moiety prior to coupling to the self-immolative moiety.

In certain embodiments, the self-immolative linker L² is —NH—(CH₂)₄—C(═O)— or —NH—(CH₂)₃—C(═O)—.

In certain embodiments, the self-immolative linker L² is p-aminobenzyloxycarbonyl (PABC).

In certain embodiments, the self-immolative linker L² is 2,4-bis(hydroxymethyl)aniline.

Other exemplary self-immolative linkers that are readily adapted for use in the present invention are taught in, for example, U.S. Pat. No. 7,754,681, WO2012074693A1, U.S. Pat. No. 9,089,614, EP1732607A2, WO2015038426A1 (all of which are incorporated by reference), Walther et al. “Prodrugs in medicinal chemistry and enzyme prodrug therapies” Adv Drug Deliv Rev. 2017 Sep. 1; 118:65-77, and Tranoy-Opalinski et al. “Design of self-immolative linkers for tumour-activated prodrug therapy”, Anticancer Agents Med Chem. 2008 August; 8(6):618-37; the teachings of each of which are incorporated by reference herein.

c. Drug Moiety

A wide range of drug entities can be used as the drug moiety, DM, of the subject binder drug conjugates.

In certain embodiments, the free drug moiety is an immunomodulator—which includes drug moieties acting as immune activating agents and/or inducers of an innate immunity pathway response. In certain embodiments, the free drug moiety induces the production of IFN-α. In certain embodiments, the free drug moiety induces the production of proinflammatory cytokines. In certain embodiments, the free drug moiety induces the production of IL-1β. In certain embodiments, the free drug moiety induces the production of IL-18.

In certain embodiments, the free drug moiety promotes the expansion and survival of effector cells including NK, γδ T, and CD8+ T cells.

In certain embodiments, the free drug moiety induces macrophage pyroptosis.

(i) Exemplary Immuno-DASH Inhibitors

In certain embodiments, the immuno-DASH inhibitor for use in the method of the present invention are represented by the general formula;

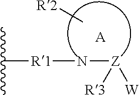

wherein
A represents a 4-8 membered heterocycle including the N and the Ca carbon;
Z represents C or N;
W represents —CN, CH═NR5,

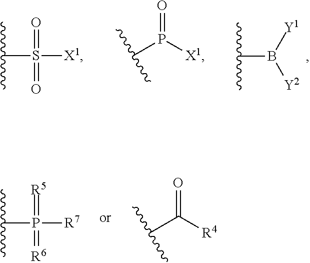

R′1 represents a C-terminally linked amino acid residue or amino acid analog, or a C-terminally linked peptide or peptide analog, the amine terminus of which forms a covalent with L1, or if L1 is a bond then with the substrate recognition sequence;
R′2 is absent or represents one or more substitutions to the ring A, each of which can independently be a halogen, a lower alkyl, a lower alkenyl, a lower alkynyl, a carbonyl (such as a carboxyl, an ester, a formate, or a ketone), a thiocarbonyl (such as a thioester, a thioacetate, or a thioformate), an amino, an acylamino, an amido, a cyano, a nitro, an azido, a sulfate, a sulfonate, a sulfonamido, —(CH₂)ₘ—R7, —(CH₂)ₘ—OH, —(CH₂)ₘ—O-lower alkyl, —(CH₂)ₘ—O-lower alkenyl, —(CH2)ₙ—O—(CH₂)ₘ—R7, —(CH₂)ₘ—SH, —(CH₂)ₘ—S-lower alkyl, —(CH₂)ₘ—S-lower alkenyl, —(CH2)ₙ—S—(CH₂)ₘ—R7;
if X is N, R′3 represents hydrogen, if X is C, R′3 represents hydrogen or a halogen, a lower alkyl, a lower alkenyl, a lower alkynyl, a carbonyl (such as a carboxyl, an ester, a formate, or a ketone), a thiocarbonyl (such as a thioester, a thioacetate, or a thioformate), an amino, an acylamino, an amido, a cyano, a nitro, an azido, a sulfate, a sulfonate, a sulfonamido, —(CH₂)ₘ—R7, —(CH₂)ₘ—OH, —(CH₂)ₘ—O-lower alkyl, —(CH₂)ₘ—O-lower alkenyl, —(CH₂)ₙ—O—(CH₂)ₘ—R7, —(CH₂)ₘ—SH, —(CH₂)ₘ—S-lower alkyl, —(CH₂)ₘ—S-lower alkenyl, —(CH₂)ₙ—S—(CH₂)ₘ—R7;
R4 represents a hydrogen, a lower alkyl, a lower alkenyl, a lower alkynyl, —(CH₂)ₘ—R3, —(CH₂)ₙ—OH, —(CH₂)ₙ—O-lower alkyl, —(CH₂)ₙ—O-alkenyl, —(CH₂)ₙ O-alkynyl, —(CH₂)ₙ—O—(CH₂)ₘ—R7, —(CH₂)ₙ—SH, —(CH₂)ₙ S-lower alkyl, —(CH₂)ₙ—S-lower alkenyl, —(CH₂)ₙ—S-lower alkynyl, —(CH₂)ₙ—S—(CH₂)ₘ—R3, —C(O)C(O)NH₂, or —C(O)C(O)OR8;
R5 represents H, an alkyl, an alkenyl, an alkynyl, —C(X1)(X2)X3, —(CH₂)ₘ—R7, —(CH₂)ₙ—OH, —(CH₂)ₙ—O-alkyl, —(CH₂)ₙ—O-alkenyl, —(CH₂)ₙ—O-alkynyl, —(CH₂)ₙ—O—(CH₂)ₘ—R7, —(CH₂)ₙ—SH, —(CH₂)ₙ—S-alkyl, —(CH₂)ₙ—S-alkenyl, —(CH₂)ₙ—S-alkynyl, —(CH₂)ₙ—S—(CH₂)ₘ—R7, —C(O)C(O)NH₂, or —C(O)C(O)OR′7;
R6 represents hydrogen, a halogen, a alkyl, a alkenyl, a alkynyl, an aryl, —(CH₂)ₘ—R7, —(CH₂)ₘ—OH, —(CH₂)ₘ—O-lower alkyl, —(CH₂)ₘ—O-lower alkenyl, —(CH₂)ₙ O—(CH₂)ₘ—R7, —(CH₂)ₘ—SH, —(CH₂)ₘ—S-lower alkyl, —(CH₂)ₘ—S-lower alkenyl, —(CH₂)ₙ S—(CH₂)ₘ—R7,
R7 represents, for each occurrence, a substituted or unsubstituted aryl, aralkyl, cycloalkyl, cycloalkenyl, or heterocycle;
R′7 represents, for each occurrence, hydrogen, or a substituted or unsubstituted alkyl, alkenyl, aryl, aralkyl, cycloalkyl, cycloalkenyl, or heterocycle; and
Y1 and Y2 can independently or together be OH, or a group capable of being hydrolyzed to a hydroxyl group, including cyclic derivatives where Y1 and Y2 are connected via a ring having from 5 to 8 atoms in the ring structure (such as pinacol or the like),
R50 represents O or S;
R51 represents N₃, SH₂, NH₂, NO₂ or O—R′7;
R52 represents hydrogen, a lower alkyl, an amine, OR′7, or a pharmaceutically acceptable salt, or R51 and R52 taken together with the phosphorous atom to which they are attached complete a heterocyclic ring having from 5 to 8 atoms in the ring structure
X1 represents a halogen;
X2 and X3 each represent a hydrogen or a halogen
m is zero or an integer in the range of 1 to 8; and
n is an integer in the range of 1 to 8.

In preferred embodiments, the ring A is a 5, 6 or 7 membered ring, e.g., represented by the formula

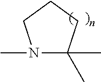

and more preferably a 5 or 6 membered ring (i.e., n is 1 or 2, though n may also be 3 or 4).

The ring may, optionally, be further substituted.

In preferred embodiments, W represents

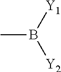

In preferred embodiments, R′1 is

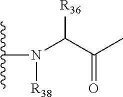

wherein R36 is a small hydrophobic group, e.g., a lower alkyl or a halogen and R38 is hydrogen, or R36 and R37 together form a 4-7 membered heterocycle including the N and the Cα carbon, as defined for A above.

In preferred embodiments, R′2 is absent, or represents a small hydrophobic group such as a lower alkyl or a halogen.

In preferred embodiments, R′3 is a hydrogen, or a small hydrophobic group such as a lower alkyl or a halogen.

In preferred embodiments, R′5 is a hydrogen, or a halogenated lower alkyl.

In preferred embodiments, X1 is a fluorine, and X2 and X3, if halogens, are fluorine.

Also deemed as equivalents are any compounds which can be hydrolytically converted into any of the aforementioned compounds including boronic acid esters and halides, and carbonyl equivalents including acetals, hemiacetals, ketals, and hemiketals, and cyclic dipeptide analogs.

In certain preferred embodiments, the subject method utilizes, as a immuno-DASH inhibitor, a boronic acid analogs of an amino acid. For example, the present invention contemplates the use of boro-prolyl derivatives in the subject method. Exemplary boronic acid derived inhibitors of the present invention are represented by the general formula:

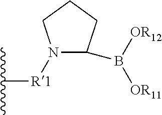

wherein
R′1 represents a C-terminally linked amino acid residue or amino acid analog, or a C-terminally linked peptide or peptide analog, the amine terminus of which forms a covalent with L1, or if L1 is a bond then with the substrate recognition sequence; and
R11 and R12 each independently represents hydrogen, a alkyl, or a pharmaceutically acceptable salt, or R11 and R12 taken together with the O—B—O atoms to which they are attached complete a heterocyclic ring having from 5 to 8 atoms in the ring structure.

In certain embodiments, the immuno-DASH inhibitor is a peptide or peptidomimetic including a prolyl group or analog thereof in the P1 specificity position, and a nonpolar (and preferably hydrophobic) amino acid in the P2 specificity position, e.g., a nonpolar amino acid such as alanine, leucine, isoleucine, valine, proline, phenylalanine, tryptophan or methionine, or an analog thereof. In other embodiments, the P2 position an amino acid with charged sidechain, such as Arginine, Lysine, Aspartic acid or Glutamic Acid. For example, the immuno-DASH inhibitor may include an Ala-Pro or Val-Pro dipeptide sequence or equivalent thereof, and be represented in the general formulas:

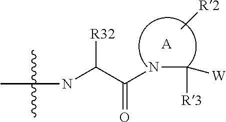

In preferred embodiments, the ring A is a 5, 6 or 7 membered ring, e.g., represented by the formula

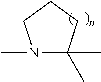

In certain preferred embodiments, R32 is a small hydrophobic group, e.g., a lower alkyl or a halogen.

In certain preferred embodiments, R32 is—lower alkyl-guanidine, -lower-alkyl-amine, lower-alkyl-C(O)OH, such as —(CH₂)ₘ—NH—C(═N)(NH₂), —(CH₂)ₘ—NH₂ or —(CH₂)ₘ—COOH, where m is 1-6, and preferably 1-3.

In preferred embodiments, R′2 is absent, or represents a small hydrophobic group such as a lower alkyl or a halogen.

In preferred embodiments, R′3 is a hydrogen, or a small hydrophobic group such as a lower alkyl or a halogen.

Another aspect of the invention relates to the immuno-DASH inhibitor represented by formula III, or a pharmaceutical salt thereof:

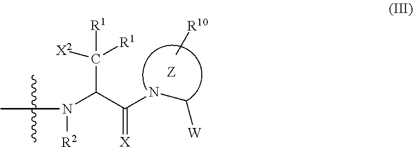

wherein
ring Z represents a 4-10 membered heterocycle including the N and the Cα carbon;
W represents —CN, CH═NR4, a functional group which reacts with an active site residue of the target, or

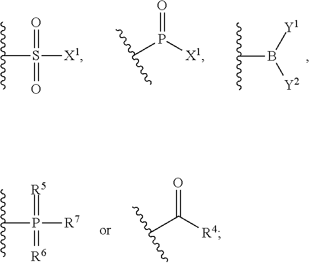

7X is O or S;
X2 is H, a halogen, or a lower alkyl;
Y1 and Y2 are independently OH, or together with the boron atom to which they are attached represent a group that is hydrolysable to a boronic acid, or together with the boron atom to which they are attached form a 5-8 membered ring that is hydrolysable to a boronic acid;
R1 represents, independently for each occurrence, a halogen, a lower alkyl, a lower alkenyl, a lower alkynyl, a carbonyl, a thiocarbonyl, an amino, an acylamino, an amido, a cyano, a nitro, an azido, a sulfate, a sulfonate, a sulfonamido, —CF₃, —(CH₂)ₘ—R3, —(CH₂)ₘOH, —(CH₂)ₘ—O-lower alkyl, —(CH₂)ₘ—O-lower alkenyl, —(CH₂)ₙ O—(CH₂)ₘ—R3, —(CH₂)ₘ—SH, —(CH₂)ₘ—S-lower alkyl, —(CH₂)ₘ—S-lower alkenyl, or —(CH₂)ₙ—S—(CH₂)ₘ—R3;
R2 represents, for each occurrence, hydrogen, lower alkyl, lower alkynyl, —(CH₂)ₘ—R3, —C(═O)-alkyl, —C(═O)-alkenyl, —C(═O)-alkynyl, or —C(═O)—(CH₂)ₘ—R3;
R3 represents, for each occurrence, hydrogen, or a substituted or unsubstituted lower alkyl, lower alkenyl, aryl, aralkyl, cycloalkyl, cycloalkenyl, or heterocycle;
R4 represents a hydrogen, a lower alkyl, a lower alkenyl, a lower alkynyl, —(CH₂)ₘ—R3, —(CH₂)ₙ OH, —(CH₂)ₙ—O-lower alkyl, —(CH₂)ₙ—O-alkenyl, —(CH₂)ₙ O-alkynyl, —(CH₂)ₙ—O—(CH₂)ₘ—R7, —(CH₂)ₙ—SH, —(CH₂)ₙ S-lower alkyl, —(CH₂)ₙ—S-lower alkenyl, —(CH₂)ₙ—S-lower alkynyl, —(CH₂)ₙ S—(CH₂)ₘ—R3, —C(O)C(O)NH₂, or —C(O)C(O)OR8;
R5 represents O or S;
R6 represents N₃, SH, NH₂, NO₂ or OR8;
R7 represents hydrogen, a lower alkyl, an amine, OR8, or a pharmaceutically acceptable salt, or R5 and R6 taken together with the phosphorous atom to which they are attached complete a heterocyclic ring having from 5 to 8 atoms in the ring structure;
R8 represents, hydrogen, a substituted or unsubstituted alkyl, alkenyl, aryl, aralkyl, cycloalkyl, cycloalkenyl or heterocyclyl;
R10 is absent or represents one to three substitutions to the ring Z to which they are appended, each of which can independently be a halogen, a lower alkyl, a lower alkenyl, a lower alkynyl, a carbonyl (such as a carboxyl, an ester, a formate, or a ketone), a thiocarbonyl (such as a thioester, a thioacetate, or a thioformate), an amino, an acylamino, an amido, a cyano, an isocyano, a thiocyanato, an isothiocyanato, a cyanato, a nitro, an azido, a sulfate, a sulfonate, a sulfonamido, lower alkyl-C(O)OH, —O-lower alkyl-C(O)OH, -guanidinyl; —(CH₂)ₘ—R7, —(CH₂)ₘ—OH, —(CH₂)ₘ—O-lower alkyl, —(CH₂)ₘ—O-lower alkenyl, —(CH₂)ₙ—O—(CH₂)ₘ—R3, —(CH₂)ₘ—SH, —(CH₂)ₘ—S-lower alkyl, —(CH₂)ₘ—S-lower alkenyl, —(CH₂)ₙ—S—(CH₂)ₘ—R3;
n is 0, 1, 2, or 3; and
m is 0, 1, 2, or 3.

Another aspect of the invention relates to the immuno-DASH inhibitor represented by formula IV, or a pharmaceutical salt thereof:

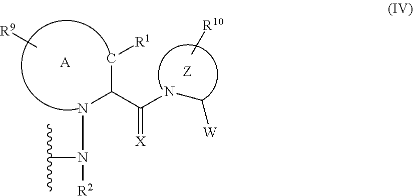

wherein
ring A represents a 3-10 membered ring structure including the N;
ring Z represents a 4-10 membered heterocycle including the N and the Cα carbon;
W represents —CN, —CH═NR4, a functional group which reacts with an active site residue of the target, or

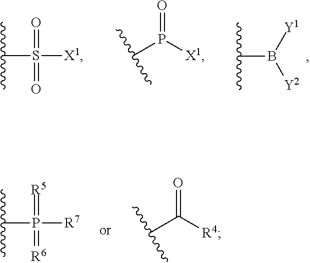

### X is O or S;

X1 represents a halogen;
Y1 and Y2 are independently OH, or together with the boron atom to which they are attached represent a group that is hydrolysable to a boronic acid, or together with the boron atom to which they are attached form a 5-8 membered ring that is hydrolysable to a boronic acid;
R1 represents a halogen, a lower alkyl, a lower alkenyl, a lower alkynyl, a carbonyl, a thiocarbonyl, an amino, an acylamino, an amido, a cyano, a nitro, an azido, a sulfate, a sulfonate, a sulfonamido, —CF3, —(CH2)m-R3, —(CH2)mOH, —(CH₂)m-O-lower alkyl, —(CH2)m-O-lower alkenyl, —(CH2)n—O—(CH2)m-R3, —(CH2)m-SH, —(CH2)m-S-lower alkyl, —(CH2)m-S-lower alkenyl, or (CH2)n-S—(CH2)m-R3;
R2 represents, for each occurrence, hydrogen, lower alkyl, lower alkynyl, —(CH₂)m-R3, —C(═O)-alkyl, —C(═O)-alkenyl, —C(═O)-alkynyl, or —C(═O)—(CH₂)m-R3;
R3 represents, for each occurrence, hydrogen, or a substituted or unsubstituted lower alkyl, lower alkenyl, aryl, aralkyl, cycloalkyl, cycloalkenyl, or heterocycle;
R4 represents a hydrogen, a lower alkyl, a lower alkenyl, a lower alkynyl, —(CH2)m-R3, —(CH2)n—OH, —(CH2)n—O-lower alkyl, —(CH2)n—O-alkenyl, —(CH2)n—O-alkynyl, —(CH2)n—O—(CH2)m-R7, —(CH2)n-SH, —(CH2)n-S-lower alkyl, —(CH2)n-S-lower alkenyl, —(CH2)n-S-lower alkynyl, —(CH2)n-S—(CH2)m-R3, —C(O)C(O)NH2, or —C(O)C(O)OR8;
R5 represents O or S;
R6 represents N₃, SH, NH₂, NO₂ or OR8;
R7 represents hydrogen, a lower alkyl, an amine, OR8, or a pharmaceutically acceptable salt, or R5 and R6 taken together with the phosphorous atom to which they are attached complete a heterocyclic ring having from 5 to 8 atoms in the ring structure;
R8 represents, hydrogen, a substituted or unsubstituted alkyl, alkenyl, aryl, aralkyl, cycloalkyl, cycloalkenyl or heterocyclyl;
R9 and R10, each independently, are absent or represents one to three substitutions to the ring A or to the ring Z to which they are appended, each of which can independently be a halogen, a lower alkyl, a lower alkenyl, a lower alkynyl, a carbonyl (such as a carboxyl, an ester, a formate, or a ketone), a thiocarbonyl (such as a thioester, a thioacetate, or a thioformate), an amino, an acylamino, an amido, a cyano, an isocyano, a thiocyanato, an isothiocyanato, a cyanato, a nitro, an azido, a sulfate, a sulfonate, a sulfonamido, —(CH2)m-R7, —(CH2)m-OH, —(CH2)m-O-lower alkyl, —(CH2)m-O-lower alkenyl, —(CH2)n—O—(CH2)m-R3, —(CH2)m-SH, —(CH2)m-S-lower alkyl, —(CH2)m S-lower alkenyl, —(CH2)n-S—(CH2)m-R3;
n is 0, 1, 2, or 3; and
m is 0, 1, 2, or 3.

In certain preferred embodiments, the immuno-DASH inhibitor is a boronic acid inhibitor of the DASH enzymes DPP8 and DPP9 (and optionally also DPP-4 and/or FAP).

In certain preferred embodiments, the immuno-DASH inhibitor is a dipeptide boronic acid inhibitor of the DASH enzymes DPP8 and DPP9 (and optionally also DPP-4 and/or FAP). In certain preferred embodiments, the immuno-DASH inhibitor the dipeptide boronic acid has a proline or proline analog in the P1 position. The subject immuno-DASH inhibitors can mediate tumor regression by immune-mediated mechanisms. The subject immuno-DASH inhibitors induce macrophage pyroptosis, and directly or indirectly have such activities as immunogenic modulation, sensitize tumor cells to antigen-specific CTL killing, alter immune-cell subsets and function, accelerate T cell priming via modulation of dendritic cell trafficking, and invoke a general T-cell mediated antitumor activity.

In certain embodiments, the subject combination of immuno-DASH inhibitor and PD-1 inhibitor can be administered as part of a therapy involving one or more other chemotherapeutic agents, immuno-oncology agents or radiation. It can also be used a part of therapy including tumor vaccines, adoptive cell therapy, gene therapy, oncolytic viral therapies and the like.

In certain embodiments, the immuno-DASH inhibitor of the present methods is represented by formula I, or a pharmaceutical salt thereof.

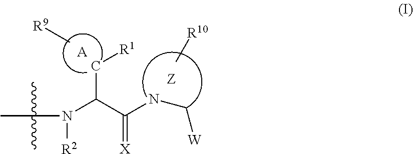

wherein
ring A represents a 3-10 membered ring structure;
ring Z represents a 4-10 membered heterocycle including the N and the Cα carbon;
W represents —CN, —CH═NR4, a functional group which reacts with an active site residue of the target, or

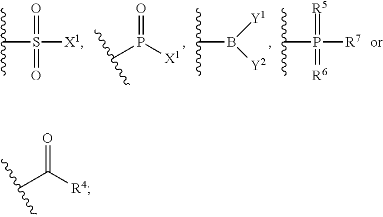

### X is O or S;

X1 represents a halogen;
Y1 and Y2 are independently OH, or together with the boron atom to which they are attached represent a group that is hydrolysable to a boronic acid, or together with the boron atom to which they are attached form a 5-8 membered ring that is hydrolysable to a boronic acid;
R1 represents a halogen, a lower alkyl, a lower alkenyl, a lower alkynyl, a carbonyl, a thiocarbonyl, an amino, an acylamino, an amido, a cyano, a nitro, an azido, a sulfate, a sulfonate, a sulfonamido, —CF3, —(CH2)m-R3, —(CH2)mOH, —(CH2)m-O-lower alkyl, —(CH2)m-O-lower alkenyl, —(CH2)n—O—(CH2)m-R3, —(CH2)m-SH, —(CH2)m-S-lower alkyl, —(CH2)m-S-lower alkenyl, or —(CH2)n-S—(CH2)m-R3;
R2 represents, for each occurrence, hydrogen, lower alkyl, lower alkynyl, —(CH2)m-R3, C(═O)-alkyl, C(═O)-alkenyl, C(═O)-alkynyl, or C(═O)—(CH2)m-R3;
R3 represents, for each occurrence, hydrogen, or a substituted or unsubstituted lower alkyl, lower alkenyl, aryl, aralkyl, cycloalkyl, cycloalkenyl, or heterocycle;
R4 represents a hydrogen, a lower alkyl, a lower alkenyl, a lower alkynyl, —(CH2)m-R3, —(CH2)n—OH, —(CH2)n—O-lower alkyl, —(CH2)n—O-alkenyl, —(CH2)n-O-alkynyl, —(CH2)n-O—(CH2)m-R7, —(CH2)n-SH, —(CH2)n-S-lower alkyl, —(CH2)n-S-lower alkenyl, —(CH2)n-S-lower alkynyl, —(CH2)n-S—(CH2)m-R3, —C(O)C(O)NH₂, or C(O)C(O)OR8;
R5 represents O or S;
R6 represents N₃, SH, NH₂, NO₂ or OR8;
R7 represents hydrogen, a lower alkyl, an amine, OR8, or a pharmaceutically acceptable salt, or R5 and R6 taken together with the phosphorous atom to which they are attached complete a heterocyclic ring having from 5 to 8 atoms in the ring structure;
R8 represents, hydrogen, a substituted or unsubstituted alkyl, alkenyl, aryl, aralkyl, cycloalkyl, cycloalkenyl or heterocyclyl;
R9 and R10, each independently, are absent or represents one, two, or three substitutions to the ring A or to the ring Z to which they are appended, each of which can independently be a halogen, a lower alkyl, a lower alkenyl, a lower alkynyl, a carbonyl (such as a carboxyl, an ester, a formate, or a ketone), a thiocarbonyl (such as a thioester, a thioacetate, or a thioformate), an amino, an acylamino, an amido, a cyano, an isocyano, a thiocyanato, an isothiocyanato, a cyanato, a nitro, an azido, a sulfate, a sulfonate, a sulfonamido, lower alkyl-C(O)OH, —O-lower alkyl-C(O)OH, -guanidinyl; —(CH2)m-R7, —(CH2)m-OH, —(CH2)m-O-lower alkyl, —(CH2)m-O-lower alkenyl, —(CH2)n-O—(CH2)m-R3, —(CH2)m-SH, —(CH2)m-S-lower alkyl, —(CH2)m-S-lower alkenyl, —(CH2)n-S μ(CH2)m-R3;
n is 0, 1, 2, or 3; and
m is 0, 1, 2, or 3.

In certain embodiments, the immuno-DASH inhibitor of Formula I is represented in Formula Ia, or is a pharmaceutical salt thereof:

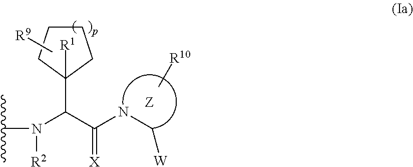

wherein X, W, Z, R¹, R², R⁹ and R¹⁰ are as defined above for Formula I, and p is 1, 2 or 3.

In certain preferred embodiments of Ia: R¹ is a lower alkyl; R⁹ is absent, or independently for each occurrence, is a lower alkyl, —OH, —NH₂, —N₃, -lower alkyl-C(O)OH, —O-lower alkyl, —O-lower alkyl-C(O)OH, -guanidinyl; X is O; each R² is hydrogen, R¹⁰ is absent, or represents a single substitution of —OH, —NH₂, —CN or —N₃; and W is —B(OH)₂ or —CN (and more preferably —B(OH)2).

In certain embodiments, the immuno-DASH inhibitor of Formula I is represented in Formula Ib, or is a pharmaceutical salt thereof:

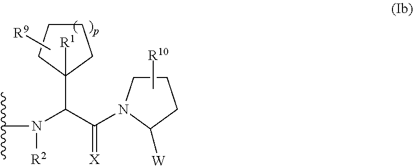

wherein X, W, R¹, R², R⁹ and R¹⁰ are as defined above for Formula I, and p is 1, 2 or 3.

In certain preferred embodiments of Ib: R¹ is a lower alkyl; R⁹ is absent, or independently for each occurrence, is a lower alkyl, —OH, —NH₂, —N₃, -lower alkyl-C(O)OH, —O-lower alkyl, —O-lower alkyl-C(O)OH, -guanidinyl; X is O; each R² is hydrogen, R¹⁰ is absent, or represents a single substitution of —OH, —NH₂, —CN or —N₃; and W is —B(OH)₂ or —CN (and more preferably —B(OH)₂).

In certain embodiments, the immuno-DASH inhibitor of Formula I is represented in Formula Ic, or is a pharmaceutical salt thereof:

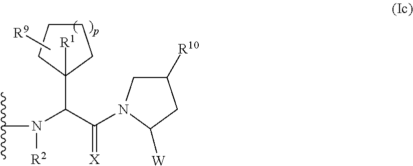

wherein X, W, R¹, R², R⁹ and R¹⁰ are as defined above for Formula I, and p is 1, 2 or 3.

In certain preferred embodiments of Ic: R¹ is a lower alkyl; R⁹ is absent, or independently for each occurrence, is a lower alkyl, —OH, —NH₂, —N₃, -lower alkyl-C(O)OH, —O-lower alkyl, —O-lower alkyl-C(O)OH, -guanidinyl; X is O; each R² is hydrogen, R¹⁰ is absent, or represents a single substitution of —OH, —NH₂, —CN or —N₃; and W is —B(OH)₂ or —CN (and more preferably —B(OH)₂).

In some embodiments, the immuno-DASH inhibitor is represented by:

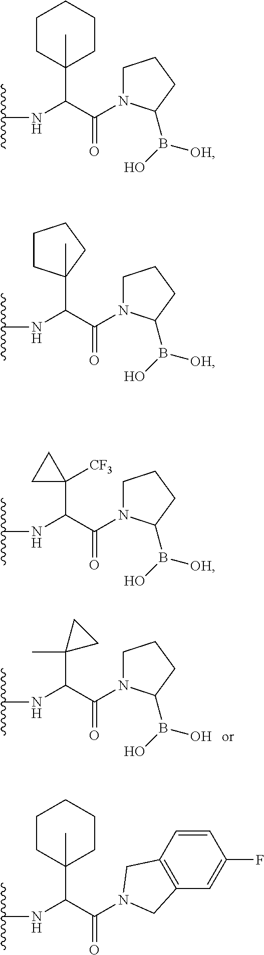

Another aspect of the invention relates to the immuno-DASH inhibitor represented by formula II, or a pharmaceutical salt thereof:

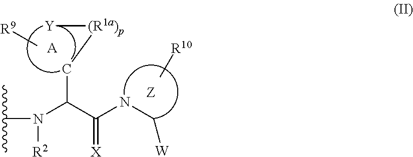

wherein
ring A, along with each occurrence of R^{1a}, represents a 7-12 membered polycyclic ring structure;
ring Z represents a 4-10 membered heterocycle including the N and the Cα carbon;
W represents —CN, CH═NR⁴, a functional group which reacts with an active site residue of the target, or

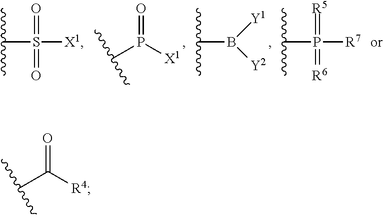

### X is O or S;

X¹ represents a halogen;

### Y is C or N;

Y¹ and Y² are independently OH, or together with the boron atom to which they are attached represent a group that is hydrolysable to a boronic acid, or together with the boron atom to which they are attached form a 5-8 membered ring that is hydrolysable to a boronic acid;
R1a represents a lower alkyl, —(CH₂)m-, —(CH₂)m-O—(CH₂)m-; —(CH₂)m-N—(CH₂)m-; or —(CH₂)m-S—(CH₂)m;
R² represents, for each occurrence, hydrogen, lower alkyl, lower alkynyl, —(CH₂)m-R³, —C(═O)-alkyl, —C(═O)-alkenyl, —C(═O)-alkynyl, or —C(═O)—(CH₂)ₘ—R³;
R³ represents, for each occurrence, hydrogen, or a substituted or unsubstituted lower alkyl, lower alkenyl, aryl, aralkyl, cycloalkyl, cycloalkenyl, or heterocycle;
R⁴ represents a hydrogen, a lower alkyl, a lower alkenyl, a lower alkynyl, —(CH₂)ₘ—R³, —(CH₂)ₙ OH, —(CH₂)ₙ—O-lower alkyl, —(CH₂)ₙ—O-alkenyl, —(CH₂)ₙ—O-alkynyl, —(CH₂)ₙ O (CH₂)ₘ—R⁷, —(CH₂)ₙ SH, —(CH₂)ₙ S-lower alkyl, —(CH₂)ₙ S-lower alkenyl, —(CH₂)ₙ—S-lower alkynyl, —(CH₂)ₙ—S—(CH₂)ₘ—R³, —C(O)C(O)NH₂, or C(O)C(O)OR⁸;
R⁵ represents O or S;
R⁶ represents N₃, SH, NH₂, NO₂ or OR⁸;
R⁷ represents hydrogen, a lower alkyl, an amine, OR⁸, or a pharmaceutically acceptable salt, or R⁵ and R⁶ taken together with the phosphorous atom to which they are attached complete a heterocyclic ring having from 5 to 8 atoms in the ring structure;
R⁸ represents, hydrogen, a substituted or unsubstituted alkyl, alkenyl, aryl, aralkyl, cycloalkyl, cycloalkenyl or heterocyclyl;
R⁹ and R¹⁰, each independently, are absent or represents one, two, or three substitutions to the ring A or to the ring Z to which they are appended, each of which can independently be a halogen, a lower alkyl, a lower alkenyl, a lower alkynyl, a carbonyl (such as a carboxyl, an ester, a formate, or a ketone), a thiocarbonyl (such as a thioester, a thioacetate, or a thioformate), an amino, an acylamino, an amido, a cyano, an isocyano, a thiocyanato, an isothiocyanato, a cyanato, a nitro, an azido, a sulfate, a sulfonate, a sulfonamido, lower alkyl-C(O)OH, —O-lower alkyl-C(O)OH, -guanidinyl; —(CH₂)ₘ—R⁷, —(CH₂)ₘ—OH, —(CH₂)ₘ—O-lower alkyl, —(CH₂)ₘ—O-lower alkenyl, —(CH₂)ₙ—O—(CH₂)ₘ—R³, —(CH₂)ₘ—SH, —(CH₂)ₘ—S-lower alkyl, —(CH₂)ₘ—S-lower alkenyl, —(CH₂)ₙ—S—(CH₂)ₘ—R³;
n is 0, 1, 2, or 3;
m is 0, 1, 2, or 3; and
p is 1, 2, or 3.

In certain embodiments, the immuno-DASH inhibitor of Formula II is represented in Formula IIa, or is a pharmaceutical salt thereof:

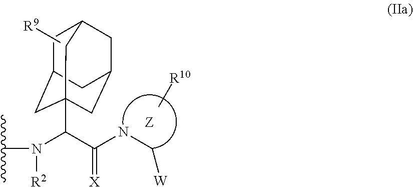

wherein X, W, Z, R², R⁹ and R¹⁰ are as defined above for Formula II.

In certain preferred embodiments of IIa: R⁹, independently for each occurrence, is a lower alkyl, —OH, —NH₂, —N₃, -lower alkyl-C(O)OH, —O-lower alkyl, —O-lower alkyl-C(O)OH, -guanidinyl; X is O; each R² is hydrogen, R¹⁰ is absent, or represents a single substitution of —OH, —NH₂, —CN or —N₃; and W is —B(OH)₂ or —CN (and more preferably —B(OH)2).

In certain embodiments, the immuno-DASH inhibitor of Formula II is represented in Formula IIb, or is a pharmaceutical salt thereof:

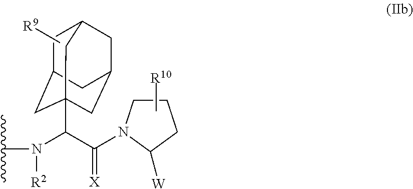

wherein X, W, R², R⁹ and R¹⁰ are as defined above for Formula II.

In certain preferred embodiments of IIb: R⁹, independently for each occurrence, is a lower alkyl, —OH, —NH₂, —N₃, -lower alkyl-C(O)OH, —O-lower alkyl, —O-lower alkyl-C(O)OH, -guanidinyl; X is O; each R² is hydrogen, R¹⁰ is absent, or represents a single substitution of —OH, —NH₂, —CN or —N₃; and W is —B(OH)₂ or —CN (and more preferably —B(OH)₂).

In certain embodiments, the immuno-DASH inhibitor of Formula II is represented in Formula IIc, or is a pharmaceutical salt thereof:

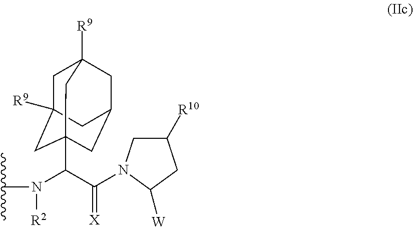

wherein X, W, R², R⁹ and R¹⁰ are as defined above for Formula II.

In certain preferred embodiments of IIc: R⁹, independently for each occurrence, is a lower alkyl, —OH, —NH₂, —N₃, -lower alkyl-C(O)OH, —O-lower alkyl, —O-lower alkyl-C(O)OH, -guanidinyl; X is O; each R² is hydrogen, R¹⁰ is absent, or represents a single substitution of —OH, —NH₂, —CN or —N₃; and W is —B(OH)₂ or —CN (and more preferably —B(OH)₂).

In certain embodiments, the immuno-DASH inhibitor of Formula II is represented in Formula IId, or is a pharmaceutical salt thereof:

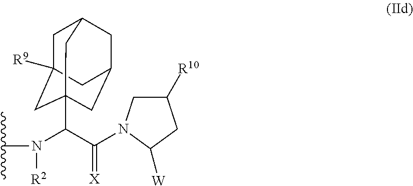

wherein X, W, R², R⁹ and R¹⁰ are as defined above for Formula II.

In certain preferred embodiments of IId: R⁹, independently for each occurrence, is a lower alkyl, —OH, —NH₂, —N₃, -lower alkyl-C(O)OH, —O-lower alkyl, —O-lower alkyl-C(O)OH, -guanidinyl; X is O; each R² is hydrogen, R¹⁰ is absent, or represents a single substitution of —OH, —NH₂, —CN or —N₃; and W is —B(OH)₂ or —CN (and more preferably —B(OH)₂).

In certain embodiments, the immuno-DASH inhibitor of Formula II is represented in Formula IIe, or is a pharmaceutical salt thereof:

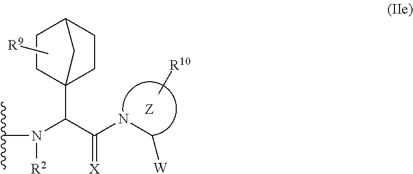

wherein X, W, Z, R², R⁹ and R¹⁰ are as defined above for Formula II.

In certain preferred embodiments of IIe: R⁹, independently for each occurrence, is a lower alkyl, —OH, —NH₂, —N₃, -lower alkyl-C(O)OH, —O-lower alkyl, —O-lower alkyl-C(O)OH, -guanidinyl; X is O; each R² is hydrogen, R¹⁰ is absent, or represents a single substitution of —OH, —NH₂, —CN or —N₃; Z is a pyrrolidine or piperidine ring (and more preferably a pyrrolidine ring); and W is —B(OH)₂ or —CN (and more preferably —B(OH)₂).

In some embodiments, the immuno-DASH inhibitor is one of the following:

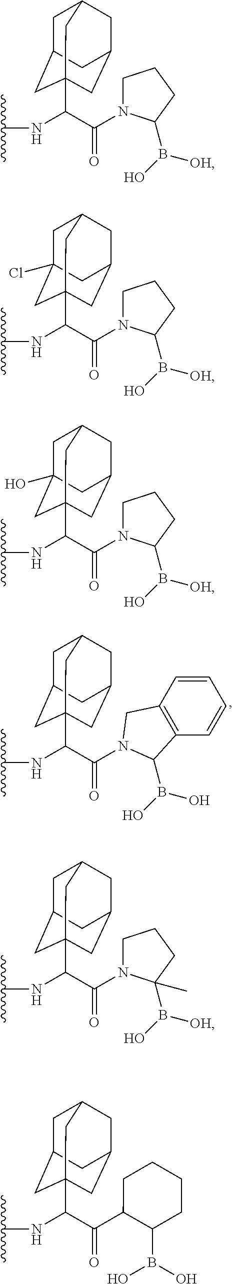 [SMILES: *NC(C(=O)N1CCCC1B(O)O)C12CC3CC(CC(C3)C1)C2 ; *NC(C(=O)N1CCCC1B(O)O)C12CC3CC(CC(Cl)(C3)C1)C2 ; *NC(C(=O)N1CCCC1B(O)O)C12CC3CC(CC(O)(C3)C1)C2 ; *NC(C(=O)N1Cc2ccccc2C1B(O)O)C12CC3CC(CC(C3)C1)C2 ; *NC(C(=O)N1CCCC1(C)B(O)O)C12CC3CC(CC(C3)C1)C2 ; *NC(C(=O)C1CCCCC1B(O)O)C12CC3CC(CC(C3)C1)C2]

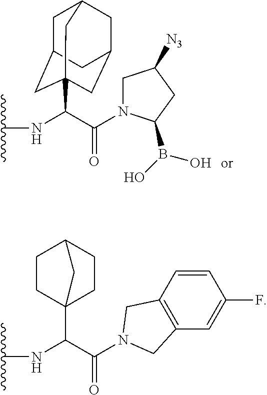 [SMILES: *N[C@H](C(=O)N1C[C@@H](N=[N+]=[N-])C[C@H]1B(O)O)C12CC3CC(CC(C3)C1)C2 ; *NC(C(=O)N1Cc2ccc(F)cc2C1)C12CCC(CC1)C2]

(ii) Exemplary STING Agonists

Non-limiting examples of STING agonists include agonists represented in the one of the general formulas

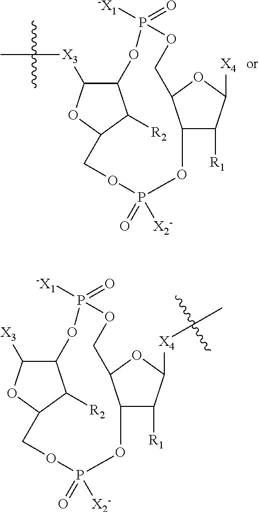

wherein
X₁ and X₂ are, independently, O or S, and preferably are the same (O,O or S,S);
X₃ and X₄ are, independently, a purine, such as a guanine or guanine analog, or a pymridine, and wherein the wavy lines indicate covalent attachment site to L1, or where L1 is a bond, to the substrate recognition sequence,
R₁ and R₂ are, independently, H, hydroxyl, a halogen (preferably F or Cl) or an optionally substituted straight chain alkyl of from 1 to 18 carbons and from 0 to 3 heteroatoms, an optionally substituted alkenyl of from 1-9 carbons, an optionally substituted alkynyl of from 1-9 carbons, or an optionally substituted aryl, wherein substitution(s), when present, may be independently selected from the group consisting of C₁₋₆, alkyl straight or branched chain, benzyl, halogen, trihalomethyl, C₁₋₆alkoxy, —NO₂, —NH₂, —OH, ═O, —COOR′ or —OR′, wherein R₁ and R₂ are not both H,
R¹ is H or lower alkyl, —CH₂OH, or —CONH₂.

In certain embodiments, the STING agonist is represented in one of the formula:

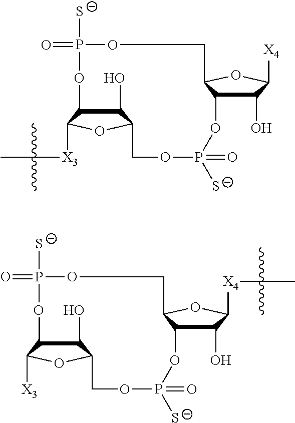

In the STING agonist structures above, X₃ and X₄ may each independently be, for example, 9-purine, 9-adenine, 9-guanine, 9-hypoxanthine, 9-xanthine, 9-uric acid, or 9-isoguanine. provided that one of X₃ or X₄ includes a functional group with which L² shares a bond if L² is a self immolative linker, or a functional group with which DM shares a bond if L² is (that) a bond.

X₃ and X₁ nay be identical or different.

In some embodiments, the STING agonists may be provided in the form of predominantly Rp,Rp or Rp,Sp stereoisomers. In some embodiments, the STING agonists may be provided in the form of predominantly Rp,Rp stereoisomers.

Exemplary STING agonists include:

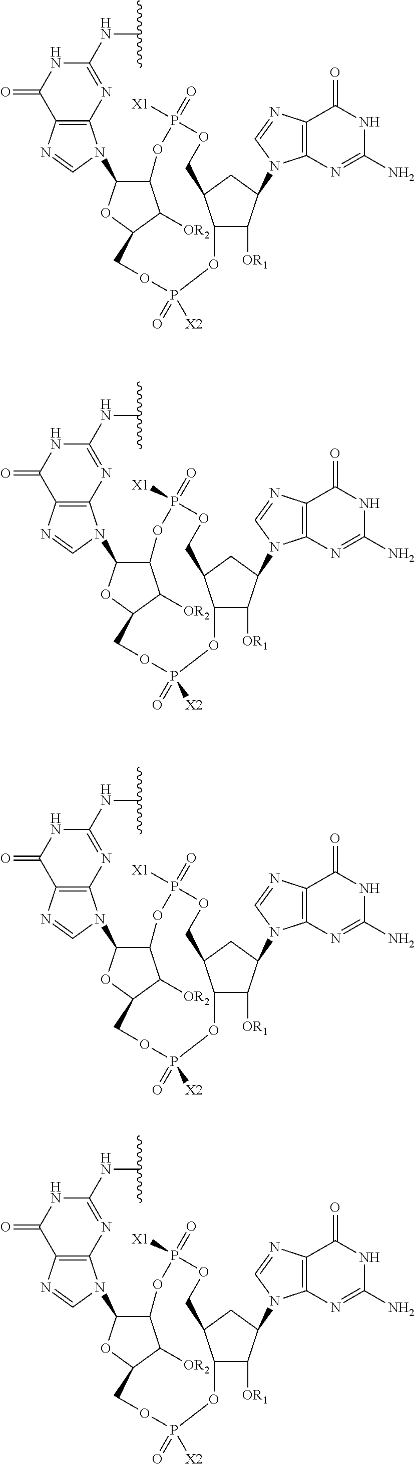

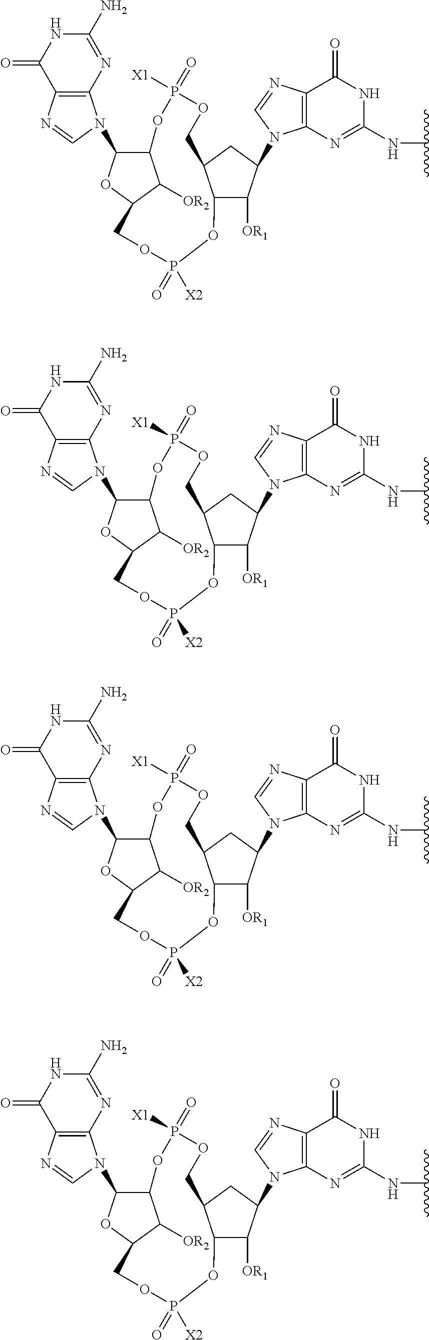

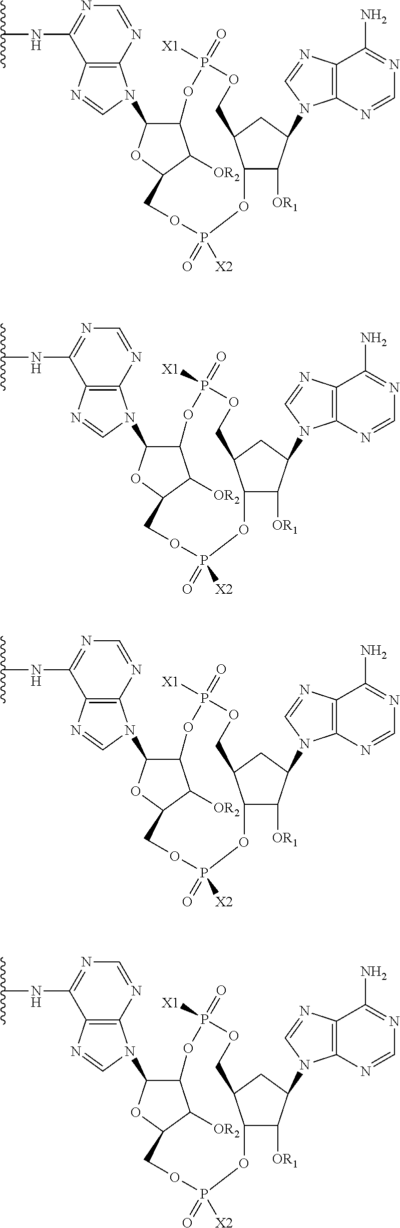

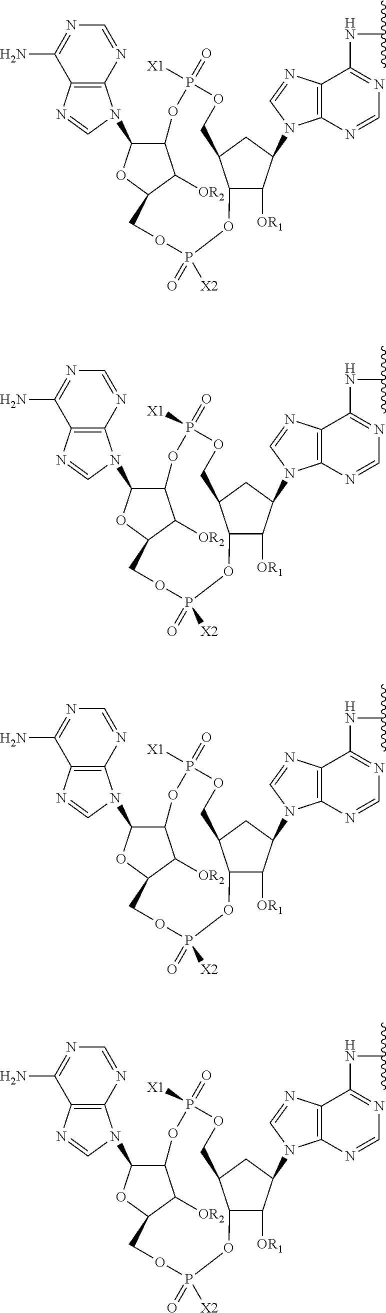

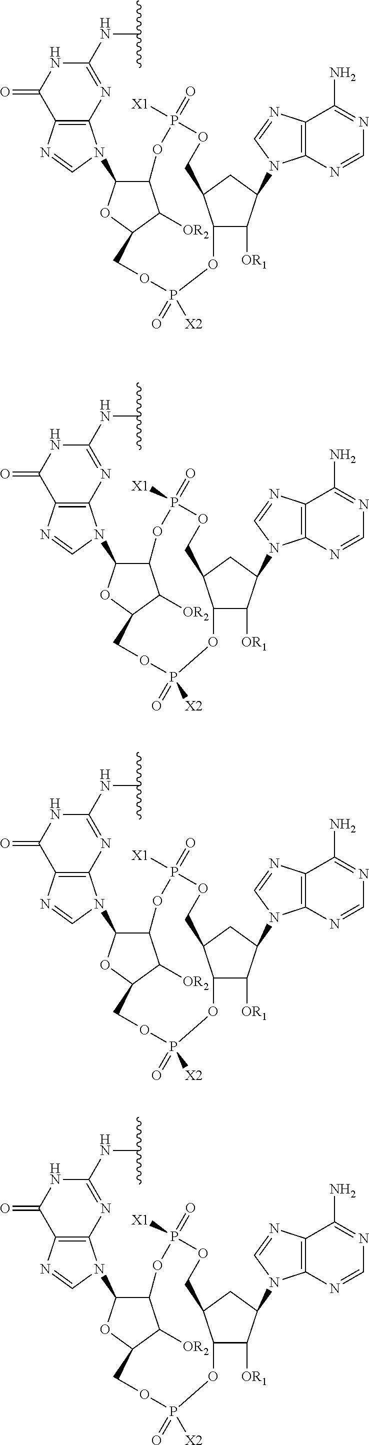

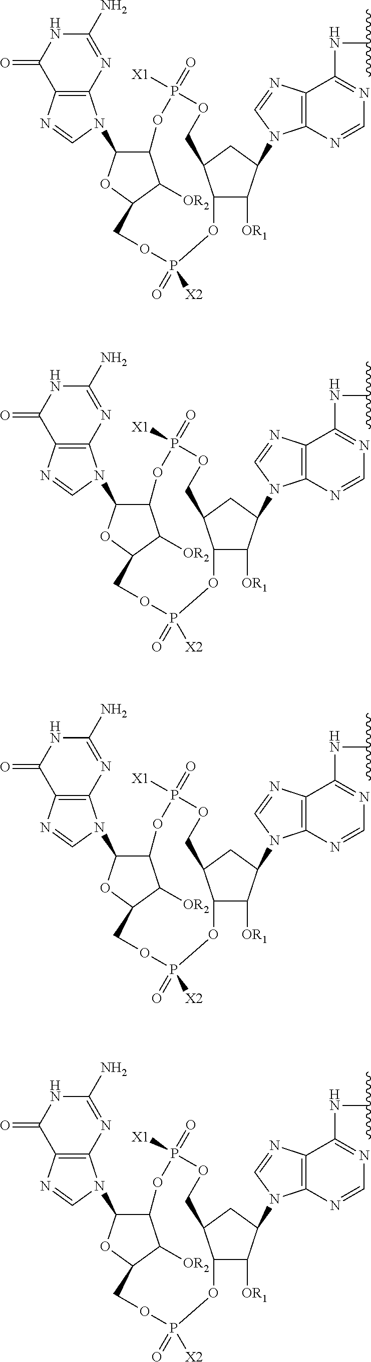

In certain embodiments, the STING agonist is represented in one of the following structures”

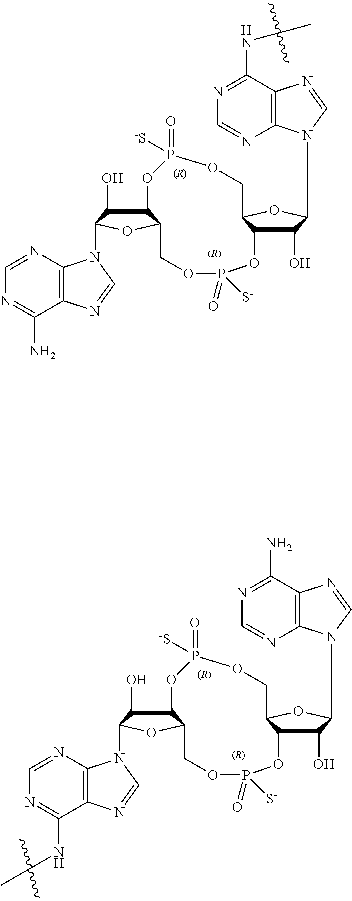

Still another STING agonist that can be used as Drug Moiety in the present binder conjugates is

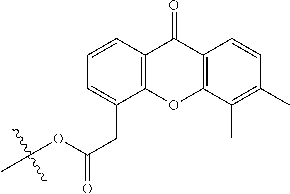 [SMILES: *OC(=O)Cc1cccc2c(=O)c3ccc(C)c(C)c3oc12]

Still other exemplary STING agonists that can be readily adapted for use as the Drug Moiety in the conjugates of the present invention are taught, merely to illustrate, in PCT Publications WO2017123669A1, and WO2015077354A1, and US Patent Publication US20150056224A1 (each of which is hereby incorporated by reference).

It will also be appreciated by those skilled in the art that, particularly with the use of a self-immolative linker, the STING agonist can be coupled to the linker though functional groups other than amines as shown above, such as through free hydroxyl groups for example.

(iii) Exemplary TLR Agonists

Examples of the “Toll-like receptor (TLR) agonist” include, but are not limited to, TLR1/2 agonists, TLR2 agonists, TLR3 agonists (e.g., PolyI:C), TLR4 agonists (e.g., S-type lipopolysaccharide, paclitaxel, lipid A, and monophosphoryl lipid A), TLR5 agonists (e.g., flagellin), TLR6/2 agonists (e.g., MALP-2), TLR7 agonist, TLR7/8 agonists (e.g., gardiquimod, imiquimod, loxoribine, and resiquimod (R848)), TLR7/9 agonists (e.g., hydroxychloroquine sulfate), TLR8 agonists (e.g., motolimod (VTX-2337)), TLR9 agonists (e.g., CpG-ODN), and TLR11 agonists (e.g., profilin).

Exemplary TRL agonists that can be used as the Drug Moiety in the binder conjugates of the present invention include S-27609, CL307, UC-IV150, imiquimod, gardiquimod, resiquimod, motolimod, VTS-1463GS-9620, GSK2245035, TMX-101, TMX-201, TMX-202, isatoribine, AZD8848, MEDI9197, 3M-051, 3M-852, 3M-052, 3M-854A, S-34240, KU34B, or CL663, or as appropriate, analogs thereof with appropriate functional groups for directed linkage and release from the substrate recognition sequence or by linkage to a self immolative linker.

Exemplary agonists of TRLs, particularly TRL7 agonists, TRL8 agonists and TRL7/8 agonists include:

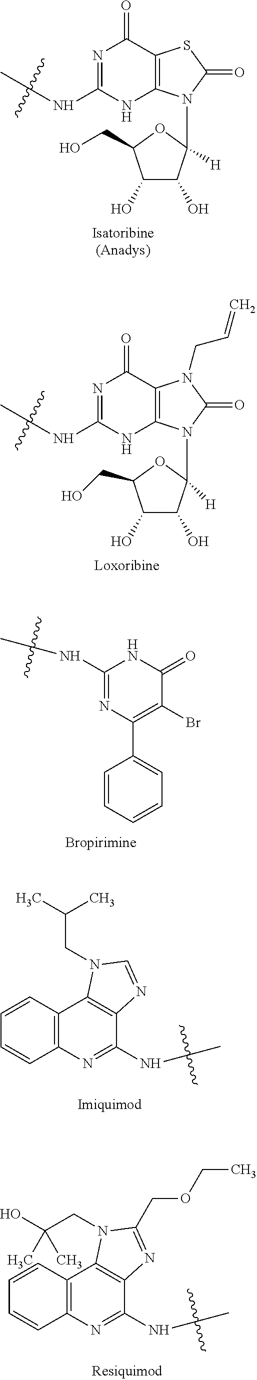 [SMILES: *Nc1nc(=O)c2sc(=O)n([C@@H]3O[C@H](CO)[C@@H](O)[C@H]3O)c2[nH]1 ; *Nc1nc(=O)c2c([nH]1)n([C@@H]1O[C@H](CO)[C@@H](O)[C@H]1O)c(=O)n2CC=C ; *Nc1nc(-c2ccccc2)c(Br)c(=O)[nH]1 ; *Nc1nc2ccccc2c2c1ncn2CC(C)C ; *Nc1nc2ccccc2c2c1nc(COCC)n2CC(C)(C)O]

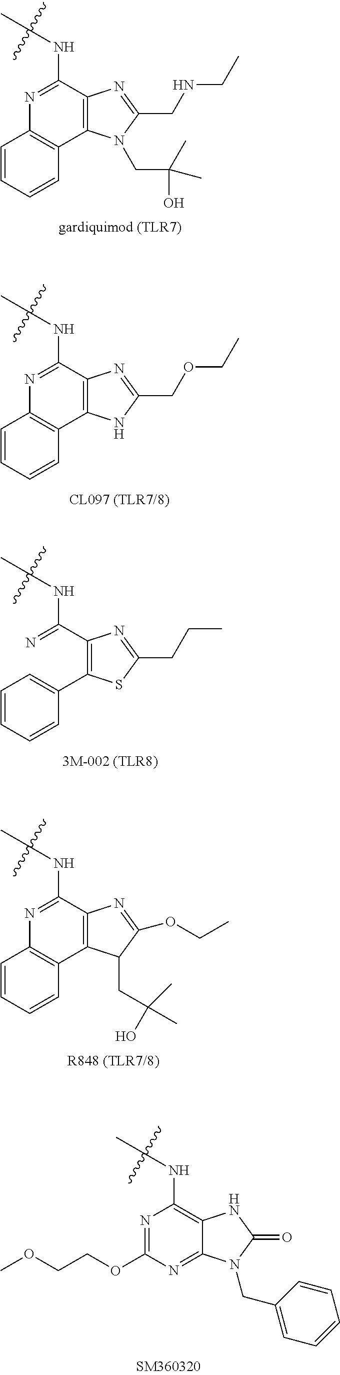

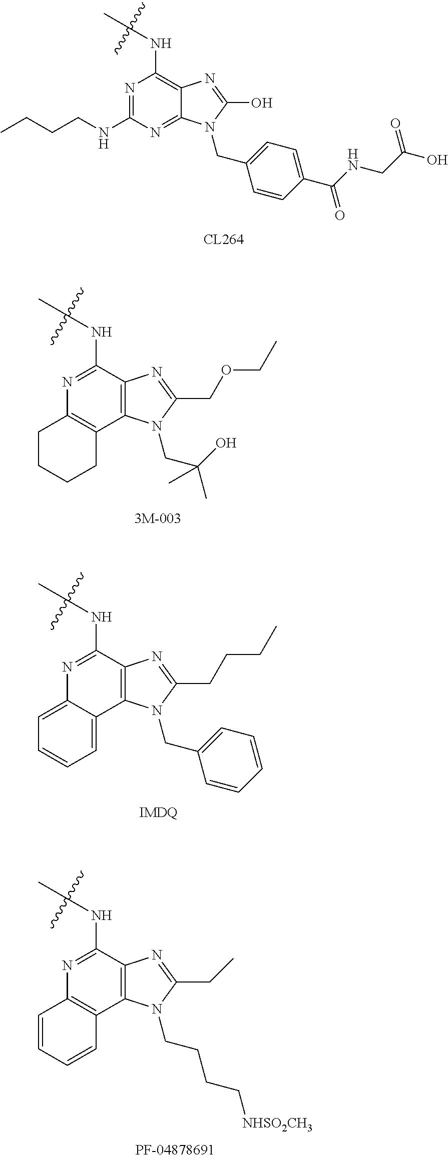

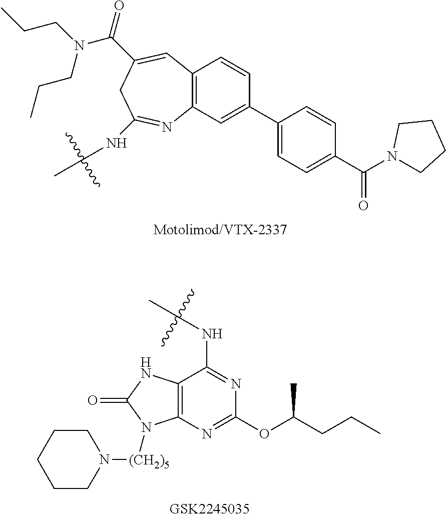

In certain embodiments, the Drug Moiety is a TRL7/8 agonist represented in the general formula

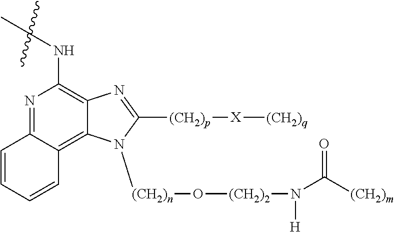

wherein
X is CH₂, O, S or N, preferably CH₂, O or N, and more preferably CH₂ or O;
n is 0 (direct bond from N to O), or an integer from 1 to 5, preferably 1 or 2;
z is an integer from 1 to 5;
m is an integer from 1 to 20, preferably from 1 to 16;
p is 0 (direct bond from ring to X), or an integer from 1 to 5, preferably 1 or 2; and
q is an integer from 1 to 5, preferably 1 or 2.

For instance, the TRL agonist is a TRL7/8 agonist such as one of

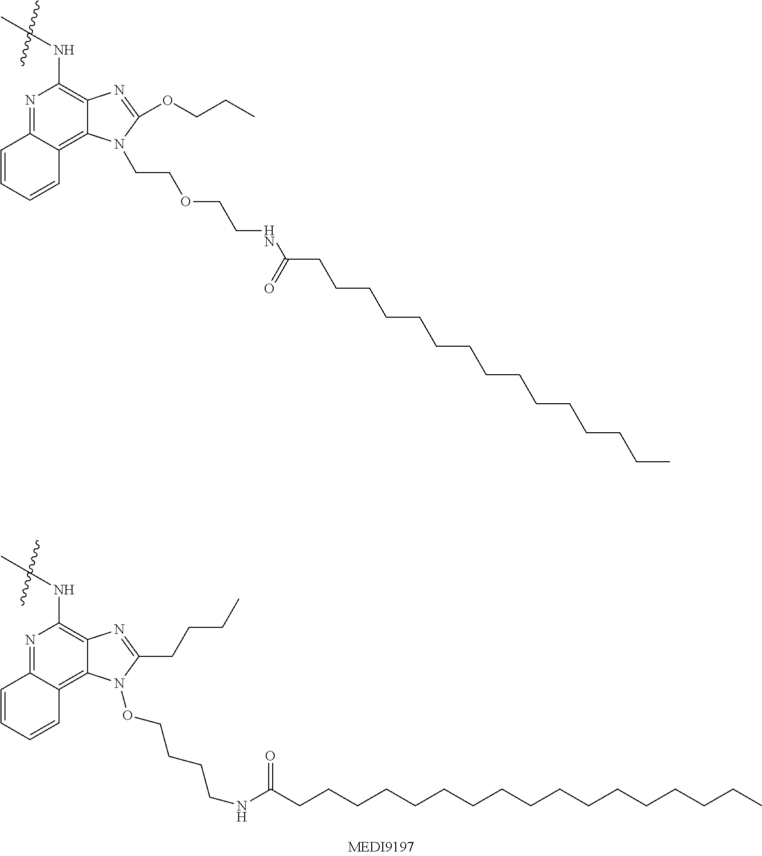 [SMILES: *Nc1nc2ccccc2c2c1nc(OCCC)n2CCOCCNC(=O)CCCCCCCCCCCCCCC ; *Nc1nc2ccccc2c2c1nc(CCCC)n2OCCCCNC(=O)CCCCCCCCCCCCCCCCC]

Publication No. WO2008135791, WO2016141092 also describe classes of imidazoquinoline compounds having immuno-modulating properties which act via TLR7.

Other exemplary TRL agonists that be readily adapted for use as the Drug Moiety of the binder conjugates of the present invention are disclosed in, for example, Yoo et al. “Structure-activity relationships in Toll-like receptor 7 agonistic 1H-imidazo[4,5-c]pyridines” Org. Biomol. Chem., 2013, 11, 6526-6545; Fletcher et al. “Masked oral prodrugs of Toll-like receptor 7 agonists: a new approach for the treatment of infectious disease”, 2006 Current opinion in investigational drugs (London, England). 7. 702-708; and Pryde et al. “The discovery of a novel prototype small molecule TLR7 agonist for the treatment of hepatitis C virus infection” Med. Chem. Commun., 2011, 2, 185-189.

It will also be appreciated by those skilled in the art that, particularly with the use of a self-immolative linker, the TRL agonists can be coupled to the linker though functional groups other than amines as shown above, such as through free hydroxyl groups for example.

(iv) Exemplary RIG-1 Agonists

The conjugate of any one of the preceding embodiments, wherein said immune-stimulatory agonist is a RIG-I agonist, wherein the RIG-I agonist is KIN700, KIN1148, KIN600, KIN500, KIN100, KIN101, KIN400, KIN2000, or SB-9200.

(v) Exemplary Anthracyclines

In certain embodiments, the drug moiety is an anthracycline or derivative thereof, preferably doxorubicin or other analogs that are able to induce immunogenic cell death of tumor cells.

Anthracyclines and analogs thereof specifically include, without limitation, doxorubicin, daunorubicin, epirubicin, idarubicin, pirarubicin, valrubicin, aclarubicin, mitoxantrone, actinomycin, bleomycin, plicamycin, and mitomycin. For example, the anthracycline moiety can be represented by the formula

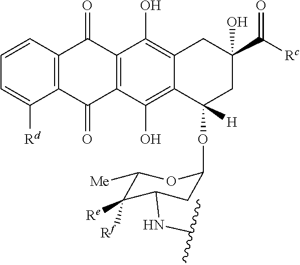

wherein,
R^{c} represents (C₁-C₆)alkyl, (C₁-C₆)hydroxyalkyl, or (C₁-C₆)alkanoyloxy(C₁-C₆)alkyl, in particular methyl, hydroxymethyl, diethoxyacetoxymethyl, or butyryloxymethyl;
R^{d} represents hydrogen, hydroxyl, or (C₁-C₆)alkoxy, in particular methoxy; one of R^{e} and R^{f} represents a hydrogen atom; and the other represents a hydrogen atom or a hydroxy or tetrahydropyrany-2-yloxy (OTHP) group.

(vi) Exemplary Proteasome Inhibitors

In certain embodiments, the drug moiety is a proteasome inhibitor. Exemplary proteasome inhibitors include

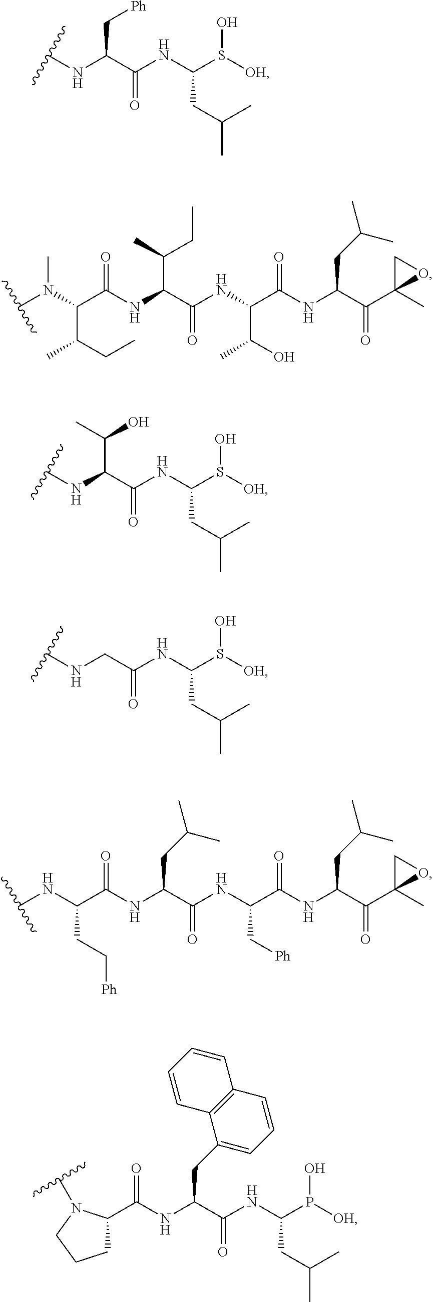

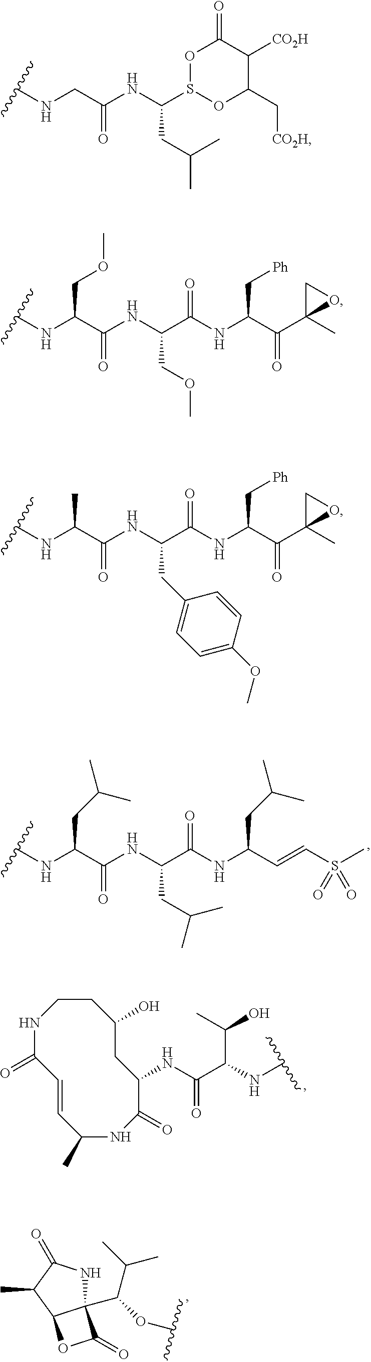

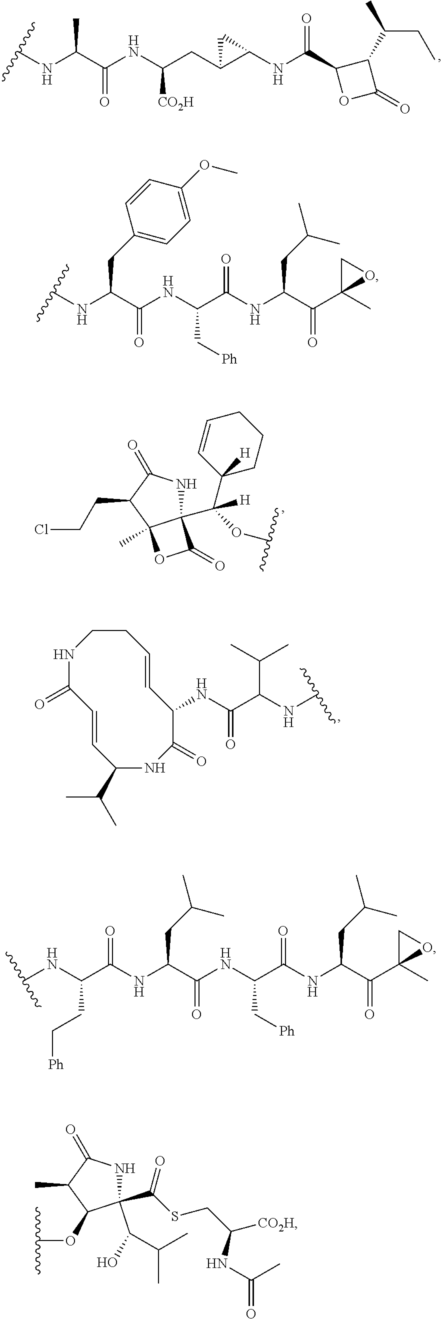

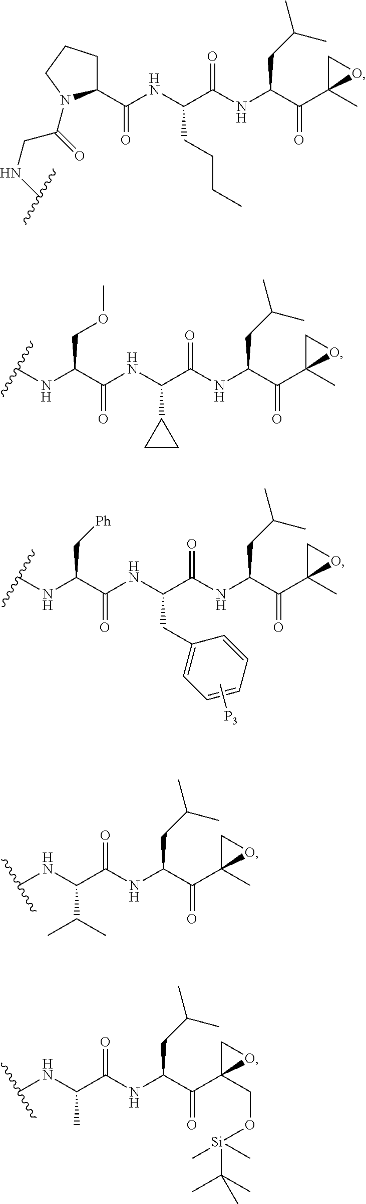

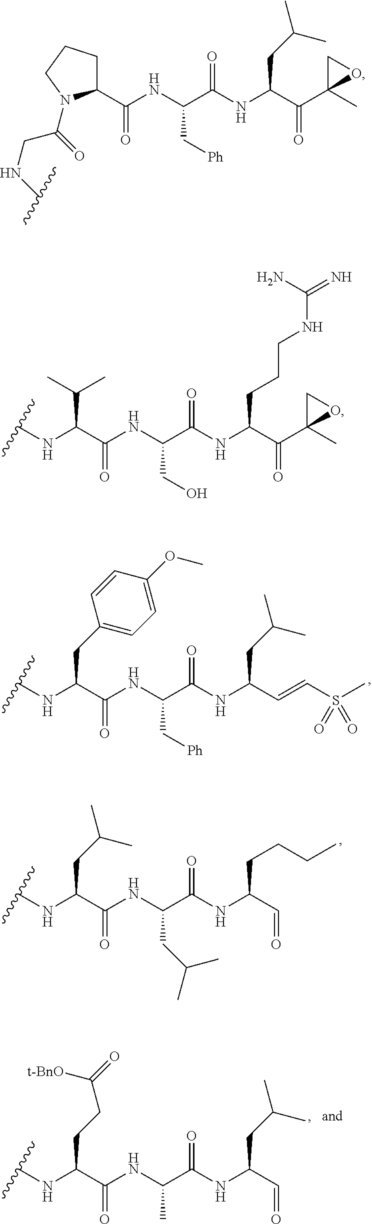

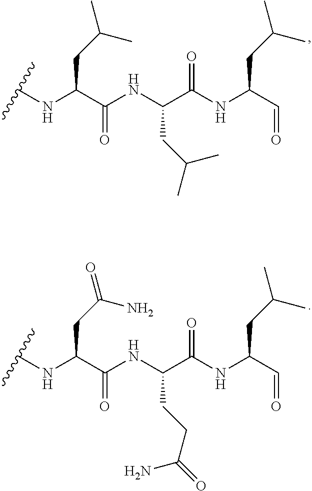 [SMILES: *N[C@@H](CC(C)C)C(=O)N[C@@H](CC(C)C)C(=O)N[C@H](C=O)CC(C)C ; *N[C@@H](CC(N)=O)C(=O)N[C@@H](CCC(N)=O)C(=O)N[C@H](C=O)CC(C)C]

d. Cell Binding Moieties

In certain embodiments, the disease tissue is a tumor. In certain embodiments, the Cell Binding Moiety of the binder-drug conjugate is selected to bind to a cell surface protein on a tumor cell. In other embodiments, the Cell Binding Moiety of the binder-drug conjugate is selected to bind to a cell surface protein on a macrophage, monocyte derived suppressor cells (MDSC), dendritic cells, fiboblasts, T-cells, NK cell, Mast Cells, Granulocytes, Eiosinophils and B-cells.

In certain embodiments, the Cell Binding Moiety of the binder-drug conjugate is selected such that when the binder-drug conjugate is bound with the surface feature on the target cell it has an internalization half-time of at least 6 hours, more preferably at least 10, 12, 14, 16, 18, 20, 24, 36, 48, 60, 75 or even 100 hours.

In certain embodiments, the Cell Binding Moiety of the binder-drug conjugate to bind a cell surface protein selectively expressed or upregulated by the target cell in the disease tissue relative to normal cells from a healthy state of the tissue. For instance, the protein is detectable on the surface of the target cells at levels 2 fold higher than normal cells from the tissue, even more preferably levels at least 5, 10, 20, 30, 40, 50, 75, 100, 250, 500 or even 1000-fold higher than normal cells from the tissue.

In certain embodiments, the Cell Binding Moiety of the binder-drug conjugate is selected to bind to a cell surface protein selectively expressed or upregulated by the target cell in the disease tissue relative to cells from other tissues, particularly cells from critical organs. For instance, the protein is detectable on the surface of the target cells at levels 2 fold higher than cells from other tissues, even more preferably levels at least 5, 10, 20, 30, 40, 50, 75, 100, 250, 500 or even 1000-fold higher than cells from other tissues.

In certain embodiments, the Cell Binding Moiety of the binder-drug conjugate is selected to bind to a checkpoint protein and preferably the Cell Binding Moiety is an antagonist of that checkpoint. Examples of checkpoint proteins include those selected from the group consisting of CTLA-4, PD-1, LAG-3, BTLA, KIR, TIM-3, PD-L1, PD-L2, B7-H3, B7-H4, HVEM, GAL9, CD160, VISTA, BTNL2, TIGIT, PVR, BTN1A1, BTN2A2, BTN3A2 and CSF-1R, more preferably CTLA-4, PD-1, LAG-3, TIM-3, BTLA, VISTA, HVEM, TIGIT, PVR, PD-L1 and CD160.

In certain embodiments, the Cell Binding Moiety of the binder-drug conjugate is selected to bind a co-stimulatory receptor and the Cell Binding Moiety is a costimulatory agonist of the receptor. Examples include the surface feature being a cotimulatory receptor or ligand selected from the group consisting of 4-1BB, 4-1BB-L, OX40, OX40-L, GITR, CD28, CD40, CD40-L, ICOS, ICOS-L, LIGHT, and CD27, more preferably 4-1BB, OX40, GITR, CD40 and ICOS.

In certain embodiments, the Cell Binding Moiety is an antibody, such as a humanized antibody, a human antibody, or a chimeric antibody, or comprises an antigen-binding portion thereof that binds the cell surface feature, such as Fab, F(ab)2, F(ab′), F(ab′)2, F(ab′)3, Fd, Fv, disulfide linked Fv, dAb or sdAb (or nanobody), CDR, scFv, (scFv)2, di-scFv, bi-scFv, tascFv (tandem scFv), AVIBODY (e.g., diabody, triabody, tetrabody), T-cell engager (BiTE), scFv-Fc, Fcab, mAb2, small modular immunopharmaceutical (SMIP), Genmab/unibody or duobody, V-NAR domain, IgNAR, minibody, IgGACH2, DVD-Ig, probody, intrabody, or a multispecificity antibody.

In other embodiments, the Cell Binding Moiety is non-antibody scaffold, such as selected from the group consisting of Affibodies, Affimers, Affilins, Anticalins, Atrimers, Avimer, DARPins, FN3 scaffolds (e.g. Adnectins and Centyrins), Fynomers, Kunitz domains, Nanofitin, Pronectins, OBodies, tribodies, Avimers, bicyclic peptides and Cys-knots.

(i) PD-L1 Binding Affimers

In certain embodiments, the Cell Binding Moiety is an affimer that binds to PD-L1. An affimer is a scaffold based on stefin A, meaning that it has a sequence which is derived from stefin A, preferably a mammalian stefin A, and more preferably a human stefin A. One aspect of the application provides affimers which bind PD-L1 (also referred to as “anti-PD-L1 affimers”) comprising an affimer in which one or more of the solvent accessible loops from the wild-type stefin A protein with amino acid sequences to provide an affimer having the ability to bind PD-L1, preferably selectively, and preferably with Kd of 10⁻⁶M or less.

In certain embodiments, the anti-PD-L1 affimer is derived from the wild-type human stefin A protein having a backbone sequence and in which one or both of loop 2 [designated (Xaa)ₙ] and loop 4 [designated (Xaa)ₘ] are replaced with alternative loop sequences (Xaa)ₙ and (Xaa)ₘ, to have the general formula (i)

FR1-(Xaa)ₙ-FR2-(Xaa)ₘ-FR3 (I)

wherein
FR1 is a polypeptide sequence represented by MIPGGLSEAK PATPEIQEIV DKVKPQLEEK TNETYGKLEA VQYKTQVLA (SEQ ID No. 1) or a polypeptide sequence having at least 70% homology thereto;
FR2 is a polypeptide sequence represented by GTNYYIKVRA GDNKYMHLKV FKSL (SEQ ID No. 2) or a polypeptide sequence having at least 70% homology thereto;
FR3 is a polypeptide sequence represented by EDLVLTGYQV DKNKDDELTG F (SEQ ID No. 3) or a polypeptide sequence having at least 70% homology thereto; and
Xaa, individually for each occurrence, is an amino acid residue, n and m are each, independently, an integer from 3 to 20.

In certain embodiments, FR1 is a polypeptide sequence having at least 80%, 85%, 90%, 95% or even 98% homology with SEQ ID No. 1. In certain embodiments, FR1 is a polypeptide sequence having at least 80%, 85%, 90%, 95% or even 98% identity with SEQ ID No. 1; In certain embodiments, FR2 is a polypeptide sequence having at least 80%, 85%, 90%, 95% or even 98% homology with SEQ ID No. 2. In certain embodiments, FR2 is a polypeptide sequence having at least 80%, 85%, 90%, 95% or even 98% identity with SEQ ID No. 2; In certain embodiments, FR3 is a polypeptide sequence having at least 80%, 85%, 90%, 95% or even 98% homology with SEQ ID No. 3. In certain embodiments, FR3 is a polypeptide sequence having at least 80%, 85%, 90%, 95% or even 98% identity with SEQ ID No. 3.

For those embodiments in which at least one drug-conjugate moiety is appended to the affimer sequence through a thiol side chain of a cysteine introduced into the affimer sequence, the cysteine will preferably be provided in a portion of the affimer sequence regions corresponding to FR1, FR2 and/or FR3, and more preferably with a replacement to an amino acid residue in the affimer the side chain of which is solvent accessible and is not involved in hydrogen bonding with other portions of the affimer. In general, cysteines will not be introduced into the loops (Xaa)ₙ or (Xaa)ₘ.

In certain embodiments, the anti-PD-L1 affimer has an amino acid sequence represented in the general formula (SEQ ID No. 4):

MIP-Xaa1-GLSEAKPATPEIQEIVDKVKPQLEEKTNETYGKLEAVQYKT
QVLA-(Xaa)ₙ-Xaa2-TNYYIKVRAGDNKYMHLKVF-Xaa3-Xaa4-
Xaa5-(Xaa)ₘ-Xaa6-D-Xaa7-VLTGYQVDKNKDDELTGF

wherein
Xaa, individually for each occurrence, is an amino acid residue; n and m are each, independently, an integer from 3 to 20; Xaa1 is Gly, Ala, Val, Arg, Lys, Asp, or Glu, more preferably Gly, Ala, Arg or Lys, and more even more preferably Gly or Arg; Xaa2 is Gly, Ala, Val, Ser or Thr, more preferably Gly or Ser; Xaa3 is Arg, Lys, Asn, Gln, Ser, Thr, more preferably Arg, Lys, Asn or Gln, and even more preferably Lys or Asn; Xaa4 is Gly, Ala, Val, Ser or Thr, more preferably Gly or Ser; Xaa5 is Ala, Val, Ile, Leu, Gly or Pro, more preferably Ile, Leu or Pro, and even more preferably Leu or Pro; Xaa6 is Gly, Ala, Val, Asp or Glu, more preferably Ala, Val, Asp or Glu, and even more preferably Ala or Glu; and Xaa7 is Ala, Val, Ile, Leu, Arg or Lys, more preferably Ile, Leu or Arg, and even more preferably Leu or Arg.

For those embodiments in which at least one drug-conjugate moiety is appended to the affimer sequence through a thiol side chain of a cysteine introduced into the affimer sequence, the cysteine will preferably be provided in a portion of the affimer sequence other than with the loop sequences (Xaa)ₙ or (Xaa)ₘ. Accordingly, the SEQ ID No. 4 may include from 1 to cysteines in place of amino acid residues at varying positions of that sequence.

For instance, the anti-PD-L1 affimer can have an amino acid sequence represented in the general formula (SEQ ID No. 5):

MIPRGLSEAKPATPEIQEIVDKVKPQLEEKTNETYGKLEAVQYKTQVLA-
(Xaa)ₙ-STNYYIKVRAGDNKYMHLKVFNGP-(Xaa)ₘ-ADRVLTGYQVD
KNKDDELTGF

wherein Xaa, individually for each occurrence, is an amino acid residue; n and m are each, independently, an integer from 3 to 20.

In certain embodiments, n is 3 to 15, 3 to 12, 3 to 9, 3 to 7, 5 to 7, 5 to 9, 5 to 12, 5 to 15, 7 to 12 or 7 to 9.

In certain embodiments, m is 3 to 15, 3 to 12, 3 to 9, 3 to 7, 5 to 7, 5 to 9, 5 to 12, 5 to 15, 7 to 12 or 7 to 9.

In certain embodiments, Xaa, independently for each occurrence, is an amino acid that can be added to a polypeptide by recombinant expression in a prokaryotic or eukaryotic cell, and even more preferably one of the 20 naturally occurring amino acids.

For those embodiments in which at least one drug-conjugate moiety is appended to the affimer sequence through a thiol side chain of a cysteine introduced into the affimer sequence, the cysteine will preferably be provided in a portion of the affimer sequence other than with the loop sequences (Xaa)ₙ or (Xaa)ₘ. Accordingly, the SEQ ID No. 5 may include from 1 to cysteines in place of amino acid residues at varying positions of that sequence.

In certain embodiments of the above sequences and formulas, (Xaa)ₙ is an amino acid sequence represented in the general formula (II)

-aa1-aa2-aa3-Gly-Pro-aa4-aa5-Trp-aa6- (II)

wherein
aa1 represents an amino acid residue with a basic sidechain, more preferably Lys, Arg or His, and even more preferably Lys or Arg;
aa2 represents an amino acid residue, preferably an amino acid residue with a neutral polar or non-polar sidechain or a charged (acidic or basic) sidechain, more preferably a small aliphatic sidechain, a neutral polar side chain or a basic or acid side chain, even more preferably Ala, Pro, Ile, Gln, Thr, Asp, Glu, Lys, Arg or His, and even more preferably Ala, Gln, Asp or Glu;
aa3 represents an amino acid residue with an aromatic or basic sidechain, preferably Phe, Tyr, Trp, Lys, Arg or His, more preferably Phe, Tyr, Trp, and even more preferably His or Tyr, Trp or His;
aa4 represents an amino acid residue with a neutral polar or non-polar sidechain or a charged (acidic or basic) sidechain; preferably a neutral polar sidechain or a charged (acidic or basic) sidechain; more preferably Ala, Pro, Ile, Gln, Thr, Asp, Glu, Lys, Arg or His, and even more preferably Gln, Lys, Arg, His, Asp or Glu;
aa5 represents an amino acid residue with a neutral polar or a charged (acidic or basic) or a small aliphatic or an aromatic sidechain; preferably a neutral polar sidechain or a charged sidechain; more preferably Ser, Thr, Asn, Gln, Asp, Glu, Arg or His, and even more preferably Ser, Asn, Gln, Asp, Glu or Arg; and
aa6 represents an amino acid residue with an aromatic or acid sidechain, preferably Phe, Tyr, Trp, Asp or Glu; more preferably Trp or Asp; and even more preferably Trp.

In certain embodiments of the above sequences and formulas, (Xaa)ₙ is an amino acid sequence represented in the general formula (III)

-aa1-aa2-aa3-Phe-Pro-aa4-aa5-Phe-Trp- (III)

wherein
aa1 represents an amino acid residue with a basic sidechain or aromatic sidechain, preferably Lys, Arg, His, Ser, Thr, Asn or Gln, more preferably Lys, Arg, His, Asn or Gln, and even more preferably Lys or Asn;
aa2 represents an amino acid residue, preferably an amino acid residue with a neutral polar or non-polar sidechain or a charged (acidic or basic) sidechain, more preferably a small aliphatic sidechain, a neutral polar side chain or a basic or acid side chain, even more preferably Ala, Pro, Ile, Gln, Thr, Asp, Glu, Lys, Arg or His, and even more preferably Ala, Gln, Asp or Glu;
aa3 represents an amino acid residue with an aromatic or basic sidechain, preferably Phe, Tyr, Trp, Lys, Arg or His, more preferably Phe, Tyr, Trp or His, and even more preferably Tyr, Trp or His;
aa4 represents an amino acid residue with a neutral polar or non-polar sidechain or a charged (acidic or basic) sidechain; preferably a neutral polar sidechain or a charged (acidic or basic) sidechain; more preferably Ala, Pro, Ile, Gln, Thr, Asp, Glu, Lys, Arg or His, and even more preferably Gln, Lys, Arg, His, Asp or Glu; and
aa5 represents an amino acid residue with a neutral polar or a charged (acidic or basic) or a small aliphatic or an aromatic sidechain; preferably a neutral polar sidechain or a charged sidechain; more preferably Ser, Thr, Asn, Gln, Asp, Glu, Arg or His, and even more preferably Ser, Asn, Gln, Asp, Glu or Arg.

In certain embodiments of the above sequences and formulas, (Xaa)ₙ is an amino acid sequence selected from SEQ ID Nos. 6 to 40, or an amino acid sequence having at least 80%, 85%, 90%, 95% or even 98% homology with a sequence selected from SEQ ID Nos. 6 to 40. In certain embodiments, (Xaa)ₙ is an amino acid sequence having at least 80%, 85%, 90%, 95% or even 98% identity with a sequence selected from SEQ ID No. 6 to 40.

| Loop 2 sequences | SEQ ID No. |
|---|---|
| KAWGPKQWW | 6 |
| KPYGPRDWD | 7 |
| KEYGPEEWW | 8 |
| HAYGPRDWD | 9 |
| KDHGPIAWW | 10 |
| NKHFHQRFW | 11 |
| NKHFPIHFW | 12 |
| HEFGPAEWD | 13 |
| NAHFPQSFW | 14 |
| KEHGPDSWW | 15 |
| NQHFPHSFW | 16 |
| NAHFGPRFW | 17 |
| NTWFPESFW | 18 |
| NQHFPQSFW | 19 |
| KQYGPDDWW | 20 |
| KDWGPSNWW | 21 |
| KQFGPKDWW | 22 |
| NHHFPKRFW | 23 |
| YRHFPQWH | 24 |
| NIHFPPNFW | 25 |
| YTHFPQWT | 26 |
| NDHFPHTFW | 27 |
| NQHFPSYFW | 28 |
| NQYFPPHFW | 29 |
| KKHFPASFW | 30 |
| KKFFPKHFW | 31 |
| KLHFPRSFW | 32 |
| YKHFPPNFW | 33 |
| EEHFPFQFW | 34 |
| KPHFPDNFW | 35 |
| YQYFPDQFN | 36 |
| VQWFPRSFW | 37 |
| AAHFPEHFW | 38 |
| REGRQDWVL | 39 |
| WVPFPHQQL | 40 |

In certain embodiments of the above sequences and formulas, (Xaa)ₘ is an amino acid sequence represented in the general formula (IV)

-aa7-aa8-aa9-aa10-aa11-aa12-aa13-aa14-aa15- (IV)

wherein
aa7 represents an amino acid residue with neutral polar or non-polar sidechain or an acidic sidechain; preferably Gly, Ala, Val, Pro, Trp, Gln, Ser, Asp or Glu, and even more preferably Gly, Ala, Trp, Gln, Ser, Asp or Glu;
aa8 represents an amino acid residue, preferably an amino acid residue with a neutral polar or non-polar sidechain or a charged (acidic or basic) sidechain or aromatic sidechain, more preferably a charged (acidic or basic) sidechain, more preferably Asp, Glu, Lys, Arg, His, Gln, Ser, Thr, Asn, Ala, Val, Pro, Gly, Tyr or Phe, and even more preferably Asp, Glu, Lys, Arg, His or Gln;
aa9 represents an amino acid residue, preferably an amino acid residue with a neutral polar or non-polar sidechain or a charged (acidic or basic) sidechain or aromatic sidechain, more preferably a neutral polar side chain or an acid side chain, more preferably Gln, Ser, Thr, Asn, Asp, Glu, Arg, Lys, Gly, Leu, Pro or Tyr, and even more preferably Gln, Thr or Asp;
aa10 represents an amino acid residue, preferably an amino acid residue with a neutral polar or non-polar sidechain or a charged (acidic or basic) sidechain or aromatic sidechain, more preferably a neutral polar side chain or a basic or acid side chain, more preferably Asp, Glu, Arg, His, Lys, Ser, Gln, Asn, Ala, Leu, Tyr, Trp, Pro or Gly, and even more preferably Asp, Glu, His, Gln, Asn, Leu, Trp or Gly;
aa11 represents an amino acid residue, preferably an amino acid residue with a neutral polar sidechain or a charged (acidic or basic) sidechain or a nonpolar aliphatic sidechain or an aromatic sidechain, more preferably a neutral polar side chain or a basic or acid side chain, more preferably Asp, Glu, Ser, Thr, Gln, Arg, Lys, His, Val, Ile, Tyr or Gly and even more preferably Asp, Glu, Ser, Thr, Gln, Lys or His;
aa12 represents an amino acid residue, preferably an amino acid residue with a neutral polar sidechain or a charged (acidic or basic) sidechain or a nonpolar aliphatic sidechain or an aromatic sidechain, more preferably a an acid side chain, more preferably Asp, Glu, Ser, Thr, Gln, Asn, Lys, Arg, Val, Leu, Ile, Trp, Tyr, Phe or Gly and even more preferably Asp, Glu, Ser, Tyr, Trp, Arg or Lys;
aa13 represents an amino acid residue, preferably an amino acid residue with a neutral polar sidechain or a charged (acidic or basic) sidechain or a nonpolar aliphatic sidechain or an aromatic sidechain, more preferably a an acid side chain, more preferably Ser, Thr, Gln, Asn, Val, Ile, Leu, Gly, Pro, Asp, Glu, His, Arg, Trp, Tyr or Phe and even more preferably Ser, Thr, Gln, Asn, Val, Ile, Leu, Gly, Asp or Glu;
aa14 represents an amino acid residue, preferably an amino acid residue with a neutral polar sidechain or a charged (acidic or basic) sidechain, more preferably Ala, Ile, Trp, Pro, Asp, Glu, Arg, Lys, His, Ser, Thr, Gln or Asn and even more preferably Ala, Pro, Asp, Glu, Arg, Lys, Ser, Gln or Asn; and
aa15 represents an amino acid residue, preferably an amino acid residue with a neutral polar or neutral non-polar sidechain or a charged (acidic or basic) sidechain, more preferably His, Arg, Lys, Asp, Ser, Thr, Gln, Asn, Ala, Val, Leu, Gly or Phe and even more preferably His, Arg, Lys, Asp, Ser, Thr, Gln or Asn.

In certain embodiments of the above sequences and formulas, (Xaa)ₘ is an amino acid sequence selected from SEQ ID Nos. 41 to 75, or an amino acid sequence having at least 80%, 85%, 90%, 95% or even 98% homology with a sequence selected from SEQ ID Nos. 41 to 75. In certain embodiments, (Xaa)ₘ is an amino acid sequence having at least 80%, 85%, 90%, 95% or even 98% identity with a sequence selected from SEQ ID No. 41 to 75.

| Loop 4 sequences | SEQ ID No. |
|---|---|
| GRTIQ | 41 |
| EPQLDTSPI | 42 |
| GDYEQVLIH | 43 |
| PADHVLEEA | 44 |
| EDTNTDGAL | 45 |
| GQSWDQRRQ | 46 |
| SKSPIDLPF | 47 |
| DPQDVYLNQ | 48 |
| GSLHSFGST | 49 |
| QEKNQWVEE | 50 |
| QKNYEEDPH | 51 |
| WDGHKRFAD | 52 |
| DDNQERQEH | 53 |
| AVTQEDQAV | 54 |
| EVDWKYQDH | 55 |
| VDDKTLSKD | 56 |
| QGQGKDPSQ | 57 |
| GHQSEVQHS | 58 |
| TGTSIWNQD | 59 |
| GVHDSLQGYDA | 60 |
| QKGQKIDKF | 61 |
| DDELHDTRH | 62 |
| ATTGDEWDR | 63 |
| SHPHSNHTS | 64 |
| WRTDYKYEE | 65 |
| NDPHDSVPH | 66 |
| GQQRENEQE | 67 |
| GERQQDDAN | 68 |
| AYREGSQWT | 69 |
| EFYDHGIIQ | 70 |
| ENEATRDQH | 71 |
| GYDHEDNRG | 72 |
| QPADMSAEF | 73 |
| WVPFPHQQL | 74 |
| REGRQDWVL | 75 |

In certain embodiments, the anti-PD-L1 affimer has an amino acid sequence selected from SEQ ID Nos. 76 to 84, or an amino acid sequence having at least 70%, 75% 80%, 85%, 90%, 95% or even 98% homology with a sequence selected from SEQ ID Nos. 76 to 84. In certain embodiments, the anti-PD-L1 affimer has an amino acid sequence having at least 70%, 75% 80%, 85%, 90%, 95% or even 98% identity with a sequence selected from SEQ ID No. 76 to 84.

For those embodiments in which at least one drug-conjugate moiety is appended to the affimer sequence through a thiol side chain of a cysteine introduced into the affimer sequence, the cysteine will preferably be provided in a portion of the affimer sequence other than with the loop sequences (Xaa)ₙ or (Xaa)ₘ. Accordingly, the anti-PD-L1 affimer will have a sequence that varies from SEQ ID No. 76 to 84 by at least the inclusion of from 1 to 5 cysteines in place of amino acid residues at varying positions of that sequence, though preferably not in the Loop 2 or Loop 4 sequence.

| Exemplary anti-PD-L1 | |
|---|---|
| Affimer Sequences | SEQ ID No. |
| MIPRGLSEAK PATPEIQEIV DKVKPQLEEK | 76 |
| TNETYGKLEA VQYKTQVLAK EHGPDSWWST | |
| NYYIKVRAGD NKYMHLKVFN GPQEKNQWVE | |
| EADRVLTGYQ VDKNKDDELT GF | |
| MIPRGLSEAK PATPEIQEIV DKVKPQLEEK | 77 |
| TNETYGKLEA VQYKTQVLAK EYGPEEWWST | |
| NYYIKVRAGD NKYMHLKVFN GPGDYEQVLI | |
| HADRVLTGYQ VDKNKDDELT GF | |
| MIPRGLSEAK PATPEIQEIV DKVKPQLEEK | 78 |
| TNETYGKLEA VQYKTQVLAK DHGPIAWWST | |
| NYYIKVRAGD NKYMHLKVFN GPEDTNTDGA | |
| LADRVLTGYQ VDKNKDDELT GF | |
| MIPRGLSEAK PATPEIQEIV DKVKPQLEEK | 79 |
| TNETYGKLEA VQYKTQVLAK DWGPSNWWST | |
| NYYIKVRAGD NKYMHLKVFN GPVDDKTLSK | |
| DADRVLTGYQ VDKNKDDELT GF | |
| MIPRGLSEAK PATPEIQEIV DKVKPQLEEK | 80 |
| TNETYGKLEA VQYKTQVLAN TWFPESFWST | |
| NYYIKVRAGD NKYMHLKVFN GPDDNQERQE | |
| HADRVLTGYQ VDKNKDDELT GF | |
| MIPRGLSEAK PATPEIQEIV DKVKPQLEEK | 81 |
| TNETYGKLEA VQYKTQVLAK PYGPRDWDST | |
| NYYIKVRAGD NKYMHLKVFN GPEPQLDTSP | |
| IADRVLTGYQ VDKNKDDELT GF | |
| MIPRGLSEAK PATPEIQEIV DKVKPQLEEK | 82 |
| TNETYGKLEA VQYKTQVLAH AYGPRDWDST | |
| NYYIKVRAGD NKYMHLKVFN GPPADHVLEE | |
| AADRVLTGYQ VDKNKDDELT GF | |
| MIPRGLSEAK PATPEIQEIV DKVKPQLEEK | 83 |
| TNETYGKLEA VQYKTQVLAA AHFPEHFWST | |
| NYYIKVRAGD NKYMHLKVFN GPQPADMSAE | |
| FADRVLTGYQ VDKNKDDELT GF | |
| MIPRGLSEAK PATPEIQEIV DKVKPQLEEK | 84 |
| TNETYGKLEA VQYKTQVLAR EGRQDWVLST | |
| NYYIKVRAGD NKYMHLKVFN GPWVPFPHQQ | |
| LADRVLTGYQ VDKNKDDELT GF | |

In certain embodiments, the anti-PD-L1 affimer has an amino acid sequence that is encoded by a nucleic acid having a coding sequence corresponding to nucleotides 1-336 of one of SEQ ID Nos. 85 to 92, or an amino acid sequence that can be encoded by a nucleic acid having a coding sequence at least 70%, 75% 80%, 85%, 90%, 95% or even 98% identical with nucleotides 1-336 of one of SEQ ID Nos. 85 to 92, or an amino acid sequence that can be encoded by a nucleic acid having a coding sequence that hybridizes nucleotides 1-336 of one of SEQ ID Nos. 85 to 92 under stringent conditions (such as in the presence of 6× sodium chloride/sodium citrate (SSC) at 45° C. followed by a wash in 0.2×SSC at 65° C.

For those embodiments in which at least one drug-conjugate moiety is appended to the affimer sequence through a thiol side chain of a cysteine introduced into the affimer sequence, the cysteine will preferably be provided in a portion of the affimer sequence other than with the loop sequences (Xaa)ₙ or (Xaa)ₘ. Accordingly, the anti-PD-L1 affimer will have a sequence that varies from amino acid sequences encoded by SEQ ID No. 85 to 92 by at least the inclusion of from 1 to 5 cysteines in place of amino acid residues at varying positions of that sequence, though preferably not in the Loop 2 or Loop 4 sequence.

| | SEQ |
|---|---|
| Exemplary anti-PD-L1 Affimer | ID |
| Coding Sequences | No. |
| ATGATCCCGCGTGGCCTGTCTGAAGCTAAACCAGCAACTCCGG | 85 |
| AAATTCAAGAGATCGTCGATAAGGTGAAACCGCAGCTGGAAG | |
| AGAAAACGAACGAAACCTACGGTAAGCTGGAAGCGGTCCAGT | |
| ACAAAACCCAAGTGCTAGCAAAAGATTGGGGTCCATCTAACT | |
| GGTGGTCCACCAACTATTACATTAAGGTTCGTGCCGGTGACAA | |
| TAAGTATATGCACCTGAAAGTGTTCAACGGCCCGGTTGATGAT | |
| AAAACCCTGTCTAAAGATGCGGACCGTGTTCTGACCGGTTACC | |
| AGGTTGACAAGAACAAAGATGACGAGCTGACGGGTTTCGCGG | |
| CCGCGGGTCATCACCACCACCACCATTAG | |
| ATGATCCCGCGTGGCCTGTCTGAAGCTAAACCAGCAACTCCGG | 86 |
| AAATTCAAGAGATCGTCGATAAGGTGAAACCGCAGCTGGAAG | |
| AGAAAACGAACGAAACCTACGGTAAGCTGGAAGCGGTCCAGT | |
| ACAAAACCCAAGTGCTAGCAAAAGATCATGGTCCAATCGCAT | |
| GGTGGTCCACCAACTATTACATTAAGGTTCGTGCCGGTGACAA | |
| TAAGTATATGCACCTGAAAGTGTTCAACGGCCCGGAAGATAC | |
| CAACACCGATGGTGCACTGGCGGACCGTGTTCTGACCGGTTAC | |
| CAGGTTGACAAGAACAAAGATGACGAGCTGACGGGTTTCGCG | |
| GCCGCGGGTCATCACCACCACCACCATTAG | |
| ATGATCCCGCGTGGCCTGTCTGAAGCTAAACCAGCAACTCCGG | 87 |
| AAATTCAAGAGATCGTCGATAAGGTGAAACCGCAGCTGGAAG | |
| AGAAAACGAACGAAACCTACGGTAAGCTGGAAGCGGTCCAGT | |
| ACAAAACCCAAGTGCTAGCAAAACCATACGGTCCACGTGATT | |
| GGGATTCCACCAACTATTACATTAAGGTTCGTGCCGGTGACAA | |
| TAAGTATATGCACCTGAAAGTGTTCAACGGCCCGGAACCACA | |
| GCTGGATACCTCTCCAATCGCGGACCGTGTTCTGACCGGTTAC | |
| CAGGTTGACAAGAACAAAGATGACGAGCTGACGGGTTTCGCG | |
| GCCGCGGGTCATCACCACCACCACCATTAG | |
| ATGATCCCGCGTGGCCTGTCTGAAGCTAAACCAGCAACTCCGG | 88 |
| AAATTCAAGAGATCGTCGATAAGGTGAAACCGCAGCTGGAAG | |
| AGAAAACGAACGAAACCTACGGTAAGCTGGAAGCGGTCCAGT | |
| ACAAAACCCAAGTGCTAGCAAACACCTGGTTTCCAGAATCTTT | |
| TTGGTCCACCAACTATTACATTAAGGTTCGTGCCGGTGACAAT | |
| AAGTATATGCACCTGAAAGTGTTCAACGGCCCGGATGATAAC | |
| CAGGAACGTCAGGAACATGCGGACCGTGTTCTGACCGGTTAC | |
| CAGGTTGACAAGAACAAAGATGACGAGCTGACGGGTTTCGCG | |
| GCCGCGGGTCATCACCACCACCACCATTAG | |
| ATGATCCCGCGTGGCCTGTCTGAAGCTAAACCAGCAACTCCGG | 89 |
| AAATTCAAGAGATCGTCGATAAGGTGAAACCGCAGCTGGAAG | |
| AGAAAACGAACGAAACCTACGGTAAGCTGGAAGCGGTCCAGT | |
| ACAAAACCCAAGTGCTAGCACGTGAAGGTCGTCAGGATTGGG | |
| TTCTGTCCACCAACTATTACATTAAGGTTCGTGCCGGTGACAA | |
| TAAGTATATGCACCTGAAAGTGTTCAACGGCCCGTGGGTTCCA | |
| TTTCCACATCAGCAGCTGGCGGACCGTGTTCTGACCGGTTACC | |
| AGGTTGACAAGAACAAAGATGACGAGCTGACGGGTTTCGCGG | |
| CCGCGGGTCATCACCACCACCACCATTAG | |
| ATGATCCCGCGTGGCCTGTCTGAAGCTAAACCAGCAACTCCGG | 90 |
| AAATTCAAGAGATCGTCGATAAGGTGAAACCGCAGCTGGAAG | |
| AGAAAACGAACGAAACCTACGGTAAGCTGGAAGCGGTCCAGT | |
| ACAAAACCCAAGTGCTAGCACATGCATACGGTCCACGTGATT | |
| GGGATTCCACCAACTATTACATTAAGGTTCGTGCCGGTGACAA | |
| TAAGTATATGCACCTGAAAGTGTTCAACGGCCCGCCAGCAGA | |
| TCATGTTCTGGAAGAAGCAGCGGACCGTGTTCTGACCGGTTAC | |
| CAGGTTGACAAGAACAAAGATGACGAGCTGACGGGTTTCGCG | |
| GCCGCGGGTCATCACCACCACCACCATTAG | |
| ATGATCCCGCGTGGCCTGTCTGAAGCTAAACCAGCAACTCCGG | 91 |
| AAATTCAAGAGATCGTCGATAAGGTGAAACCGCAGCTGGAAG | |
| AGAAAACGAACGAAACCTACGGTAAGCTGGAAGCGGTCCAGT | |
| ACAAAACCCAAGTGCTAGCAAAAGAATACGGTCCAGAAGAAT | |
| GGTGGTCCACCAACTATTACATTAAGGTTCGTGCCGGTGACAA | |
| TAAGTATATGCACCTGAAAGTGTTCAACGGCCCGGGTGATTAC | |
| GAACAGGTTCTGATCCATGCGGACCGTGTTCTGACCGGTTACC | |
| AGGTTGACAAGAACAAAGATGACGAGCTGACGGGTTTCGCGG | |
| CCGCGGGTCATCACCACCACCACCATTAG | |
| ATGATCCCGCGTGGCCTGTCTGAAGCTAAACCAGCAACTCCGG | 92 |
| AAATTCAAGAGATCGTCGATAAGGTGAAACCGCAGCTGGAAG | |
| AGAAAACGAACGAAACCTACGGTAAGCTGGAAGCGGTCCAGT | |
| ACAAAACCCAAGTGCTAGCAGCTGCTCATTTCCCGGAACATTT | |
| CTGGTCCACCAACTATTACATTAAGGTTCGTGCCGGTGACAAT | |
| AAGTATATGCACCTGAAAGTGTTCAACGGCCCGCAGCCGGCT | |
| GATATGTCTGCTGAATTCGCGGACCGTGTTCTGACCGGTTACC | |
| AGGTTGACAAGAACAAAGATGACGAGCTGACGGGTTTCCTGC | |
| AGGCGGCCGCGCACCACCACCACCACCACTG | |

Furthermore, minor modifications may also include small deletions or additions—beyond the loop 2 and loop 4 inserts described above—to the stefin A or stefin A derived sequences disclosed herein, such as addition or deletion of up to 10 amino acids relative to stefin A or the stefin A derived Affimer polypeptide.

In certain embodiments, the PD-L1 binding Affimer polypeptide binds human PD-L1 as a monomer with a dissociation constant (K_{D}) of about 1 μM or less, about 100 nM or less, about 40 nM or less, about 20 nM or less, about 10 nM or less, about 1 nM or less, or about 0.1 nM or less.

In certain embodiments, the PD-L1 binding Affimer polypeptide portion binds human PD-L1 as a monomer with an off rate constant (K_{off}), such as measured by Biacore, of about 10⁻³ s⁻¹ (i.e., unit of 1/second) or slower; of about 10⁻⁴ S or slower or even of about 10⁻⁵ s⁻¹ or slower.

In certain embodiments, the PD-L1 binding Affimer polypeptide portion binds human PD-L1 as a monomer with an association constant (Kₒₙ), such as measured by Biacore, of at least about 10³ M⁻¹s⁻¹ or faster; at least about 10⁴ M⁻¹s⁻¹ or faster; at least about 10⁵ M⁻¹s⁻¹ or faster; or even at least about 10⁶ M⁻¹s⁻¹ or faster.

In certain embodiments, the PD-L1 binding Affimer polypeptide portion binds human PD-L1 as a monomer with an IC50 in a competitive binding assay with human PD-1 of 1 μM or less, about 100 nM or less, about 40 nM or less, about 20 nM or less, about 10 nM or less, about 1 nM or less, or about 0.1 nM or less.

Fusions Proteins—General

In some embodiments, the affimer polypeptides may further comprise an additional insertion, substitution or deletion that modulates biological activity of the affimer polypeptide. For example, the additions, substitutions or deletions may modulate one or more properties or activities of modified affimer. For example, the additions, substitutions or deletions may modulate affinity for the affimer polypeptide, e.g., for binding to and inhibiting PD-1, modulate the circulating half-life, modulate the therapeutic half-life, modulate the stability of the affimer polypeptide, modulate cleavage by proteases, modulate dose, modulate release or bio-availability, facilitate purification, decrease deamidation, improve shelf-life, or improve or alter a particular route of administration. Similarly, affimer polypeptides may comprise protease cleavage sequences, reactive groups, antibody-binding domains (including but not limited to, FLAG or poly-His) or other affinity based sequences (including but not limited to, FLAG, poly-His, GST, etc.) or linked molecules (including but not limited to, biotin) that improve detection, purification or other traits of the polypeptide.

In some instances, these additional sequences are added to one end and/or the other of the affimer polypeptide in the form of a fusion protein. Accordingly, in certain aspects of the invention the Binder-drug conjugate is a fusion protein having at least one affimer polypeptide sequence and one or more heterologous polypeptide sequences (“fusion domain” herein). A fusion domain may be selected so as to confer a desired property, such as secretion from a cell or retention on the cell surface (i.e., for Encoded Affimers), to serve as substrate or other recognition sequences for post-translational modifications, to create multimeric structures aggregating through protein-protein interactions, to alter (often to extend) serum half-life, or to alter tissue localization or tissue exclusion and other ADME properties—merely as examples.

For example, some fusion domains are particularly useful for isolation and/or purification of the fusion proteins, such as by affinity chromatography. Well known examples of such fusion domains that facilitate expression or purification include, merely to illustrate, affinity tags such as polyhistidine (i.e., a His₆ tag), Strep II tag, streptavidin-binding peptide (SBP) tag, calmodulin-binding peptide (CBP), glutathione S-transferase (GST), maltose-binding protein (MBP), S-tag, HA tag, c-Myc tag, thioredoxin, protein A and protein G.

In order for the Affimer to be secreted if made recombinantly, it will generally contain a signal sequence that directs the transport of the protein to the lumen of the endoplasmic reticulum and ultimately to be secreted (or retained on the cell surface if a transmembrane domain or other cell surface retention signal). Signal sequences (also referred to as signal peptides or leader sequences) are located at the N-terminus of nascent polypeptides. They target the polypeptide to the endoplasmic reticulum and the proteins are sorted to their destinations, for example, to the inner space of an organelle, to an interior membrane, to the cell outer membrane, or to the cell exterior via secretion. Most signal sequences are cleaved from the protein by a signal peptidase after the proteins are transported to the endoplasmic reticulum. The cleavage of the signal sequence from the polypeptide usually occurs at a specific site in the amino acid sequence and is dependent upon amino acid residues within the signal sequence.

In some embodiments, the signal peptide is about 5 to about 40 amino acids in length (such as about 5 to about 7, about 7 to about 10, about 10 to about 15, about 15 to about 20, about to about 25, or about 25 to about 30, about 30 to about 35, or about 35 to about 40 amino acids in length).

In some embodiments, the signal peptide is a native signal peptide from a human protein. In other embodiments, the signal peptide is a non-native signal peptide. For example, in some embodiments, the non-native signal peptide is a mutant native signal peptide from the corresponding native secreted human protein, and can include one or more (such as 2, 3, 4, 5, 6, 7, 8, 9, or 10 or more) substitutions insertions or deletions.

In some embodiments, the signal peptide is a signal peptide or mutant thereof from a non-IgSF protein family, such as a signal peptide from an immunoglobulin (such as IgG heavy chain or IgG-kappa light chain), a cytokine (such as interleukin-2 (IL-2), or CD33), a serum albumin protein (e.g. HSA or albumin), a human azurocidin preprotein signal sequence, a luciferase, a trypsinogen (e.g. chymotrypsinogen or trypsinogen) or other signal peptide able to efficiently secrete a protein from a cell. Exemplary signal peptides include, but are not limited to:

| Native Protein | Signal Sequence |
|---|---|
| HSA | MKWVTFISLLFLFSSAYS |
| Ig kappa light chain | MDMRAPAGIFGFLLVLFPGYRS |
| Human azurocidin | MTRLTVLALLAGLLASSRA |
| preprotein | |
| IgG heavy chain | MELGLSWIFLLAILKGVQC |
| IgG heavy chain | MELGLRWVFLVAILEGVQC |
| IgG heavy chain | MKHLWFFLLLVAAPRWVLS |
| IgG heavy chain | MDWTWRILFLVAAATGAHS |
| IgG heavy chain | MDWTWRFLFVVAAATGVQS |
| IgG heavy chain | MEFGLSWLFLVAILKGVQC |
| IgG heavy chain | MEFGLSWVFLVALFRGVQC |
| IgG heavy chain | MDLLHKNMKHLWFFLLLVAAPRW |
| | VLS |
| IgG Kappa light | MDMRVPAQLLGLLLLWLSGARC |
| IgG Kappa light | MKYLLPTAAAGLLLLAAQPAMA |
| Gaussia luciferase | MGVKVLFALICIAVAEA |
| Human albumin | MKWVTFISLLFLFSSAYS |
| Human chymotrypsinogen | MAFLWLLSCWALLGTTFG |
| Human interleukin-2 | MQLLSCIALILALV |
| Human trypsinogen-2 | MNLLLILTFVAAAVA |
| Human CD33 | MPLLLLLPLLWAGALA |
| Prolactin | MDSKGSSQKGSRLLLLLVVSNLL |
| | LCQGVVS |
| Human tPA | MDAMKRGLCCVLLLCGAVFVSPS |
| Synthetic/Consensus | MLLLLLLLLLLALALA |
| Synthetic/Consensus | MWWRLWWLLLLLLLLWPMVWA |

The subject fusion proteins may also include one or more linkers separating heterologous protein sequences or domains—i.e., separating cell binding moieties where more than one is included in a binder drug conjugate. As used herein, the term “linker” refers to a linker amino acid sequence inserted between a first polypeptide (e.g., an affimer) and a second polypeptide (e.g., a second affimer, an Fc region, a receptor trap, albumin, etc). Empirical linkers designed by researchers are generally classified into 3 categories according to their structures: flexible linkers, rigid linkers, and in vivo cleavable linkers. Besides the basic role in linking the functional domains together (as in flexible and rigid linkers) or releasing free functional domain in vivo (as in in vivo cleavable linkers), linkers may offer many other advantages for the production of fusion proteins, such as improving biological activity, increasing expression yield, and achieving desirable pharmacokinetic profiles. Linkers should not adversely affect the expression, secretion, or bioactivity of the fusion protein. Linkers should not be antigenic and should not elicit an immune response.

Suitable linkers are known to those of skill in the art and often include mixtures of glycine and serine residues and often include amino acids that are sterically unhindered. Other amino acids that can be incorporated into useful linkers include threonine and alanine residues. Linkers can range in length, for example from 1-50 amino acids in length, 1-22 amino acids in length, 1-10 amino acids in length, 1-5 amino acids in length, or 1-3 amino acids in length. In some embodiments, the linker may comprise a cleavage site. In some embodiments, the linker may comprise an enzyme cleavage site, so that the second polypeptide may be separated from the first polypeptide.

In certain preferred embodiments, the linker can be characterized as flexible. Flexible linkers are usually applied when the joined domains require a certain degree of movement or interaction. They are generally composed of small, non-polar (e.g. Gly) or polar (e.g. Ser or Thr) amino acids. See, for example, Argos P. (1990) “An investigation of oligopeptides linking domains in protein tertiary structures and possible candidates for general gene fusion” J Mol Biol. 211:943-958. The small size of these amino acids provides flexibility and allows for mobility of the connecting functional domains. The incorporation of Ser or Thr can maintain the stability of the linker in aqueous solutions by forming hydrogen bonds with the water molecules, and therefore reduces the unfavorable interaction between the linker and the protein moieties. The most commonly used flexible linkers have sequences consisting primarily of stretches of Gly and Ser residues (“GS” linker). An example of the most widely used flexible linker has the sequence of (Gly-Gly-Gly-Gly-Ser)ₙ. By adjusting the copy number “n”, the length of this GS linker can be optimized to achieve appropriate separation of the functional domains, or to maintain necessary inter-domain interactions. Besides the GS linkers, many other flexible linkers have been designed for recombinant fusion proteins. As These flexible linkers are also rich in small or polar amino acids such as Gly and Ser, but can contain additional amino acids such as Thr and Ala to maintain flexibility, as well as polar amino acids such as Lys and Glu to improve solubility.

In certain preferred embodiments, the linker can be characterized as rigid. While flexible linkers have the advantage to connect the functional domains passively and permitting certain degree of movements, the lack of rigidity of these linkers can be a limitation in certain fusion protein embodiments, such as in expression yield or biological activity. The ineffectiveness of flexible linkers in these instances was attributed to an inefficient separation of the protein domains or insufficient reduction of their interference with each other. Under these situations, rigid linkers have been successfully applied to keep a fixed distance between the domains and to maintain their independent functions.

Many natural linkers exhibited α-helical structures. The α-helical structure was rigid and stable, with intra-segment hydrogen bonds and a closely packed backbone. Therefore, the stiff α-helical linkers can act as rigid spacers between protein domains. George et al. (2002) “An analysis of protein domain linkers: their classification and role in protein folding” Protein Eng. 15(11):871-9. In general, rigid linkers exhibit relatively stiff structures by adopting α-helical structures or by containing multiple Pro residues. Under many circumstances, they separate the functional domains more efficiently than the flexible linkers.

The length of the linkers can be easily adjusted by changing the copy number to achieve an optimal distance between domains. As a result, rigid linkers are chosen when the spatial separation of the domains is critical to preserve the stability or bioactivity of the fusion proteins. In this regard, alpha helix-forming linkers with the sequence of (EAAAK)ₙ have been applied to the construction of many recombinant fusion proteins. Another type of rigid linkers has a Pro-rich sequence, (XP)ₙ, with X designating any amino acid, preferably Ala, Lys, or Glu.

Merely to illustrate, exemplary linkers include:

| | |
|---|---|
| Flexible | (GGGGS)ₙ (i.e., n = 1-6) |
| Flexible | (Gly)₈ |
| Flexible | (Gly)₆ |
| Flexible | KESGSVSSEQLAQFRSLD |
| Flexible | EGKSSGSGSESKST |
| Flexible | GSAGSAAGSGEF |
| Rigid | (EAAAK)ₙ (i.e., n = 1-6) |
| Rigid | A(EAAAK)₄ALEA(EAAAK)₄A |
| Rigid | PAPAP |
| Rigid | AEAAAKEAAAKA |
| Rigid | (Ala-Pro)n (10 to 34 aa) |

Other linkers that may be used in the subject fusion proteins include, but are not limited to, SerGly, GGSG, GSGS, GGGS, S(GGS)ₙ where n is 1-7, GRA, poly(Gly), poly(Ala), GGGSGGG, ESGGGGVT, LESGGGGVT, GRAQVT, WRAQVT, and ARGRAQVT. The hinge regions of the Fc fusions described below may also be considered linkers.

Still other modifications that can be made to the affimer polypeptide sequence itself or to a flanking polypeptide moiety provided as part of a fusion protein is one or more sequences that are sites for post-translational modifications by enzymes. These can include, but are not limited to, glycosylation, acetylation, acylation, lipid-modification, palmitoylation, palmitate addition, phosphorylation, glycolipid-linkage modification, and the like.

Engineering PK and ADME Properties

In certain embodiment, the binder-drug conjugate may not have a half-life and/or PK profile that is optimal for the route of administration, such as parenteral therapeutic dosing. The term “half-life” refers to the amount of time it takes for a substance, such as a binder-drug conjugate of the present invention, to lose half of its pharmacologic or physiologic activity or concentration. Biological half-life can be affected by elimination, excretion, degradation (e.g., enzymatic) of the substance, or absorption and concentration in certain organs or tissues of the body. In some embodiments, biological half-life can be assessed by determining the time it takes for the blood plasma concentration of the substance to reach half its steady state level (“plasma half-life”). To address this shortcoming, there are a variety of general strategies for prolongation of half-life that have been used in the case of other protein therapeutics, including the incorporation of half-life extending moieties as part of the Binder-drug conjugate.

The term “half-life extending moiety” refers to a pharmaceutically acceptable moiety, domain, or molecule covalently linked (“conjugated” or “fused”) to the affimer polypeptide to form the Binder-drug conjugates described herein, optionally via a non-naturally encoded amino acid, directly or via a linker, that prevents or mitigates in vivo proteolytic degradation or other activity-diminishing modification of the affimer polypeptide, increases half-life, and/or improves or alters other pharmacokinetic or biophysical properties including but not limited to increasing the rate of absorption, reducing toxicity, improving solubility, reducing protein aggregation, increasing biological activity and/or target selectivity of the modified affimer polypeptide, increasing manufacturability, and/or reducing immunogenicity of the modified affimer polypeptide, compared to a comparator such as an unconjugated form of the modified affimer polypeptide. The term “half-life extending moiety” includes non-proteinaceous, half-life extending moieties, such as a water soluble polymer such as polyethylene glycol (PEG) or discrete PEG, hydroxyethyl starch (HES), a lipid, a branched or unbranched acyl group, a branched or unbranched C8-C30 acyl group, a branched or unbranched alkyl group, and a branched or unbranched C8-C30 alkyl group; and proteinaceous half-life extending moieties, such as serum albumin, transferrin, adnectins (e.g., albumin-binding or pharmacokinetics extending (PKE) adnectins), Fc domain, and unstructured polypeptide, such as XTEN and PAS polypeptide (e.g. conformationally disordered polypeptide sequences composed of the amino acids Pro, Ala, and/or Ser), and a fragment of any of the foregoing. An examination of the crystal structure of an affimer and its interaction with its target, such as the anti-PD-L1 affimer complex with PD-1 shown in the Figures, can indicate which certain amino acid residues have side chains that are fully or partially accessible to solvent.

In certain embodiments, the half-life extending moiety extends the half-life of the resulting Binder-drug conjugate circulating in mammalian blood serum compared to the half-life of the protein that is not so conjugated to the moiety (such as relative to the Affimer polypeptide alone). In some embodiments, half-life is extended by greater than or greater than about 1.2-fold, 1.5-fold, 2.0-fold, 3.0-fold, 4.0-fold., 5.0-fold, or 6.0-fold. In some embodiments, half-life is extended by more than 6 hours, more than 12 hours, more than 24 hours, more than 48 hours, more than 72 hours, more than 96 hours or more than 1 week after in vivo administration compared to the protein without the half-life extending moiety.

As means for further exemplification, half-life extending moieties that can be used in the generation of Binder-drug conjugates of the invention include:
Genetic fusion of the pharmacologically affimer sequence to a naturally long-half-life protein or protein domain (e.g., Fc fusion, transferrin [Tf] fusion, or albumin fusion. See, for example, Beck et al. (2011) “Therapeutic Fc-fusion proteins and peptides as successful alternatives to antibodies. MAbs. 3:1-2; Czajkowsky et al. (2012) “Fc-fusion proteins: new developments and future perspectives. EMBO Mol Med. 4:1015-28; Huang et al. (2009) “Receptor-Fc fusion therapeutics, traps, and Mimetibody technology” Curr Opin Biotechnol. 2009; 20:692-9; Keefe et al. (2013) “Transferrin fusion protein therapies: acetylcholine receptor-transferrin fusion protein as a model. In: Schmidt S, editor. Fusion protein technologies for biopharmaceuticals: applications and challenges. Hoboken: Wiley; p. 345-56; Weimer et al. (2013) “Recombinant albumin fusion proteins. In: Schmidt S, editor. Fusion protein technologies for biopharmaceuticals: applications and challenges. Hoboken: Wiley; 2013. p. 297-323; Walker et al. (2013) “Albumin-binding fusion proteins in the development of novel long-acting therapeutics. In: Schmidt S, editor. Fusion protein technologies for biopharmaceuticals: applications and challenges. Hoboken: Wiley; 2013. p. 325-43.
Genetic fusion of the pharmacologically affimer sequence to an inert polypeptide, e.g., XTEN (also known as recombinant PEG or “rPEG”), a homoamino acid polymer (HAP; HAPylation), a proline-alanine-serine polymer (PAS; PASylation), or an elastin-like peptide (ELP; ELPylation). See, for example, Schellenberger et al. (2009) “A recombinant polypeptide extends the in vivo half-life of peptides and proteins in a tunable manner. Nat Biotechnol. 2009; 27:1186-90; Schlapschy et al. Fusion of a recombinant antibody fragment with a homo-amino-acid polymer: effects on biophysical properties and prolonged plasma half-life. Protein Eng Des Sel. 2007; 20:273-84; Schlapschy (2013) PASylation: a biological alternative to PEGylation for extending the plasma halflife of pharmaceutically active proteins. Protein Eng Des Sel. 26:489-501. Floss et al. (2012) “Elastin-like polypeptides revolutionize recombinant protein expression and their biomedical application. Trends Biotechnol. 28:37-45. Floss et al. “ELP-fusion technology for biopharmaceuticals. In: Schmidt S, editor. Fusion protein technologies for biopharmaceuticals: application and challenges. Hoboken: Wiley; 2013. p. 372-98.
Increasing the hydrodynamic radius by chemical conjugation of the pharmacologically active peptide or protein to repeat chemical moieties, e.g., to PEG (PEGylation) or hyaluronic acid. See, for example, Caliceti et al. (2003) “Pharmacokinetic and biodistribution properties of poly(ethylene glycol)-protein conjugates” Adv Drug Delivery Rev. 55:1261-77; Jevsevar et al. (2010) PEGylation of therapeutic proteins. Biotechnol J 5:113-28; Kontermann (2009) “Strategies to extend plasma half-lives of recombinant antibodies” BioDrugs. 23:93-109; Kang et al. (2009) “Emerging PEGylated drugs” Expert Opin Emerg Drugs. 14:363-80; and Mero et al. (2013) “Conjugation of hyaluronan to proteins” Carb Polymers. 92:2163-70.
Significantly increasing the negative charge of fusing the pharmacologically active peptide or protein by polysialylation; or, alternatively, (b) fusing a negatively charged, highly sialylated peptide (e.g., carboxy-terminal peptide [CTP; of chorionic gonadotropin (CG) b-chain]), known to extend the halflife of natural proteins such as human CG b-subunit, to the biological drug candidate. See, for example, Gregoriadis et al. (2005) “Improving the therapeutic efficacy of peptides and proteins: a role for polysialic acids” Int J Pharm. 2005; 300:125-30; Duijkers et al. “Single dose pharmacokinetics and effects on follicular growth and serum hormones of a long-acting recombinant FSH preparation (FSHCTP) in healthy pituitary-suppressed females” (2002) Hum Reprod. 17:1987-93; and Fares et al. “Design of a longacting follitropin agonist by fusing the C-terminal sequence of the chorionic gonadotropin beta subunit to the follitropin beta subunit” (1992) Proc Natl Acad Sci USA. 89:4304-8. 35; and Fares “Half-life extension through O-glycosylation.
Binding non-covalently, via attachment of a peptide or protein-binding domain to the bioactive protein, to normally long-half-life proteins such as HSA, human IgG, transferrin or fibronectin. See, for example, Andersen et al. (2011) “Extending half-life by indirect targeting of the neonatal Fc receptor (FcRn) using a minimal albumin binding domain” J Biol Chem. 286:5234-41; O'Connor-Semmes et al. (2014) “GSK2374697, a novel albumin-binding domain antibody (albudAb), extends systemic exposure of extendin-4: first study in humans-PK/PD and safety” Clin Pharmacol Ther. 2014; 96:704-12. Sockolosky et al. (2014) “Fusion of a short peptide that binds immunoglobulin G to a recombinant protein substantially increases its plasma half-life in mice” PLoS One. 2014; 9:e102566.

Classical genetic fusions to long-lived serum proteins offer an alternative method of half-life extension distinct from chemical conjugation to PEG or lipids. Two major proteins have traditionally been used as fusion partners: antibody Fc domains and human serum albumin (HSA). Fc fusions involve the fusion of peptides, proteins or receptor exodomains to the Fc portion of an antibody. Both Fc and albumin fusions achieve extended half-lives not only by increasing the size of the peptide drug, but both also take advantage of the body's natural recycling mechanism: the neonatal Fc receptor, FcRn. The pH-dependent binding of these proteins to FcRn prevents degradation of the fusion protein in the endosome. Fusions based on these proteins can have half-lives in the range of 3-16 days, much longer than typical PEGylated or lipidated peptides. Fusion to antibody Fc domains can improve the solubility and stability of the peptide or protein drug. An example of a peptide Fc fusion is dulaglutide, a GLP-1 receptor agonist currently in late-stage clinical trials. Human serum albumin, the same protein exploited by the fatty acylated peptides is the other popular fusion partner. Albiglutide is a GLP-1 receptor agonist based on this platform. A major difference between Fc and albumin is the dimeric nature of Fc versus the monomeric structure of HSA leading to presentation of a fused peptide as a dimer or a monomer depending on the choice of fusion partner. The dimeric nature of an Affimer-Fc fusion can produce an avidity effect if the Affimer target, such as PD-L1 on tumour cells, are spaced closely enough together or are themselves dimers. This may be desirable or not depending on the target.

Fc Fusions

In some embodiments, the affimer polypeptide may be part of a fusion protein with an immunoglobulin Fc domain (“Fc domain”), or a fragment or variant thereof, such as a functional Fc region. In this context, an Fc fusion (“Fc-fusion”), such as a binder-drug conjugate created as an Affimer-Fc fusion protein, is a polypeptide comprising one or more affimer sequences covalently linked through a peptide backbone (directly or indirectly) to an Fc region of an immunoglobulin. An Fc-fusion may comprise, for example, the Fc region of an antibody (which facilitates effector functions and pharmacokinetics) and an affimer sequence as part of the same polypeptide. An immunoglobulin Fc region may also be linked indirectly to one or more affimers. Various linkers are known in the art and can optionally be used to link an Fc to a polypeptide including an affimer sequence to generate an Fc-fusion. In certain embodiments, Fc-fusions can be dimerized to form Fc-fusion homodimers, or using non-identical Fe domains, to form Fc-fusion heterodimers.

There are several reasons for choosing the Fc region of human antibodies for use in generating the subject Binder-drug conjugates as affimer fusion proteins. The principle rationale is to produce a stable protein, large enough to demonstrate a similar pharmacokinetic profile compared with those of antibodies, and to take advantage of the properties imparted by the Fc region; this includes the salvage neonatal FcRn receptor pathway involving FcRn-mediated recycling of the fusion protein to the cell surface post endocytosis, avoiding lysosomal degradation and resulting in release back into the bloodstream, thus contributing to an extended serum half-life. Another obvious advantage is the Fe domain's binding to Protein A, which can simplify downstream processing during production of the Binder-drug conjugate and permit generation of highly pure preparation of the Binder-drug conjugate.

In general, an Fe domain will include the constant region of an antibody excluding the first constant region immunoglobulin domain. Thus, Fe domain refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cy2 and Cy3 and the hinge between Cy1 and Cy2. Although the boundaries of the Fe domain may vary, the human IgG heavy chain Fc region is usually defined to comprise residues C226 or P230 to its carboxyl-terminus, wherein the numbering is according to the EU index as set forth in Kabat (Kabat et al., Sequences of Proteins of Immunological Interest, 5th Ed. Public Health Service, NIH, Bethesda, Md. (1991)). Fc may refer to this region in isolation, or this region in the context of a whole antibody, antibody fragment, or Fc fusion protein. Polymorphisms have been observed at a number of different Fc positions and are also included as Fe domains as used herein.

In certain embodiments, the Fc As used herein, a “functional Fc region” refers to an Fe domain or fragment thereof which retains the ability to bind FcRn. A functional Fc region binds to FcRn, but does not possess effector function. The ability of the Fc region or fragment thereof to bind to FcRn can be determined by standard binding assays known in the art. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions can be assessed using various assays known in the art for evaluating such antibody effector functions.

In an exemplary embodiment, the Fc domain is derived from an IgG1 subclass, however, other subclasses (e.g., IgG2, IgG3, and IgG4) may also be used. An exemplary sequence of a human IgG1 immunoglobulin Fc domain which can be used is:

(SEQ ID No. 93)
DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVS
LTCLVKGFYPSDIAVEWESNGQPENNYKTTPPVLDSDGSFFLYSKLTV
DKSRWQQGNVFSCSVMHEALHNHYTQKSLSLSPGK

In some embodiments, the Fc region used in the fusion protein may comprise the hinge region of an Fc molecule. An exemplary hinge region comprises the core hinge residues spanning positions 1-16 (i.e., DKTHTCPPCPAPELLG) of the exemplary human IgG1 immunoglobulin Fc domain sequence provided above. In certain embodiments, the affimer-containing fusion protein may adopt a multimeric structure (e.g., dimer) owing, in part, to the cysteine residues at positions 6 and 9 within the hinge region of the exemplary human IgG1 immunoglobulin Fc domain sequence provided above. In other embodiments, the hinge region as used herein, may further include residues derived from the CH1 and CH2 regions that flank the core hinge sequence of the exemplary human IgG1 immunoglobulin Fc domain sequence provided above. In yet other embodiments, the hinge sequence may comprise or consist of GSTHTCPPCPAPELLG or EPKSCDKTHTCPPCPAPELLG.

In some embodiments, the hinge sequence may include one or more substitutions that confer desirable pharmacokinetic, biophysical, and/or biological properties. Some exemplary hinge sequences include:

EPKSCDKTHTCPPCPAPELLGGPS
EPKSSDKTHTCPPCPAPELLGGPS;
EPKSSDKTHTCPPCPAPELLGGSS;
EPKSSGSTHTCPPCPAPELLGGSS;
DKTHTCPPCPAPELLGGPS
and
DKTHTCPPCPAPELLGGSS.

In one embodiment, the residue P at position 18 of the exemplary human IgG1 immunoglobulin Fe domain sequence provided above may be replaced with S to ablate Fc effector function; this replacement is exemplified in hinges having the sequences EPKSSDKTHTCPPCPAPELLGGSS, EPKSSGSTHTCPPCPAPELLGGSS, and DKTHTCPPCPAPELLGGSS. In another embodiment, the residues DK at positions 1-2 of the exemplary human IgG1 immunoglobulin Fc domain sequence provided above may be replaced with GS to remove a potential clip site; this replacement is exemplified in the sequence EPKSSGSTHTCPPCPAPELLGGSS. In another embodiment, the C at the position 103 of the heavy chain constant region of human IgG1 (i.e., domains CH₁—CH₃), may be replaced with S to prevent improper cysteine bond formation in the absence of a light chain; this replacement is exemplified by EPKSSDKTHTCPPCPAPELLGGPS, EPKSSDKTHTCPPCPAPELLGGSS, and EPKSSGSTHTCPPCPAPELLGGSS.

In some embodiments, the Fc is a mammalian Fc such as a human Fc, including Fc domains derived from IgG1, IgG2, IgG3 or IgG4. The Fc region may possess at least about 80%, 85%, 90%, 95%, 96%, 97%, 98%, or 99% sequence identity with a native Fc region and/or with an Fc region of a parent polypeptide. In some embodiments, the Fc region may have at least about 90% sequence identity with a native Fc region and/or with an Fc region of a parent polypeptide.

In some embodiments, the Fc domain comprises an amino acid sequence selected from SEQ ID NOs: 93, or an Fc sequence from the examples provided by SEQ ID Nos. 94-106. It should be understood that the C-terminal lysine of an Fc domain is an optional component of a fusion protein comprising an Fc domain. In some embodiments, the Fc domain comprises an amino acid sequence selected from SEQ ID NOs: 93-106, except that the C-terminal lysine thereof is omitted. In some embodiments, the Fc domain comprises the amino acid sequence of SEQ ID NO: 93. In some embodiments, the Fc domain comprises the amino acid sequence of SEQ ID NOs: 93 except the C-terminal lysine thereof is omitted.

| | |
|---|---|
| hIgG1a_191 | DKTHTCPPCPAPELLGGPSVFLFPPKPKDTLMISRTPEVTCVVVD |
| [A subtype] | VSHEDPEVKFNWYVDGVEVHNAKTKPREEQYNSTYRVVSVLTVLH |
| (SEQ ID No. 94) | QDWLNGKEYKCKVSNKALPAPIEKTISKAKGQPREPQVYTLPPSR |
| | DELTKNQVSLTCLVKGFYPSDIAVEWESNGQPENNYKTTPPVLDS |
| | DGSFFLYSKLTVDKSRWQQGNVFSCSVMHEALHNHYTQKSLSLSP |
| | GK |
| hIgG1a_189 | DKTHTCPPCPAPELLGGPSVFLFPPKPKDTLMISRTPEVTCVVVD |
| [hIgG1a_191 sans “GK” | VSHEDPEVKFNWYVDGVEVHNAKTKPREEQYNSTYRVVSVLTVLH |
| on C term; A subtype] | QDWLNGKEYKCKVSNKALPAPIEKTISKAKGQPREPQVYTLPPSR |
| (SEQ ID No. 95) | DELTKNQVSLTCLVKGFYPSDIAVEWESNGQPENNYKTTPPVLDS |
| | DGSFFLYSKLTVDKSRWQQGNVFSCSVMHEALHNHYTQKSLSLSP |
| hIgG1a_191b | DKTHTCPPCPAPELLGGPSVFLFPPKPKDTLMISRTPEVTCVVVD |
| [A/F subtype] | VSHEDPEVKFNWYVDGVEVHNAKTKPREEQYNSTYRVVSVLTVLH |
| (SEQ ID No. 96) | QDWLNGKEYKCKVSNKALPAPIEKTISKAKGQPREPQVYTLPPSR |
| | DEMTKNQVSLTCLVKGFYPSDIAVEWESNGQPENNYKTTPPVLDS |
| | DGSFFLYSKLTVDKSRWQQGNVFSCSVMHEALHNHYTQKSLSLSP |
| | GK |
| hIgG1f_1.1_191 | DKTHTCPPCPAPEAEGAPSVFLFPPKPKDTLMISRTPEVTCVVVD |
| [Contains 5 point | VSHEDPEVKFNWYVDGVEVHNAKTKPREEQYNSTYRVVSVLTVLH |
| mutations to alter | QDWLNGKEYKCKVSNKALPSSIEKTISKAKGQPREPQVYTLPPSR |
| ADCC function, F | EEMTKNQVSLTCLVKGFYPSDIAVEWESNGQPENNYKTTPPVLDS |
| subtype] | DGSFFLYSKLTVDKSRWQQGNVFSCSVMHEALHNHYTQKSLSLSP |
| (SEQ ID No. 97) | GK |
| hIgG1f_1.1_186 | EPKSSDKTHTCPPCPAPEAEGAPSVFLFPPKPKDTLMISRTPEVT |
| [Contains 5 point | CVVVDVSHEDPEVKFNWYVDGVEVHNAKTKPREEQYNSTYRVVSV |
| mutations to alter ADCC | LTVLHQDWLNGKEYKCKVSNKALPSSIEKTISKAKGQPREPQVYT |
| function and C225S | LPPSREEMTKNQVSLTCLVKGFYPSDIAVEWESNGQPENNYKTTP |
| (Edlemen numbering); | PVLDSDGSFFLYSKLTVDKSRWQQGNVFSCSVMHEALHNHYTQKS |
| F subtype | LSLSPGK |
| (SEQ ID No. 98) | |
| hIgG1a_(N297G)_191 | DKTHTCPPCPAPELLGGPSVFLFPPKPKDTLMISRTPEVTCVVVD |
| [A subtype] | VSHEDPEVKFNWYVDGVEVHNAKTKPREEQYGSTYRVVSVLTVLH |
| (SEQ ID No. 99) | QDWLNGKEYKCKVSNKALPAPIEKTISKAKGQPREPQVYTLPPSR |
| | DELTKNQVSLTCLVKGFYPSDIAVEWESNGQPENNYKTTPPVLDS |
| | DGSFFLYSKLTVDKSRWQQGNVFSCSVMHEALHNHYTQKSLSLSP |
| | GK |
| hIgG1a_190 | DKTHTCPPCPAPELLGGPSVFLFPPKPKDTLMISRTPEVTCVVVD |
| [hIgG1a_190 sans “K” | VSHEDPEVKFNWYVDGVEVHNAKTKPREEQYNSTYRVVSVLTVLH |
| on C term; A subtype] | QDWLNGKEYKCKVSNKALPAPIEKTISKAKGQPREPQVYTLPPSR |
| (SEQ ID No. 100) | DELTKNQVSLTCLVKGFYPSDIAVEWESNGQPENNYKTTPPVLDS |
| | DGSFFLYSKLTVDKSRWQQGNVFSCSVMHEALHNHYTQKSLSLSP |
| | G |
| hIgG1a_(N297Q)_191 | DKTHTCPPCPAPELLGGPSVFLFPPKPKDTLMISRTPEVTCVVVD |
| [A subtype] | VSHEDPEVKFNWYVDGVEVHNAKTKPREEQYQSTYRVVSVLTVLH |
| (SEQ ID No. 101) | QDWLNGKEYKCKVSNKALPAPIEKTISKAKGQPREPQVYTLPPSR |
| | DELTKNQVSLTCLVKGFYPSDIAVEWESNGQPENNYKTTPPVLDS |
| | DGSFFLYSKLTVDKSRWQQGNVFSCSVMHEALHNHYTQKSLSLSP |
| | GK |
| hIgG1a_(N297S)_191 | DKTHTCPPCPAPELLGGPSVFLFPPKPKDTLMISRTPEVTCVVVD |
| [A subtype] | VSHEDPEVKFNWYVDGVEVHNAKTKPREEQYSSTYRVVSVLTVLH |
| (SEQ ID No. 102) | QDWLNGKEYKCKVSNKALPAPIEKTISKAKGQPREPQVYTLPPSR |
| | DELTKNQVSLTCLVKGFYPSDIAVEWESNGQPENNYKTTPPVLDS |
| | DGSFFLYSKLTVDKSRWQQGNVFSCSVMHEALHNHYTQKSLSLSP |
| | GK |
| hIgG1a_(N297A)_191 | DKTHTCPPCPAPELLGGPSVFLFPPKPKDTLMISRTPEVTCVVVD |
| [A subtype] | VSHEDPEVKFNWYVDGVEVHNAKTKPREEQYASTYRVVSVLTVLH |
| (SEQ ID No. 103) | QDWLNGKEYKCKVSNKALPAPIEKTISKAKGQPREPQVYTLPPSR |
| | DELTKNQVSLTCLVKGFYPSDIAVEWESNGQPENNYKTTPPVLDS |
| | DGSFFLYSKLTVDKSRWQQGNVFSCSVMHEALHNHYTQKSLSLSP |
| | GK |
| hIgG1a_(N297H)_191 | DKTHTCPPCPAPELLGGPSVFLFPPKPKDTLMISRTPEVTCVVVD |
| [A subtype] | VSHEDPEVKFNWYVDGVEVHNAKTKPREEQYHSTYRVVSVLTVLH |
| (SEQ ID No. 104) | QDWLNGKEYKCKVSNKALPAPIEKTISKAKGQPREPQVYTLPPSR |
| | DELTKNQVSLTCLVKGFYPSDIAVEWESNGQPENNYKTTPPVLDS |
| | DGSFFLYSKLTVDKSRWQQGNVFSCSVMHEALHNHYTQKSLSLSP |
| | GK |
| hIgG4 | DKRVESKYGPPCPSCPAPEFLGGPSVFLFPPKPKDTLMISRTPEV |
| (SEQ ID No. 105) | TCVVVDVSQEDPEVQFNWYVDGVEVHNAKTKPREEQFNSTYRVVS |
| | VLTVLHQDWLNGKEYKCKVSNKGLPSSIEKTISKAKGQPREPQVY |
| | TLPPSQEEMTKNQVSLTCLVKGFYPSDIAVEWESNGQPENNYKTT |
| | PPVLDSDGSFFLYSRLTVDKSRWQEGNVFSCSVMHEALHNHYTQK |
| | SLSLSLGK |
| hIgG4_(S241P) | DKRVESKYGPPCPPCPAPEFLGGPSVFLFPPKPKDTLMISRTPEV |
| (SEQ ID No. 106) | TCVVVDVSQEDPEVQFNWYVDGVEVHNAKTKPREEQFNSTYRVVS |
| | VLTVLHQDWLNGKEYKCKVSNKGLPSSIEKTISKAKGQPREPQVY |
| | TLPPSQEEMTKNQVSLTCLVKGFYPSDIAVEWESNGQPENNYKTT |
| | PPVLDSDGSFFLYSRLTVDKSRWQEGNVFSCSVMHEALHNHYTQK |
| | SLSLSLGK |

Exemplary Fc fusions of a PD-L1 binding Affimer with an Fe are provided in the Examples and Figures, demonstrating that the affimer sequence can be placed at either the N-terminal or C-terminal end of the Fc domain, and may be attached directly or the fusion protein may have other polypeptide sequences intervening between the Fc domain and the affimer polypeptide sequence. In the illustrated examples, an unstructured (flexible) linker, (Gly₄Ser)ₙ, is used with PD-L1 Binding Affimer “251” (SEQ ID No. 84) and the Fc domain of human IgG1 (SEQ ID No. 93) with the hinge region being EPKSCDKTHTCPPCPAPELLG. The constructs both included the CD33 secretion signal sequence MPLLLLLPLLWAGALA which is cleaved from mature versions of the protein.

| | |
|---|---|
| PD-L1 251 Fc1 | MPLLLLLPLLWAGALAIPRGLSEAKPATPE |
| (N-term Affimer) | IQEIVDKVKPQLEEKTGETYGKLEAVQYKT |
| (SEQ ID No. 108) | QVLAREGRQDWVLSTNYYIKVRAGDNKYMH |
| | LKVFNGPWVPFPHQQLADRVLTGYQVDKNK |
| | DDELTGFAAAGGGGSGGGGSGGGGSGGGGS |
| | EPKSCDKTHTCPPCPAPELLGGPSVFLFPP |
| | KPKDTLMISRTPEVTCVVVDVSHEDPEVKF |
| | NWYVDGVEVHNAKTKPREEQYNSTYRVVSV |
| | LTVLHQDWLNGKEYKCKVSNKALPAPIEKT |
| | ISKAKGQPREPQVYTLPPSREEMTKNQVSL |
| | TCLVKGFYPSDIAVEWESNGQPENNYKTTP |
| | PVLDSDGSFFLYSKLTVDKSRWQQGNVFSC |
| | SVMHEALHNHYTQKSLSLSPGK |
| PD-L1 251 Fc1 | MPLLLLLPLLWAGALAEPKSCDKTHTCPPC |
| (C-term Affimer) | PAPELLGGPSVFLFPPKPKDTLMISRTPEV |
| (SEQ ID No. 110) | TCVVVDVSHEDPEVKFNWYVDGVEVHNAKT |
| | KPREEQYNSTYRVVSVLTVLHQDWLNGKEY |
| | KCKVSNKALPAPIEKTISKAKGQPREPQVY |
| | TLPPSREEMTKNQVSLTCLVKGFYPSDIAV |
| | EWESNGQPENNYKTTPPVLDSDGSFFLYSK |
| | LTVDKSRWQQGNVFSCSVMHEALHNHYTQK |
| | SLSLSPGKGGGGSGGGGSGGGGSGGGGSIP |
| | RGLSEAKPATPEIQEIVDKVKPQLEEKTGE |
| | TYGKLEAVQYKTQVLAREGRQDWVLSTNYY |
| | IKVRAGDNKYMHLKVENGPWVPFPHQQLAD |
| | RVLTGYQVDKNKDDELTGFAAA |

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enables these cytotoxic effector cells to bind specifically to an antigen-bearing target cell and subsequently kill the target cell with cytotoxins.

In certain embodiments, the fusion protein includes an Fc domain sequence for which the resulting Binder-drug conjugate has no (or reduced) ADCC and/or complement activation or effector functionality. For example, the Fc domain may comprise a naturally disabled constant region of IgG2 or IgG4 isotype or a mutated IgG1 constant region. Examples of suitable modifications are described in EP0307434. One example comprises the substitutions of alanine residues at positions 235 and 237 (EU index numbering).

In other embodiments, the fusion protein includes an Fc domain sequence for which the resulting Binder-drug conjugate will retain some or all Fc functionality for example will be capable of one or both of ADCC and CDC activity, as for example if the fusion protein comprises the Fc domain from human IgG1 or IgG3. Levels of effector function can be varied according to known techniques, for example by mutations in the CH2 domain, for example wherein the IgG1 CH2 domain has one or more mutations at positions selected from 239 and 332 and 330, for example the mutations are selected from S239D and I332E and A330L such that the antibody has enhanced effector function, and/or for example altering the glycosylation profile of the antigen-binding protein of the invention such that there is a reduction in fucosylation of the Fc region.

Albumin Fusion

In other embodiments, the Binder-drug conjugate is a fusion protein comprising, in addition to at least one affimer sequence, an albumin sequence or an albumin fragment. In other embodiments, the Binder-drug conjugate is conjugated to the albumin sequence or an albumin fragment through chemical linkage other than incorporation into the polypeptide sequence including the affimer. In some embodiments, the albumin, albumin variant, or albumin fragment is human serum albumin (HSA), a human serum albumin variant, or a human serum albumin fragment. Albumin serum proteins comparable to HSA are found in, for example, cynomolgus monkeys, cows, dogs, rabbits and rats. Of the non-human species, bovine serum albumin (BSA) is the most structurally similar to HSA. See, e.g., Kosa et al., (2007) J Pharm Sci. 96(11):3117-24. The present disclosure contemplates the use of albumin from non-human species, including, but not limited to, albumin sequence derived from cyno serum albumin or bovine serum albumin.

Mature HSA, a 585 amino acid polypeptide (approx. 67 kDa) having a serum half-life of about 20 days, is primarily responsible for the maintenance of colloidal osmotic blood pressure, blood pH, and transport and distribution of numerous endogenous and exogenous ligands. The protein has three structurally homologous domains (domains I, II and III), is almost entirely in the alpha-helical conformation, and is highly stabilized by 17 disulphide bridges. In certain preferred embodiments, the Binder-drug conjugate can be an albumin fusion protein including one or more affimer polypeptide sequences and the sequence for mature human serum albumin (SEQ ID No. 111) or a variant or fragment thereof which maintains the PK and/or biodistribution properties of mature albumin to the extent desired in the fusion protein.

(SEQ ID No. 111)
DAHKSEVAHRFKDLGEENFKALVLIAFAQYLQQCPFEDHVKLVNEV
TEFAKTCVADESAENCDKSLHTLFGDKLCTVATLRETYGEMADCCA
KQEPERNECFLQHKDDNPNLPRLVRPEVDVMCTAFHDNEETFLKKY
LYEIARRHPYFYAPELLFFAKRYKAAFTECCQAADKAACLLPKLDEL
RDEGKASSAKQRLKCASLQKFGERAFKAWAVARLSQRFPKAEFAEV
SKLVTDLTKVHTECCHGDLLECADDRADLAKYICENQDSISSKLKEC
CEKPLLEKSHCIAEVENDEMPADLPSLAADFVESKDVCKNYAEAKD
VFLGMFLYEYARRHPDYSVVLLLRLAKTYETTLEKCCAAADPHECY
AKVFDEFKPLVEEPQNLIKQNCELFEQLGEYKFQNALLVRYTKKVPQ
VSTPTLVEVSRNLGKVGSKCCKHPEAKRMPCAEDYLSVVLNQLCVL
HEKTPVSDRVTKCCTESLVNRRPCFSALEVDETYVPKEFNAETFTFH
ADICTLSEKERQIKKQTALVELVKHKPKATKEQLKAVMDDFAAFVE
KCCKADDKETCFAEEGKKLVAASQAALGL

The albumin sequence can be set off from the affimer polypeptide sequence or other flanking sequences in the Binder-drug conjugate by use of linker sequences as described above.

While unless otherwise indicated, reference herein to “albumin” or to “mature albumin” is meant to refer to HSA. However, it is noted that full-length HSA has a signal peptide of 18 amino acids (MKWVTFISLLFLFSSAYS) followed by a pro-domain of 6 amino acids (RGVFRR); this 24 amino acid residue peptide may be referred to as the pre-pro domain. The Affimer-HSA fusion proteins can be expressed and secreted using the HSA pre-pro-domain in the recombinant proteins coding sequence. Alternatively, the affimer-HSA fusion can be expressed and secreted through inclusion of other secretion signal sequences, such as described above.

In alternative embodiments, rather than provided as part of a fusion protein with the affimer polypeptide, the serum albumin polypeptide can be covalently coupled to the affimer-containing polypeptide through a bond other than a backbone amide bond, such as cross-linked through chemical conjugation between amino acid sidechains on each of the albumin polypeptide and the affimer-containing polypeptide.

Albumin Binding Domain

In certain embodiments, the Binder-drug conjugate can include a serum-binding moiety—either as part of a fusion protein (if also a polypeptide) with the affimer polypeptide sequence or chemically conjugated through a site other than being part of a contiguous polypeptide chain.

In certain embodiments, the serum-binding polypeptide is an albumin binding moiety. Albumin contains multiple hydrophobic binding pockets and naturally serves as a transporter of a variety of different ligands such as fatty acids and steroids as well as different drugs. Furthermore, the surface of albumin is negatively charged making it highly water-soluble.

The term “albumin binding moiety” as used herein refers to any chemical group capable of binding to albumin, i.e. has albumin binding affinity. Albumin binds to endogenous ligands such as fatty acids; however, it also interacts with exogenous ligands such as warfarin, penicillin and diazepam. As the binding of these drugs to albumin is reversible the albumin-drug complex serves as a drug reservoir that can enhance the drug biodistribution and bioavailability. Incorporation of components that mimic endogenous albumin-binding ligands, such as fatty acids, has been used to potentiate albumin association and increase drug efficacy.

In certain embodiments, a chemical modification method that can be applied in the generation of the subject Binder-drug conjugates to increase protein half-life is lipidation, which involves the covalent binding of fatty acids to peptide side chains. Originally conceived of and developed as a method for extending the half-life of insulin, lipidation shares the same basic mechanism of half-life extension as PEGylation, namely increasing the hydrodynamic radius to reduce renal filtration. However, the lipid moiety is itself relatively small and the effect is mediated indirectly through the non-covalent binding of the lipid moiety to circulating albumin. One consequence of lipidation is that it reduces the water-solubility of the peptide but engineering of the linker between the peptide and the fatty acid can modulate this, for example by the use of glutamate or mini PEGs within the linker. Linker engineering and variation of the lipid moeity can affect self-aggregation which can contribute to increased half-life by slowing down biodistribution, independent of albumin. See, for example, Jonassen et al. (2012) Pharm Res. 29(8):2104-14.

Other examples of albumin binding moieties for use in the generation of certain Binder-drug conjugates include albumin-binding (PKE2) adnectins (See WO2011140086 “Serum Albumin Binding Molecules”, WO2015143199 “Serum albumin-binding Fibronectin Type III Domains” and WO2017053617 “Fast-off rate serum albumin binding fibronectin type iii domains”), the albumin binding domain 3 (ABD3) of protein G of Streptococcus strain G148, and the albumin binding domain antibody GSK2374697 (“AlbudAb”) or albumin binding nanobody portion of ATN-103 (Ozoralizumab).

PEGylation, XTEN, PAS and Other Polymers

A wide variety of macromolecular polymers and other molecules can be linked to the affimer containing polypeptides of the present disclosure to modulate biological properties of the resulting Binder-drug conjugate, and/or provide new biological properties to the Binder-drug conjugate. These macromolecular polymers can be linked to the affimer containing polypeptide via a naturally encoded amino acid, via a non-naturally encoded amino acid, or any functional substituent of a natural or non-natural amino acid, or any substituent or functional group added to a natural or non-natural amino acid. The molecular weight of the polymer may be of a wide range, including but not limited to, between about 100 Da and about 100,000 Da or more. The molecular weight of the polymer may be between about 100 Da and about 100,000 Da, including but not limited to, 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, and 100 Da. In some embodiments, the molecular weight of the polymer is between about 100 Da and about 50,000 Da. In some embodiments, the molecular weight of the polymer is between about 100 Da and about 40,000 Da. In some embodiments, the molecular weight of the polymer is between about 1,000 Da and about 40,000 Da. In some embodiments, the molecular weight of the polymer is between about 5,000 Da and about 40,000 Da. In some embodiments, the molecular weight of the polymer is between about 10,000 Da and about 40,000 Da.

For this purpose, various methods including pegylation, polysialylation, HESylation, glycosylation, or recombinant PEG analogue fused to flexible and hydrophilic amino acid chain (500 to 600 amino acids) have been developed (See Chapman, (2002) Adv Drug Deliv Rev. 54. 531-545; Schlapschy et al., (2007) Prot Eng Des Sel. 20, 273-283; Contermann (2011) Curr Op Biotechnol. 22, 868-876; Jevsevar et al., (2012) Methods Mol Biol. 901, 233-246).

Examples of polymers include but are not limited to polyalkyl ethers and alkoxy-capped analogs thereof (e.g., polyoxyethylene glycol, polyoxyethylene/propylene glycol, and methoxy or ethoxy-capped analogs thereof, especially polyoxyethylene glycol, the latter is also known as polyethylene glycol or PEG); discrete PEG (dPEG); polyvinylpyrrolidones; polyvinylalkyl ethers; polyoxazolines, polyalkyl oxazolines and polyhydroxyalkyl oxazolines; polyacrylamides, polyalkyl acrylamides, and polyhydroxyalkyl acrylamides (e.g., polyhydroxypropylmethacrylamide and derivatives thereof); polyhydroxyalkyl acrylates; polysialic acids and analogs thereof; hydrophilic peptide sequences; polysaccharides and their derivatives, including dextran and dextran derivatives, e.g., carboxymethyldextran, dextran sulfates, aminodextran; cellulose and its derivatives, e.g., carboxymethyl cellulose, hydroxyalkyl celluloses; chitin and its derivatives, e.g., chitosan, succinyl chitosan, carboxymethylchitin, carboxymethylchitosan; hyaluronic acid and its derivatives; starches; alginates; chondroitin sulfate; albumin; pullulan and carboxymethyl pullulan; polyaminoacids and derivatives thereof, e.g., polyglutamic acids, polylysines, polyaspartic acids, polyaspartamides; maleic anhydride copolymers such as: styrene maleic anhydride copolymer, divinylethyl ether maleic anhydride copolymer; polyvinyl alcohols; copolymers thereof, terpolymers thereof; mixtures thereof; and derivatives of the foregoing.

The polymer selected may be water soluble so that the Binder-drug conjugate to which it is attached does not precipitate in an aqueous environment, such as a physiological environment. The water soluble polymer may be any structural form including but not limited to linear, forked or branched. Typically, the water soluble polymer is a poly(alkylene glycol), such as poly(ethylene glycol) (PEG), but other water soluble polymers can also be employed. By way of example, PEG is used to describe certain embodiments of this disclosure. For therapeutic use of the Binder-drug conjugate, the polymer may be pharmaceutically acceptable.

The term “PEG” is used broadly to encompass any polyethylene glycol molecule, without regard to size or to modification at an end of the PEG, and can be represented as linked to the affimer containing polypeptide by the formula:

XO—(CH₂CH₂O)ₙ—CH₂CH₂—

or

XO—(CH₂CH₂O)ₙ—

where n is 2 to 10,000 and X is H or a terminal modification, including but not limited to, a C1-4 alkyl, a protecting group, or a terminal functional group. In some cases, a PEG used in the polypeptides of the disclosure terminates on one end with hydroxy or methoxy, i.e., X is H or CH₃ (“methoxy PEG”).

It is noted that the other end of the PEG, which is shown in the above formulas by a terminal “—” may attach to the affimer containing polypeptide via a naturally-occurring or non-naturally encoded amino acid. For instance, the attachment may be through an amide, carbamate or urea linkage to an amine group (including but not limited to, the epsilon amine of lysine or the N-terminus) of the polypeptide. Alternatively, the polymer is linked by a maleimide linkage to a thiol group (including but not limited to, the thiol group of cysteine)—which in the case of attachment to the affimer polypeptide sequence per se requires altering a residue in the affimer sequence to a cysteine.

The number of water soluble polymers linked to the affimer-containing polypeptide (i.e., the extent of PEGylation or glycosylation) can be adjusted to provide an altered (including but not limited to, increased or decreased) pharmacologic, pharmacokinetic or pharmacodynamic characteristic such as in vivo half-life in the resulting Binder-drug conjugate. In some embodiments, the half-life of the resulting Binder-drug conjugate is increased at least about 10, 20, 30, 40, 50, 60, 70, 80, 90 percent, 2-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 50-fold, or at least about 100-fold over an unmodified polypeptide.

Another variation of polymer system useful to modify the PK or other biological properties of the resulting Binder-drug conjugate are the use of unstructured, hydrophilic amino acid polymers that are functional analogs of PEG, particularly as part of a fusion protein with the affimer polypeptide sequence. The inherent biodegradability of the polypeptide platform makes it attractive as a potentially more benign alternative to PEG. Another advantage is the precise molecular structure of the recombinant molecule in contrast to the polydispersity of PEG. Unlike HSA and Fc peptide fusions, in which the three-dimensional folding of the fusion partner needs to be maintained, the recombinant fusions to unstructured partners can, in many cases, be subjected to higher temperatures or harsh conditions such as HPLC purification.

One of the more-advanced of this class of polypeptides is termed XTEN (Amunix) and is 864 amino acids long and comprised of six amino acids (A, E, G, P, S and T). See Schellenberger et al. “A recombinant polypeptide extends the in vivo half-life of peptides and proteins in a tunable manner” 2009 Nat Biotechnol. 27(12):1186-90. Enabled by the biodegradable nature of the polymer, this is much larger than the 40 KDa PEGs typically used and confers a concomitantly greater half-life extension. The fusion of XTEN to the affimer containing polypeptide should result in halflife extension of the final Binder-drug conjugate by 60- to 130-fold over the unmodified polypeptide.

A second polymer based on similar conceptual considerations is PAS (XL-Protein GmbH). Schlapschy et al. “PASYlation: a biological alternative to PEGylation for extending the plasma half-life of pharmaceutically active proteins” 2013 Protein Eng Des Sel. 26(8):489-501. A random coil polymer comprised of an even more restricted set of only three small uncharged amino acids, proline, alanine and serine. AS with Fc, HAS and XTEN, the PAS modification can be genetically encoded with the affimer polypeptide sequence to produce an inline fusion protein when expressed.

Multispecific Fusion Proteins

In certain embodiments, the Binder-drug conjugate is a multi-specific polypeptide including, for example, a first anti-PD-L1 affimer polypeptide and at least one additional binding domain. The additional binding domain may be a polypeptide sequence selected from amongst, to illustrate, a second affimer polypeptide sequence (which may be the same or different than the first affimer polypeptide sequence), an antibody or fragment thereof or other antigen binding polypeptide, a ligand binding portion of a receptor (such as a receptor trap polypeptide), a receptor-binding ligand (such as a cytokine, growth factor or the like), engineered T-cell receptor, an enzyme or catalytic fragment thereof, or other polypeptide sequence that confers some

In certain embodiments, the Binder-drug conjugate includes one or more additional affimer polypeptide sequence that are also directed to PD-L1. The additional anti-PD-L1 affimers may be the same or different (or a mixture thereof) as the first anti-PD-L1 affimer polypeptide in order to create a multi-specific affimer fusion protein. The Binder-drug conjugates can bind the same or overlapping sites on PD-L1, or can bind two different sites such that the Binder-drug conjugate can simultaneously bind two sites on the same PD-L1 protein (biparatopic) or more than two sites (multiparatopic).

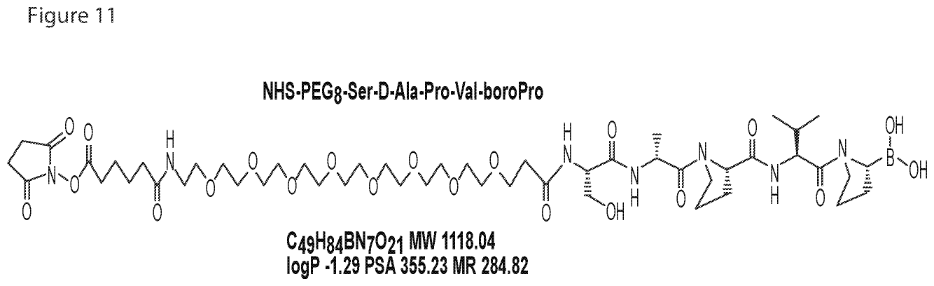

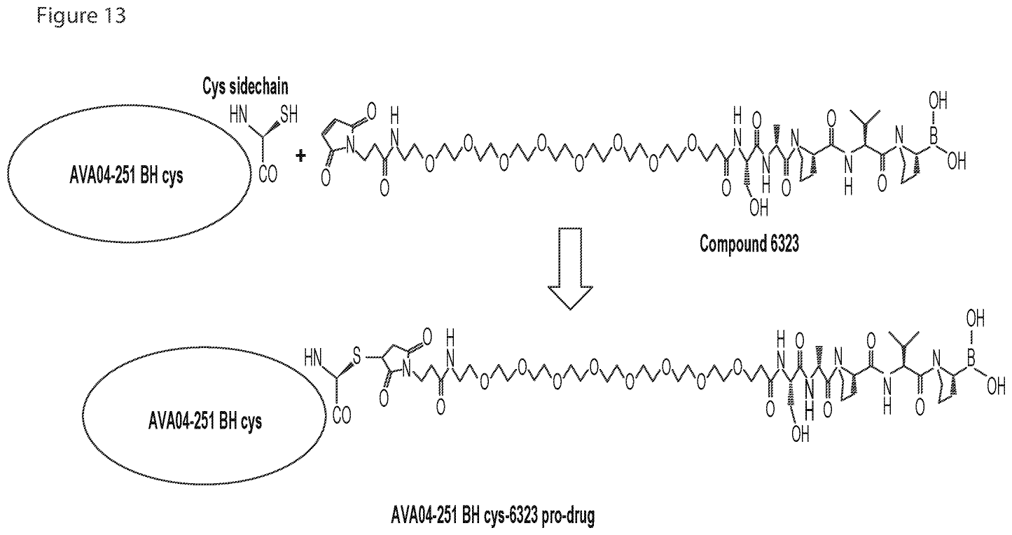

In certain embodiments, the Binder-drug conjugate includes one or more antigen binding sites from an antibody. The resulting Binder-drug conjugate can be a single chain including both the anti-PD-L1 affimer and the antigen binding site (such as in the case of an scFV), or can be a multimeric protein complex such as in antibody assembled with heavy and/or light chains to which the sequence of the anti-PD-L1 antibody has also been fused. An exemplary affimer/antibody fusion of this format is the Ipilimumab-AVA04-141 bispecific antibody shown in FIG. 11A, which is divalent for each of CTLA-4 and PD-L1. Another is the Bevacizumab-AVA04-251 bispecific antibody shown in FIG. 13A, which is divalent for each of VEGF-A and PD-L1.

In the case of the illustrated Ipilimumab-AVA04-141 bispecific antibody, the anti-PD-L1 affimer polypeptide is provided as an in-line fusion at the C-terminal end of the heavy chain of the anti-CTLA-4 antibody, where the heavy chain (including the secretion signal sequence MPLLLLLPLLWAGALA which can be removed, and a Gly₄-Ser repeat linker) has the affimer fusion sequence:

(SEQ ID No. 112)
MPLLLLLPLLWAGALAQVQLVESGGGVVQPGRSLRLSCAASGFTFSS
YTMHWVRQAPGKGLEWVTFISYDGNNKYYADSVKGRFTISRDNSKN
TLYLQMNSLRAEDTAIYYCARTGWLGPFDYWGQGTLVTVSSASTKG
PSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPK
SCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQD
WLNGKEYKCKVSNKALPAPIEKTISKAKGQPREPQVYTLPPSRDELTK
NQVSLTCLVKGFYPSDIAVEWESNGQPENNYKTTPPVLDSDGSFFLYS
KLTVDKSRWQQGNVFSCSVMHEALHNHYTQKSLSLSPGKGGGGSGG
GGSGGGGSIPRGLSEAKPATPEIQEIVDKVKPQLEEKTNETYGKLEAV
QYKTQVLAAAHFPEHFWSTNYYIKVRAGDNKYMHLKVFNGPQPAD
MSAEFADRVLTGYQVDKNKDDELTGF

And the light chain (including the secretion signal sequence MPLLLLLPLLWAGALA which can be removed) has the sequence of the native Ipilimumab antibody:

(SEQ ID No. 113)
MPLLLLLPLLWAGALAEIVLTQSPGTLSLSPGERATLSCRASQSVGSS
YLAWYQQKPGQAPRLLIYGAFSRATGIPDRFSGSGSGTDFTLTISRLE
PEDFAVYYCQQYGSSPWTFGQGTKVEIKRTVAAPSVFIFPPSDEQLKS
GTASVVCLLNNFYPREAKVQWKVDNALQSGNSQESVTEQDSKDST
YSLSSTLTLSKADYEKHKVYACEVTHQGLSSPVTKSFNRGEC

Likewise, in the case of the illustrated Bevacizumab-AVA04-251 bispecific antibody, the anti-PD-L1 affimer polypeptide is provided as an in-line fusion at the C-terminal end of the heavy chain of the anti-VEGF-A antibody, where the heavy chain (including the secretion signal sequence MPLLLLLPLLWAGALA which can be removed and a flexible Gly₄-Ser repeat linker) has the affimer fusion sequence:

(SEQ ID No. 114)
MPLLLLLPLLWAGALAEVQLVESGGGLVQPGGSLRLSCAASGYTFTN
YGMNWVRQAPGKGLEWVGWINTYTGEPTYAADFKRRFTFSLDTSKS
TAYLQMNSLRAEDTAVYYCAKYPHYYGSSHWYFDVWGQGTLVTVS
SASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVD
KKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVL
TVLHQDWLNGKEYKCKVSNKALPAPIEKTISKAKGQPREPQVYTLPP
SREEMTKNQVSLTCLVKGFYPSDIAVEWESNGQPENNYKTTPPVLDS
DGSFFLYSKLTVDKSRWQQGNVFSCSVMHEALHNHYTQKSLSLSPG
GGGGSGGGGSGGGGSIPRGLSEAKPATPEIQEIVDKVKPQLEEKTGET
YGKLEAVQYKTQVLAREGRQDWVLSTNYYIKVRAGDNKYMHLKVF
NGPWVPFPHQQLADRVLTGYQVDKNKDDELTGF

And the light chain (including the secretion signal sequence MPLLLLLPLLWAGALA which can be removed) has the sequence of the native Bevacizumab antibody:

(SEQ ID No. 115)
MPLLLLLPLLWAGALADIQMTQSPSSLSASVGDRVTITCSASQDISNY
LNWYQQKPGKAPKVLIYFTSSLHSGVPSRFSGSGSGTDFTLTISSLQP
EDFATYYCQQYSTVPWTFGQGTKVEIKRTVAAPSVFIFPPSDEQLKS
GTASVVCLLNNFYPREAKVQWKVDNALQSGNSQESVTEQDSKDST
YSLSSTLTLSKADYEKHKVYACEVTHQGLSSPVTKSFNRGEC

To further illustrate the flexibility in formatting the affimers of the present invention provide, a version of the Bevacizumab-AVA04-251 bispecific antibody was also generated in which the light chain was the same as above but the heavy chain included a rigid linker between the antibody heavy chain and anti-PD-L1 affimer, where the heavy chain (including the secretion signal sequence MPLLLLLPLLWAGALA which can be removed and a rigid A(EAAAK)₃ linker) has the affimer fusion sequence:

(SEQ ID No. 116)
MPLLLLLPLLWAGALAEVQLVESGGGLVQPGGSLRLSCAASGYTFTN
YGMNWVRQAPGKGLEWVGWINTYTGEPTYAADFKRRFTFSLDTSKS
TAYLQMNSLRAEDTAVYYCAKYPHYYGSSHWYFDVWGQGTLVTVS
SASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVD
KKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVL
TVLHQDWLNGKEYKCKVSNKALPAPIEKTISKAKGQPREPQVYTLPP
SREEMTKNQVSLTCLVKGFYPSDIAVEWESNGQPENNYKTTPPVLDS
DGSFFLYSKLTVDKSRWQQGNVFSCSVMHEALHNHYTQKSLSLSPG
KAEAAAKEAAAKEAAAKIPRGLSEAKPATPEIQEIVDKVKPQLEEKT
GETYGKLEAVQYKTQVLAREGRQDWVLSTNYYIKVRAGDNKYMHL
KVFNGPWVPFPHQQLADRVLTGYQVDKNKDDELTGF

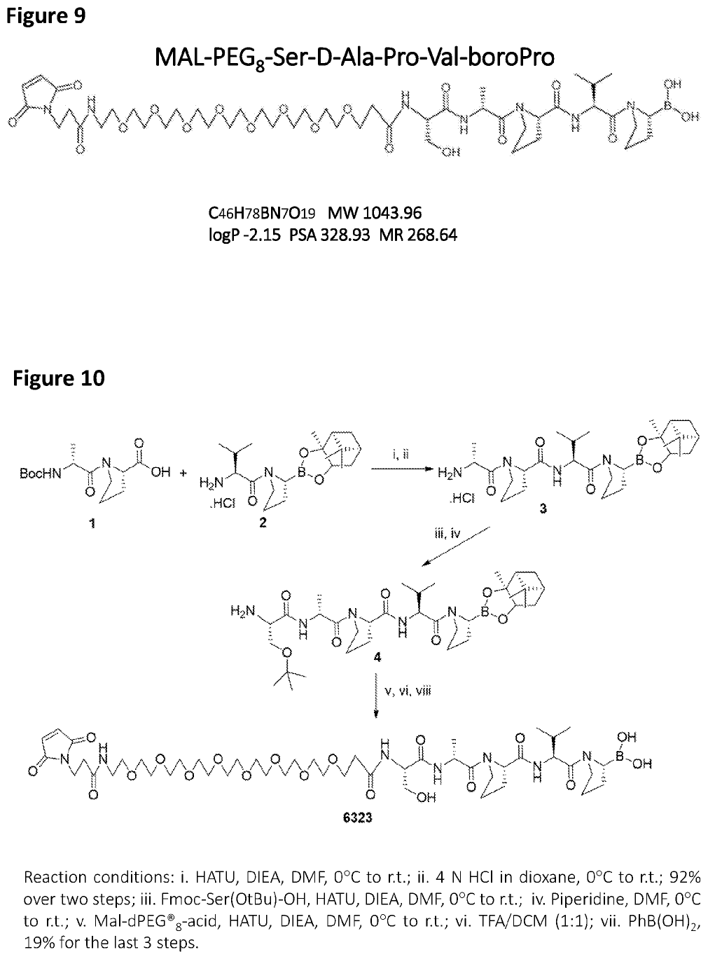

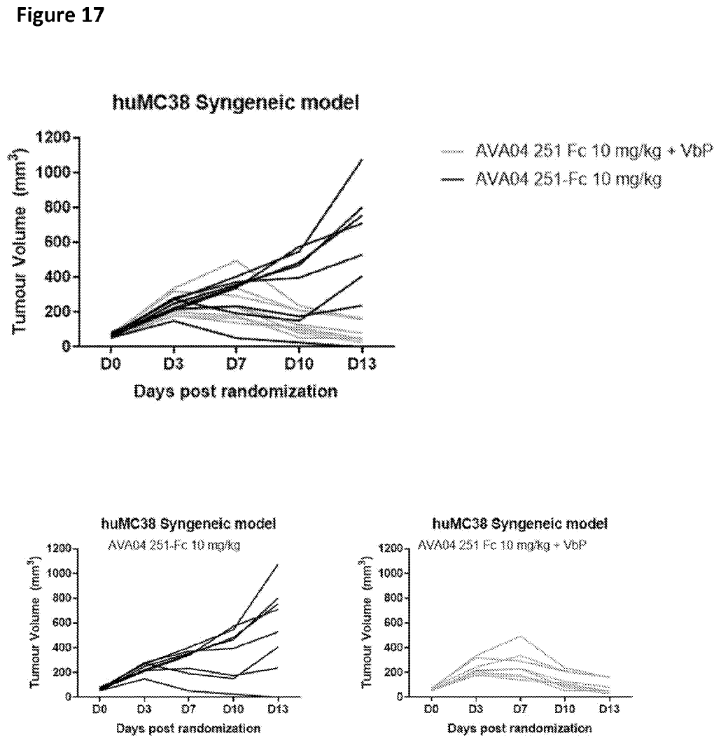

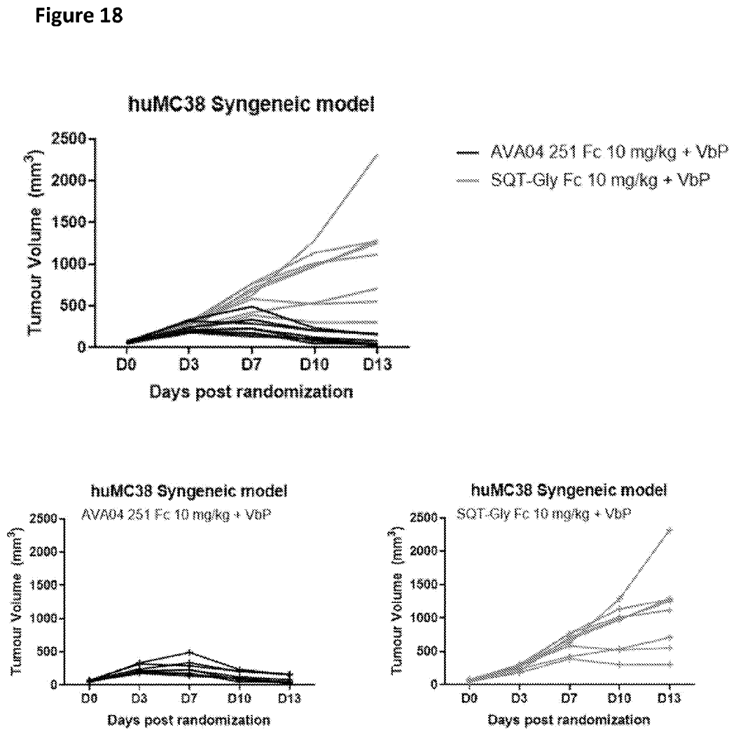

As will be apparent to those skilled in the art and illustrated in FIG. 17, the anti-PD-L1 affimer polypeptide sequence can be added at either of the N-terminal or C-terminal ends of the heavy or light chain of the antibody, or combinations/permuations thereof. Moreover, as shown in FIG. 9 in the context of multimeric affimers, more than one affimer sequence can be included to an any given antibody chain.

In some embodiments with respect to a multi-specific Binder-drug conjugate comprising a full-length immunoglobulin, the fusion of the affimer polypeptide sequence to the antibody will preserve the Fc function of the Fc region of the immunoglobulin. For instance, in certain embodiments, the Binder-drug conjugate will be capable of binding, via its Fc portion, to the Fc receptor of Fc receptor-positive cells. In some further embodiments, the Binder-drug conjugate may activate the Fc receptor-positive cell by binding to the Fc receptor-positive cell, thereby initiating or increasing the expression of cytokines and/or co-stimulatory antigens. Furthermore, the Binder-drug conjugate may transfer at least a second activation signal required for physiological activation of the T cell to the T cell via the co-stimulatory antigens and/or cytokines.

In some embodiments, resulted from the binding of its Fc portion to other cells that express Fc receptors present on the surface of effector cells from the immune system, such as immune cells, hepatocytes, and endothelial cells, the Binder-drug conjugate may possess antibody-dependent cellular cytotoxicity (ADCC) function, a mechanism of cell-mediated immune defense whereby an effector cell of the immune system actively lyses a target cell, whose membrane-surface antigen has been bound by an antibody, and therefore, trigger tumor cell death via ADCC. In some further embodiments, the Binder-drug conjugate is capable of demonstrating ADCC function.

As described above, apart from the Fc-mediated cytotoxicity, the Fc portion may contribute to maintaining the serum levels of the Binder-drug conjugate, critical for its stability and persistence in the body. For example, when the Fc portion binds to Fc receptors on endothelial cells and on phagocytes, the Binder-drug conjugate may become internalized and recycled back to the blood stream, enhancing its half-life within the body.

Exemplary targets of the additional affimer polypeptides include, but are not limited to, another immune checkpoint protein, and immune co-stimulatory receptor (particularly if the additional affimer(s) can agonize the co-stimulatory receptor), a receptor, a cytokine, a growth factor, or a tumor-associated antigen, mere to illustrate.

Where the Binder-drug conjugate is an affimer/antibody fusion protein, the immunoglobulin portion of the, for example, may be an immunoglobulin is a monoclonal antibody against CD20, CD30, CD33, CD38, CD52, VEGF, VEGF receptors, EGFR or Her2/neu. A few illustrative examples for such immunoglobulins include an antibody comprised within any of the following: trastuzumab, panitumumab, cetuximab, obinutuzumab, rituximab, pertuzumab, alemtuzumab, bevacizumab, tositumomab, ibritumomab, ofatumumab, brentuximab and gemtuzumab.

In certain embodiments, the anti-PD-L1 affimer polypeptide is part of a binder-drug conjugate that includes one more binding domains that inhibit an immune checkpoint molecule, such as expressed on a T-cell, including but not limited to PD-1, PD-L2, CTLA-4, NKG2A, KIR, LAG-3, TIM-3, CD96, VISTA, or TIGIT.

In certain embodiments, the anti-PD-L1 affimer polypeptide is part of a binder-drug conjugate that includes one more binding domains that agonizes an immune co-stimulatory molecule, such as expressed on a T-cell, including but not limited to CD28, ICOS, CD137, OX40, GITR, CD27, CD30, HVEM, DNAM-1 or CD28H.

In certain embodiments, the anti-PD-L1 affimer polypeptide is part of a binder-drug conjugate that includes one more ligand agonists of immune co-stimulatory molecules, such as an agonist ligand for CD28, ICOS, CD137, OX40, GITR, CD27, CD30, HVEM, DNAM-1 or CD28H.

By combining the PD-L1 inhibitory activity of the anti-PD-L1 affimer with binding domains that block of one or several of inhibitory immune checkpoints and/or activate one or more of immune costimulatory pathways, the multi-specific Binder-drug conjugates can rescue otherwise exhausted anti-tumor T cells, enhance anti-tumor immunity and, thereby, enlists positive responses in cancer patients. In some further embodiments, dual blockade by the Binder-drug conjugate of coordinately expressed immune-checkpoint proteins can produce additive or synergistic anti-tumor activities.

In certain embodiments, the anti-PD-L1 affimer polypeptide is part of a binder-drug conjugate that includes one more binding domains that inhibit a soluble immune suppressing molecule, such as a binding domain that binds to the soluble immune suppressing molecules (such as a receptor trap) or a binding domain that binds to the corresponding cognate receptor and prevents ligand activation of the receptor, including but not limited to antagonists of PGE2, TGF-β, VEGF, CCL2, IDO, CSF1, IL-10, IL-13, IL-23, adenosine, or STAT3 activators. In certain instances, the Binder-drug conjugate includes a VEGF Receptor Trap domain, such as the VEGF binding receptor domain of Aflibercept. In another example, the Binder-drug conjugate includes a TGF-β Receptor Trap domain, such as the TGF-3 binding receptor domain of MSB0011359C.

In certain embodiments, the anti-PD-L1 affimer polypeptide is part of a binder-drug conjugate that includes one more binding domains that bind to a protein upregulated in the tumor microenvironment, i.e., a tumor associated antigen, such as upregulated on tumor cells in the tumor, or macrophage, fibroblasts, T-cells or other immune cells that infiltrate the tumor.

In certain embodiments, the anti-PD-L1 affimer polypeptide is part of a binder-drug conjugate that includes one more binding domains that bind to a protein selected from the groups consisting of CEACAM-1, CEACAM-5, BTLA, LAIR1, CD160, 2B4, TGFR, B7-H3, B7-H4, CD40, CD40L, CD47, CD70, CD80, CD86, CD94, CD137, CD137L, CD226, Galectin-9, GITRL, HHLA2, ICOS, ICOSL, LIGHT, MHC class I or II, NKG2a, NKG2d, OX4OL, PVR, SIRP□, TCR, CD20, CD30, CD33, CD38, CD52, VEGF, VEGF receptors, EGFR, Her2/neu, ILT1, ILT2, ILT3, ILT4, ILT5, ILT6, ILT7, ILT8, KIR2DL1, KIR2DL2, KIR2DL3, KIR2DL4, KIR2DL5A, KIR2DL5B, KIR3DL1, KIR3DL2, KIR3DL3, NKG2A, NKG2C, NKG2E or TSLP.

(ii) Other PD-L1 Binders

In some embodiments, the cell binding moiety is a PD-L1 binding antagonist that inhibits the binding of PD-L1 to both PD-1 and B7-1. In some embodiments, PD-L1 binding antagonist is an anti-PD-L1 antibody. In some embodiments, the anti-PD-L1 antibody is a monoclonal antibody. In some embodiments, the anti-PDL1 antibody is an antibody fragment, such as selected from the group consisting of Fab, Fab′-SH, Fv, scFv, and (Fab′)2 fragments. In some embodiments, the anti-PD-L1 antibody is a humanized antibody or a human antibody. In some embodiments, the PD-L1 binding antagonist is selected from the group consisting of: YW243.55.S70, MPDL3280A, MDX-1105, and MEDI4736.

In certain embodiments, the cell binding moiety is an anti-PD-L1 antibody or fragment thereof comprising a heavy chain variable region comprising the amino acid sequence of

EVQLVESGGGLVQPGGSLRLSCAASGFTFSDSWIHWVRQAPGKGLEW
VAWISPYGGSTYYADSVKGRFTISADTSKNTAYLQMNSLRAEDTAVY
YCARRHWPGGFDYWGQGTLVTVSS
Or
EVQLVESGGGLVQPGGSLRLSCAASGFTFSDSWIHWVRQAPGKGLEW
VAWISPYGGSTYYADSVKGRFTISADTSKNTAYLQMNSLRAEDTAVY
YCARRHWPGGFDYWGQGTLVTVSSASTK

and a light chain variable region comprising the amino acid sequence of

DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKR.

Other human and humanized antibodies, and fragments thereof, are well know in the art and can be readily adapted for use in the present invention.

### IV. Methods of Use and Pharmaceutical Compositions

The Binder-drug conjugates of the invention are useful in a variety of applications including, but not limited to, therapeutic treatment methods, such as immunotherapy for cancer. In certain embodiments, Binder-drug conjugates described herein are useful for activating, promoting, increasing, and/or enhancing an immune response, inhibiting tumor growth, reducing tumor volume, inducing tumor regression, increasing tumor cell apoptosis, and/or reducing the tumorigenicity of a tumor. In certain embodiments, the polypeptides or agents of the invention are also useful for immunotherapy against pathogens, such as viruses. In certain embodiments, the Binder-drug conjugates described herein are useful for inhibiting viral infection, reducing viral infection, increasing virally-infected cell apoptosis, and/or increasing killing of virus-infected cells. The methods of use may be in vitro, ex vivo, or in vivo methods.

The present invention provides methods for activating an immune response in a subject using a binder-drug conjugate. In some embodiments, the invention provides methods for promoting an immune response in a subject using a binder-drug conjugate described herein. In some embodiments, the invention provides methods for increasing an immune response in a subject using a binder-drug conjugate. In some embodiments, the invention provides methods for enhancing an immune response in a subject using a binder-drug conjugate. In some embodiments, the activating, promoting, increasing, and/or enhancing of an immune response comprises increasing cell-mediated immunity. In some embodiments, the activating, promoting, increasing, and/or enhancing of an immune response comprises increasing Th1-type responses. In some embodiments, the activating, promoting, increasing, and/or enhancing of an immune response comprises increasing T-cell activity. In some embodiments, the activating, promoting, increasing, and/or enhancing of an immune response comprises increasing CD4+ T-cell activity. In some embodiments, the activating, promoting, increasing, and/or enhancing of an immune response comprises increasing CD8+ T-cell activity. In some embodiments, the activating, promoting, increasing, and/or enhancing of an immune response comprises increasing CTL activity. In some embodiments, the activating, promoting, increasing, and/or enhancing of an immune response comprises increasing NK cell activity. In some embodiments, the activating, promoting, increasing, and/or enhancing of an immune response comprises increasing T-cell activity and increasing NK cell activity. In some embodiments, the activating, promoting, increasing, and/or enhancing of an immune response comprises increasing CU activity and increasing NK cell activity. In some embodiments, the activating, promoting, increasing, and/or enhancing of an immune response comprises inhibiting or decreasing the suppressive activity of Treg cells. In some embodiments, the activating, promoting, increasing, and/or enhancing of an immune response comprises inhibiting or decreasing the suppressive activity of MDSCs. In some embodiments, the activating, promoting, increasing, and/or enhancing of an immune response comprises increasing the number of the percentage of memory T-cells. In some embodiments, the activating, promoting, increasing, and/or enhancing of an immune response comprises increasing long-term immune memory function. In some embodiments, the activating, promoting, increasing, and/or enhancing of an immune response comprises increasing long-term memory. In some embodiments, the activating, promoting, increasing, and/or enhancing of an immune response comprises no evidence of substantial side effects and/or immune-based toxicities. In some embodiments, the activating, promoting, increasing, and/or enhancing of an immune response comprises no evidence of cytokine release syndrome (CRS) or a cytokine storm. In some embodiments, the immune response is a result of antigenic stimulation. In some embodiments, the antigenic stimulation is a tumor cell. In some embodiments, the antigenic stimulation is cancer. In some embodiments, the antigenic stimulation is a pathogen. In some embodiments, the antigenic stimulation is a virally-infected cell.

In vivo and in vitro assays for determining whether a binder-drug conjugate activates, or inhibits an immune response are known in the art.

In some embodiments, a method of increasing an immune response in a subject comprises administering to the subject a therapeutically effective amount of a binder-drug conjugate described herein, wherein the a binder-drug conjugate binds human PD-L1.

In some embodiments, a method of increasing an immune response in a subject comprises administering to the subject a therapeutically effective amount of a binder-drug conjugate described herein, wherein the Binder-drug conjugate is an affimer-containing antibody or receptor trap fusion polypeptide including an affimer polypeptide that specifically binds to PD-L1

In certain embodiments of the methods described herein, a method of activating or enhancing a persistent or long-term immune response to a tumor comprises administering to a subject a therapeutically effective amount of a binder-drug conjugate which binds human PD-L1. In some embodiments, a method of activating or enhancing a persistent immune response to a tumor comprises administering to a subject a therapeutically effective amount of a binder-drug conjugate described herein, wherein the Binder-drug conjugate is a affimer-containing antibody or receptor trap fusion polypeptide including an affimer polypeptide that specifically binds to PD-L1.

In certain embodiments of the methods described herein, a method of inhibiting tumor relapse or tumor regrowth comprises administering to a subject a therapeutically effective amount of a binder-drug conjugate which binds human PD-L1. In some embodiments, a method of inhibiting tumor relapse or tumor regrowth comprises administering to a subject a therapeutically effective amount of a binder-drug conjugate described herein, wherein the Binder-drug conjugate is a affimer-containing antibody or receptor trap fusion polypeptide including an affimer polypeptide that specifically binds to PD-L1.

In some embodiments, the tumor expresses or overexpresses a tumor antigen that is targeted by an additional binding entity provided in the Binder-drug conjugate along with the anti-PD-L1 affimer polypeptide, i.e., where the Binder-drug conjugate is a bispecific or multispecific agent.

In certain embodiments, the method of inhibiting growth of a tumor comprises administering to a subject a therapeutically effective amount of a binder-drug conjugate described herein. In certain embodiments, the subject is a human. In certain embodiments, the subject has a tumor, or the subject had a tumor which was removed.

In some embodiments, the tumor is a solid tumor. In certain embodiments, the tumor is a tumor selected from the group consisting of: colorectal tumor, pancreatic tumor, lung tumor, ovarian tumor, liver tumor, breast tumor, kidney tumor, prostate tumor, neuroendocrine tumor, gastrointestinal tumor, melanoma, cervical tumor, bladder tumor, glioblastoma, and head and neck tumor. In certain embodiments, the tumor is a colorectal tumor. In certain embodiments, the tumor is an ovarian tumor. In some embodiments, the tumor is a lung tumor. In certain embodiments, the tumor is a pancreatic tumor. In certain embodiments, the tumor is a melanoma tumor. In some embodiments, the tumor is a bladder tumor.

To further illustrate, the subject Binder-drug conjugates can be used to treat patients suffering from cancer, such as osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer (e.g., non-small cell lung cancer), gastric cancer, colorectal cancer, cervical cancer, synovial sarcoma, head and neck cancer, squamous cell carcinoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, carcinoid cancer or liver cancer, breast cancer or gastric cancer. In an embodiment of the invention, the cancer is metastatic cancer, e.g., of the varieties described above.

In certain embodiments, the cancer is a hematologic cancer. In some embodiment, the cancer is selected from the group consisting of: acute myelogenous leukemia (AML), Hodgkin lymphoma, multiple myeloma, T-cell acute lymphoblastic leukemia (T-ALL), chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), and cutaneous T-cell lymphoma (CTCL).

The present invention also provides pharmaceutical compositions comprising a binder-drug conjugate described herein and a pharmaceutically acceptable vehicle. In some embodiments, the pharmaceutical compositions find use in immunotherapy. In some embodiments, the pharmaceutical compositions find use in immuno-oncology. In some embodiments, the compositions find use in inhibiting tumor growth. In some embodiments, the pharmaceutical compositions find use in inhibiting tumor growth in a subject (e.g., a human patient). In some embodiments, the compositions find use in treating cancer. In some embodiments, the pharmaceutical compositions find use in treating cancer in a subject (e.g., a human patient).

Formulations are prepared for storage and use by combining a purified Binder-drug conjugate of the present invention with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition.

In some embodiments, a binder-drug conjugate described herein is lyophilized and/or stored in a lyophilized form. In some embodiments, a formulation comprising a binder-drug conjugate described herein is lyophilized.

Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22.sup.nd Edition, 2012, Pharmaceutical Press, London.).

The pharmaceutical compositions of the present invention can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intraarterial, intratumoral, subcutaneous, intraperitoneal, intramuscular (e.g., injection or infusion), or intracranial (e.g., intrathecal or intraventricular).

The therapeutic formulation can be in unit dosage form. Such formulations include tablets, pills, capsules, powders, granules, solutions or suspensions in water or non-aqueous media, or suppositories. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and diluents (e.g., water). These can be used to form a solid preformulation composition containing a homogeneous mixture of a compound of the present invention, or a non-toxic pharmaceutically acceptable salt thereof. The solid preformulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol and cellulose acetate.

The Binder-drug conjugates described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22.sup.nd Edition, 2012, Pharmaceutical Press, London.

In certain embodiments, pharmaceutical formulations include a binder-drug conjugate of the present invention complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In certain embodiments, sustained-release preparations comprising Binder-drug conjugates described herein can be produced. Suitable examples of sustained-release preparations include semi-permeable matrices of solid hydrophobic polymers containing a binder-drug conjugate, where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

In certain embodiments, in addition to administering a binder-drug conjugate described herein, the method or treatment further comprises administering at least one additional immune response stimulating agent. In some embodiments, the additional immune response stimulating agent includes, but is not limited to, a colony stimulating factor (e.g., granulocyte-macrophage colony stimulating factor (GM-CSF), macrophage colony stimulating factor (M-CSF), granulocyte colony stimulating factor (G-CSF), stem cell factor (SCF)), an interleukin (e.g., IL-1, IL2, IL-3, IL-7, IL-12, IL-15, IL-18), a checkpoint inhibitor, an antibody that blocks immunosuppressive functions (e.g., an anti-CTLA-4 antibody, anti-CD28 antibody, anti-CD3 antibody), a toll-like receptor (e.g., TLR4, TLR7, TLR9), or a member of the B7 family (e.g., CD80, CD86). An additional immune response stimulating agent can be administered prior to, concurrently with, and/or subsequently to, administration of the Binder-drug conjugate. Pharmaceutical compositions comprising a binder-drug conjugate and the immune response stimulating agent(s) are also provided. In some embodiments, the immune response stimulating agent comprises 1, 2, 3, or more immune response stimulating agents.

In certain embodiments, in addition to administering a binder-drug conjugate described herein, the method or treatment further comprises administering at least one additional therapeutic agent. An additional therapeutic agent can be administered prior to, concurrently with, and/or subsequently to, administration of the Binder-drug conjugate. Pharmaceutical compositions comprising a binder-drug conjugate and the additional therapeutic agent(s) are also provided. In some embodiments, the at least one additional therapeutic agent comprises 1, 2, 3, or more additional therapeutic agents.

Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the Binder-drug conjugate. Combination therapy may decrease the likelihood that resistant cancer cells will develop. In some embodiments, combination therapy comprises a therapeutic agent that affects the immune response (e.g., enhances or activates the response) and a therapeutic agent that affects (e.g., inhibits or kills) the tumor/cancer cells.

In some embodiments of the methods described herein, the combination of a binder-drug conjugate described herein and at least one additional therapeutic agent results in additive or synergistic results. In some embodiments, the combination therapy results in an increase in the therapeutic index of the Binder-drug conjugate. In some embodiments, the combination therapy results in an increase in the therapeutic index of the additional therapeutic agent(s). In some embodiments, the combination therapy results in a decrease in the toxicity and/or side effects of the Binder-drug conjugate. In some embodiments, the combination therapy results in a decrease in the toxicity and/or side effects of the additional therapeutic agent(s).

Useful classes of therapeutic agents include, for example, anti-tubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, anti-folates, anti-metabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an alkylating agent, an antimetabolite, an antimitotic, a topoisomerase inhibitor, or an angiogenesis inhibitor.

Therapeutic agents that may be administered in combination with the Binder-drug conjugate described herein include chemotherapeutic agents. Thus, in some embodiments, the method or treatment involves the administration of a binder-drug conjugate of the present invention in combination with a chemotherapeutic agent or in combination with a cocktail of chemotherapeutic agents. Treatment with a binder-drug conjugate can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book, 4.sup.th Edition, 2008, M. C. Perry, Editor, Lippincott, Williams & Wilkins, Philadelphia, Pa.

Chemotherapeutic agents useful in the present invention include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON); and anti-androgens such as flutamide, nilutamide, bicalutamide, leuprolide, and goserelin; and pharmaceutically acceptable salts, acids or derivatives of any of the above. In certain embodiments, the additional therapeutic agent is cisplatin. In certain embodiments, the additional therapeutic agent is carboplatin.

In certain embodiments of the methods described herein, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these. In some embodiments, the additional therapeutic agent is irinotecan.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In certain embodiments, the additional therapeutic agent is gemcitabine.

In certain embodiments of the methods described herein, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (nab-paclitaxel; ABRAXANE), DHA-paclitaxel, or PG-paclitaxel. In certain alternative embodiments, the antimitotic agent comprises a vinca alkaloid, such as vincristine, vinblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1. In certain embodiments, the additional therapeutic agent is paclitaxel. In certain embodiments, the additional therapeutic agent is nab-paclitaxel.

In some embodiments of the methods described herein, an additional therapeutic agent comprises an agent such as a small molecule. For example, treatment can involve the combined administration of a binder-drug conjugate of the present invention with a small molecule that acts as an inhibitor against tumor-associated antigens including, but not limited to, EGFR, HER2 (ErbB2), and/or VEGF. In some embodiments, a binder-drug conjugate of the present invention is administered in combination with a protein kinase inhibitor selected from the group consisting of: gefitinib (IRESSA), erlotinib (TARCEVA), sunitinib (SUTENT), lapatanib, vandetanib (ZACTIMA), AEE788, CI-1033, cediranib (RECENTIN), sorafenib (NEXAVAR), and pazopanib (GW786034B). In some embodiments, an additional therapeutic agent comprises an mTOR inhibitor.

In certain embodiments of the methods described herein, the additional therapeutic agent is a small molecule that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an inhibitor of the Hippo pathway. In some embodiments, the additional therapeutic agent is an inhibitor of the mTOR/AKR pathway. In some embodiments, the additional therapeutic agent is an inhibitor of the RSPO/LGR pathway.

In some embodiments of the methods described herein, an additional therapeutic agent comprises a biological molecule, such as an antibody. For example, treatment can involve the combined administration of a binder-drug conjugate of the present invention with antibodies against tumor-associated antigens including, but not limited to, antibodies that bind EGFR, HER2/ErbB2, and/or VEGF. In certain embodiments, the additional therapeutic agent is an antibody specific for a cancer stem cell marker. In some embodiments, the additional therapeutic agent is an antibody that binds a component of the Notch pathway. In some embodiments, the additional therapeutic agent is an antibody that binds a component of the Wnt pathway. In certain embodiments, the additional therapeutic agent is an antibody that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an antibody that inhibits.beta.-catenin signaling. In certain embodiments, the additional therapeutic agent is an antibody that is an angiogenesis inhibitor (e.g., an anti-VEGF or VEGF receptor antibody). In certain embodiments, the additional therapeutic agent is bevacizumab (AVASTIN), ramucirumab, trastuzumab (HERCEPTIN), pertuzumab (OMNITARG), panitumumab (VECTIBIX), nimotuzumab, zalutumumab, or cetuximab (ERBITUX).

In some embodiments of the methods described herein, the additional therapeutic agent is an antibody that modulates the immune response. In some embodiments, the additional therapeutic agent is an anti-PD-1 antibody, an anti-LAG-3 antibody, an anti-CTLA-4 antibody, an anti-TIM-3 antibody, or an anti-TIGIT antibody.

Furthermore, treatment with a binder-drug conjugate described herein can include combination treatment with other biologic molecules, such as one or more cytokines (e.g., lymphokines, interleukins, tumor necrosis factors, and/or growth factors) or can be accompanied by surgical removal of tumors, removal of cancer cells, or any other therapy deemed necessary by a treating physician. In some embodiments, the additional therapeutic agent is an immune response stimulating agent.

In some embodiments of the methods described herein, the Binder-drug conjugate can be combined with a growth factor selected from the group consisting of: adrenomedullin (AM), angiopoietin (Ang), BMPs, BDNF, EGF, erythropoietin (EPO), FGF, GDNF, G-CSF, GM-CSF, GDF9, HGF, HDGF, IGF, migration-stimulating factor, myostatin (GDF-8), NGF, neurotrophins, PDGF, thrombopoietin, TGF-□, TGF-□, TNF-□, VEGF, P1GF, IL-1, IL-2, IL-3, IL-4, IL-5, IL-6, IL-7, IL-12, IL-15, and IL-18.

In some embodiments of the methods described herein, the additional therapeutic agent is an immune response stimulating agent. In some embodiments, the immune response stimulating agent is selected from the group consisting of granulocyte-macrophage colony stimulating factor (GM-CSF), macrophage colony stimulating factor (M-CSF), granulocyte colony stimulating factor (G-CSF), interleukin 3 (IL-3), interleukin 12 (IL-12), interleukin 1 (IL-1), interleukin 2 (IL-2), B7-1 (CD80), B7-2 (CD86), 4-1BB ligand, anti-CD3 antibody, anti-CTLA-4 antibody, anti-TIGIT antibody, anti-PD-1 antibody, anti-LAG-3 antibody, and anti-TIM-3 antibody.

In some embodiments of the methods described herein, an immune response stimulating agent is selected from the group consisting of: a modulator of PD-1 activity, a modulator of PD-L2 activity, a modulator of CTLA-4 activity, a modulator of CD28 activity, a modulator of CD80 activity, a modulator of CD86 activity, a modulator of 4-1BB activity, an modulator of OX40 activity, a modulator of KIR activity, a modulator of Tim-3 activity, a modulator of LAG3 activity, a modulator of CD27 activity, a modulator of CD40 activity, a modulator of GITR activity, a modulator of TIGIT activity, a modulator of CD20 activity, a modulator of CD96 activity, a modulator of IDO1 activity, a cytokine, a chemokine, an interferon, an interleukin, a lymphokine, a member of the tumor necrosis factor (TNF) family, and an immunostimulatory oligonucleotide.

In some embodiments of the methods described herein, an immune response stimulating agent is selected from the group consisting of: a PD-1 antagonist, a PD-L2 antagonist, a CTLA-4 antagonist, a CD80 antagonist, a CD86 antagonist, a KIR antagonist, a Tim-3 antagonist, a LAG3 antagonist, a TIGIT antagonist, a CD20 antagonist, a CD96 antagonist, and/or an IDO1 antagonist.

In some embodiments of the methods described herein, the PD-1 antagonist is an antibody that specifically binds PD-1. In some embodiments, the antibody that binds PD-1 is KEYTRUDA (MK-3475), pidilizumab (CT-011), nivolumab (OPDIVO, BMS-936558, MDX-1106), MEDI0680 (AMP-514), REGN2810, BGB-A317, PDR-001, or STI-A1110. In some embodiments, the antibody that binds PD-1 is described in PCT Publication WO 2014/179664, for example, an antibody identified as APE2058, APE1922, APE1923, APE1924, APE 1950, or APE1963, or an antibody containing the CDR regions of any of these antibodies. In other embodiments, the PD-1 antagonist is a fusion protein that includes PD-L2, for example, AMP-224. In other embodiments, the PD-1 antagonist is a peptide inhibitor, for example, AUNP-12.

In some embodiments, the CTLA-4 antagonist is an antibody that specifically binds CTLA-4. In some embodiments, the antibody that binds CTLA-4 is ipilimumab (YERVOY) or tremelimumab (CP-675,206). In some embodiments, the CTLA-4 antagonist a CTLA-4 fusion protein, for example, KAHR-102.

In some embodiments, the LAG3 antagonist is an antibody that specifically binds LAG3. In some embodiments, the antibody that binds LAG3 is IMP701, IMP731, BMS-986016, LAG525, and GSK2831781. In some embodiments, the LAG3 antagonist includes a soluble LAG3 receptor, for example, IMP321.

In some embodiments, the KIR antagonist is an antibody that specifically binds KIR. In some embodiments, the antibody that binds KIR is lirilumab.

In some embodiments, an immune response stimulating agent is selected from the group consisting of: a CD28 agonist, a 4-1BB agonist, an OX40 agonist, a CD27 agonist, a CD80 agonist, a CD86 agonist, a CD40 agonist, and a GITR agonist. p In some embodiments, the OX40 agonist includes OX40 ligand, or an OX40-binding portion thereof. For example, the OX40 agonist may be MEDI6383. In some embodiments, the OX40 agonist is an antibody that specifically binds OX40. In some embodiments, the antibody that binds OX40 is MEDI6469, MEDI0562, or MOXR0916 (RG7888). In some embodiments, the OX40 agonist is a vector (e.g., an expression vector or virus, such as an adenovirus) capable of expressing OX40 ligand. In some embodiments the OX40-expressing vector is Delta-24-RGDOX or DNX2401.

In some embodiments, the 4-1BB (CD137) agonist is a binding molecule, such as an anticalin. In some embodiments, the anticalin is PRS-343. In some embodiments, the 4-1BB agonist is an antibody that specifically binds 4-1BB. In some embodiments, antibody that binds 4-1BB is PF-2566 (PF-05082566) or urelumab (BMS-663513).

In some embodiments, the CD27 agonist is an antibody that specifically binds CD27. In some embodiments, the antibody that binds CD27 is varlilumab (CDX-1127).

In some embodiments, the GITR agonist comprises GITR ligand or a GITR-binding portion thereof. In some embodiments, the GITR agonist is an antibody that specifically binds GITR. In some embodiments, the antibody that binds GITR is TRX518, MK-4166, or INBRX-110.

In some embodiments, immune response stimulating agents include, but are not limited to, cytokines such as chemokines, interferons, interleukins, lymphokines, and members of the tumor necrosis factor (TNF) family. In some embodiments, immune response stimulating agents include immunostimulatory oligonucleotides, such as CpG dinucleotides.

In some embodiments, an immune response stimulating agent includes, but is not limited to, anti-PD-1 antibodies, anti-PD-L2 antibodies, anti-CTLA-4 antibodies, anti-CD28 antibodies, anti-CD80 antibodies, anti-CD86 antibodies, anti-4-1BB antibodies, anti-OX40 antibodies, anti-KIR antibodies, anti-Tim-3 antibodies, anti-LAG3 antibodies, anti-CD27 antibodies, anti-CD40 antibodies, anti-GITR antibodies, anti-TIGIT antibodies, anti-CD20 antibodies, anti-CD96 antibodies, or anti-IDO1 antibodies.

In particular embodiments, the Binder-drug conjugates disclosed herein may be used alone, or in association with radiation therapy.

In particular embodiments, the Binder-drug conjugates disclosed herein may be used alone, or in association with targeted therapies. Examples of targeted therapies include: hormone therapies, signal transduction inhibitors (e.g., EGFR inhibitors, such as cetuximab (Erbitux) and erlotinib (Tarceva)); HER2 inhibitors (e.g., trastuzumab (Herceptin) and pertuzumab (Perjeta)); BCR-ABL inhibitors (such as imatinib (Gleevec) and dasatinib (Sprycel)); ALK inhibitors (such as crizotinib (Xalkori) and ceritinib (Zykadia)); BRAF inhibitors (such as vemurafenib (Zelboraf) and dabrafenib (Tafinlar)), gene expression modulators, apoptosis inducers (e.g., bortezomib (Velcade) and carfilzomib (Kyprolis)), angiogenesis inhibitors (e.g., bevacizumab (Avastin) and ramucirumab (Cyramza), monoclonal antibodies attached to toxins (e.g., brentuximab vedotin (Adcetris) and ado-trastuzumab emtansine (Kadcyla)).

In particular embodiments, the Binder-drug conjugates of the invention may be used in combination with an anti-cancer therapeutic agent or immunomodulatory drug such as an immunomodulatory receptor inhibitor, e.g., an antibody or antigen-binding fragment thereof that specifically binds to the receptor.

In an embodiment of the invention, an anti-PD-1 antibody or antigen-binding fragment thereof of the invention is in association with a Tim-3 pathway antagonist, preferably as part of a pharmaceutical composition.

In an embodiment of the invention, an anti-PD-1 antibody or antigen-binding fragment thereof of the invention is in association with a Vista pathway antagonist, preferably as part of a pharmaceutical composition.

In an embodiment of the invention, an anti-PD-1 antibody or antigen-binding fragment thereof of the invention is in association with a BTLA pathway antagonist, preferably as part of a pharmaceutical composition.

In an embodiment of the invention, an anti-PD-1 antibody or antigen-binding fragment thereof of the invention is in association with a LAG-3 pathway antagonist, preferably as part of a pharmaceutical composition.

In an embodiment of the invention, an anti-PD-1 antibody or antigen-binding fragment thereof of the invention is in association with a TIGIT pathway antagonist, preferably as part of a pharmaceutical composition.

In an embodiment of the invention, an anti-PD-1 antibody or antigen-binding fragment thereof of the invention is in association with an anti-PDL1 antibody

In an embodiment of the invention, an anti-PD-1 antibody or antigen-binding fragment thereof of the invention is in association with BMS-936559, MSB0010718C or MPDL3280A), preferably as part of a pharmaceutical composition.

In an embodiment of the invention, an anti-PD-1 antibody or antigen-binding fragment thereof of the invention is in association with an anti-CTLA4 antibody, preferably as part of a pharmaceutical composition.

In an embodiment of the invention, an anti-PD-1 antibody or antigen-binding fragment thereof of the invention is in association with an anti-CS1 antibody, preferably as part of a pharmaceutical composition.

In an embodiment of the invention, an anti-PD-1 antibody or antigen-binding fragment thereof of the invention is in association with an anti-KIR2DL1/2/3 antibody, preferably as part of a pharmaceutical composition.

In an embodiment of the invention, an anti-PD-1 antibody or antigen-binding fragment thereof of the invention is in association with an anti-CD137 antibody, preferably as part of a pharmaceutical composition.

In an embodiment of the invention, an anti-PD-1 antibody or antigen-binding fragment thereof of the invention is in association with an anti-GITR antibody, preferably as part of a pharmaceutical composition.

In an embodiment of the invention, an anti-PD-1 antibody or antigen-binding fragment thereof of the invention is in association with an anti-PD-L2 antibody, preferably as part of a pharmaceutical composition.

In an embodiment of the invention, an anti-PD-1 antibody or antigen-binding fragment thereof of the invention is in association with an anti-ILT1 antibody, preferably as part of a pharmaceutical composition.

In an embodiment of the invention, an anti-PD-1 antibody or antigen-binding fragment thereof of the invention is in association with an anti-ILT2 antibody, preferably as part of a pharmaceutical composition.

In an embodiment of the invention, an anti-PD-1 antibody or antigen-binding fragment thereof of the invention is in association with an anti-ILT3 antibody, preferably as part of a pharmaceutical composition.

In an embodiment of the invention, an anti-PD-1 antibody or antigen-binding fragment thereof of the invention is in association with an anti-ILT4 antibody, preferably as part of a pharmaceutical composition.

In an embodiment of the invention, an anti-PD-1 antibody or antigen-binding fragment thereof of the invention is in association with an anti-ILT5 antibody, preferably as part of a pharmaceutical composition.

In an embodiment of the invention, an anti-PD-1 antibody or antigen-binding fragment thereof of the invention is in association with an anti-ILT6 antibody, preferably as part of a pharmaceutical composition.

In an embodiment of the invention, an anti-PD-1 antibody or antigen-binding fragment thereof of the invention is in association with an anti-ILT7 antibody, preferably as part of a pharmaceutical composition.

In an embodiment of the invention, an anti-PD-1 antibody or antigen-binding fragment thereof of the invention is in association with an anti-ILT8 antibody, preferably as part of a pharmaceutical composition.

In an embodiment of the invention, an anti-PD-1 antibody or antigen-binding fragment thereof of the invention is in association with an anti-CD40 antibody, preferably as part of a pharmaceutical composition.

In an embodiment of the invention, an anti-PD-1 antibody or antigen-binding fragment thereof of the invention is in association with an anti-OX40 antibody, preferably as part of a pharmaceutical composition.

In an embodiment of the invention, an anti-PD-1 antibody or antigen-binding fragment thereof of the invention is in association with an anti-KIR2DL1 antibody, preferably as part of a pharmaceutical composition.

In an embodiment of the invention, an anti-PD-1 antibody or antigen-binding fragment thereof of the invention is in association with an anti-KIR2DL2/3 antibody, preferably as part of a pharmaceutical composition.

In an embodiment of the invention, an anti-PD-1 antibody or antigen-binding fragment thereof of the invention is in association with an anti-KIR2DL4 antibody, preferably as part of a pharmaceutical composition.

In an embodiment of the invention, an anti-PD-1 antibody or antigen-binding fragment thereof of the invention is in association with an anti-KIR2DL5A antibody, preferably as part of a pharmaceutical composition.

In an embodiment of the invention, an anti-PD-1 antibody or antigen-binding fragment thereof of the invention is in association with an anti-KIR2DL5B antibody, preferably as part of a pharmaceutical composition.

In an embodiment of the invention, an anti-PD-1 antibody or antigen-binding fragment thereof of the invention is in association with an anti-KIR3DL1 antibody, preferably as part of a pharmaceutical composition.

In an embodiment of the invention, an anti-PD-1 antibody or antigen-binding fragment thereof of the invention is in association with an anti-KIR3DL2 antibody, preferably as part of a pharmaceutical composition.

In an embodiment of the invention, an anti-PD-1 antibody or antigen-binding fragment thereof of the invention is in association with an anti-KIR3DL3 antibody, preferably as part of a pharmaceutical composition.

In an embodiment of the invention, an anti-PD-1 antibody or antigen-binding fragment thereof of the invention is in association with an anti-NKG2A antibody, preferably as part of a pharmaceutical composition.

In an embodiment of the invention, an anti-PD-1 antibody or antigen-binding fragment thereof of the invention is in association with an anti-NKG2C antibody, preferably as part of a pharmaceutical composition.

In an embodiment of the invention, an anti-PD-1 antibody or antigen-binding fragment thereof of the invention is in association with an anti-ICOS antibody, preferably as part of a pharmaceutical composition.

In an embodiment of the invention, an anti-PD-1 antibody or antigen-binding fragment thereof of the invention is in association with an anti-SIRP.alpha. antibody, preferably as part of a pharmaceutical composition.

In an embodiment of the invention, an anti-PD-1 antibody or antigen-binding fragment thereof of the invention is in association with an anti-CD47 antibody, preferably as part of a pharmaceutical composition.

In an embodiment of the invention, an anti-PD-1 antibody or antigen-binding fragment thereof of the invention is in association with an anti-4-1 BB antibody, preferably as part of a pharmaceutical composition.

In an embodiment of the invention, an anti-PD-1 antibody or antigen-binding fragment thereof of the invention is in association with an anti-IL-10 antibody, preferably as part of a pharmaceutical composition.

In an embodiment of the invention, an anti-PD-1 antibody or antigen-binding fragment thereof of the invention is in association with an anti-TSLP antibody, preferably as part of a pharmaceutical composition.

In an embodiment of the invention, an anti-PD-1 antibody or antigen-binding fragment thereof of the invention is in association with IL-10 or PEGylated IL-10, preferably as part of a pharmaceutical composition.

In an embodiment of the invention, an anti-PD-1 antibody or antigen-binding fragment thereof of the invention is in association with an anti-APRIL antibody, preferably as part of a pharmaceutical composition.

In an embodiment of the invention, an anti-PD-1 antibody or antigen-binding fragment thereof of the invention is in association with an anti-CD27 antibody, preferably as part of a pharmaceutical composition.

In an embodiment of the invention, a binder-drug conjugate of the invention is in association with a STING agonist, preferably as part of a pharmaceutical composition. The cyclic-dinucleotides (CDNs) cyclic-di-AMP (produced by Listeria monocytogenes and other bacteria) and its analogs cyclic-di-GMP and cyclic-GMP-AMP are recognized by the host cell as a pathogen associated molecular pattern (PAMP), which bind to the pathogen recognition receptor (PRR) known as Stimulator of INterferon Genes (STING). STING is an adaptor protein in the cytoplasm of host mammalian cells which activates the TANK binding kinase (TBK1)-IRF3 and the NF-.kappa.B signaling axis, resulting in the induction of IFN-.beta. and other gene products that strongly activate innate immunity. It is now recognized that STING is a component of the host cytosolic surveillance pathway, that senses infection with intracellular pathogens and in response induces the production of IFN-β of the invention is in association with a STING agonist, leading to the development of an adaptive protective pathogen-specific immune response consisting of both antigen-specific CD4+ and CD8+ T cells as well as pathogen-specific antibodies. U.S. Pat. Nos. 7,709,458 and 7,592,326; PCT Publication Nos. WO2007/054279, WO2014/093936, WO2014/179335, WO2014/189805, WO2015/185565, WO2016/096174, WO2016/145102, WO2017/027645, WO2017/027646, and WO2017/075477 (all of which are incorporated by reference); and Yan et al., Bioorg. Med. Chem Lett. 18:5631-4, 2008.

In an embodiment of the invention, a binder-drug conjugate of the invention is administered in conjunction with one or more vaccines intended to stimulate an immune response to one or more predetermined antigens. The antigen(s) may be administered directly to the individual, or may be expressed within the individual from, for example, a tumor cell vaccine (e.g., GVAX) which may be autologous or allogenic, a dendritic cell vaccine, a DNA vaccine, an RNA vaccine, a viral-based vaccine, a bacterial or yeast vaccine (e.g., a Listeria monocytogenes or Saccharomyces cerevisiae), etc. See, e.g., Guo et al., Adv. Cancer Res. 2013; 119: 421-475; Obeid et al., Semin Oncol. 2015 August; 42(4): 549-561. Examples of target antigens that may find use in the invention are listed in the following Table 4. The target antigen may also be a fragment or fusion polypeptide comprising an immunologically active portion of the antigens listed in the table. This list is not meant to be limiting.

In an embodiment of the invention, a binder-drug conjugate of the invention is administered in association with one or more antiemetics including, but not limited to: casopitant (GlaxoSmithKline), Netupitant (MGI-Helsinn) and other NK-1 receptor antagonists, palonosetron (sold as Aloxi by MGI Pharma), aprepitant (sold as Emend by Merck and Co.; Rahway, N.J.), diphenhydramine (sold as Benadryl by Pfizer; New York, N.Y.), hydroxyzine (sold as Atarax by Pfizer; New York, N.Y.), metoclopramide (sold as Reglan by AH Robins Co; Richmond, Va.), lorazepam (sold as Ativan by Wyeth; Madison, N.J.), alprazolam (sold as Xanax by Pfizer; New York, N.Y.), haloperidol (sold as Haldol by Ortho-McNeil; Raritan, N.J.), droperidol (Inapsine), dronabinol (sold as Marinol by Solvay Pharmaceuticals, Inc.; Marietta, Ga.), dexamethasone (sold as Decadron by Merck and Co.; Rahway, N.J.), methylprednisolone (sold as Medrol by Pfizer; New York, N.Y.), prochlorperazine (sold as Compazine by Glaxosmithkline; Research Triangle Park, N.C.), granisetron (sold as Kytril by Hoffmann-La Roche Inc.; Nutley, N.J.), ondansetron (sold as Zofran by Glaxosmithkline; Research Triangle Park, N.C.), dolasetron (sold as Anzemet by Sanofi-Aventis; New York, N.Y.), tropisetron (sold as Navoban by Novartis; East Hanover, N.J.).

Other side effects of cancer treatment include red and white blood cell deficiency. Accordingly, in an embodiment of the invention, a binder-drug conjugate is administered in association with an agent which treats or prevents such a deficiency, such as, e.g., filgrastim, PEG-filgrastim, erythropoietin, epoetin alfa or darbepoetin alfa.

In an embodiment of the invention, a binder-drug conjugate of the invention is administered in association with anti-cancer radiation therapy. For example, in an embodiment of the invention, the radiation therapy is external beam therapy (EBT): a method for delivering a beam of high-energy X-rays to the location of the tumor. The beam is generated outside the patient (e.g., by a linear accelerator) and is targeted at the tumor site. These X-rays can destroy the cancer cells and careful treatment planning allows the surrounding normal tissues to be spared. No radioactive sources are placed inside the patient's body. In an embodiment of the invention, the radiation therapy is proton beam therapy: a type of conformal therapy that bombards the diseased tissue with protons instead of X-rays. In an embodiment of the invention, the radiation therapy is conformal external beam radiation therapy: a procedure that uses advanced technology to tailor the radiation therapy to an individual's body structures. In an embodiment of the invention, the radiation therapy is brachytherapy: the temporary placement of radioactive materials within the body, usually employed to give an extra dose—or boost—of radiation to an area.

In certain embodiments of the methods described herein, the treatment involves the administration of a binder-drug conjugate of the present invention in combination with anti-viral therapy. Treatment with a binder-drug conjugate can occur prior to, concurrently with, or subsequent to administration of antiviral therapy. The anti-viral drug used in combination therapy will depend upon the virus the subject is infected with.

Combined administration can include co-administration, either in a single pharmaceutical formulation or using separate formulations, or consecutive administration in either order but generally within a time period such that all active agents can exert their biological activities simultaneously.

It will be appreciated that the combination of a binder-drug conjugate described herein and at least one additional therapeutic agent may be administered in any order or concurrently. In some embodiments, the Binder-drug conjugate will be administered to patients that have previously undergone treatment with a second therapeutic agent. In certain other embodiments, the Binder-drug conjugate and a second therapeutic agent will be administered substantially simultaneously or concurrently. For example, a subject may be given a binder-drug conjugate while undergoing a course of treatment with a second therapeutic agent (e.g., chemotherapy). In certain embodiments, a binder-drug conjugate will be administered within 1 year of the treatment with a second therapeutic agent. In certain alternative embodiments, a binder-drug conjugate will be administered within 10, 8, 6, 4, or 2 months of any treatment with a second therapeutic agent. In certain other embodiments, a binder-drug conjugate will be administered within 4, 3, 2, or 1 weeks of any treatment with a second therapeutic agent. In some embodiments, a binder-drug conjugate will be administered within 5, 4, 3, 2, or 1 days of any treatment with a second therapeutic agent. It will further be appreciated that the two (or more) agents or treatments may be administered to the subject within a matter of hours or minutes (i.e., substantially simultaneously).

For the treatment of a disease, the appropriate dosage of a binder-drug conjugate of the present invention depends on the type of disease to be treated, the severity and course of the disease, the responsiveness of the disease, whether the Binder-drug conjugate is administered for therapeutic or preventative purposes, previous therapy, the patient's clinical history, and so on, all at the discretion of the treating physician. The Binder-drug conjugate can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of an individual agent. The administering physician can determine optimum dosages, dosing methodologies, and repetition rates. In certain embodiments, dosage is from 0.01 μg to 100 mg/kg of body weight, from 0.1 μg to 100 mg/kg of body weight, from 1 μg to 100 mg/kg of body weight, from 1 mg to 100 mg/kg of body weight, 1 mg to 80 mg/kg of body weight from 10 mg to 100 mg/kg of body weight, from 10 mg to 75 mg/kg of body weight, or from 10 mg to 50 mg/kg of body weight. In certain embodiments, the dosage of the Binder-drug conjugate is from about 0.1 mg to about 20 mg/kg of body weight. In some embodiments, the dosage of the Binder-drug conjugate is about 0.1 mg/kg of body weight. In some embodiments, the dosage of the Binder-drug conjugate is about 0.25 mg/kg of body weight. In some embodiments, the dosage of the Binder-drug conjugate is about 0.5 mg/kg of body weight. In some embodiments, the dosage of the Binder-drug conjugate is about 1 mg/kg of body weight. In some embodiments, the dosage of the Binder-drug conjugate is about 1.5 mg/kg of body weight. In some embodiments, the dosage of the Binder-drug conjugate is about 2 mg/kg of body weight. In some embodiments, the dosage of the Binder-drug conjugate is about 2.5 mg/kg of body weight. In some embodiments, the dosage of the Binder-drug conjugate is about 5 mg/kg of body weight. In some embodiments, the dosage of the Binder-drug conjugate is about 7.5 mg/kg of body weight. In some embodiments, the dosage of the Binder-drug conjugate is about 10 mg/kg of body weight. In some embodiments, the dosage of the Binder-drug conjugate is about 12.5 mg/kg of body weight. In some embodiments, the dosage of the Binder-drug conjugate is about 15 mg/kg of body weight. In certain embodiments, the dosage can be given once or more daily, weekly, monthly, or yearly. In certain embodiments, the Binder-drug conjugate is given once every week, once every two weeks, once every three weeks, or once every four weeks.

In some embodiments, a binder-drug conjugate may be administered at an initial higher “loading” dose, followed by one or more lower doses. In some embodiments, the frequency of administration may also change. In some embodiments, a dosing regimen may comprise administering an initial dose, followed by additional doses (or “maintenance” doses) once a week, once every two weeks, once every three weeks, or once every month. For example, a dosing regimen may comprise administering an initial loading dose, followed by a weekly maintenance dose of, for example, one-half of the initial dose. Or a dosing regimen may comprise administering an initial loading dose, followed by maintenance doses of, for example one-half of the initial dose every other week. Or a dosing regimen may comprise administering three initial doses for 3 weeks, followed by maintenance doses of, for example, the same amount every other week.

As is known to those of skill in the art, administration of any therapeutic agent may lead to side effects and/or toxicities. In some cases, the side effects and/or toxicities are so severe as to preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or if possible, suffer from the unpleasant side effects associated with the therapeutic agent.

In some embodiments, the dosing schedule may be limited to a specific number of administrations or “cycles”. In some embodiments, the Binder-drug conjugate is administered for 3, 4, 5, 6, 7, 8, or more cycles. For example, the Binder-drug conjugate is administered every 2 weeks for 6 cycles, the Binder-drug conjugate is administered every 3 weeks for 6 cycles, the Binder-drug conjugate is administered every 2 weeks for 4 cycles, the Binder-drug conjugate is administered every 3 weeks for 4 cycles, etc. Dosing schedules can be decided upon and subsequently modified by those skilled in the art.

Thus, the present invention provides methods of administering to a subject the polypeptides or agents described herein comprising using an intermittent dosing strategy for administering one or more agents, which may reduce side effects and/or toxicities associated with administration of a binder-drug conjugate, chemotherapeutic agent, etc. In some embodiments, a method for treating cancer in a human subject comprises administering to the subject a therapeutically effective dose of a binder-drug conjugate in combination with a therapeutically effective dose of a chemotherapeutic agent, wherein one or both of the agents are administered according to an intermittent dosing strategy. In some embodiments, the intermittent dosing strategy comprises administering an initial dose of a binder-drug conjugate to the subject, and administering subsequent doses of the Binder-drug conjugate about once every 2 weeks. In some embodiments, the intermittent dosing strategy comprises administering an initial dose of a binder-drug conjugate to the subject, and administering subsequent doses of the Binder-drug conjugate about once every 3 weeks. In some embodiments, the intermittent dosing strategy comprises administering an initial dose of a binder-drug conjugate to the subject, and administering subsequent doses of the Binder-drug conjugate about once every 4 weeks. In some embodiments, the Binder-drug conjugate is administered using an intermittent dosing strategy and the chemotherapeutic agent is administered weekly.

In certain embodiments, the invention also provides methods for treating subjects using a binder-drug conjugate of the invention, wherein the subject suffers from a viral infection. In one embodiment, the viral infection is infection with a virus selected from the group consisting of human immunodeficiency virus (HIV), hepatitis virus (A, B, or C), herpes virus (e.g., VZV, HSV-I, HAV-6, HSV-II, and CMV, Epstein Barr virus), adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus or arboviral encephalitis virus.

In an embodiment, the invention provides methods for treating subjects using a binder-drug conjugate thereof of the invention, wherein the subject suffers from a bacterial infection. In one embodiment, the bacterial infection is infection with a bacterium selected from the group consisting of Chlamydia, rickettsial bacteria, mycobacteria, staphylococci, streptococci, pneumonococci, meningococci and gonococci, klebsiella, proteus, serratia, pseudomonas, Legionella, Corynebacterium diphtheriae, Salmonella, bacilli, Vibrio cholerae, Clostridium tetan, Clostridium botulinum, Bacillus anthricis, Yersinia pestis, Mycobacterium leprae, Mycobacterium lepromatosis, and Borriella.

In an embodiment, the invention provides methods for treating subjects using a binder-drug conjugate of the invention, wherein the subject suffers from a fungal infection. In one embodiment, the fungal infection is infection with a fungus selected from the group consisting of Candida (albicans, krusei, glabrata, tropicalis, etc.), Cryptococcus neoformans, Aspergillus (fumigatus, niger, etc.), Genus Mucorales (mucor, absidia, rhizopus), Sporothrix schenkii, Blastomyces dermatitidis, Paracoccidioides brasiliensis, Coccidioides immitis and Histoplasma capsulatum.

In an embodiment, the invention provides methods for treating subjects using a binder-drug conjugate of the invention, wherein the subject suffers from a parasitic infection. In one embodiment, the parasitic infection is infection with a parasite selected from the group consisting of Entamoeba histolytica, Balantidium coli, Naegleria fowleri, Acanthamoeba, Giardia lambia, Cryptosporidium, Pneumocystis carinii, Plasmodium vivax, Babesia microti, Trypanosoma brucei, Trypanosoma cruzi, Leishmania donovani, Toxoplasma gondii and Nippostrongylus brasiliensis.

a. PGE2 Inhibitors

In certain embodiments, the binder-drug conjugate is administered in combination with an agent that inhibits PGE2 production. The process of PGE2 synthesis involves phospholipase A2 (PLA2) family members that mobilize arachidonic acid from cellular membranes, cyclooxygenases (constitutively-active COX1 and inducible COX2) that convert arachidonic acid into prostaglandin H2 (PGH2), and prostaglandin E synthase (PGES), needed for the final formulation of PGE2. While the rate of PGE2 synthesis and the resulting inflammatory process can be affected by additional factors, such as local availability of AA, in most physiologic conditions, the rate of PGE2 synthesis is controlled by local expression and activity of COX2.

In other embodiments, the subject binder-drug conjugate is administered in combination with agents which promote PGE2 degradation. The rate of PGE2 degradation is controlled by 15-hydroxyprostaglandin dehydrogenase (15-PGDH), suggesting that in addition to the rate of PGE2 synthesis, also the rate of PGE2 decay constitutes a target for therapeutic intervention in the subject binder-drug conjugate combinations.

In still other embodiments, the subject binder-drug conjugate is administered in combination with agents that reduce PGE2 responsiveness. Four different PGE2 receptors are EPi, EP2, EP3 and EP4. The signaling through the two Gs-coupled receptors, EP2 and EP4, is mediated by the adenylate cyclase-triggered cAMP/PKA/CREB pathway, mediating the dominant aspects of the anti-inflammatory and suppressive activity of PGE2. While EP2 is believed to signal in a largely cAMP-dependent fashion, EP4 also activates the PI3K-dependent ERK1/2 pathway. However, both EP2 and EP4 have been shown to activate the GSK3/β-catenin pathway. The expression of EP2 and the resulting responsiveness to PGE2 can be suppressed by hyper-methylation, as observed in patients with idiopathic lung fibrosis. These observations raise the possibility that, in addition to the regulation of PGE2 production and its degradation, the regulation of PGE2 responsiveness at the level of expression of individual PGE2 receptors can also contribute to the pathogenesis of human disease and be exploited in their therapy. In support, the use of synthetic inhibitors, preferentially affecting EP2, EP3, or EP4 signaling, allow for differential suppression of different aspects of PGE2 activity.

Agents which reduce PGE2 responsiveness also include prostaglandin (PG) signaling inhibitors. Prostaglandins signal through numerous receptors, with the key immunosuppressive effects being mediated by the activation of adenylate cyclase, the resulting elevation of the intracellular cyclic (c)AMP, PKA and the downstream activation of the PKA/CREB pathway.

Another level of interference with the PG responsiveness includes the interference with their binging to PG receptors. In case of PGE2, the two key cAMP-activating receptors are EP2 and EP4, for which a number of specific inhibitors exist.

The increase of cAMP levels induced by prostaglandins or other factors can be prevented by phosphodiesterases (PDEs; currently known 6 types, PDE1-PDE5 and PDE10, which reduce the levels of intracellular cAMP). PDEs can be controlled by phosphodiesterase inhibitors, which include such substances as xanthines (caffeine, aminophylline, IBMX, pentoxyphylline, theobromine, theophylline, or paraxanthine), which all increase the levels of intracellular cAMP, and the more selective synthetic and natural factors, including vinpocetine, cilostazol, inaminone, cilostazol, mesembrine, rolipram, ibudilast, drotaverine, piclamilast, sildafenil, tadalafil, verdenafil, or papaverine.

Furthermore, interference with PGE2 signaling (or with the signaling of other cAMP-elevating factors, such as histamine, of beta-adrenergic agonists) can be achieved by the inhibition of downstream signals of cAMP, such as PKA or CREB.

(a) Cyclooxygenase Inhibitors

In certain preferred embodiments, the subject binder-drug conjugate is administered in combination with one or more prostaglandin (PG) synthesis inhibitors. Factor which inhibit the synthesis of PGs in general or the synthesis of a specific type of PGs. PG synthesis inhibitors include nonselective inhibitors of COX-1 and COX-2, the two key enzymes in the PG synthesis pathway, and selective inhibitors of COX-2, which are believed to be more specific to COX-2 and less toxic. The examples of non-selective PG inhibitors include aspirin, indomethacin, or ibuprofen (Advil, Motrin). The examples of COX-2-selective inhibitors include Celecoxib (Celebrex) and rofecoxib (Vioxx). The example of COX-1-specific inhibitor is sulindac (Clinoril). Other drugs that suppress prostaglandin synthesis include steroids (example: hydrocortisone, cortisol, prednisone, or dexamethasone) and acetaminophen (Tylenol, Panadol), commonly used as anti-inflammatory, antipyretic and analgesic drugs. Examples of the most commonly used selective COX2 inhibitors include celecoxib, alecoxib, valdecoxib, and rofecoxib.

Examples of the most commonly used non-selective COX 1 and COX2 inhibitors include: acetylsalicylic acid (aspirin) and other salicylates, acetaminophen (Tylenol), ibuprofen (Advil, Motrin, Nuprin, Rufen), naproxen (Naprosyn, Aleve), nabumetone (Relafen), or diclofenac (Cataflam).

A component of the present invention is a Cox-2 inhibitor. The terms “cyclooxygenase-2 inhibitor”, or “Cox-2 inhibitor”, which can be used interchangeably herein, embrace compounds which inhibit the Cox-2 enzyme regardless of the degree of inhibition of the Cox-1 enzyme, and include pharmaceutically acceptable salts of those compounds. Thus, for purposes of the present invention, a compound is considered a Cox-2 inhibitor irrespective of whether the compound inhibits the Cox-2 enzyme to an equal, greater, or lesser degree than the Cox-1 enzyme.

In one embodiment of the present invention, it is preferred that the Cox-2 inhibitor compound is a non-steroidal anti-inflammatory drug (NSAID). Therefore, preferred materials that can serve as the Cox-2 inhibitor of the present invention include non-steroidal anti-inflammatory drug compounds, a pharmaceutically acceptable salt thereof, or a pure (−) or (+) optical isomeric form thereof.

Examples of NSAID compounds that are useful in the present invention include acemetacin, acetyl salicylic acid, alclofenac, alminoprofen, azapropazone, benorylate, benoxaprofen, bucloxic acid, carprofen, choline magnesium trisalicylate, clidanac, clopinac, dapsone, diclofenac, diflunisal, droxicam, etodolac, fenoprofen, fenbufen, fenclofenec, fentiazac, floctafenine, flufenisal, flurbiprofen, (r)-flurbiprofen, (s)-flurbiprofen, furofenac, feprazone, flufenamic acid, fluprofen, ibufenac, ibuprofen, indometacin, indomethacin, indoprofen, isoxepac, isoxicam, ketoprofen, ketorolac, miroprofen, piroxicam, meloxicam, mefenamic, mefenamic acid, meclofenamic acid, meclofen, nabumetone, naproxen, niflumic acid, oxaprozin, oxipinac, oxyphenbutazone, phenylbutazone, podophyllotoxin derivatives, proglumetacin, piprofen, pirprofen, prapoprofen, salicylic acid, salicylate, sudoxicam, suprofen, sulindac, tenoxicam, tiaprofenic acid, tiopinac, tioxaprofen, tolfenamic acid, tolmetin, zidometacin, zomepirac, and 2-fluoro-a-methyl[1,1′-biphenyl]-4-acetic acid, 4-(nitrooxy)butyl ester.

In a preferred embodiment, the Cox-2 inhibitor is a Cox-2 selective inhibitor. The term “Cox-2 selective inhibitor” embraces compounds which selectively inhibit the Cox-2 enzyme over the Cox-1 enzyme, and also include pharmaceutically acceptable salts and prodrugs of those compounds. In certain embodiments, the PGE2 antagonist is not indomethacin.

In practice, the selectivity of a Cox-2 inhibitor varies depending upon the condition under which the test is performed and on the inhibitors being tested. However, for the purposes of this specification, the selectivity of a Cox-2 inhibitor can be measured as a ratio of the in vitro or in vivo IC50 value for inhibition of Cox-1, divided by the IC50 value for inhibition of Cox-2 (Cox-1 IC50/Cox-2 IC50). A Cox-2 selective inhibitor is any inhibitor for which the ratio of Cox-1 IC50 to Cox-2 IC50 is greater than 1. In preferred embodiments, this ratio is greater than 2, more preferably greater than 5, yet more preferably greater than 10, still more preferably greater than 50, and more preferably still greater than 100.

As used herein, the term “IC50” refers to the concentration of a compound that is required to produce 50% inhibition of cyclooxygenase activity. Preferred Cox-2 selective inhibitors of the present invention have a Cox-2 IC50 of less than about 1 μM, more preferred of less than about 0.5 μM, and even more preferred of less than about 0.2 μM.

Preferred Cox-2 selective inhibitors have a Cox-1 IC50 of greater than about 1 μM, and more preferably of greater than 20 μM. Such preferred selectivity may indicate an ability to reduce the incidence of common NSAID-induced side effects.

Also included within the scope of the present invention are compounds that act as prodrugs of Cox-2-selective inhibitors. As used herein in reference to Cox-2 selective inhibitors, the term “prodrug” refers to a chemical compound that can be converted into an active Cox-2 selective inhibitor by metabolic or simple chemical processes within the body of the subject. One example of a prodrug for a Cox-2 selective inhibitor is parecoxib, which is a therapeutically effective prodrug of the tricyclic Cox-2 selective inhibitor valdecoxib. An example of a preferred Cox-2 selective inhibitor prodrug is sodium parecoxib. A class of prodrugs of Cox-2 inhibitors is described in U.S. Pat. No. 5,932,598 (incorporated by reference).

The Cox-2 selective inhibitor of the present invention can be, for example, the Cox-2 selective inhibitor meloxicam, (CAS registry number 71125-38-7), or a pharmaceutically acceptable salt or prodrug thereof.

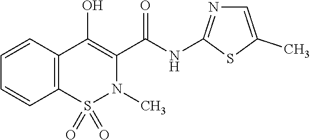 [SMILES: Cc1cnc(NC(=O)C2=C(O)c3ccccc3S(=O)(=O)N2C)s1]

In another embodiment of the invention the Cox-2 selective inhibitor can be the Cox-2 selective inhibitor RS 57067, 6-[[5-(4-chlorobenzoyl)-1,4-dimethyl-1H-pyrrol-2-yl]methyl]-3(2H)-pyridazinone, (CAS registry number 179382-91-3), or a pharmaceutically acceptable salt or prodrug thereof.

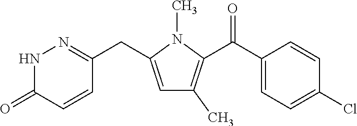 [SMILES: Cc1cc(Cc2ccc(=O)[nH]n2)n(C)c1C(=O)c1ccc(Cl)cc1]

Other examples include:

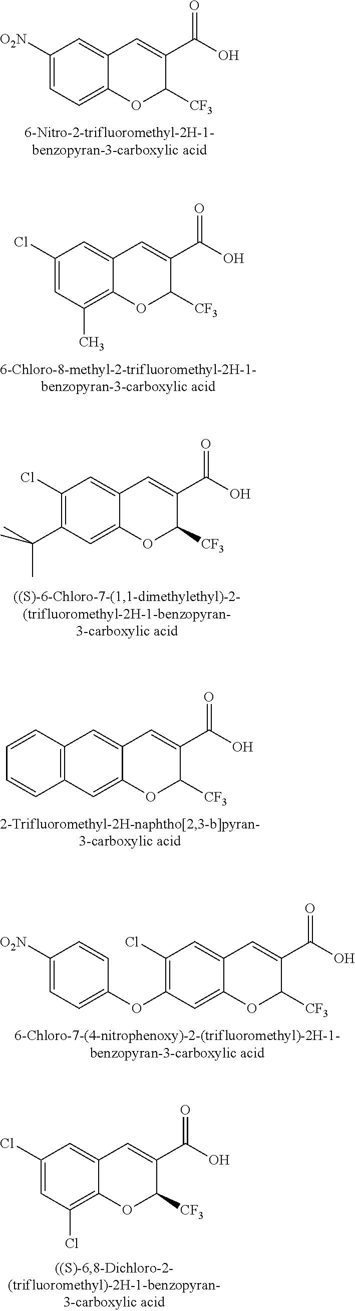 [SMILES: O=C(O)C1=Cc2cc([N+](=O)[O-])ccc2OC1C(F)(F)F ; Cc1cc(Cl)cc2c1OC(C(F)(F)F)C(C(=O)O)=C2 ; CC(C)(C)c1cc2c(cc1Cl)C=C(C(=O)O)[C@@H](C(F)(F)F)O2 ; O=C(O)C1=Cc2cc3ccccc3cc2OC1C(F)(F)F ; O=C(O)C1=Cc2cc(Cl)c(Oc3ccc([N+](=O)[O-])cc3)cc2OC1C(F)(F)F ; O=C(O)C1=Cc2cc(Cl)cc(Cl)c2O[C@@H]1C(F)(F)F]

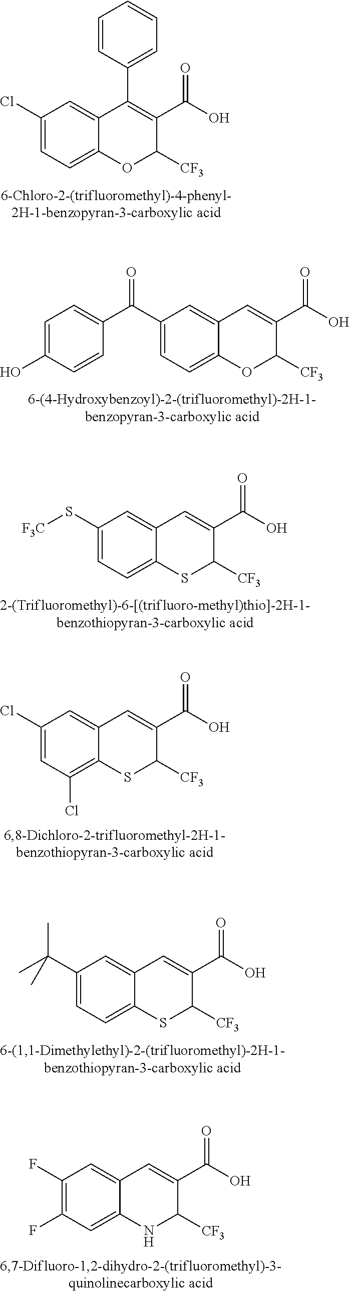 [SMILES: O=C(O)C1=C(c2ccccc2)c2cc(Cl)ccc2OC1C(F)(F)F ; O=C(O)C1=Cc2cc(C(=O)c3ccc(O)cc3)ccc2OC1C(F)(F)F ; O=C(O)C1=Cc2cc(SC(F)(F)F)ccc2SC1C(F)(F)F ; O=C(O)C1=Cc2cc(Cl)cc(Cl)c2SC1C(F)(F)F ; CC(C)(C)c1ccc2c(c1)C=C(C(=O)O)C(C(F)(F)F)S2 ; O=C(O)C1=Cc2cc(F)c(F)cc2NC1C(F)(F)F]

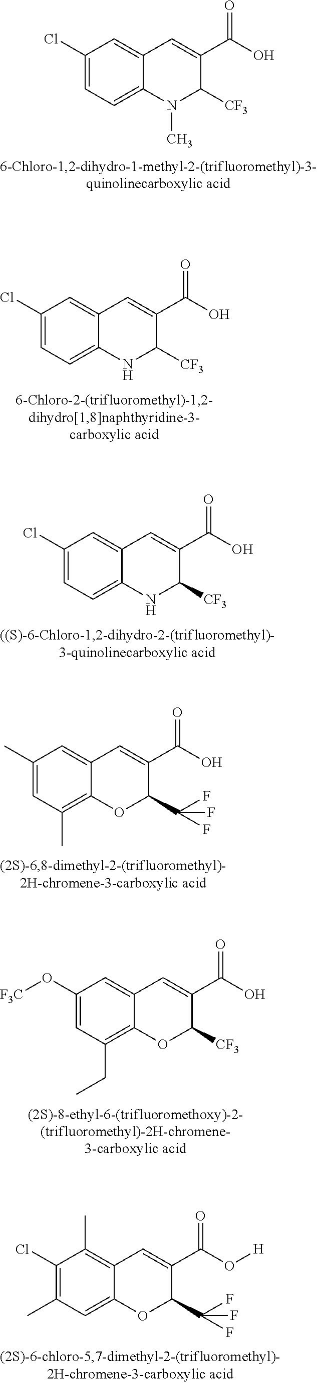 [SMILES: CN1c2ccc(Cl)cc2C=C(C(=O)O)C1C(F)(F)F ; O=C(O)C1=Cc2cc(Cl)ccc2NC1C(F)(F)F ; O=C(O)C1=Cc2cc(Cl)ccc2N[C@@H]1C(F)(F)F ; Cc1cc(C)c2c(c1)C=C(C(=O)O)[C@@H](C(F)(F)F)O2 ; CCc1cc(OC(F)(F)F)cc2c1O[C@H](C(F)(F)F)C(C(=O)O)=C2 ; Cc1cc2c(c(C)c1Cl)C=C(C(=O)O)[C@@H](C(F)(F)F)O2]

In preferred embodiments the chromene Cox-2 inhibitor is selected from (S)-6-chloro-7-(1,1-dimethylethyl)-2-(trifluoromethyl)-2H-1-benzopyran-3-carboxylic acid, (2S)-6,8-dimethyl-2-(trifluoromethyl)-2H-chromene-3-carboxylic acid, (2S)-6-chloro-8-methyl-2-(trifluoromethyl)-2H-chromene-3-carboxylic acid, (2S)-8-ethyl-6-(trifluoromethoxy)-2-(trifluoromethyl)-2H-chromene-3-carboxylic acid, (S)-6,8-dichloro-2-(trifluoromethyl)-2H-1-benzopyran-3-carboxylic acid, (2S)-6-chloro-5,7-dimethyl-2-(trifluoromethyl)-2H-chromene-3-carboxylic acid, and mixtures thereof.

In a preferred embodiment of the invention the Cox-2 inhibitor can be selected from the class of tricyclic Cox-2 selective inhibitors represented by the general structure of:

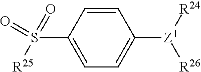

wherein:
Z¹ is selected from the group consisting of partially unsaturated or unsaturated heterocyclyl and partially unsaturated or unsaturated carbocyclic rings;
R²⁴ is selected from the group consisting of heterocyclyl, cycloalkyl, cycloalkenyl and aryl, wherein R²⁴ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R²⁵ is selected from the group consisting of methyl or amino; and
R²⁶ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl;
or a prodrug thereof.

In a preferred embodiment of the invention the Cox-2 selective inhibitor represented by the above formula is selected from the group of compounds which includes celecoxib (B-21), valdecoxib (B-22), deracoxib (B-23), rofecoxib (B-24), etoricoxib (MK-663; B-25), JTE-522 (B-26), or prodrugs thereof.

Additional information about selected examples of the Cox-2 selective inhibitors discussed above can be found as follows: celecoxib (CAS RN 169590-42-5, C-2779, SC-58653, and in U.S. Pat. No. 5,466,823 (incorporated by reference)); deracoxib (CAS RN 169590-41-4); rofecoxib (CAS RN 162011-90-7); compound B-24 (U.S. Pat. No. 5,840,924); compound B-26 (WO 00/25779 (incorporated by reference)); and etoricoxib (CAS RN 202409-33-4, MK-663, SC-86218, and in WO 98/03484 (incorporated by reference)).

Structural Formula

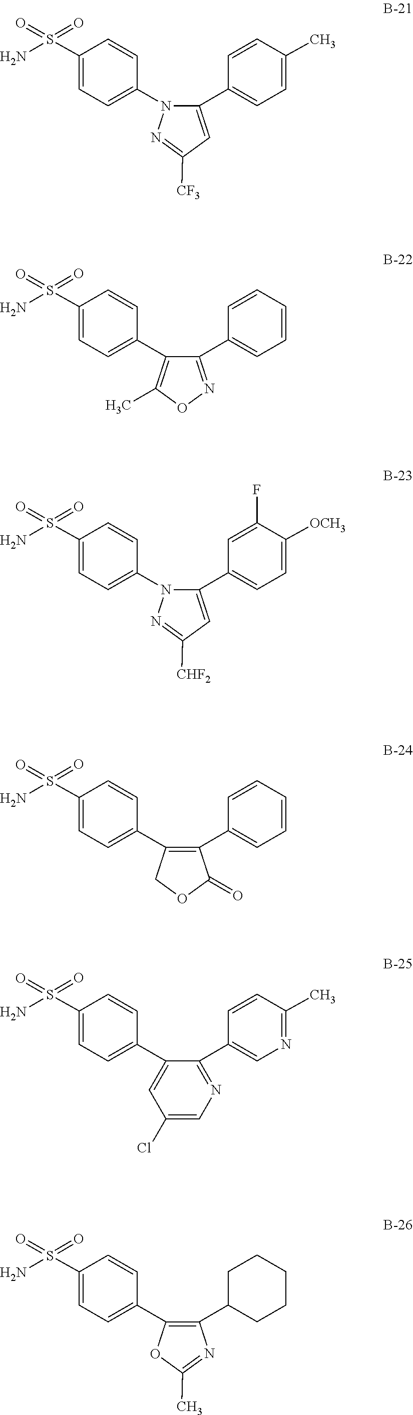 [SMILES: Cc1ccc(-c2cc(C(F)(F)F)nn2-c2ccc(S(N)(=O)=O)cc2)cc1 ; Cc1onc(-c2ccccc2)c1-c1ccc(S(N)(=O)=O)cc1 ; COc1ccc(-c2cc(C(F)F)nn2-c2ccc(S(N)(=O)=O)cc2)cc1F ; NS(=O)(=O)c1ccc(C2=C(c3ccccc3)C(=O)OC2)cc1 ; Cc1ccc(-c2ncc(Cl)cc2-c2ccc(S(N)(=O)=O)cc2)cn1 ; Cc1nc(C2CCCCC2)c(-c2ccc(S(N)(=O)=O)cc2)o1]

In a more preferred embodiment of the invention, the Cox-2 selective inhibitor is selected from the group consisting of celecoxib, rofecoxib and etoricoxib.

In a preferred embodiment, parecoxib (See, U.S. Pat. No. 5,932,598 (incorporated by reference)), having the structure shown in B-27, and which is a therapeutically effective prodrug of the tricyclic Cox-2 selective inhibitor valdecoxib, B-22, (See, U.S. Pat. No. 5,633,272 (incorporated by reference)), may be advantageously employed as the Cox-2 inhibitor of the present invention.

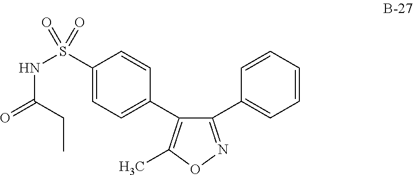 [SMILES: CCC(=O)NS(=O)(=O)c1ccc(-c2c(-c3ccccc3)noc2C)cc1]

A preferred form of parecoxib is sodium parecoxib.

Another tricyclic Cox-2 selective inhibitor useful in the present invention is the compound ABT-963, having the formula B-28 shown below, that has been previously described in International Publication Number WO 00/24719.

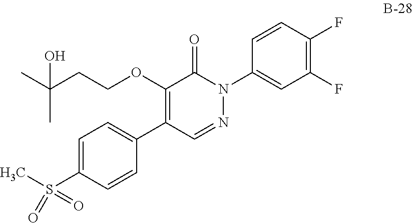 [SMILES: CC(C)(O)CCOc1c(-c2ccc(S(C)(=O)=O)cc2)cnn(-c2ccc(F)c(F)c2)c1=O]

(b) Cytosolic Phospholipases A2 (cPLA2) Inhibitors

In certain embodiments, the PGE2 inhibitor is an inhibitor of cytosolic phospholipases A2 (cPLA2), such as, merely to illustrate, arachidonyl trifluoromethyl ketone,

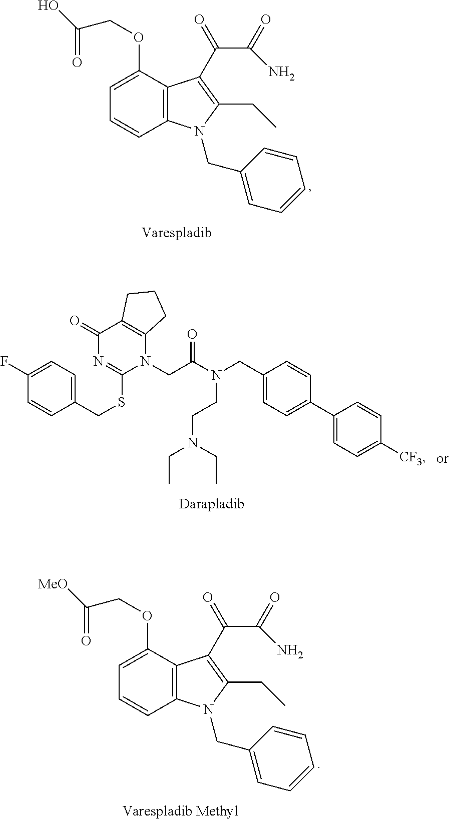 [SMILES: CCc1c(C(=O)C(N)=O)c2c(OCC(=O)O)cccc2n1Cc1ccccc1 ; CCN(CC)CCN(Cc1ccc(-c2ccc(C(F)(F)F)cc2)cc1)C(=O)Cn1c(SCc2ccc(F)cc2)nc(=O)c2c1CCC2 ; "or" ; CCc1c(C(=O)C(N)=O)c2c(OCC(=O)OC)cccc2n1Cc1ccccc1]

### VI. Certain Examples

AVA04-251 Fc
(SEQ ID No. 117)
IPRGLSEAKPATPEIQEIVDKVKPQLEEKTGETYGKLEAVQYKTQVLAR
EGRQDWVLSTNYYIKVRAGDNKYMHLKVFNGPWVPFPHQQLADRVLTGY
QVDKNKDDELTGFAAAGGGGSGGGGSGGGGSGGGGSEPKSCDKTHTCPP
CPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFS
CSVMHEALHNHYTQKSLSLSPGK
AVA04-182 Fc
(SEQ ID No. 118)
IPRGLSEAKPATPEIQEIVDKVKPQLEEKTGETYGKLEAVQYKTQVLAF
ALPEFEYMSTNYYIKVRAGDNKYMHLKVFNGPPMIRRKNEVADRVLTGY
QVDKNKDDELTGFLHAAAGGGGSGGGGSGGGGSGGGGSEPKSCDKTHTC
PPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNV
FSCSVMHEALHNHYTQKSLSLSPGK
AVA04-251 BH cys
(SEQ ID No. 119)
MIPRGLSEAKPATPEIQEIVDKVKPQLEEKTNETYGKLEAVQYKTQVLA
REGRQDWVLSTNYYIKVRAGDNKYMHLKVFNGPWVPFPHQQLADRVLTG
YQVDKNKDDELTGFAEAAAKEAAAKEAAAKEAAAKEAAAKEAAAKMIPR
GLSEAKPATPEIQEIVDKVKPQLEEKTNETYGKLEAVQYKTQVLAREGR
QDWVLSTNYYIKVRAGDNKYMHLKVFNGPWVPFPHQQLADRVLTGYQVD
KNKDDELTGFLQAAAHHHHHHC
SQT gly Fc
(SEQ ID No. 120)
IPRGLSEAKPATPEIQEIVDKVKPQLEEKTGETYGKLEAVQYKTQVLAG
GGGGGGGGSTNYYIKVRAGDNKYMHLKVFNGPGGGGGGGGGADRVLTGY
QVDKNKDDELTGFLQAAAGGGGSGGGGSGGGGSGGGGSEPKSSDKTHTC
PPCPAPELLGGPSVFLFPPKPKDTLYITREPEVTCVVVDVSHEDPEVKF
NWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNV
FSCSVMHEALHNHYTQKSLSLSPGK

### Example 1: Selection of PD-L1 Binding Affimers from Phage Display Library

A peptide of the present invention, for example, a PD-L1 binding component, may be identified by selection from a library of affimers with two random loops, for example, generally but not exclusively of the same length of 9 amino acids.

As indicated above, the PD-L1 binding peptides of the invention were identified by selection from a phage display library comprising random loop sequences nine amino acids in length displayed in a constant affimer framework backbone based upon the sequence for Stefin A. Such selection procedures are generally known. According to such procedures, suspensions of phage are incubated with target antigen (either biotinylated antigen captured on streptavidin beads or unbiotinylated antigen captured on a plate). Unbound phage are then washed away and, subsequently, bound phage are eluted either by incubating the antigen with low pH, followed by high pH. E. coli are then infected with released, pH neutralised phage and a preparation of first round phage is obtained. The cycle is performed repeatedly, for example, two or three times and, in order to enrich for targeting phage, the stringency conditions may be increased in the later rounds of selection, for example by increasing the number of wash steps, reducing the antigen concentration, and preselecting with blocked streptavidin beads or wells coated with blocking reagent.

Following selection by successive rounds of phage amplification, PD-L1 binding clones were identified by a soluble affimer ELISA. Briefly, affimer was overexpressed from the phagemid vector, the bacterial cell lysed and the lysate used in an ELISA, detecting affimer binding to PD-L1 immobilised on a plate with a conjugated antibody to the His6 tag on the affimer.

To illustrate, selection of PD-L1 binding phage from the affimer library was carried out as described below using approximately 1×10¹¹ phage are added from a library of size approximately 6×10¹⁰ diversity.

Biotinylated antigen captured on M280 streptavidin or neutravidin beads (Thermo Scientific) Briefly, affimer was overexpressed from the phagemid vector, the bacterial cell lysed and the lysate used in an ELISA, detecting affimer binding to PD-L1 immobilised on a plate with a conjugated antibody to the His6 tag on the affimer was used for selections. Antigen was supplied in an Fc-cleaved format by R & D and was biotinylated in-house using the EZ Link Sulfo-NHS-LC Biotin kit (Pierce).

Before addition of the phage to antigen on beads, both phage and beads were blocked with 2% Marvel-PBS, each for 1 hour to reduce non-specific binding interactions. The phage were then allowed to bind the antigen for 1 hour at RT before removing unbound or weakly bound phage by washing five times with PBS-0.1% Tween 20 followed by 2 washes with PBS, eluting phage using 100 mM trietyhylamine, removing elution and neutralising with Tris buffer, then eluting a second time with 0.1M HCl, also neutralised with Tris buffer. Elutions were pooled and harvested phage were titrated as colony forming units (CFU) before amplification in E. coli TG1 cells, rescued using helper phage M13KO7 and purified before titreing and used as the input for the next round of panning. For the second and third rounds of panning, the wash stringency was increased by using more cycles of washes with PBS-Tween and reducing the antigen. Deselection steps were also introduced at round 2 and 3 by preselection of input phage with blocked beads to remove bead binders, and beads were swapped between neutravidin and streptavidin at each round to reduce streptavidin binders.

Following titration of phase from the second and third round fractions, single, well-isolated plaques were picked, soluble affimer expressed and characterised for antigen binding by crude extract ELISA. Briefly, affimer was overexpressed from the phagemid vector by IPTG induction, the bacterial cell lysed using reagent B-PER II (Thermo Scientific) and the centrifugally cleared lysate used in an ELISA, detecting affimer binding to PD-L1 immobilised on a plate with an HRP conjugated antibody (Miltenyi Biotec) to the His6 tag on the affimer, developing the ELISA using 1-step Ultra TMB-ELISA substrate (Thremo Scientific). Clones showing binding above background on negative control plates with no antigen coating were rejected as nonspecific binders. Clones showing specific binding were sequenced to identify loop sequences.

### Example 2: Binding Affinity of Anti-PD-L1 Affimer to Human Cancer Cell Line Expressing PD-L1

The Affinity of AVA04 Affimers was determined using Flow cytometry H441 cells expressing PD-L1 grown in RPMI-2640 (Sigma) containing 10% of FBS (Gibco) with Penicillin (100 U/mL, Hyclone) and Streptomycin (100 μg/mL, Hyclone) where detached from the tissue culture washed using DPBS. Cells where collected by centrifugation at 300 rpm for 5 min. The cells were resuspended in PBS and 50000 cells per wells were dispatched in a round bottom 96 well plate. Cells were washed with PBS. Affimer and controls were diluted in staining buffer (R&D) in duplicate and added on cells for staining for approximately 60 min at 4±1° C. Cells were washed and the secondary anti-Cystatin A (R&D) was diluted 1:15 in staining buffer (R&D) and added on cells for staining for approximately 40 min at 4±1° C. Cells were washed and the detection antibody A488 anti-Goat (Biolegend) was diluted 1:100 in staining buffer (R&D) and added on cells for staining for approximately 30 min at 4±1° C. Finally, cells were washed live and dead cells were stained using L/D stain Zombie Aqua (Biolegend) diluted in staining buffer for 10 min at 4±1° C. Cells were washed and fixation buffer (R&D) was added to each well for 10 min at 4±1° C. then PBS with EDTA (Lonza) was added prior reading the plate on the flow cytometer (Guava 12 HT, Millipore). Dead cells were excluded and the fluorescent Green channel (488 nm/525/30) was acquired. Results were analysed using Incyte and data were plotted using graphpad.

### Cell Binding Assay on MDA-MB-231

Cell Preparation:
   Detach MDA-MB-231 cells (ATCC) and dilute them into 0.25×10e6 cells/ml, 80 ml.
   Pipet 200 ul of cell suspension (50000 cells) into every well, 4 plates.
   Centrifuge 300 g, 5 min, discard the supernatant.
   Resuspend cells into 200 ul PBS. Centrifuge 300 g, 5 min and discard the supernatant.
Affimer and control dilution and staining
   Make Affimer and antibody dilutions according to the table (Atezolizumab, Invivogen from 3.5 nM and Affimer from 500 nM)
   On separate dilution plates:
   60 ul staining buffer on wells A1, B1, A2 and B2
   60 ul staining buffer to rows A and B wells 4 to 12
   60 ul staining buffer to rows C to H wells 2 to 12
1. 100 ul Atezolizumab (InVivogen) from 3.5 nM on wells A3 and B3 Transfer 30 ul from wells A3 and B3 to A4 and B4, etc until A12 and B12
2. Pipet 100 ul of Affmer dilutions into corresponding column 1 wells according to the table above.
   Transfer 20 ul from well 1 to 2, 2 to 3 etc until 12
   Pipet 50 ul from the dilution plate wells into corresponding wells on the assay plates with the cells and mix well.
   Incubate 60 min at +5C
Wash: Add 150 ul/well of PBS and centrifuge 300 g, 5 min. Discard the supernatant. Repeat once more
Staining with Anti Cystatin:
Pipet 50 ul/well staining buffer into wells A1 to A12, and B2 to B12
Make 22000 ul 1:15 dilution of Anti-Cystatin antibody in staining buffer: 1467 ul Ab+20533 ul staining buffer. Pipet 50 ul/well into Bi and rows C to H wells. Incubate 40 min at +5C
Wash Add 150 ul/well of PBS and centrifuge 300 g, 5 min. Discard the supernatant. Repeat once more
Staining with Secondary Ag:
   Make 5000 ul of 1:100 dilution of AF488 Anti-Human IgG (Biolegend): 50 ul
   Anti-Human IgG+4950 ul staining buffer
   Pipet 50 ul/well into rows A and B wells 2 to 12
   Make 25000 ul of 1:500 dilution of Anti-Goat AF488 (Biolegend) antibody in staining buffer: 50 ul Ab+24950 ul staining buffer.
   Pipet 50 ul/well into B1 and rows C to H wells
   Incubate 30 min at +5C
   Add 150 ul/well of PBS and centrifuge 300 g, 5 min. Discard the supernatant.
Live and Dead Staining:
   Make 25000 ul of 1:500 dilution of L/D stain Zombie Aqua (Biolegend) in staining buffer: 50 ul L/D stain+24950 ul staining buffer.
   Pipet 50 ul to every well on assay plates.
   Incubate 10 minutes at +5C
   Add 150 ul/well of PBS and centrifuge 300 g, 5 min. Discard the supernatant.
   Repeat once more
Fixation step:
   Add 100 ul/well fixation buffer
   Incubate 10 min at +5C
   Add 100 ul/well of PBS and centrifuge 300 g, 5 min. Discard the supernatant.
   Resuspend the cells into 100 ul staining buffer, store at +5C

Cell Binding Assay on H441
Cell Preparation:
   Detach H441 cells, and dilute them into 0.25×10e6 cells/ml, 85 ml.
   Pipet 200 ul of cell suspension (50000 cells) into every well, 4 plates.
   Centrifuge 300 g, 5 min, discard the supernatant.
Wash:
   Resuspend cells into 200 ul PBS with EDTA
   Centrifuge 300 g, 5 min and discard the supernatant.
Affimer and control dilution and staining
   Pipet 50 ul from the corresponding dilution plate wells into corresponding wells on the assay plates with the cells and mix well.
   Incubate 120 min at +5C
Wash:
   Add 150 ul/well of PBS with EDTA and centrifuge 300 g, 5 min. Discard the supernatant.
   Repeat once more
Staining with Anti Cystatin:
   Pipet 50 ul/well staining buffer into row A wells
   Make 18000 ul 1:15 dilution of Anti-Cystatin antibody (50 ug/ml stock) in staining buffer: 1200 ul Ab+16800 ul staining buffer.
   Pipet 50 ul/well into rows B to H wells
   No stocks in freezer, although LabGuru showed multiple. Instead, I had to use
   BAF1407 in this assay.
   Incubate 40 min at +5C
   Wash:
   Add 150 ul/well of PBS with EDTA and centrifuge 300 g, 5 min. Discard the supernatant.
   Repeat once more
Staining with Secondary Ag:
   Make 3000 ul of 1:100 dilution of AF488 Anti-Human IgG: 30 ul Anti-Human IgG+2970 ul staining buffer
   Pipet 50 ul/well into row A wells 2 to 12
   Make 18000 ul of 1:500 dilution of Anti-Goat AF488 (Biolegend) antibody in staining buffer: 36 ul Ab+18 ml staining buffer.
   Pipet 50 ul/well into rows B to H wells
   Pipet 50 ul staining buffer into A1 of all plates
   Incubate 30 min at +5C
   Add 150 ul/well of PBS with EDTA and centrifuge 300 g, 5 min. Discard the supernatant.
Live and Dead Staining:
   Make 22 ml of 1:500 dilution of L/D stain Zombie Aqua (Biolegend) in staining buffer: 44 ul L/D stain+22 ml staining buffer.
   Pipet 50 ul to every well on assay plates.
   Incubate 10 minutes at +5C
   Add 150 ul/well of PBS with EDTA and centrifuge 300 g, 5 min. Discard the supernatant.
   Repeat once more
Fixation step:
   Add 100 ul/well fixation buffer Incubate 10 min at +5C
   Add 100 ul/well of PBS with EDTA and centrifuge 300 g, 5 min. Discard the supernatant.
   Resuspend the cells into 100 ul staining buffer, store at +5C

### Example 3: Affimer Fc and In-Line Fusion Production and Characterisation

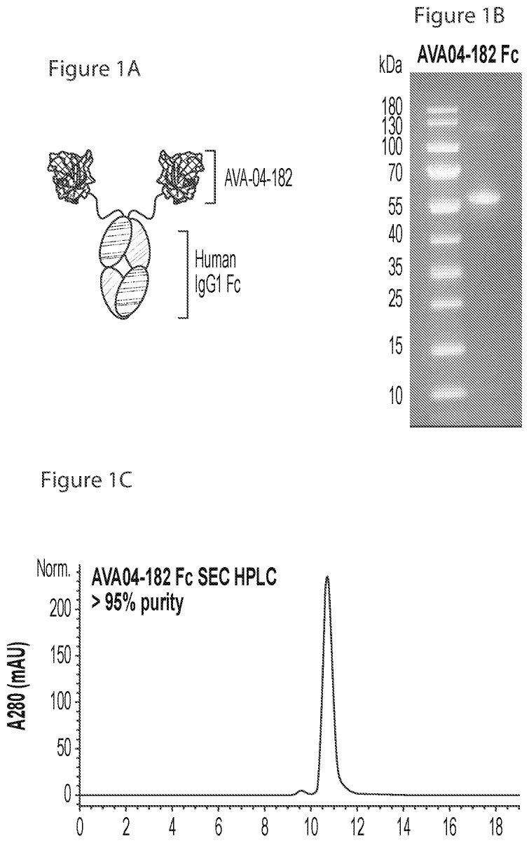

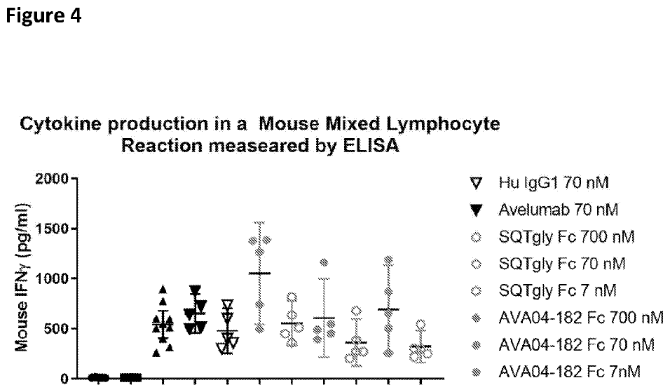

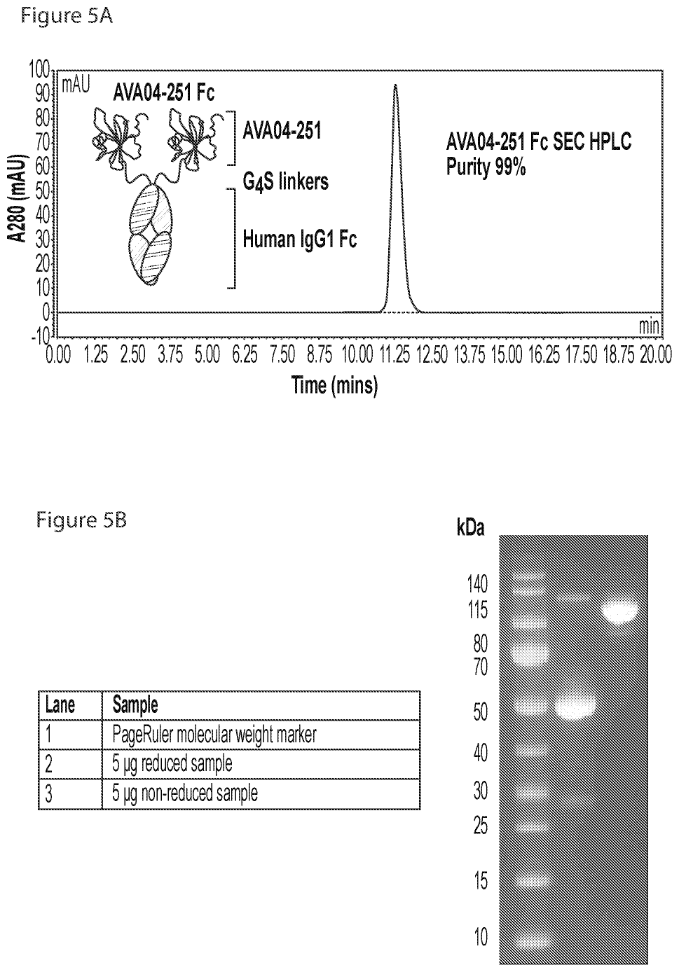

Suspension HEK cell (Expi293F cell line; Thermo) transient transfections were performed with Affimer Fc fusion constructs (AVA04-251 Fc and AVA04-182 Fc; see table above, schematic representation FIGS. 5A and 1A, respectively) using Expifectamine reagent (Thermo) following the manufacturer's protocol. Supernatant was harvested seven (7) days post-transfection by centrifuging at 20,000 g for 1 hour and filtering 0.45 μm. Protein was affinity purified using mabSelect Sure HiTrap columns on an AKTA Xpress (GE Healthcare). Resin was washed with five (5) column volumes (CV) distilled water and equilibrated with five (5) CV 1×PBS. Then, supernatant was run through at a flow rate of 5 mL/min followed by a wash with ten (10) CV 1×PBS. Bound protein was eluted in five (5) CV 0.1 M glycine pH 2.8 followed by buffer exchange into 1×PBS using Centripure desalting columns (empBiotech GmbH). A second stage purification was performed by preparative size-exclusion chromatography (SEC) using a HiLoad 26/600 Superdex 200 pg column (GE Healthcare), run in 1×PBS at 2.6 mL/min flow rate on an AKTA Xpress (GE Healthcare). Analytical SEC was carried out using a MAbPac SEC-1 (Thermo) or Yarra-3000 column (Phenomenex), run on an Ultimate 3000 HPLC (Thermo) at 0.8 mL/min in 1×PBS. AVA04-182 Fc (SEQ ID NO: 118) and AVA04-251 Fc (SEQ ID NO: 117) final protein batches showed >95% purity (FIGS. 1C and 4A, respectively). The protein yield was estimated using Nanodrop (Thermo) A280 readings and the product run on a SDS-PAGE Bolt Bis Tris plus 4-12% gel (Thermo) in Novex™ 20× Bolt™ MES SDS running buffer (Thermo) at 200V, with samples heated in reducing buffer. Protein bands on gel were stained with Quick Commassie (Generon). PageRuler prestained protein molecular weight marker (Thermo) was run on the gel to estimate the molecular weight of the fusion proteins (FIGS. 1B and 5B).

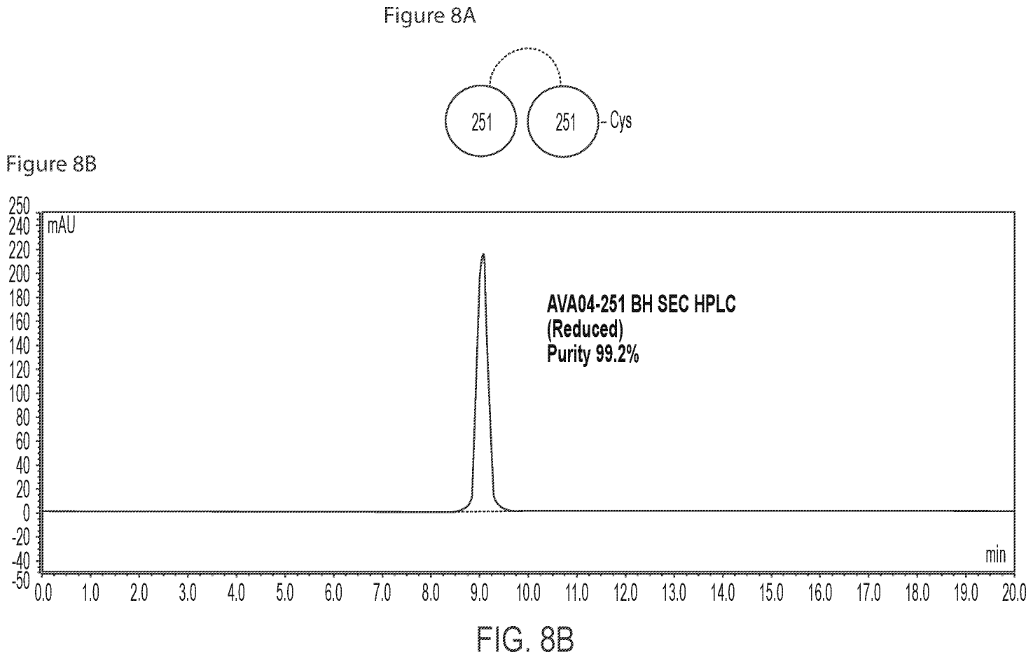

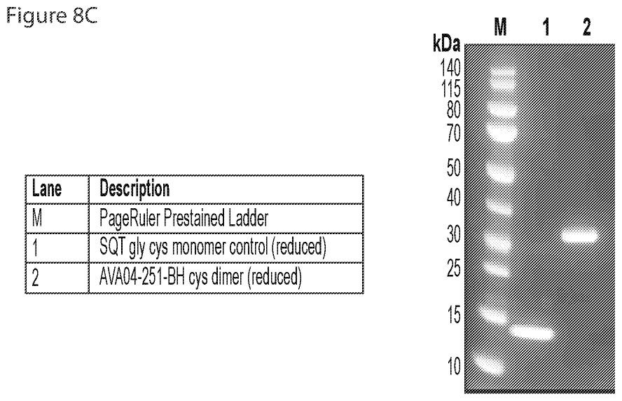

Affimer in-line fusion protein AVA04-251 BH cys (SEQ ID NO: 119, schematic representation FIG. 8A) was produced from E. coli and purified using affinity, ion-exchange and size-exclusion chromatography. The expression plasmid pD861 (Atum) was transformed into BL21 E. coli cells (Millipore) using the manufacturer's protocol. The total transformed cell mixture was plated onto LB agar plates containing 50 μg/mL kanamycin (AppliChem) and incubated at 37° C. overnight. The following day, the lawn of transformed E. coli was transferred to a sterile flask of 1× terrific broth media (Melford) & 50 μg/mL kanamycin and incubated at 30° C. shaking at 250 rpm. Expression was induced with 10 mM rhamnose (Alfa Aesar) once the cells have reached an OD600 of ˜0.8-1.0 and the culture incubated for a further 5 hours at 37° C. Cells were harvested by centrifuging and lysing the cell pellet. Affimer purification was performed using batch bind affinity purification of the His tagged protein using Nickel agarose affinity resin (Super-NiNTA500; Generon). Unbound protein was removed with five (5) CV NPI20, followed by elution of bound protein with five (5) CV of NPI400 buffer and reducing agent (50 mM sodium phosphate, 0.5 M NaCl, 0.4 M imidazole, 10 mM TCEP). Eluted protein was subsequently buffer exchanged using a cation exchange purification step based on a CM FF ion-exchange column (GE Healthcare) run in 20 mM MES pH 6, with a 0.1% triton X-114 (Sigma) wash step and eluting with a 1 M NaCl gradient. A third stage purification was performed based on preparative SEC using a HiLoad 26/600 Superdex 75 pg column (GE Healthcare) run in 1×PBS. AVA04-251 BH cys was formulated in a final reducing buffer containing 50 mM MES pH 6.0, 150 mM NaCl, 10 mM TCEP. Analytical SEC was carried out using an Accliam SEC-300 column (Thermo) run on an Ultimate 3000 HPLC (Thermo) at 0.7 mL/min in 1×PBS mobile phase. SEC HPLC and SDS-PAGE analytics show the final protein is >99% pure when reduced (FIGS. 8B and 8C, respectively).

### Example 4: Kinetic Analysis of Anti-PD-L1 Binding to Affimer Fc Fusion Proteins

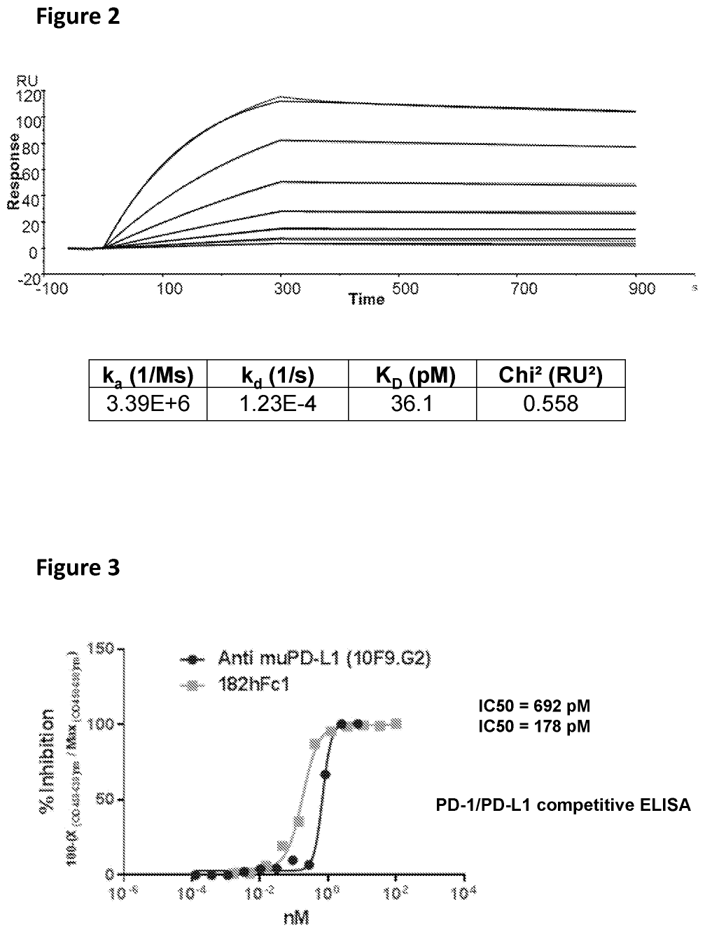

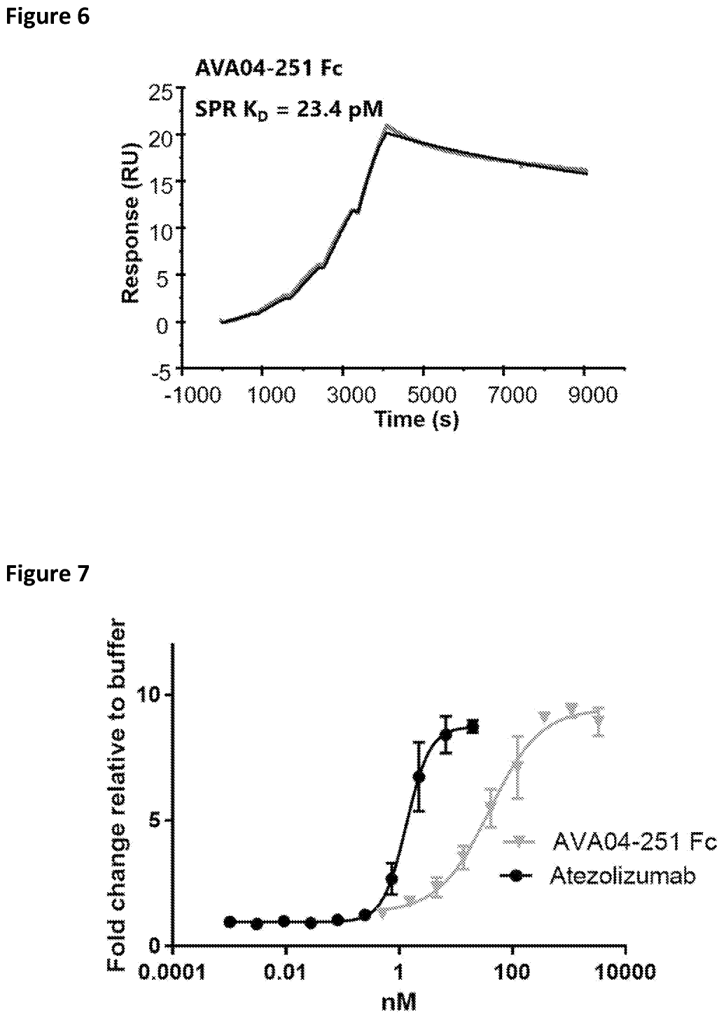

Biacore T200 kinetic analysis was performed using running buffer HBS-EP+(GE Healthcare) and a Series S sensor CM5 chip immobilized with human or mouse PD-L1 Fc (R&D Systems) in 10 mM sodium acetate pH 4.0 using amine coupling reagents (GE Healthcare). The single cycle kinetics concentration titration of Affimer Fc fusions was run at a flow rate of 30 L/min. PD-L1 Fc immobilized surface was regenerated with 3-5 mM NaOH for 20-30 seconds (GE Healthcare). The data blank was subtracted and fit to a 1:1 Langmuir binding model (BIAcore evaluation software; GE Healthcare) to calculate an apparent K_{D} value. AVA04-182 Fc fusion protein was shown to have a K_{D} of 36.1 μM using multi-cycle kinetics (FIG. 2) AVA04-251 Fc has a K_{D} of 23.4 μM measured with single-cycle kinetics (FIG. 6).

### Example 5: Competitive ELISA for Characterisation of Anti-PD-L1 Affimer

The competitive inhibition of Affimer Fc fusions was evaluated by enzyme-linked immunosorbent assay (ELISA) compared to an anti-mouse PD-L1 antibody, 10F9.G2 (FIG. 3). Human or mouse PD-1 Fc (R&D Systems) was coated at 0.5 μg/mL on the plate. Plates were washed 2 times with 150 μL of washing buffer (PBS, 0.1% Tween 20) with a plate washer and saturated with 5% casein (Sigma) in PBS for 90 minutes at room temperature (25±1° C.). Plates were washed as described previously. Affimer and controls (PD-1 Fc; blank) were then diluted in duplicate, and preincubated with 1 μg/mL of mouse PD-L1 Fc (R&D Systems) or 30 ng/mL of human PD-L1 (R&D Systems) for 30 minutes then loaded on the plate for 90 minutes at room temperature (25±1° C.). Plates were washed 3 times as described previously. Biotinylated polyclonal antibody anti-human PD-L1 (R&D Systems) was then diluted in Dilution Buffer and incubated for 90 minutes at room temperature (25±1° C.). Plates were washed 3 times as described previously and Streptavidin HRP was incubated for 30 minutes at room temperature (25±1° C.). Plates were washed and the substrate (TMB, Pierce Thermo-Scientific) was added on the plate for 10 minutes. The reaction was stopped using an acidic solution and plates were read at 450-630 nm. The IC50 was then calculated using an interpolated non-linear four-parameter fit standard curve.

### Example 6: Mouse Mixed Lymphocyte Reaction Assay of AVA04-182 Fc

A mouse mixed lymphocyte reaction (MLR) assay was performed to assess the ability of AVA04-182 Fc to modulate the T cell response. In this MLR assay, BMDC (bone marrow dendritic cells) were generated from the bone marrow of one (1) C57BL/6 mouse, cultured for seven (7) days in the presence of GM-CSF. Cells were then co-cultured with allogenic CD4+ T cells isolated from the spleen of a Balb/c mouse using negative selection. The MLR assay was performed in the presence or absence of the test products (AVA04-182 Fc, SQTgly Fc [SEQ ID NO: 120; Affimer Fc fusion without PD-L1 targeting], Avelumab and its isotype control, HuIgG1) and controls (anti-mouse PD-L1 clone 10F9.G2 and its isotype control, rat IgG2b). Test products were evaluated at three concentrations (700, 70 and 7 nM) and the controls at one concentration (70 nM). After 3 days of culture, cell culture supernatant was harvested and the secretion of interferon γ (IFNγ) and interleukin-2 (IL-2) was evaluated using ELISA. The anti-mouse PD-L1 and Avelumab induced an increase in IL-2 (data not shown) and IFNγ secretion, in comparison to their isotype controls (FIG. 4). Similarly, AVA04-182 Fc treatment led to an increase in IL-2 (data not shown) and IFNγ secretion at all concentrations tested, in comparison to SQTGly Fc (FIG. 4).

### Example 7: Activity of Anti-PD-L1 Affimer AVA04-251 Fc in a PD-1/PD-L1 Cell-Based Blockade Assay

The PD-1/PD-L1 cell-based assay (Promega) was performed according to the manufacturer's instruction. Briefly, Jurkat T cells expressing human PD-1 and a luciferase reporter driven by an NFAT response element (NFAT-RE), were co-cultured with PD-L1 aAPC/CHO-K1 cells expressing human PD-L1 and an engineered cell surface protein designed to activate cognate TCRs in an antigen-independent manner. When co-cultured, the PD-1/PD-L1 interaction inhibits TCR signaling and NFAT-RE-mediated luminescence. The addition of the anti-PD-L1 Affimer, AVA04-251 Fc, blocks the PD-1/PD-L1 interaction, releases the inhibitory signal and results in TCR activation and NFAT-RE-mediated luminescence (FIG. 7). The bioluminescent signal was detected and quantified using the Bio-Glo™ Luciferase Assay System (Promega) and signal read on a Clariostar plate reader (BMG LabTech).

### Example 8: Synthesis Protocol for 6323 (MAL-PEGs-Ser-D-Ala-Pro-Val-boroPro)

The chemical structure and synthesis scheme for maleimide 6323 (maleimide [MAL]-activated PEGs linker-Val-boroPro [VbP] pro-drug) are presented in FIGS. 9 and 10, respectively.

### Synthesis of Compound 3

HATU (0.8 g, 2.1 mmol), DIEA (0.8 mL, 4.6 mmol) and H-Val-boroPro-pn.HCl (Compound 2, 845 mg, 2.2 mmol) were added to a solution of N-Boc-D-Ala-Pro-OH (Compound 1, 572 mg, 2 mmol) in anhydrous DMF (8 mL) under ice-water bath cooling. The resulting mixture was stirred at room temperature for 2 hours and then condensed in vacuo. The residue was dissolved with ethyl acetate (100 mL), washed sequentially by 0.1 N KHSO₄ (3×20 mL), 5% NaHCO₃(3×20 mL), brine (20 mL). The organic phase was dried over anhydrous MgSO₄, filtered, and evaporated in vacuo to give N-Boc-D-Ala-L-Pro-L-Val-L-boroPro-pn which was then added to a solution of 4 N HCl in dioxane (20 mL) under ice-water cooling. The resulting mixture was stirred at room temperature for 2 hours and then condensed in vacuo. The residue was co-evaporated with dichloromethane (3×30 mL) in vacuo to completely dry. Compound 3 was thus obtained as a white powder (1.0 g, 92% over two steps).

### Synthesis of Compound 4

N-Fmoc-Ser(OtBu)-D-Ala-L-Pro-L-Val-L-boroPro-pn was prepared by coupling N-Fmoc-Ser-(OtBu)—OH and Compound 3 with the same method described above. The Fmoc was then removed by 20% of piperidine in DMF. The resulting mixture was condensed in vacuo and the residue was co-evaporated with dichloromethane (3×30 mL) in vacuo until completely dry to give the crude Compound 4 which was used directly for the next step without further purification.

### Synthesis of Compound 6323

MAL-dPEGs-Ser(OtBu)-D-Ala-L-Pro-L-Val-L-boroPro-pn was prepared by coupling MAL-dPEGs-acid with crude Compound 4 with the same method described above at 0.2 mmol scale. The OtBu was then removed by 50% of TFA in DCM. The resulting mixture was condensed in vacuo and the residue was co-evaporated with dichloromethane (3×10 mL) in vacuo until completely dry to give the crude MAL-dPEGs-Ser-D-Ala-L-Pro-L-Val-L-boroPro-pn which was further de-protected by reacting with PhB(OH)₂ in hexane-acetonitrile-water to remove the pinanediol group. The aqueous layer was separated and then purified by semi-preparative HPLC eluted with 20% to 25% acetonitrile in water (with 0.1% TFA). The desired fraction was collected and lyophilized to give Compound 6323 as a good colorless crystal (40 mg, 19% for the last three steps).

### Example 9: Synthesis Protocol for 6325 (NHS-PEGs-Ser-D-Ala-Pro-Val-boroPro)

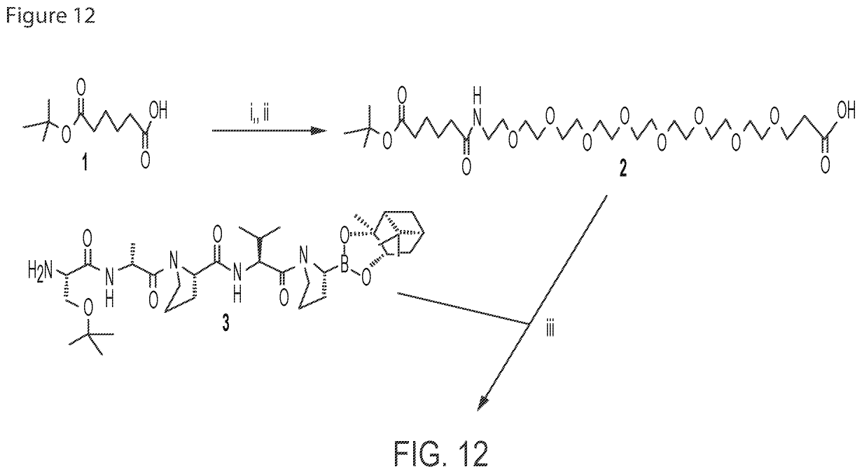

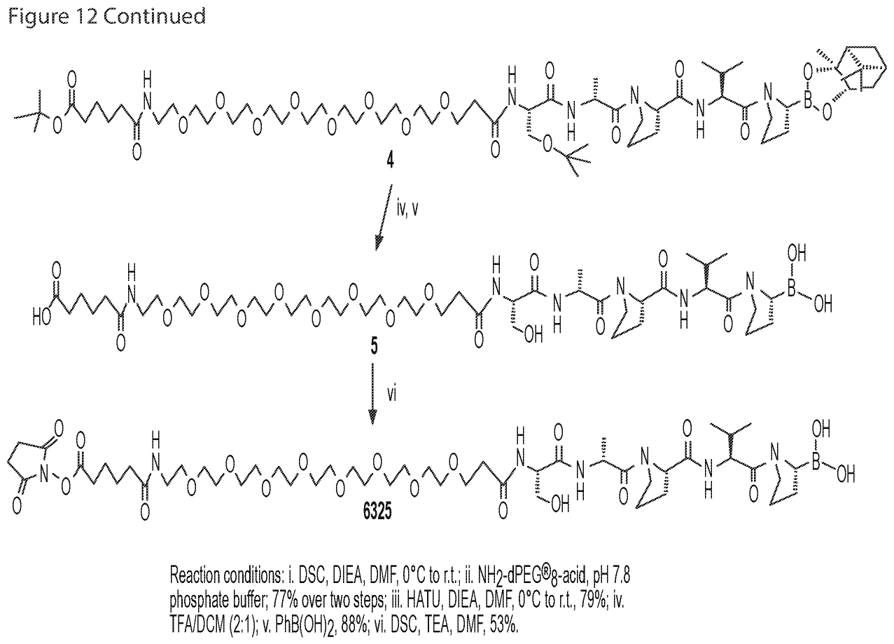

The chemical structure and synthesis scheme for 6325 (NHS-activated PEGs linker-VbP pro-drug) are presented in FIGS. 11 and 12, respectively.

### Synthesis of Compound 2

DSC (71 mg, 0.275 mmol) and DIEA (0.1 mL, 0.58 mmol) were added to a solution of Compound 1 (51 mg, 0.25 mmol) in anhydrous DMF (1.5 mL) under ice-water bath cooling. The reaction mixture was stirred at room temperature for 2 hours and then slowly added to another solution of NH₂-dPEG®s-acid (110 mg, 0.25 mmol) in a pH 7.8 phosphate buffer (5 mL) under ice-water bath cooling. The reaction mixture was stirred at room temperature for 1 hour and then purified by semi-preparative HPLC eluted with 2% to 98% acetonitrile in water (with 0.1% TFA). The desired fraction was collected and lyophilized to give Compound 2 (120 mg, 77% over two steps).

### Synthesis of Compound 4

HATU (30 mg, 0.08 mmol), DIEA (28 μL, 0.16 mmol) and Compound 3 (53 mg, 0.08 mmol; from the synthesis of Compound 6323; Example 8) were added to a solution of Compound 2 (50 mg, 0.08 mmol) in anhydrous DMF (1.5 mL) under ice-water bath cooling. The resulting mixture was stirred at room temperature for 1 hour and then purified by semi-preparative HPLC eluted with 20% to 98% acetonitrile in water (with 0.1% TFA). The desired fraction was collected and lyophilized to give Compound 4 (80 mg, 79%).

### Synthesis of Compound 5

TFA (1.0 mL) was added to a solution of Compound 4 (80 mg, 0.063 mmol) in DCM (0.5 mL) under ice-water bath cooling. The resulting mixture was stirred at room temperature for 2 hours and then was condensed in vacuo. The residue was co-evaporated with dichloromethane (3×10 mL) in vacuo to completely dry and then dissolved into a mixture of water-acetonitrile-hexane (2:1:2, 3.0 mL). PhB(OH)₂ (8 mg, 0.065 mmol) was added. The resulting mixture was stirred at room temperature for 3 hours and was then condensed in vacuo. The residue was purified by semi-preparative HPLC eluted with 20% to 98% acetonitrile in water (with 0.1% TFA). The desired fraction was collected and lyophilized to give Compound 5 (57 mg, 88%).

### Synthesis of Compound 6325

DSC (15.5 mg, 0.06 mmol) and TEA (30 μL, 0.21 mmol) were added to a solution of Compound 5 (57 mg, 0.056 mmol) in anhydrous DMF (1.5 mL) under ice-water bath cooling. The reaction mixture was stirred at room temperature for 2 hours and then purified by semi-preparative HPLC eluted with 5% to 98% acetonitrile in water (with 0.1% TFA). The desired fraction was collected and lyophilized to give Compound 6325 (24 mg, 53%) as a white powder.

### Example 10: Conjugation of Compound 6323 to AVA04-251 BH Cys Using Maleimide Chemistry

The synthesis scheme for AVA04-251 BH cys-6323 pro-drug is presented in FIG. 13.

Compound 6323 (MAL-PEGs-Ser-DAla-Pro-VbP; Example 8) was dissolved in DMSO at a concentration of 100 mM. A 1 mL sample of 1 mg/mL AVA04-251 BH cys was dialyzed overnight against 1 L of 50 mM MES, 150 mM NaCl, 1 mM TCEP, pH 6. Compound 6323 was added to AVA04-251 BH cys at a molar ratio of 100:1, respectively. The mixture was incubated at room temperature overnight (17 hours). Unconjugated Compound 6323 was removed with a 1 mL HisTrap FF column (GE Healthcare). The conjugated AVA04-251 BH cys-6323 pro-drug was eluted from the HisTrap column with 1M imidazole and dialyzed against PBS 1× (2×1 L) to remove imidazole and exchange the buffer. The concentration of the resulting solution was determined from the absorbance at 280 nm using an extinction coefficient calculated from the sequence of AVA04-251 BH cys (39151 M⁻¹ cm⁻¹; ExPASy ProtParam).

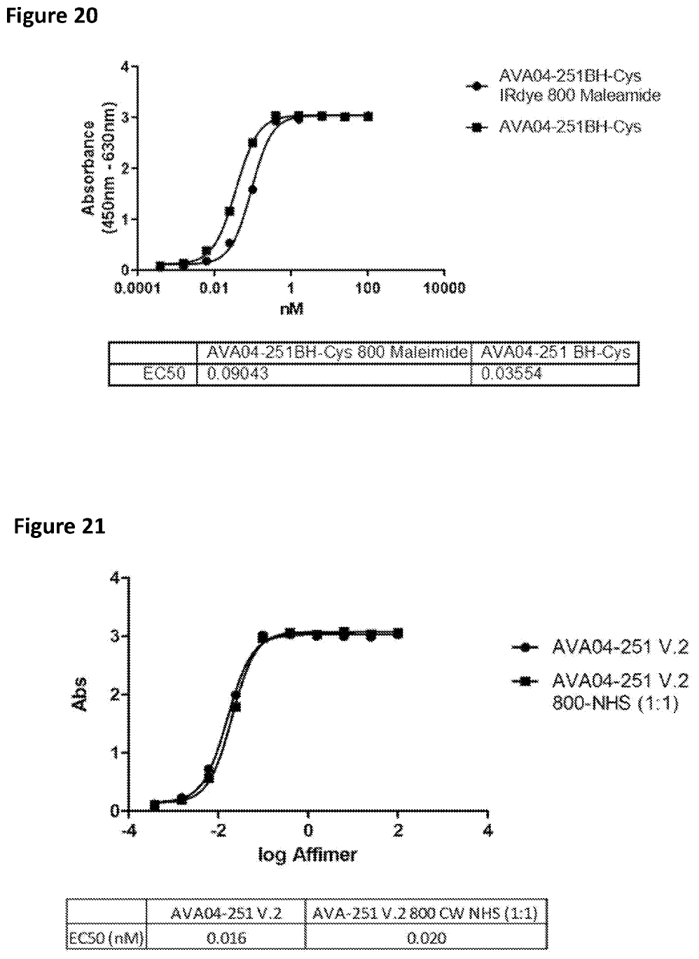

Subsequently, it was shown by binding ELISA that conjugation of AVA04-251 BH cys to IRDye 800CW with maleimide chemistry does not modify its binding capacity (FIG. 20).

Briefly, human PD-L1 Fe (R&D Systems) chimeric protein was coated onto 96 well plates at 0.5 μg/mL in carbonate buffer. After saturation with 5% casein in PBS, plates were washed and a dilution of conjugated Affimer or unconjugated control were incubated for 90 mins. Plates were then washed, a biotinylated polyclonal anti-cystatin A antibody (R&D Systems) added, and the plates incubated for 1 hour. Plates were washed and bound Affimer was detected using streptavidin-HRP. After a last washing step, TMB was added and the plate was read at 450 nM. The conjugated Affimer (AVA04-251 BH cys-800) exhibited a similar EC50 compared to the parental molecule (AVA04-251 BH cys; FIG. 20).

### Example 11: Conjugation of Compound 6325 to AVA04-182 Fc Using NHS Chemistry

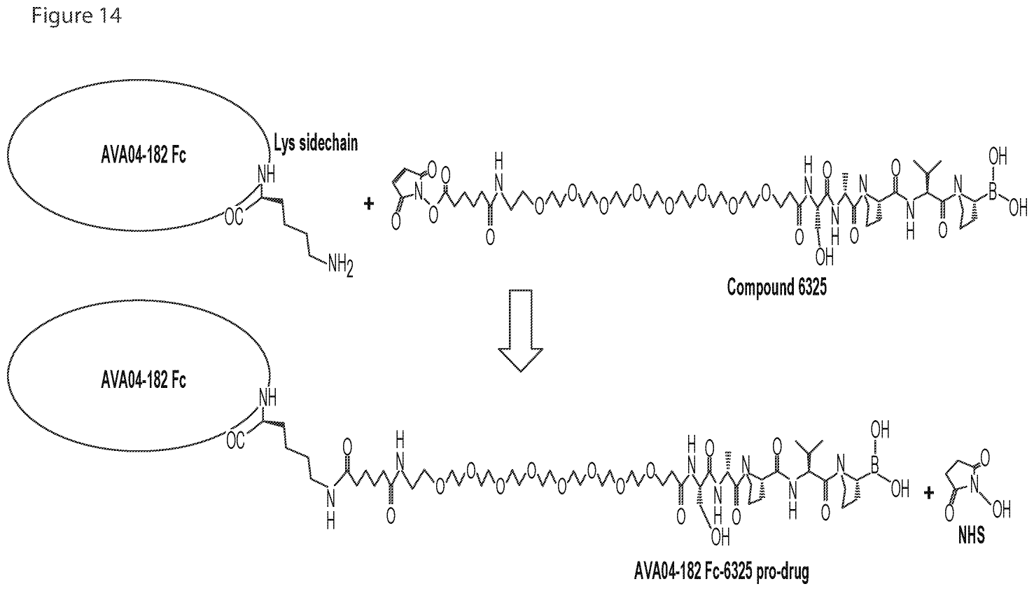

The synthesis scheme for AVA04-182 Fc-6325 pro-drug is presented in FIG. 14.

Compound 6325 (NHS-PEGs-Ser-DAla-Pro-VbP; Example 9) was dissolved in DMSO at a concentration of 100 mM. AVA04-182 Fc was diluted to 1 mg/mL with PBS. The pH was increased by addition of 1/10^{th} volume of 1 M potassium phosphate, pH 9. Compound 6325 was added to AVA04-182 Fc at a molar ratio of 4:1, respectively. The mixture was incubated at room temperature overnight (17 hours). Unconjugated Compound 6325 was removed and buffer exchanged by passing the reaction mixture over a 5 mL Zeba Spin Desalting column (Thermo Scientific, 7000 MWCO) and dialysis against 1 L of PBS. The concentration of the resulting solution was determined from the absorbance at 280 nm using an extinction coefficient calculated from the sequence of AVA04-182 Fc (92430 M⁻¹ cm⁻¹; ExPASy ProtParam).

### Example 12: Tumour Growth Inhibition Following Treatment with AVA04-182 Fc in Combination with VbP in a MB49 Syngeneic Murine Bladder Cancer Model

Mice (n=10/group, C57BL/6) were inoculated subcutaneously in the right flank with 1×10⁶ MB49 cells per animal on Day 0. AVA04-182 Fc and SQTgly Fc control (Affimer Fc fusion without PD-L1 targeting) were tested at 10 and 20 mg/kg via the IP route, 3 times over 10 days. VbP (20 μg; Tufts University) was given per os, 5 days a week for 4 weeks starting after randomization, when the mean tumor volume reached 60 mm³.

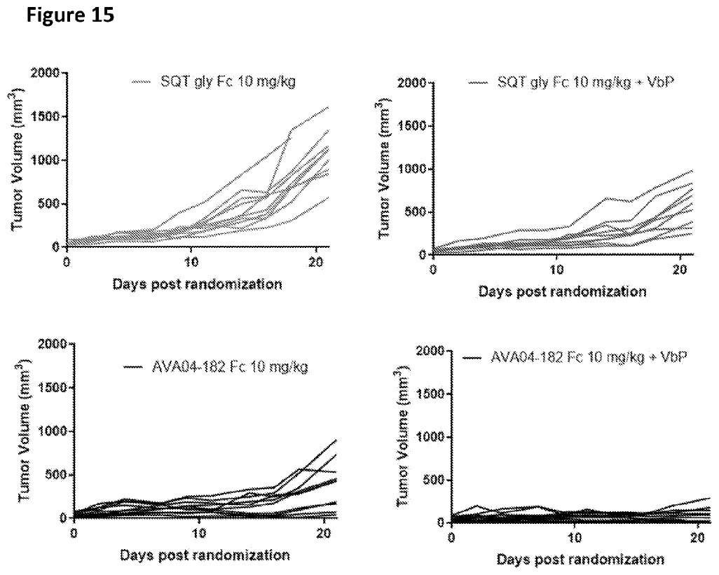

Twenty-one (21) days after initiation of the treatment, tumor growth was analyzed. Group control (SQTgly Fc) with VbP shows significant tumor growth inhibition (p<0.001, Dunnett's test) compared to SQTgly Fc alone (FIG. 15).

No significant difference was seen between monotherapies (AVA04-182 Fc or VbP), but an additive effect was observed following treatment with AVA04-182 Fc plus VbP, resulting in tumor regression in some mice from the group (p<0.01, Dunnett's test), compared to the SQTgly Fc plus VbP combination group.

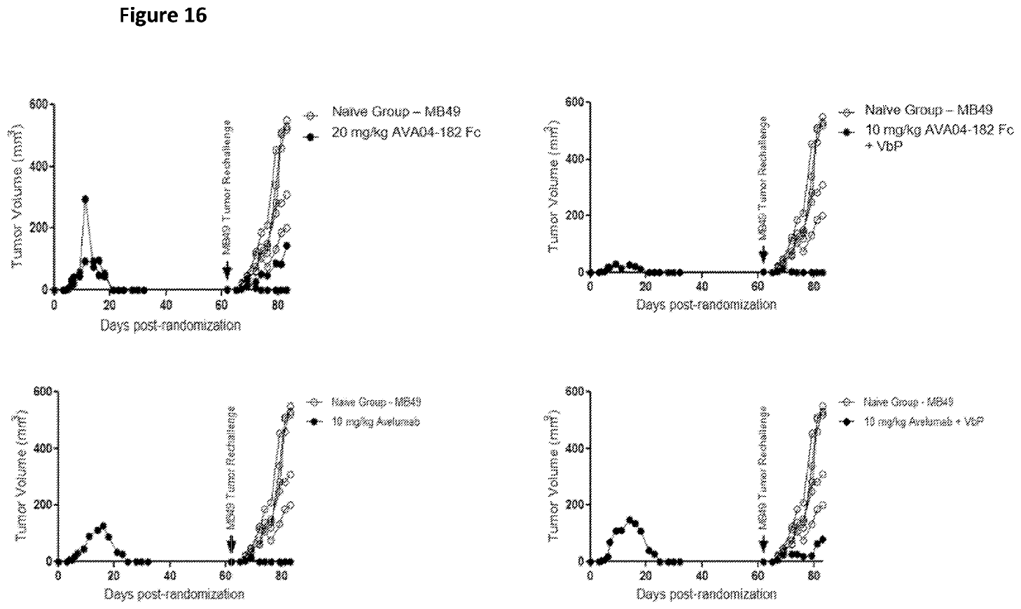

To evaluate the effect on the immune response, a rechallenge after 60 days post-inoculation with MB49 cells was performed in mice showing full regression. None of the mice developed a new tumor confirming sufficient enhancement of specific T cell activation able to trigger a memory response. As expected, naïve mice inoculated with the same culture of MB49 cells did developed tumors (FIG. 16).

### Example 13: Tumour Growth Inhibition Following Treatment with AVA04-251 Fc in Combination with VbP in a Humanised PD-L1 MC38 in a C57BL/6 Mice Syngeneic Model of Colorectal Cancer

Mice (n=8/group, C57BL/6) were inoculated subcutaneously in the right flank region with a humanized PD-L1 MC38 tumour cell line (in which the mouse PD-L1 extracellular domain was replaced by the equivalent human domain; CrownBio Inc). AVA04-251 Fc and associated control (SQTgly Fc) were injected via the IP route once tumours were >80 mm³. Treatments were administered twice a week for 3 weeks at a dose of 10 mg/kg (AVA04-251 Fc and its control, SQTgly Fc). VbP (Tufts University) was administered 5 times a week (with 2 days off) at 0.02 mg/mouse per os. Overall, tumour growth inhibition was shown for both treatments (FIG. 17). All mice treated with AVA04-251 Fc had a reduced tumour size compared to the control. Following treatment with VbP, none of the mice showed escape of tumour growth at Day 13, compared to the monotherapy.

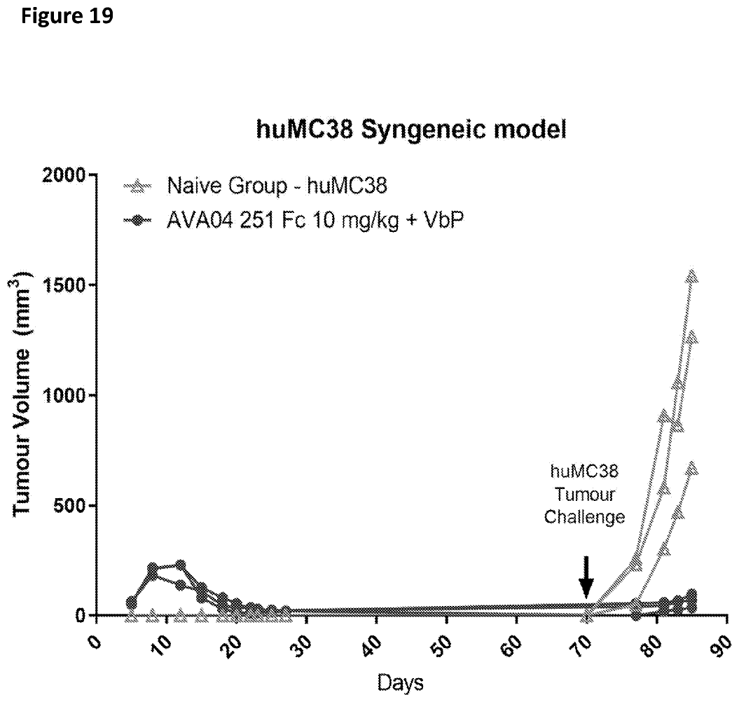

Seventy (70) days after the inoculation of the tumour, three (3) mice from the group treated with AVA04-251 Fc and VbP were inoculated a second time with the huMC38 cell line (a control group was inoculated at the same time) to assess if the immune system developed a memory response, preventing subsequent tumor growth. As shown in FIG. 19, ten (10) days after the second inoculation, tumors in the treated group was smaller than 80 mm³, while in the control group, tumors reached >750 mm³.

### Example 14: Biodistribution of AVA04-251 Fc-800 in a A375 Mouse Xenograft Model

The targeting of anti-PD-L1 Affimers to tumors expressing human PD-L1 was assessed in a mouse xenograft model based on the biodistribution of IR dye-conjugated Affimer followed over time using fluorescence imaging. AVA04-251 Fc was conjugated to IRDye800CW (LI-COR) with NHS chemistry to modify accessible amino groups on the protein. AVA04-251 Fc (1 mg/mL in PBS) was incubated with IRDye 800CW (4 mg/mL in water) at a stoichiometry of 4:1 dye:protein for 2 hours, in dark conditions, at room temperature (−23° C.). Free dye was separated from dye-conjugated Affimer (AVA04-251 Fc-800) using a 5 mL Zeba Spin Desalting Column (MWCO 7000; Pierce) according to the manufacturer's instructions. The dye:protein ratio was calculated based on the absorbance at 280 and 780 nm according to the equation:

Dye:protein ratio=(A780/□Dye)/(A280−(0.03×A780))/εprotein

Where 0.03 is the correction factor for the absorbance of IRDye 800CW at 280 nm, and εDye and ε protein are molar extinction coefficients for the dye (is 270,000 M⁻¹ cm⁻¹) and protein (115000 M⁻¹ cm⁻¹ for AVA04-251 Fc, respectively.

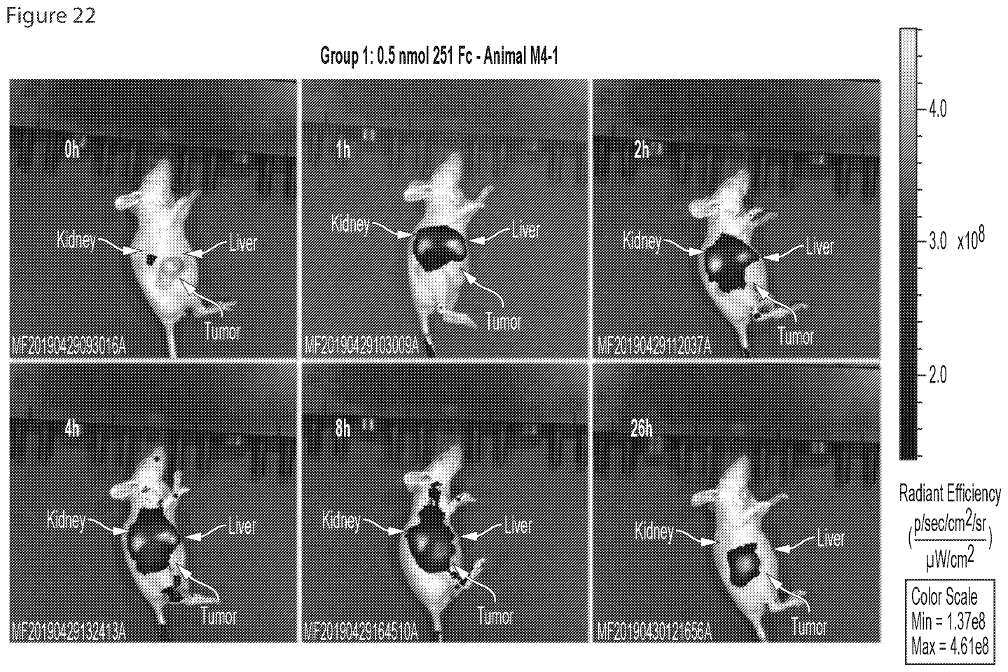

The binding of dye-conjugated AVA04-251 Fc-800 to human PD-L1 was compared to non-conjugated Affimer using a PD-L1 binding ELISA (as described in Example 10). Data indicate that dye conjugation does not impact the affinity of the AVA04-251 Fc for the PD-L1 target based on comparable EC50 values (FIG. 21). Furthermore, dye-conjugation was shown not to impact levels of higher aggregates based on SEC HPLC (Yarra 3000 column run at 0.8 mL/min in 1×PBS).

The A375 mouse xenograft model was established in female athymic nude mice (Charles River Laboratories) following subcutaneous injection of A375 cells (5×10⁶ cells [ATCC] in 100 μL sterile PBS) into the animal's flank. Tumors were monitored three (3) times per week, with the developing tumour being measured with callipers. Tumours were allowed to grow to between 500-1000 mm³ prior to intravenous administration of AVA04-251 Fc-800 (0.1 or 0.5 nmole) into the tail vein of three (3) mice. Fluorescence images were recorded with a Xenogen IVIS 200 Biophotonic Imager immediately after injection (time 0) and at 1, 2, 4, 8 and 26 hours post-dose. Time-course images from a representative animal (M4-1) administered AVA04-251 Fc-800 (0.5 nmoles), showing targeting of the anti-PD-L1 Affimer to the tumor at the 26-hour timepoint, are presented in FIG. 22. Arrows indicate the approximate locations of the kidney, liver and tumor.

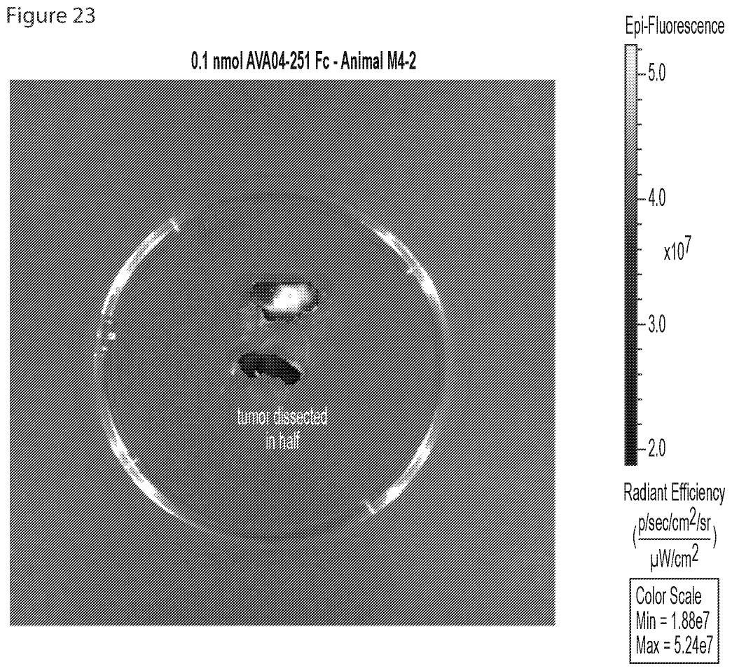

Tumor penetration of the dye-conjugated Affimer was demonstrated following dissection of a tumour following administration of AVA04-251 Fc-800 (Animal M4-2; 0.1 nmoles). The mouse was euthanized at 26-hours post-dose, the tumour removed and cut in half. The dissected tumour was imaged as previously described (FIG. 23).

### Example 15: In Vitro rhFAP(Cleavage of Affimer-Linker-VbP Pro-Drugs

The kinetics of release of biologically active VbP following cleavage of Affimer-linker-VbP pro-drugs by recombinant human fibroblast activation protein alpha (rhFAPα) was investigated based on the quantitation of released VbP over a time-course by LC-MS/MS.

Examples of synthesised MAL- and NHS-activated FAPα cleavable linker-VbP pro-drugs of varying length, based on differing numbers of ethylene glycol sub-units, are presented in Examples 8 and 9. MAL-activated linker-VbP pro-drugs (such as 6323 [PEG₈], 6324 [PEG₁₆] and 6327 [PEG₂₄]) were subsequently conjugated to the single Cys residue in Affimers including AVA04-251 BH cys. Similarly, NHS-activated linker-VbP pro-drugs (such as 6325 [PEG₈], 6326 [PEG₁₆] and 6328 [PEG₂₄]) were conjugated to free amino groups of Affimers including AVA04-182 Fc. Representative Affimer conjugation methodology is presented in Examples 10 and 11.

Affimer-linker-VbP pro-drug samples (5 μM in PBS) were incubated with rhFAPα (12 nM final concentration; R&D Systems) for 15 minutes at 37° C. The reaction was stopped by addition of TCA (5% final concentration), with the resulting precipitated protein being removed by centrifugation (6,000 g for 10 minutes). Control samples were prepared by addition of TCA before addition of rhFAPα. LC-MS/MS (Applied Biosystems 4000Qtrap mass spectrometer with Agilent 1200 HPLC) was performed with a multiple reaction monitoring method designed to specifically detect VbP. Briefly, chromatography was performed with a Zorbax Eclipse Plus C18 column (4.6×50 mm, 1.8 μm) with a linear gradient from 95:5 water:5% methanol (0.1% formic acid, 5 mM ammonium acid) to 5:95 water:methanol (0.1% formic acid, 5 mM ammonium acid) over 3 minutes. The parent ion was at 215.3 Da and the daughter ion at 126.1 Da. The internal standard was d8-Val-boroPro in water at pH 2 (parent ion 223.3 Da, daughter ion 126.1 Da).

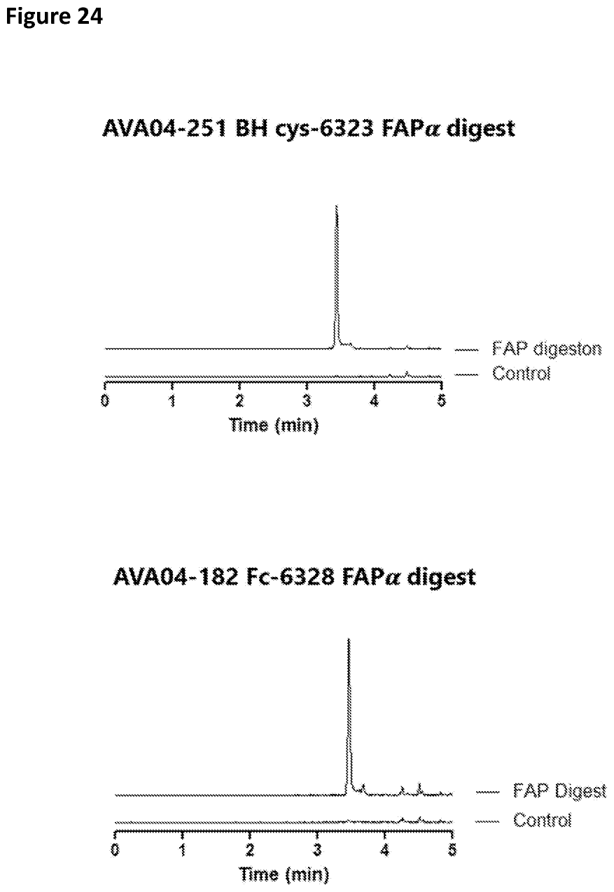

Representative LC-MS/MS chromatograms showing the release of VbP from AVA04-251 BH cys-6323 and AVA04-182 Fc-6328, incorporating the PEGs- and PEG24-linker-VbP pro-drugs, respectively, are presented in FIG. 24. Data confirm the rhFAPα catalysed release of VbP from Affimer-linker-VbP pro-drugs incorporating linker-VbP pro-drugs of varying PEG lengths and based on both MAL- and NHS-conjugation chemistry.

Subsequently, Affimer-linker-VbP pro-drug samples (5.5 μM in PBS) were incubated with rhFAPα (12 nM final concentration; R&D Systems) at 37° C. Aliquots were withdrawn immediately following addition of rhFAPα (time 0) and subsequently, following 2, 5 and 10 minutes incubation. The reaction was stopped by addition of TCA (5% final concentration), with the resulting precipitated protein being removed by centrifugation (6,000 g for 10 minutes). Released VbP was quantified by LC-MS/MS, as described previously, based on peak area interpolated against a standard curve prepared over the range 0.1-1000 ng/mL VbP (0.47-4700 nM; Tufts University).

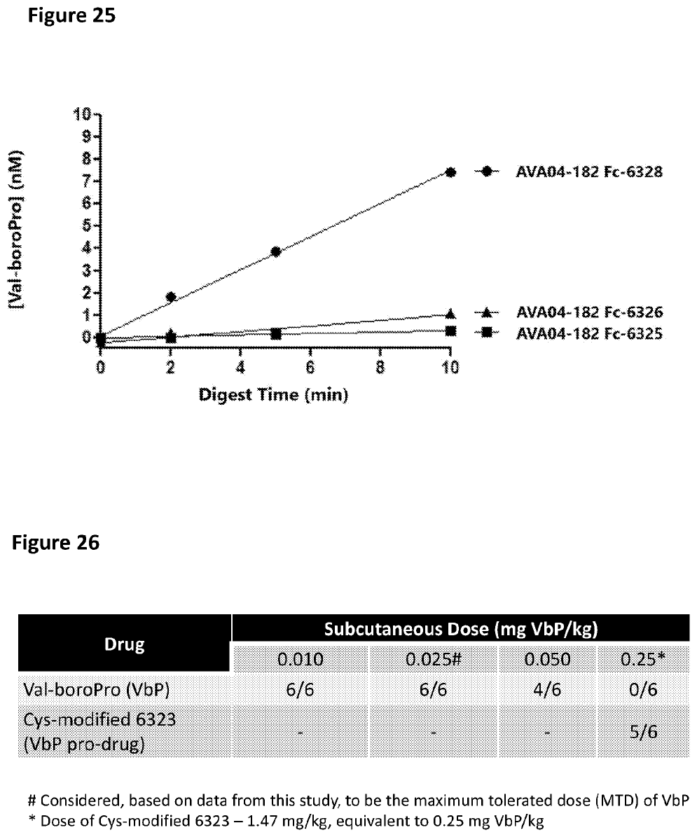

The FAPα catalysed release time-course of VbP from AVA04-182 Fc conjugated to 6325 (PEGs-linker-VbP pro-drug), 6326 (PEG16-linker-VbP pro-drug) and 6328 (PEG24-linker-VbP pro-drug) is presented in FIG. 25. Data indicate a dependency between the rate of release of VbP from the Affimer-linker-VbP pro-drugs and the length of the PEG linker, suggesting an ability to modify the VbP release kinetics depending on the desired therapeutic profile.

### Example 16: Evaluation of a Linker-VbP Pro-Drug Compared to VbP in an Acute Toxicity Study in Sprague Dawley Rats

The comparative in vivo safety of a representative linker-VbP pro-drug administered subcutaneously at a dose equivalent to 10-fold the maximum tolerated dose (MTD) of VbP was established in Sprague Dawley (SD) rats, i.e., 10 times the dose of VbP that kills at least percent of the SD rats.

Prior to administration, MAL-activated 6323 (PEGs-linker-VbP pro-drug) was conjugated to L-cysteine in order to inactivate the MAL moiety. L-cysteine (5 mg; Sigma) was dissolved in 1 mL of MAL-activated 6323 (4.8 mM in 50 mM MES, 150 mM NaCl, pH 6) to achieve an approximate stoichiometry of 10:1 L-cysteine:6323. The resulting solution was incubated at room temperature for 2-3 hours, after which time the reaction was confirmed to have reached completion by LC-MS analysis. The resulting Cys-modified 6323 was purified by preparative RP-HPLC using a Supelco Discovery C18 column with a 2:98 acetonitrile:water (0.1% TFA) gradient over 10 minutes, and was subsequently lyophilized.

Six (6) male SD rats (Charles River) were injected subcutaneously with 1.47 mg/kg Cys-modified 6323 (equivalent to 0.25 mg VbP/kg) in sterile PBS. Animals were observed for signs of toxicity at 1, 2, 4, 6, 8, and 24 hours post-dose, with the safety endpoint being the 24-hour survival ratio; the number surviving animals at 24 hours/total number treated. FIG. 26 presents comparable safety data for Cys-modified 6323 (1.47 mg/kg; equivalent to 0.25 mg VbP/kg) and VbP (Tufts University) administered subcutaneously at 0.010, 0.025 and 0.050 mg VbP/kg in sterile PBS. In this study, the MTD for VbP was considered to be 0.025 mg VbP/kg. At 24 hours, 5 of the 6 animals administered Cys-modified 6323 at a dose equivalent to 10-times the VbP MTD survived, indicating a safety margin for the VbP pro-drug relative VbP.

### Example 17: In Vitro Affimer-Linker-VbP Pro-Drug Induced Pyroptosis in the J774 Mouse Macrophage Cell Line

Affimer-linker-VbP pro-drugs are designed to be cleaved by FAPα to release VbP. VbP induces pyroptosis in macrophages by the activation of caspase-1 in the NLRP1 and CARD8 inflammasomes (1-3), but when VbP is incorporated into the pro-drug conjugate it is prevented from doing so because the amino group of valine is engaged in a peptide bond with the FAPα-cleavable linker. Pro-drug conjugates of AVA04-182 Fc were tested in an in vitro assay of pyroptosis in the presence and absence of rhFAPα in order to demonstrate that the ability of an Affimer-linker-VbP pro-drug to induce pyroptosis in macrophages is strictly dependent upon FAPα cleavage.

J774A.1 cells (mouse monocyte macrophages; ATCC) were grown in DMEM-10%-FBS in 75 cm² tissue culture flasks, harvested by scraping in PBS, resuspended in DMEM-1%-FBS, plated in 96-well plates (VWR) at a density of 5×10³ cells per well, and placed in a 5% C02 incubator at 37° C. After incubation for 24 hours, serial 10-fold dilutions of VbP, unconjugated AVA04-182 Fc, linker-VbP pro-drugs (6325, 6326 and 6328), and Affimer-linker-VbP pro-drugs (AVA04-182 Fc-6325, −6326 and −6328) were added to wells with and without addition of rhFAPα (R&D Systems) at a final concentration of 25 nM. Each reaction mixture was tested in triplicate and incubated for a further 24 hours at 37° C. Lactate dehydrogenase (LDH; a marker of pyroptosis [4]) released into culture supernatants, was measured using the CytoTox 96 Non-Radioactive Cytotoxicity Assay (Promega) according to the manufacturer's instructions. LDH concentrations were determined by absorbance (A490) measurements in a SpectraMax® M2^{e} microplate reader (Molecular Devices). Percent LDH release was calculated by subtracting the background release in wells containing DMEM-1%-FBS without cells and expressing the resulting values as percentages of the LDH released by the CytoTox 96 lysis reagent.

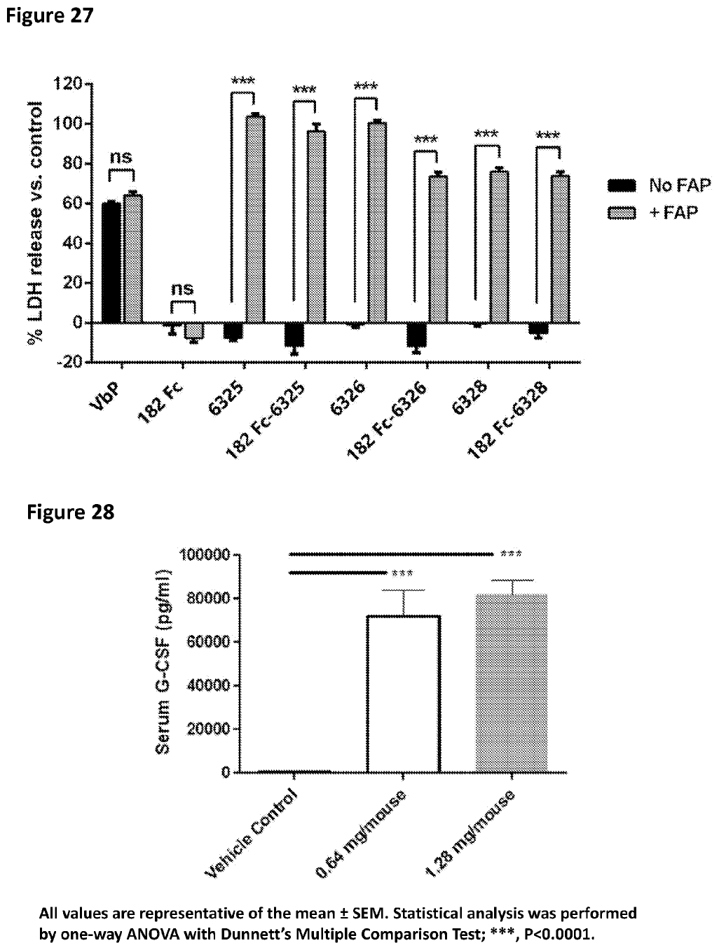

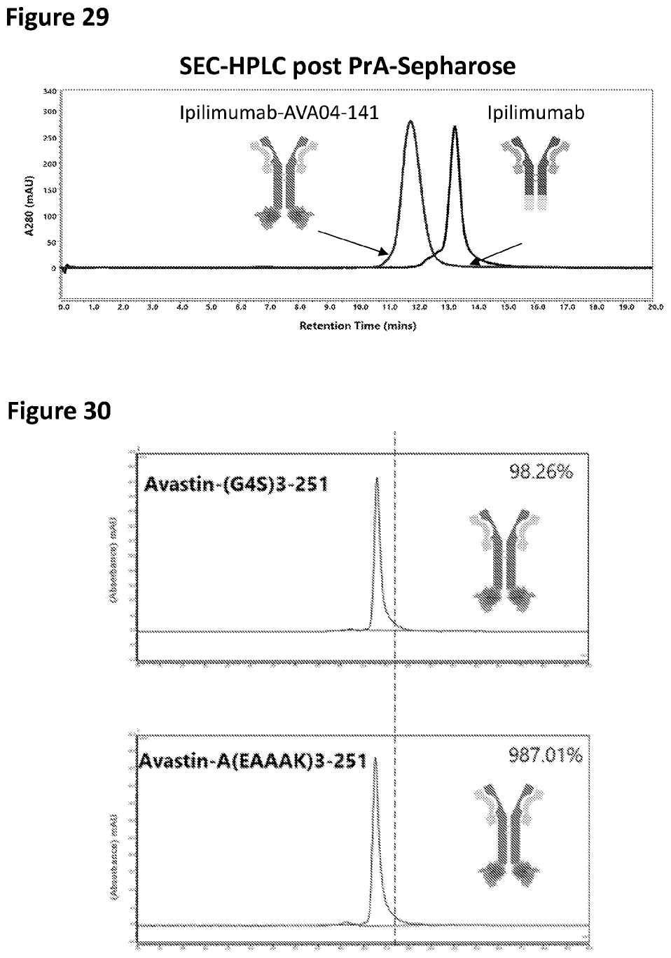

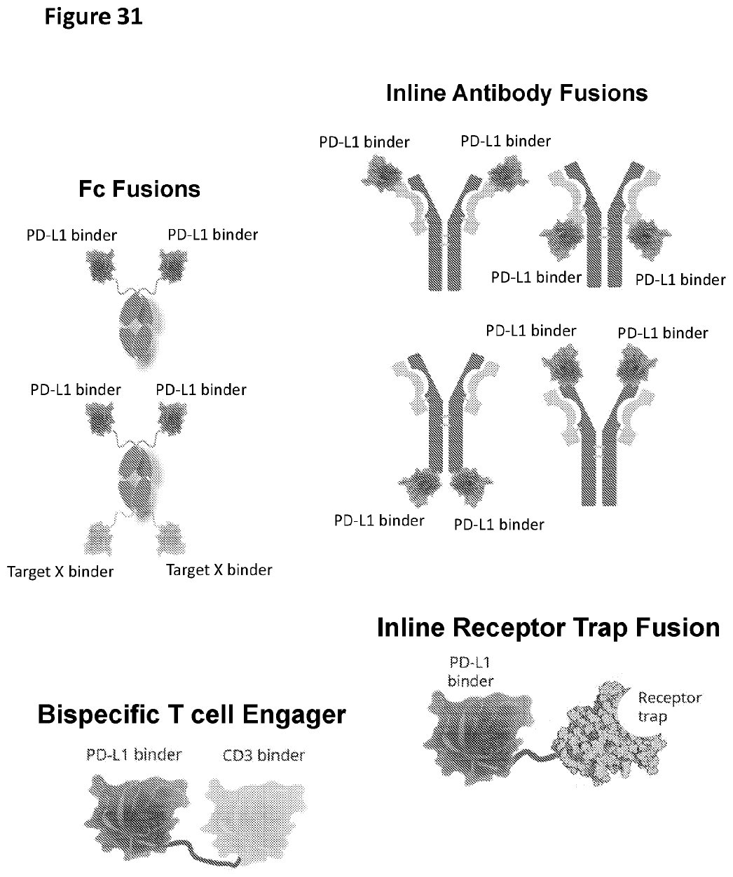

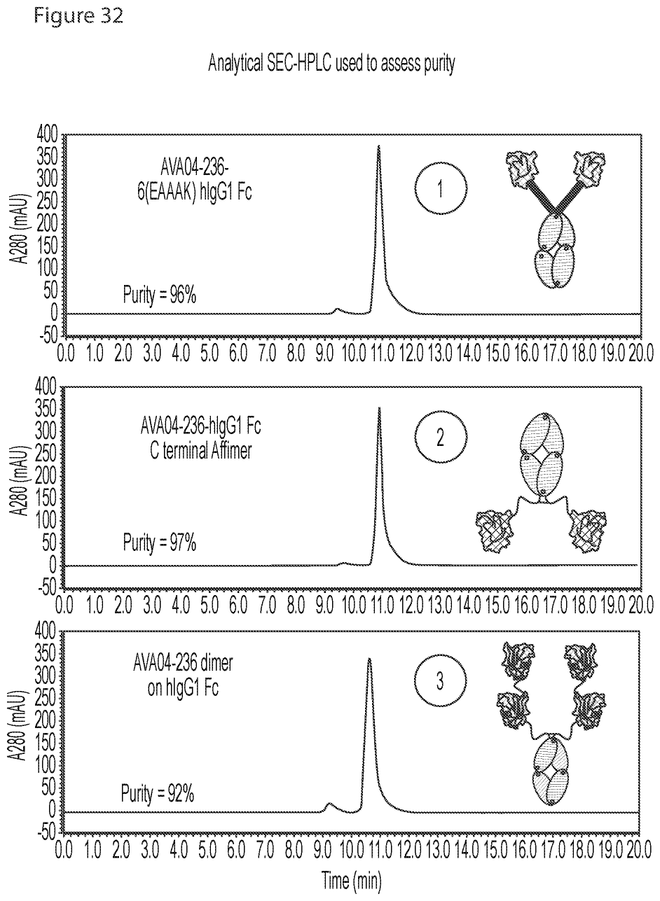

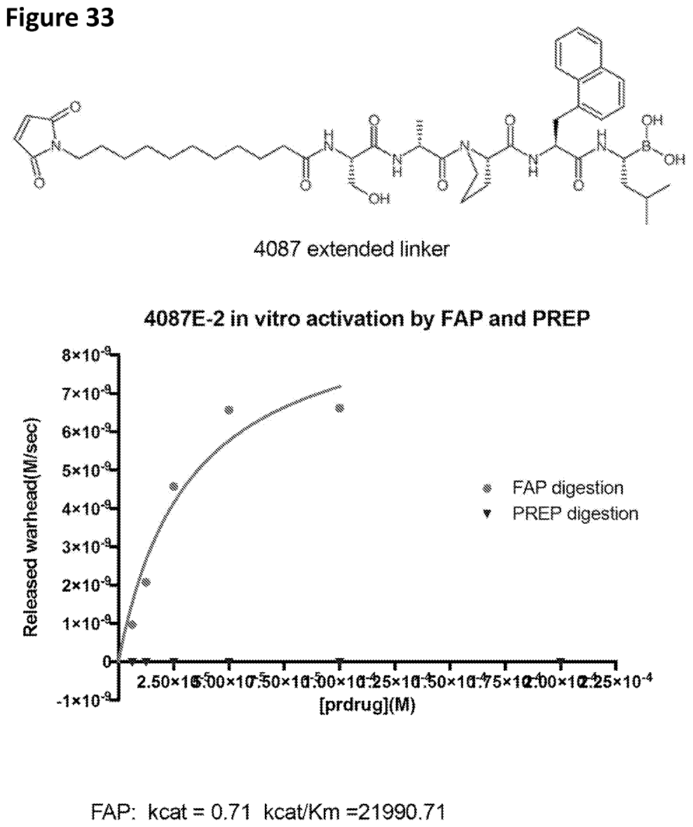

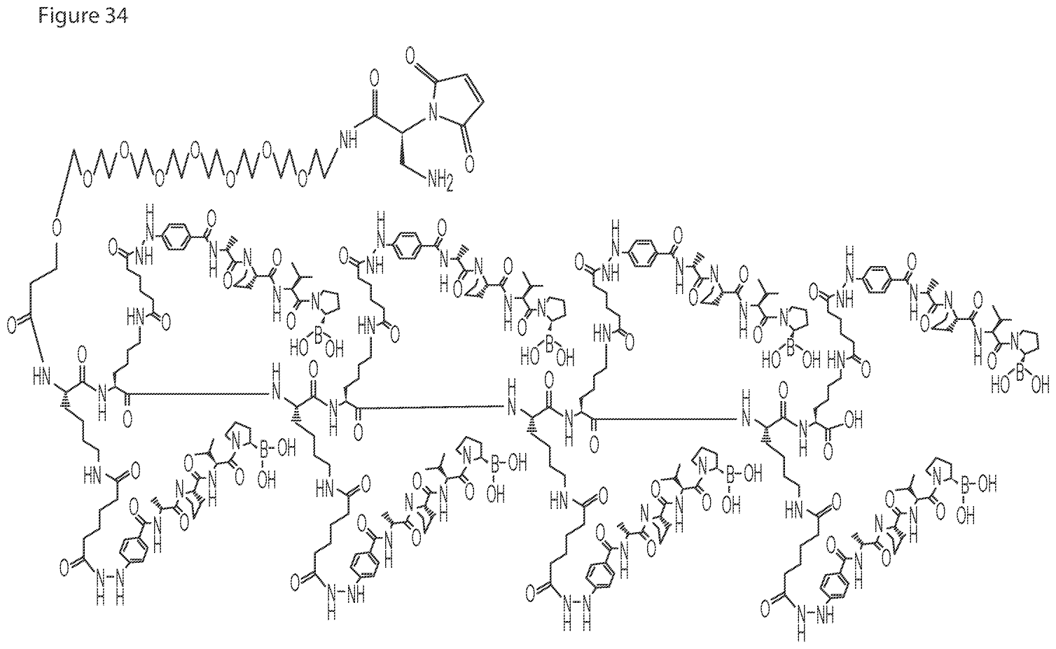

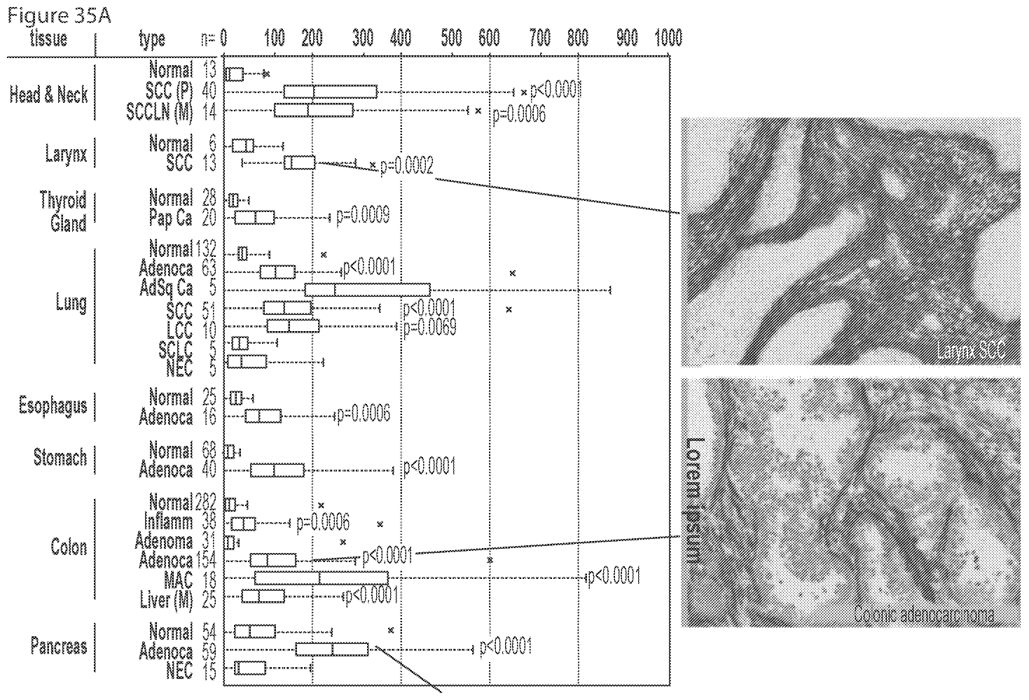

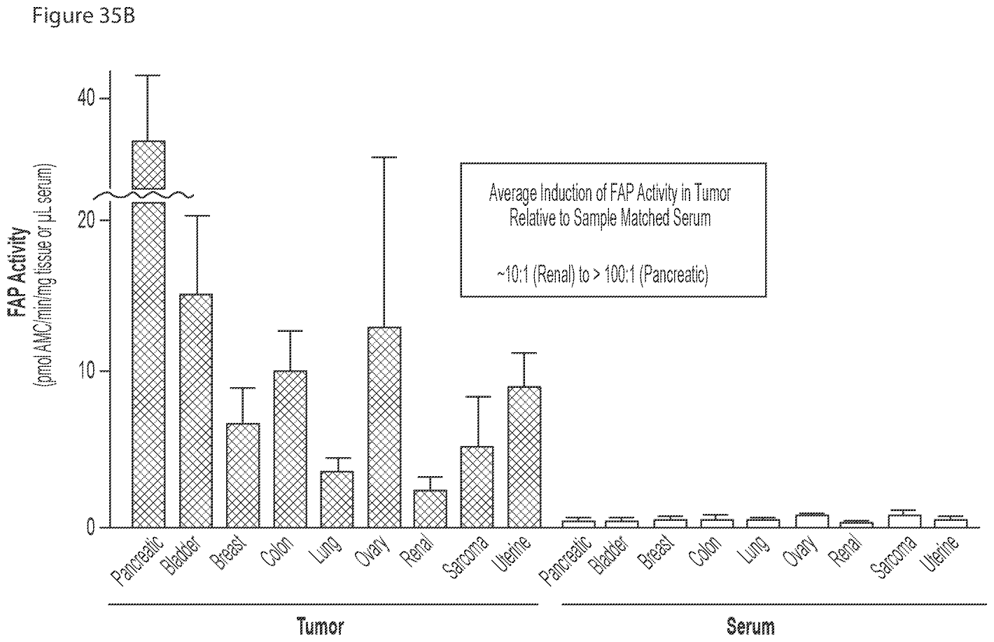

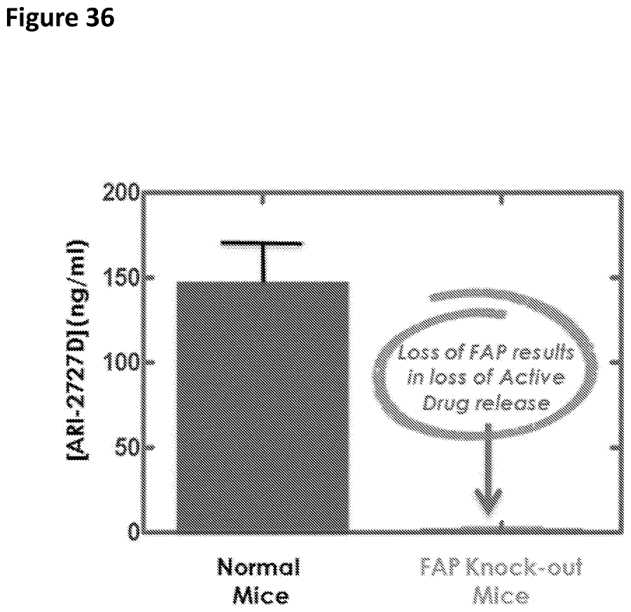

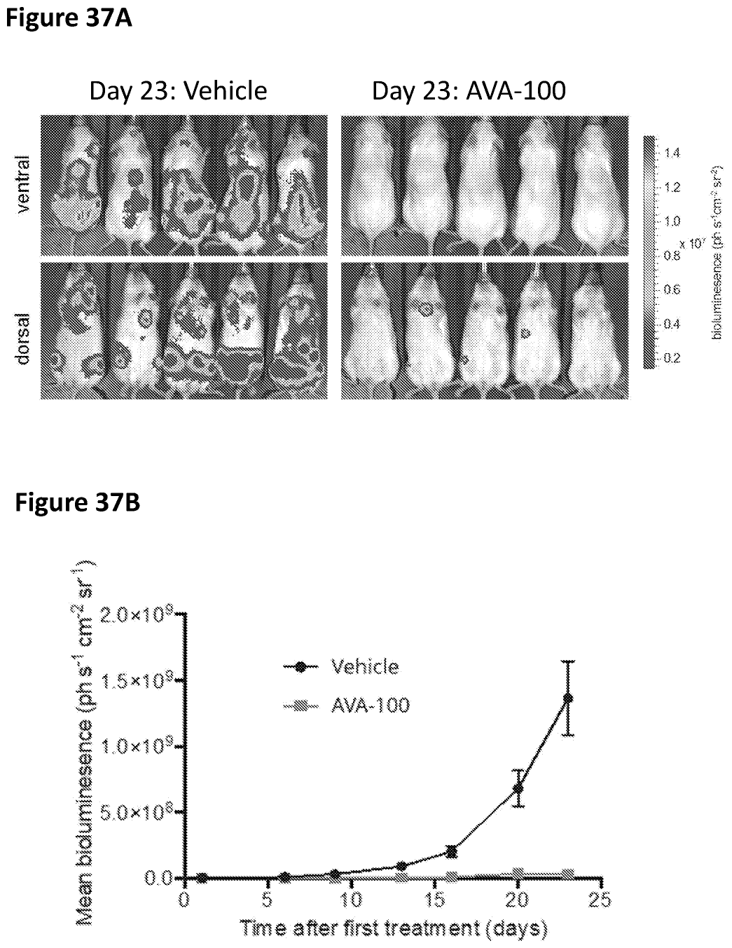

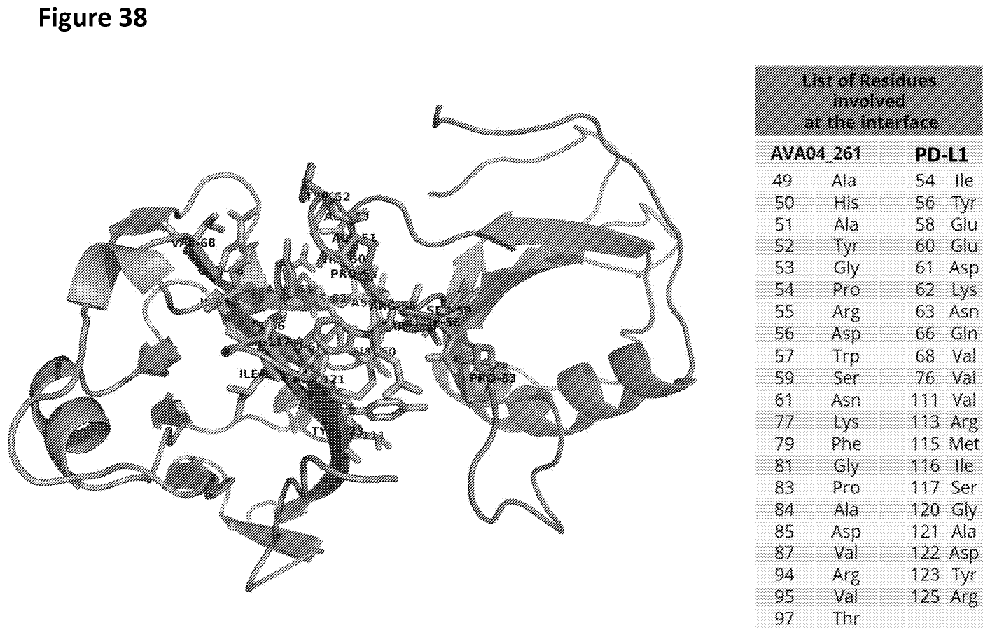

At a concentration of 1 μM, all three Affimer-linker-VbP pro-drugs exhibited significant LDH release in the presence of rhFAPα (light bars; FIG. 27), but not in its absence (dark bars; FIG. 27). This was also observed for the linker-VbP pro-drugs. As expected (5), VbP produced significant LDH release regardless of whether or not rhFAPα was present. The unconjugated Affimer did not produce LDH release. These results indicate that Affimer-linker-VbP pro-drugs release VbP in an FAPα-dependent manner, but remain biologically inactive in the absence of FAPα.

### Example 18: In Vivo Cys-Modified Linker-VbP Pro-Drug Induced G-CSF Stimulation in BALB/c Mice

In macrophages, VbP has been shown to induce the recruitment of caspase-1 into the NLRP1 inflammasome resulting in the processing of pro-caspase-1 into enzymatically active caspase-1, which then processes pro-IL-10 into the mature and biologically active form that is subsequently secreted (3, 6). Acting in both an autocrine and paracrine manner, mature IL-1β can induce the expression of various cytokines at the transcriptional level (7). In mice, the administration of VbP is associated with increased expression of cytokines, and increased serum concentration of G-CSF has been shown to be a robust marker for this effect (8, 9). The validity of G-CSF as a marker for the biological activity of VbP in vivo is supported by the complete loss of the serum G-CSF response in casp-1^{-/-} and Nlrp1b^{-/-} knockout mice (1, 5). In the Cys-modified 6323 pro-drug, VbP is attached to a FAPα-cleavable linker by a peptide bond involving the amino group of valine, and as a result VbP is biologically inactive until it is released by FAPα cleavage. The significant levels of FAPα enzymatic activity present in the tissues and blood of normal mice (10) allow the pro-drug to be tested for their ability to be activated in vivo by endogenous FAPα using serum concentration of G-CSF as a biomarker.

Cys-modified 6323 pro-drug was produced as described in Example 16. Male BALB/c mice of 7-8 weeks of age (Charles River Laboratories) were injected subcutaneously with vehicle (PBS), 1.28 or 0.64 mg/mouse Cys-modified 6323, in groups of 5 mice per treatment. Six (6) hours after dosing, blood was collected by cardiac puncture, and serum concentration of G-CSF was measured using a mouse G-CSF Quantikine ELISA kit (R&D Systems) according to the manufacturer's instructions.

At both doses tested, 1.28 and 0.64 mg/mouse, Cys-modified 6323 (grey and open bars, respectively; FIG. 28) induced significant increases in the serum concentration of G-CSF compared to vehicle treated mice (dark bars; FIG. 28). The results indicate that the FAPα-cleavable linker in 6323 is capable of being cleaved by endogenous FAPα in vivo to release biologically active VbP.

### Example 19: Illustrative Synthetic Schemes

Synthesis of the immuno-DASH inhibitors that can be incorporated in the binder-drug conjugates of the invention may involve a coupling reaction using a coupling reagent, such as HATU, etc, followed by de-protection when necessary, using, for example a reagent such as BCl₃ or HCl-PhB(OH)₂ method when necessary. Some of the target compounds were purified by RP-HPLC using Varian semi-preparative system with a Discovery C18 569226-U RP-HPLC column. The mobile phase was typically made by mixing water (0.1% TFA) with acetonitrile (0.08% TFA) in gradient concentration. The compound code, structure and characterization are shown in Table 1.

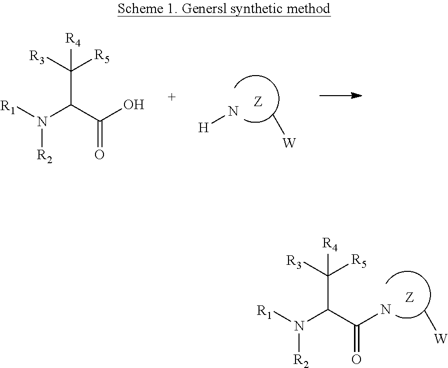

### Exampled Synthetic Procedures of Gly(1-Adamantyl)-boroPro (ARI-5544 or 3102A-2C)

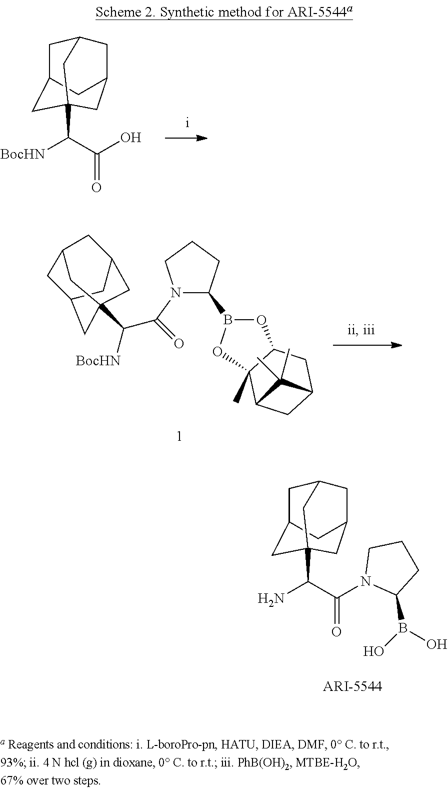

Synthesis of Gly(1-adamantyl)-boroPro (ARI-5544). A solution of 4 N HCl (g) in dioxane (5 mL, 20 mmol) was added to Compound 1 (0.86 g, 1.6 mmol) under dryice/acetone cooling and then was allowed to stir for 3 hrs at room temperature. The reaction mixture was concentrated under reduced pressure and then co-evaporated with ethyl ether (3×15 mL) to afford (+)-pinandiol protected ARI-5544) which was dissolved with a pre-cooled 0.08 N HCl (10 mL). Then, tert-Butyl methyl ether (MTBE) (10 mL) and phenylboronic acid (0.22 g, 1.7 mmol) were added. The mixture was stirred at room temperature for 3 hours and the aqueous phase was separated. The MTBE layer was extracted with 0.08 N HCl (5 mL) and the combined water extractions were washed with ether (3×10 ml). Concentrated the aqueous phase on rotovap (<30° C.) and the crude product was purified by preparative HPLC (eluents: solvent A, 0.1% TFA in water; solvent B, 0.08% TFA in acetonitrile). Collected the desired fractions and concentrated to approximately 10 mL and freeze dry to give Compound ARI-5544 as a TFA salt (0.45 g, 67% over two steps). ¹H NMR (D20): 61.60-1.75 (m, 14H), 1.85-2.15 (m, 6H), 3.07 (dd, J=11.1, 6.9 Hz, 1H), 3.46-3.52 (m, 1H), 3.76 (t, J=9.4 Hz, 1H), 3.91 (s, 1H). MS (ESI+) for C₁₆H₂₇BN₂O₃ m/z (rel intensity): 577.5 ([2× (M−H2O)+H]+, 76), 307.42 ([M+H]+, 100), 289.4 ([M−H2O+H]+, 24).

### Exampled Synthetic Procedures of 3102C

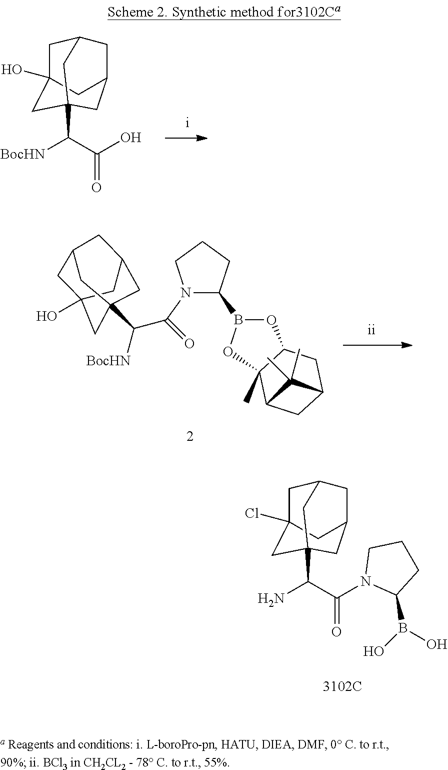

Synthesis of 3102C. Starting from N-Boc-L-3-hydroxy-1-Adamantyl-Glycine with the similar coupling reaction described above for the preparation of 1, compound 2 was prepared. This product (0.28 g, 0.5 mmol) was dissolved in dry dichloromethane (5.0 mL) and cooled to −78° C. while BCl₃ (1 M in dichloromethane, 5.0 mL) was added dropwise. The mixture was stirred at −78° C. for 1 hr, brought to room temperature and then concentrated in vacuo. The residue was partitioned between ether (5 mL) and water (5 mL). The aqueous layer was washed twice with more ether (2×5 mL), concentrated in vacuo and further purified by semipreparative RP-HPLC to give 3102C as a TFA salt (0.13 g, 55%).

### Synthesis of 5870. Synthetic Scheme: i. DAST; ii. LiOH; iii. L-boroPro-pn, HATU, DIEA; iv. BCl3

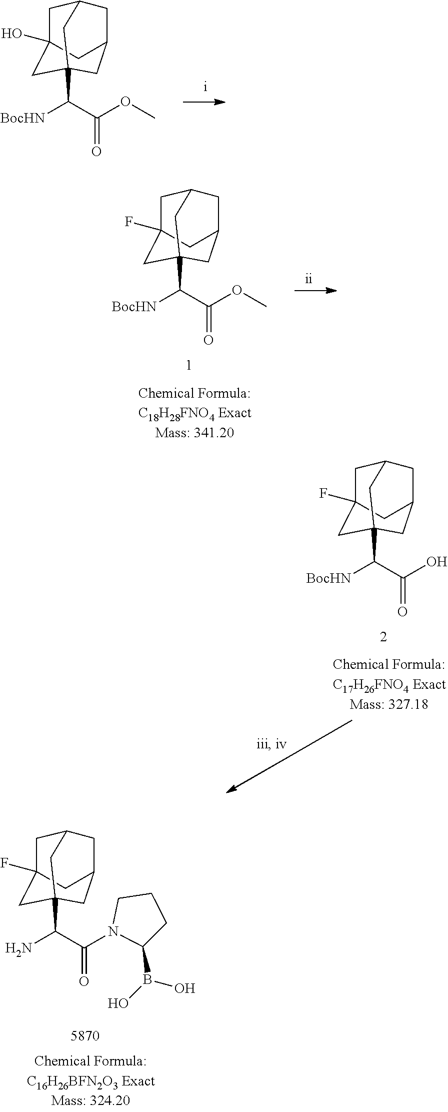 [SMILES: COC(=O)[C@@H](NC(=O)OC(C)(C)C)C12CC3CC(CC(O)(C3)C1)C2 ; COC(=O)[C@@H](NC(=O)OC(C)(C)C)C12CC3CC(CC(F)(C3)C1)C2 ; CC(C)(C)OC(=O)N[C@H](C(=O)O)C12CC3CC(CC(F)(C3)C1)C2 ; N[C@H](C(=O)N1CCC[C@H]1B(O)O)C12CC3CC(CC(F)(C3)C1)C2]

### Synthesis of 5871. Synthetic Scheme: i. Mel, K₂CO₃, DMF; ii.4 eq. DAST and high temperature; iii. LiOH; iv. L-boroPro-pn, HATU, DIEA; v. HCl then PhB(OH)₂

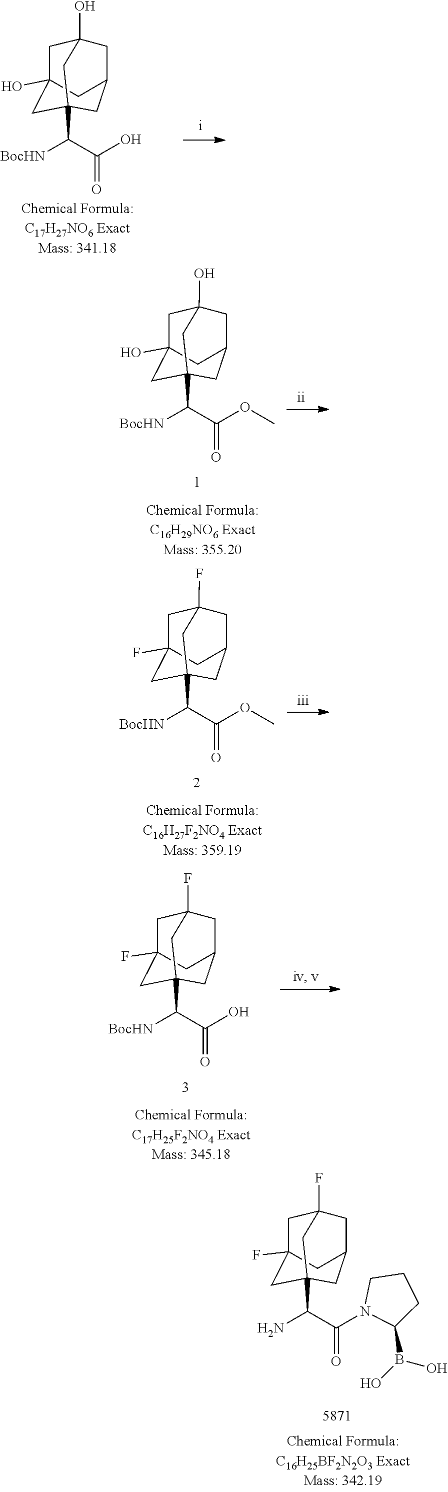 [SMILES: CC(C)(C)OC(=O)N[C@H](C(=O)O)C12CC3CC(O)(CC(O)(C3)C1)C2 ; COC(=O)[C@@H](NC(=O)OC(C)(C)C)C12CC3CC(O)(CC(O)(C3)C1)C2 ; COC(=O)[C@@H](NC(=O)OC(C)(C)C)C12CC3CC(F)(CC(F)(C3)C1)C2 ; CC(C)(C)OC(=O)N[C@H](C(=O)O)C12CC3CC(F)(CC(F)(C3)C1)C2 ; N[C@H](C(=O)N1CCC[C@H]1B(O)O)C12CC3CC(F)(CC(F)(C3)C1)C2]

### Synthesis of 5873. Synthetic Scheme: i. L-boroPro-pn, HATU, DIEA; ii. BCl₃

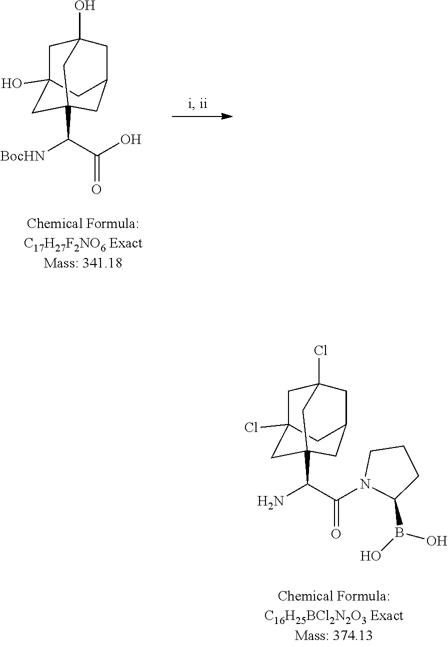 [SMILES: CC(C)(C)OC(=O)N[C@H](C(=O)O)C12CC3CC(O)(CC(O)(C3)C1)C2 ; N[C@H](C(=O)N1CCC[C@H]1B(O)O)C12CC3CC(Cl)(CC(Cl)(C3)C1)C2]

### Synthesis of 5874. Synthetic Scheme: i. 1 eq. DAST at low temperature; ii. LiOH; iii. L-boroPro-pn, HATU, DIEA; iv. HCl then PhB(OH)₂

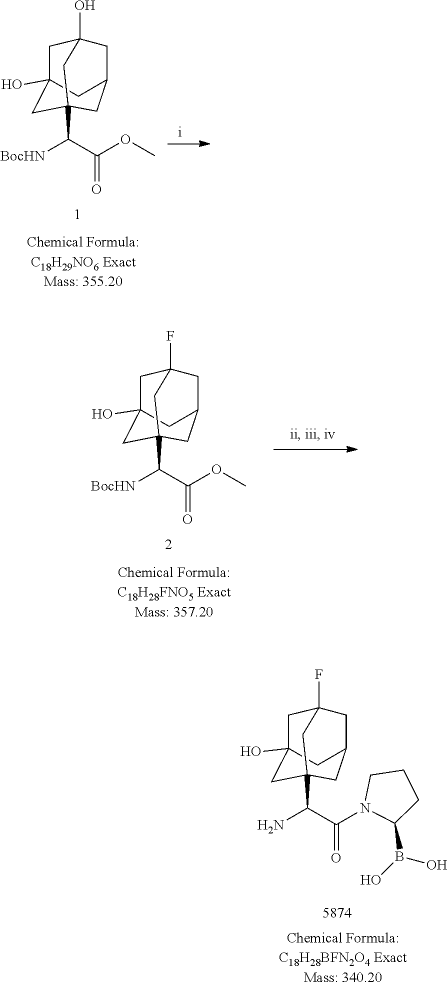 [SMILES: COC(=O)[C@@H](NC(=O)OC(C)(C)C)C12CC3CC(O)(CC(O)(C3)C1)C2 ; COC(=O)[C@@H](NC(=O)OC(C)(C)C)C12CC3CC(O)(CC(F)(C3)C1)C2 ; N[C@H](C(=O)N1CCC[C@H]1B(O)O)C12CC3CC(O)(CC(F)(C3)C1)C2]

### Synthesis of Gly(3-hydroxyl-5,7,-dimethyl-adamantyl)-boroPro. Synthetic Scheme. i. Mel, K₂CO₃; ii. TrisylN₃, KHMDS; iii. H₂/Pd—C, Boc2O; iv. KOH; v. KMnO₄; vi. L-boroPro-pn, HATU, DIEA; vii. HCl then PhB(OH)₂

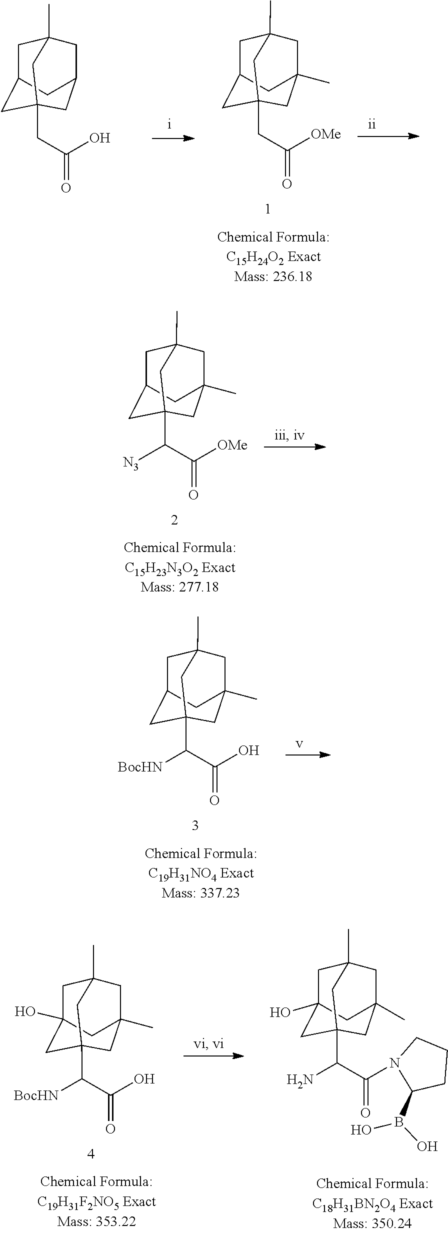 [SMILES: CC12CC3CC(C1)CC(CC(=O)O)(C3)C2 ; COC(=O)CC12CC3CC(C)(CC(C)(C3)C1)C2 ; COC(=O)C(N=[N+]=[N-])C12CC3CC(C)(CC(C)(C3)C1)C2 ; CC12CC3CC(C)(C1)CC(C(NC(=O)OC(C)(C)C)C(=O)O)(C3)C2 ; CC12CC3(C)CC(O)(C1)CC(C(NC(=O)OC(C)(C)C)C(=O)O)(C2)C3 ; CC12CC3(C)CC(O)(C1)CC(C(N)C(=O)N1CCC[C@H]1B(O)O)(C2)C3]

### Synthesis of 5879. Synthetic Scheme: i. DAST; ii. LiOH; iii. L-boroPro-pn, HATU, DIEA; iv. BCl₃

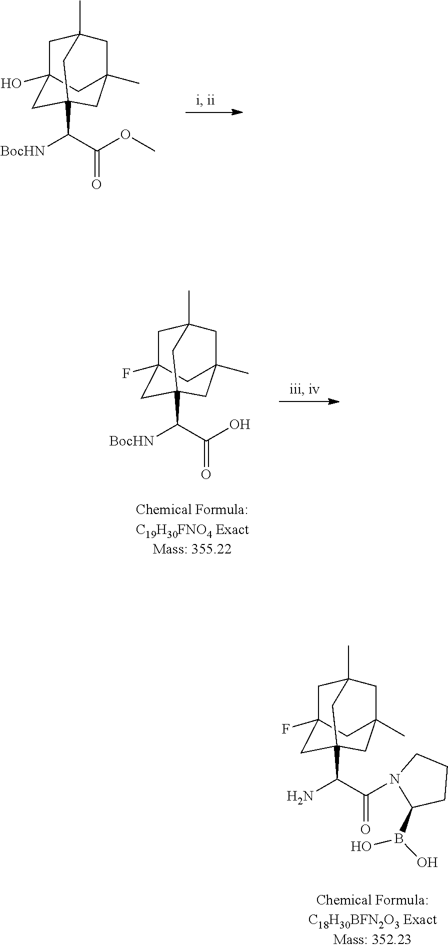 [SMILES: COC(=O)[C@@H](NC(=O)OC(C)(C)C)C12CC3(C)CC(C)(CC(O)(C3)C1)C2 ; CC12CC3(C)CC(F)(C1)CC([C@H](NC(=O)OC(C)(C)C)C(=O)O)(C2)C3 ; CC12CC3(C)CC(F)(C1)CC([C@H](N)C(=O)N1CCC[C@H]1B(O)O)(C2)C3]

### Synthesis of 5880. Synthetic Scheme: i. L-boroPro-pn, HATU, DIEA; ii. BCl₃

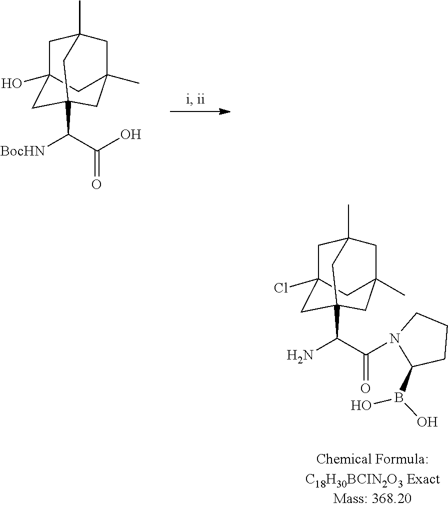 [SMILES: CC12CC3(C)CC(O)(C1)CC([C@H](NC(=O)OC(C)(C)C)C(=O)O)(C2)C3 ; CC12CC3(C)CC(Cl)(C1)CC([C@H](N)C(=O)N1CCC[C@H]1B(O)O)(C2)C3]

### Synthesis of 6067. Synthetic Scheme: i. Oxidation; ii. DAST; iii. H2/Pd—C; iv. L-boroPro-pn, HATU, DIEA; v. HCl; vi. PhB(OH)₂

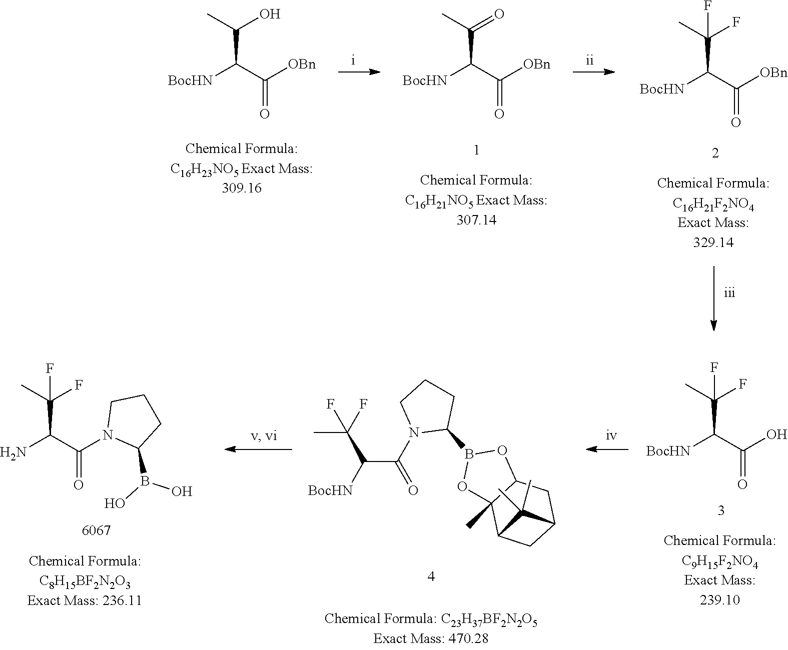

TABLE 1
Compounds code, structures, and chemical characterization

| Compound | Structure | Characterization |
|---|---|---|
| ARI-5544 (3102A-2C) | 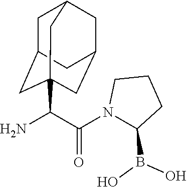 [SMILES: N[C@H](C(=O)N1CCC[C@H]1B(O)O)C12CC3CC(CC(C3)C1)C2] | ¹H NMR (D₂O): δ 1.60-1.75 (m, 13H), 1.85-2.15 (m, 6H), 3.07 (dd, J = 11.1, 6.9 Hz, 1H), 3.46-3.52 (m, 1H), 3.76 (t, J = 9.4 Hz, 1H), 3.91 (s, 1H). MS (ESI+) for C₁₆H₂₇BN₂O₃ m/z (rel intensity): 577.5 ([2 × (M − H₂O) + H]⁺, 76), 307.4 ([M + H]⁺, 100), 289.4 ([M − H₂O + H]⁺, 24). |
| 3102A-2D | 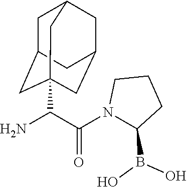 | ¹H NMR (D₂O): δ 1.56-1.75 (m, 13H), 1.95-2.10 (m, 6H), 3.05-3.10 (m, 1H), 3.50-3.60 (m, 1H), 3.65-3.75 (m, 1H), 3.89 (s, 1H). MS (ESI+) for C₁₆H₂₇BN₂O₃ m/z (rel intensity): 577.1 ([2 × (M − H₂O) + H]⁺, 65), 289.1 ([M − H₂O + H]⁺, 100). |
| 3102A | 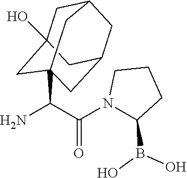 [SMILES: N[C@H](C(=O)N1CCC[C@H]1B(O)O)C12CC3CC(CC(O)(C3)C1)C2] | ¹H NMR (D₂O): δ 1.43-1.80 (m, 13H), 1.83-1.92 (m, 1H), 2.08-2.16 (m, 2H), 2.27 (s, 2H), 3.08 (dd, J = 11.2, 6.9 Hz, 1H), 3.44-3.56 (m, 1H), 3.76 (t, J = 8.5 Hz, 1H), 4.03 (s, 1H). MS (ESI+) for C₁₆H₂₇BN₂O₄ m/z (rel intensity): 609.4 ([2 × (M − H₂O) + H]⁺, 15), 323.2 ([M + H]⁺, 50), 305.2 ([M − H₂O + H]⁺, 100). |
| 3102A-2B | 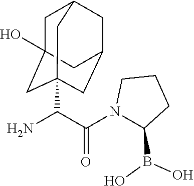 | ¹H NMR (D₂O): δ 1.30-1.80 (m, 13H), 1.85-2.10 (m, 3H), 2.24 (s, 2H), 3.04- 3.08 (m, 1H), 3.50-3.60 (m, 1H), 3.65- 3.75 (m, 1H), 4.02 (s, 1H). MS (ESI+) for C₁₆H₂₇BN₂O₄ m/z (rel intensity): 609.3 ([2 × (M − H₂O) + H]⁺, 21), 323.2 ([M + H]⁺, 7), 305.1 ([M − H₂O + H]⁺, 100). |
| 3102C | 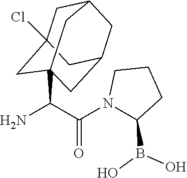 [SMILES: N[C@H](C(=O)N1CCC[C@H]1B(O)O)C12CC3CC(CC(Cl)(C3)C1)C2] | ¹H NMR (D₂O): δ 1.54-1.80 (m, 7H), 1.85- 1.95 (m, 1H), 2.00-2.21 (m, 8H), 2.27 (s, 2H), 3.09 (dd, J = 11.2, 7.0 Hz, 1H), 3.40- 3.55 (m, 1H), 3.77 (t, J = 7.7 Hz, 1H), 4.03 (s, 1H). MS (ESI+) for C₁₆H₂₆BClN₂O₃ m/z (rel intensity): 341.2 ([M + H]⁺, 50), 323.3 ([M − H₂O + H]⁺, 100). |
| 8596-1 | 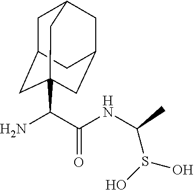 | ¹H NMR (D₂O) δ 1.18 (d, J = 7.4 Hz, 3H), 1.61-1.76 (m, 12H), 2.04 (s, 3H), 2.88 (q, J = 7.3 Hz, 1H), 3.57 (s, 1H). MS (ESI+) for C₁₄H₂₅BN₂O₃ m/z (rel intensity): 525.4 ([2 × (M − H₂O) + H]⁺, 20), 263.2 ([M − H₂O + H]⁺, 100). |
| 4268 | 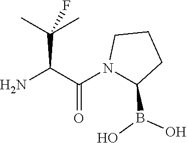 | ¹H NMR (D₂O): δ 1.29-2.09 (m, 10H), 3.05-3.15 (m, 1H), 3.45-3.60 (m, 1H), 3.70-3.80 (m, 1H), 4.49 (d, J = 11.5 Hz, 1H). MS (ESI+) for C₉H₁₈BFN₂O₃ m/z (rel intensity): 429.1 ([2 × (M − H₂O) + H]⁺, 100), 214.9 ([M − H₂O + H]⁺, 80). |
| 4175CH | 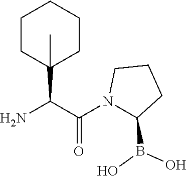 | ¹H NMR (D₂O): δ 1.09 (s, 3H), 1.40-1.75 (m, 11H), 1.80-1.95 (m, 1H), 2.00-2.15 (m, 2H), 3.06 (dd, J = 11.5, 7.0 Hz, 1H), 3.47-3.56 (m, 1H), 3.76-3.82 (m, 1H), 4.04 (s, 1H). MS (ESI+) for C₁₃H₂₅BN₂O₃ m/z (rel intensity): 501.5 ([2 × (M − H₂O) + H]⁺, 100), 269.3 ([M − H₂O + H]⁺, 50). |
| 4175CP | 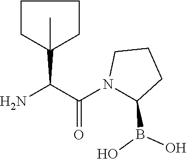 | ¹H NMR (D₂O): δ 0.95 (s, 3H), 1.30-1.80 (m, 10H), 1.95-2.05 (m, 2H), 2.95-3.05 (m, 1H), 3.35-3.70 (m, 2H), 4.08 (s, 1H). MS (ESI+) for C₁₂H₂₃BN₂O₃ m/z (rel intensity): 473.2 ([2 × (M − H₂O) + H]⁺, 34), 237.1 ([M − H₂O + H]⁺, 100). |
| 4175CP-DL | 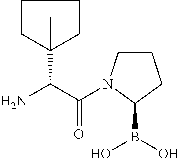 | ¹H NMR (D₂O): δ 0.97 (s, 3H), 1.30-1.60 (m, 9H), 1.90-2.00 (m, 3H), 2.95-3.05 (m, 1H), 3.30-3.60 (m, 2H), 4.06 (s, 1H). MS (ESI+) for C₁₂H₂₃BN₂O₃ m/z (rel intensity): 473.2 ([2 × (M − H₂O) + H]⁺, 66), 237.1 ([M − H₂O + H]⁺, 100). |
| 4949-1 | 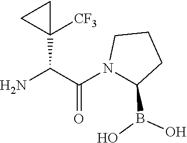 [SMILES: N[C@@H](C(=O)N1CCC[C@H]1B(O)O)C1(C(F)(F)F)CC1] | ¹H NMR (D₂O): δ 0.76-0.84 (m, 1H), 1.15- 1.25 (m, 1H), 1.36-1.45 (m, 2H), 1.75- 1.81 (m, 1H), 1.98-2.18 (m, 3H), 3.12 (t, J = 8.3 Hz, 1H), 3.50-3.70 (m, 2H). MS (ESI+) for C₁₀H₁₆BF₃N₂O₃ m/z (rel intensity): 525.2 ([2 × (M − H₂O) + H]⁺, 56), 263.1 ([M − H₂O + H]⁺, 100). |
| 4949-2 | 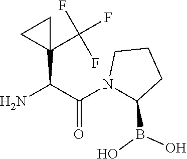 [SMILES: N[C@H](C(=O)N1CCC[C@H]1B(O)O)C1(C(F)(F)F)CC1] | ¹H NMR (D₂O): δ 1.15-1.23 (m, 1H), 1.36 (s, 3H), 1.68-2.03 (m, 2H), 2.12-2.15 (m, 2H), 3.13 (t, J = 9.3 Hz, 1H), 3.47-3.56 (m, 1H), 3.72-3.78 (m, 1H), 4.86 (s, 1H). MS (ESI+) for C₁₀H₁₆BF₃N₂O₃ m/z (rel intensity): 525.2 ([2 × (M − H₂O) + H]⁺, 50), 281.1 ([M + H]⁺, 100), 263.1 ([M − H₂O + H]⁺, 26). |
| 4367 | 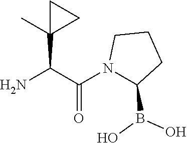 [SMILES: CC1([C@H](N)C(=O)N2CCC[C@H]2B(O)O)CC1] | ¹H NMR (D₂O): δ 0.50-0.95 (m, 4H), 1.05 (s, 3H), 1.65-1.75 (m, 1H), 1.80-1.95 (m, 1H), 2.00-2.10 (m, 2H), 3.00-3.10 (m, 1H), 3.40-3.55 (m, 1H), 3.60-3.70 (m, 2H), 3.81 (s, 1H). MS (ESI+) for C₁₀H₁₉BN₂O₃ m/z (rel intensity): 417.2 ([2 × (M − H₂O) + H]⁺, 87), 227.1 ([M + H]⁺, 45), 209.0 ([M − H₂O + H]⁺, 89). |
| 4367DL | 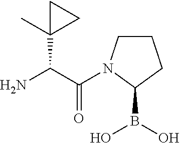 [SMILES: CC1([C@@H](N)C(=O)N2CCC[C@H]2B(O)O)CC1] | ¹H NMR (D₂O): δ 0.35-0.65 (m, 3H), 0.70- 0.85 (m, 1H), 0.99 (s, 1H), 1.09 (s, 3H), 1.65-1.75 (m, 1H), 1.80-2.10 (m, 3H), 3.00-3.10 (m, 1H), 3.40-3.55 (m, 1H), 3.60-3.75 (m, 1H), 4.01 (s, 1H). MS (ESI+) for C₁₀H₁₉BN₂O₃ m/z (rel intensity): 417.2 ([2 × (M − H₂O) + H]⁺, 70), 209.0 ([M − H₂O + H]⁺, 100). |
| 5349 | 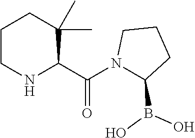 [SMILES: CC1(C)CCCN[C@@H]1C(=O)N1CCC[C@H]1B(O)O] | ¹H NMR (D₂O): δ 0.60-1.20 (m, 8H), 1.60- 2.15 (m, 6H), 3.00-3.11 (m, 2H), 3.40- 3.55 (m, 2H), 3.75-3.85 (m, 1H), 4.05- 4.30 (m, 1H). MS (ESI+) for C₁₂H₂₃BN₂O₃ m/z (rel intensity): 255.2 ([M + H]⁺, 100). |
| 5362 | 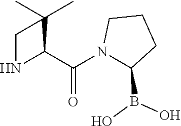 [SMILES: CC1(C)CN[C@@H]1C(=O)N1CCC[C@H]1B(O)O] | ¹H NMR (D₂O): δ 0.90-1.52 (m, 6H), 1.70- 1.80 (m, 1H), 1.90-2.15 (m, 3H), 3.07- 3.14 (m, 1H), 3.27-3.31 (m, 1H), 3.50- 3.72 (m, 2H), 3.90-3.95 (m, 1H), 5.32 (s, 1H). MS (ESI+) for C₁₀H₁₉BN₂O₃ m/z (rel intensity): 227.2 ([M + H]⁺, 100). |
| 5363 | 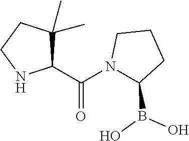 [SMILES: CC1(C)CCN[C@@H]1C(=O)N1CCC[C@H]1B(O)O] | ¹H NMR(D₂O): δ 1.05-1.10 (m, 3H), 1.25- 1.35 (m, 3H), 1.70-2.15 (m, 6H), 3.10 (dd, J = 11.0, 6.9 Hz, 1H), 3.43-3.80 (m, 4H), 4.29 (s, 1H). MS (ESI+) for C₁₁H₂₁BN₂O₃ m/z (rel intensity): 241.2 ([M + H]⁺, 100). |

### Example 20: Exemplary Immuno-DASH-Inhibitors

The following table provides, in columns 3-7, the inhibitory IC50's as determined for cell-free preparations of DPP8, DPP9, DPP4, DPP2, FAP (fibroblast activating protein) and PREP. These IC50 values (in nM) were determined following the protocol set forth in Example 4 below. Where calculated, the table also provides the intracellular IC50 (“IIC50”) for inhibition of DPP8 and DPP9 in whole cells, according to the protocol described in Example 3 below. In certain instances, the table also provides the IC50 for inducing pyroptosis of macrophages in cell culture.

| Cmpd | | | | | | | | IIC50 | |
|---|---|---|---|---|---|---|---|---|---|
| ID | Structure | DPP8 | DPP9 | DPP4 | DPP2 | FAP | PREP | DPP8/9 | Pyroptosis |
| 5544 | 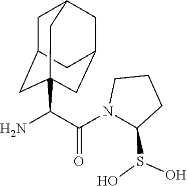 | 7.8 | 6.1 | 6.4 | 27 | 31 | 42 | 4.7 | 1.7 |
| 3102C | 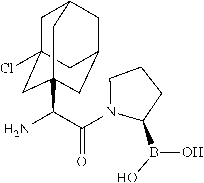 [SMILES: N[C@H](C(=O)N1CCC[C@H]1B(O)O)C12CC3CC(CC(Cl)(C3)C1)C2] | 5 | 4 | 6 | 21 | 20 | 15 | 2.7 | NA |
| 4175CH | 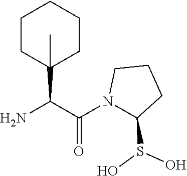 | 5.4 | 2.7 | 2.8 | 54 | 76 | 34 | 5.8 | 6.0 |
| 4175CP | 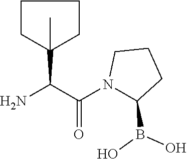 | 5.3 | 5.6 | 2.9 | 9.2 | 88 | 68 | 27 | 33 |
| 4268 | 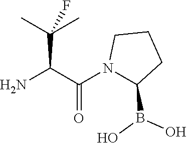 | 10 | 14 | 12 | 68 | 75 | 23 | 28 | 87 |
| 5362 | 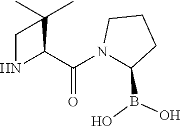 [SMILES: CC1(C)CN[C@@H]1C(=O)N1CCC[C@H]1B(O)O] | 4.5 | 2.4 | 0.5 | 67 | 58 | 1200 | 110 | NA |
| 4949 | 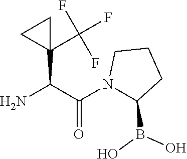 [SMILES: N[C@H](C(=O)N1CCC[C@H]1B(O)O)C1(C(F)(F)F)CC1] | 24 | 19 | 22 | 39 | 1200 | 58 | NA | 1900 |
| 3102A | 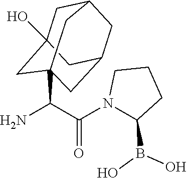 [SMILES: N[C@H](C(=O)N1CCC[C@H]1B(O)O)C12CC3CC(CC(O)(C3)C1)C2] | 10 | 7 | 7.2 | 81 | 9 | 49 | 1200 | NA |
| 5320 | 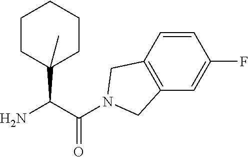 | 1.9 | 7.9 | 2070 | 53,940 | >100,000 | 89,450 | 280 | NA |
| 5349 | 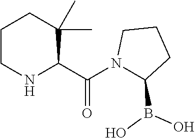 [SMILES: CC1(C)CCCN[C@@H]1C(=O)N1CCC[C@H]1B(O)O] | 250 | 3100 | 92 | 610 | 14,000 | 730 | NA | NA |
| 5363 | 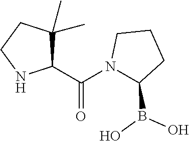 [SMILES: CC1(C)CCN[C@@H]1C(=O)N1CCC[C@H]1B(O)O] | 5.5 | 5.2 | 1.4 | 110 | 32 | 1400 | 270 | NA |
| 4367 | 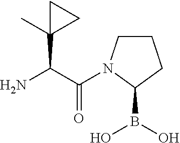 [SMILES: CC1([C@H](N)C(=O)N2CCC[C@H]2B(O)O)CC1] | 5.6 | 1.5 | 1.4 | 34 | 350 | 200 | 55 | NA |
| 5870 | 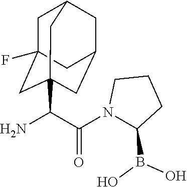 [SMILES: N[C@H](C(=O)N1CCC[C@H]1B(O)O)C12CC3CC(CC(F)(C3)C1)C2] | 9.7 | 8.4 | 9.3 | 58 | 33 | | 9.1 | |

### Example 21: Protocol for Determining the Intracellular IC₅₀ Against DPP Activity in 293T Cells

Since 293T cells express low levels of endogenous DPP8/9 but not DPP IV, DPP II, or FAP, this allows for assessment of intracellular DPP8/9 inhibition without interference from other background DPP activity. (Danilova, O. et al. (2007) Bioorg. Med. Chem. Lett. 17, 507-510; Wang, X. M. et al. (2005) Hepatology 42, 935-945) This information allows for assessment of cell penetrability of the compounds.

### Materials

293T cells (ATCC, Cat. No. CRL-11268)
RPMI 1640 cell culture media without phenol red (VWR, Cat. No. 45000-410) supplemented with 2 mM L-glutamine (VWR, Cat. No. 45000-676), 10 mM HEPES (VWR, Cat. No. 45000-690), 1 mM sodium pyruvate (VWR, Cat. No. 45000-710), 4500 mg/L glucose (VWR, Cat. No. 45001-116), 1× penicillin-streptomycin (VWR, Cat. No. 45000-652)
Inhibitor or prodrug
4000× substrate solution (100 mM Ala-Pro-AFC (Bachem, Cat. No. I-1680) in DMSO)
96-well black clear-bottom plates (BD Biosciences, Cat. No. 353948)

### Instrumentation

Plate shaker
Molecular Devices SpectraMax® M2e microplate reader

### Protocol

### Assay Setup

Trypsinize and spin down cells from a 75 cm2 or larger flask, wash with PBS and resuspend in RPMI 1640. Count the cells in the resulting suspension and adjust the volume such that it has 100,000 cells per 75 μL. Add 100 μL of RPMI 1640 alone to rows A-C of column 1 in a 96-well black clear-bottomed plate. Add 75 μL of the cell suspension to the remaining wells in columns 2-10. Equilibrate the plates at 37° C. overnight.

### Sample Preparation

1. To prepare the compound for the assay, dissolve it in either DMSO or, if cyclization is suspected, in pH 2.0 water (0.01 N HCl) to a final concentration of 100 mM.

For pH 2.0 stocks, incubate at room temperature for a minimum of four hours and up to overnight. From this, prepare a 4 mM stock in RPMI 1640. If the inhibitor is insoluble at this concentration, dilute the 100 mM stock 1:10 to 10 mM. Using this stock, prepare a 0.4 mM stock as described above. The pH of each diluted sample should be confirmed to be that of the cell culture medium (pH 7-8).

2. Prepare a dilution plate for the compounds prepared in step 3. To do so, add the 4 or 0.4 mM stocks prepared previously to row A of a 96-well plate. From this, perform 1:10 serial dilutions into RPMI 1640 down to row G as shown below. Row H should have RPMI 1640 cell culture medium alone:

3. Add 25 μL of the compound from the dilution plate prepared in step 4 to the assay plate in columns 2-10 where appropriate. Each sample should be tested in triplicate. Shake the plate briefly and allow it to incubate for two hours at 37° C.

4. During this time, the substrate should be prepared. To do so, dilute the 100 mM stock 1:400 into RPMI 1640 to its final working concentration of 250 μM.

5. After the incubation at 37° C. is complete, add 10 μL of the substrate prepared in step 5 to each well. Shake the plate briefly and allow it to incubate for 10 minutes at 37° C. Once complete, read the fluorescence at λex: 400, λem: 505.

### Data Analysis

1. Import the fluorescence values directly into Prism as the y values. For the inhibitor concentrations, which are the x values, be sure to divide the concentrations in the dilution plate by 4 to account for their dilution in the assay. The x values must be converted into log values prior to their importation into Prism. The concentration for the no inhibitor wells (row H) should be entered as −14 (equal to 10-14 M).

2. Once the values have been entered, under “Analyze”, choose “Nonlinear regression (curve fit)”. At the subsequent prompt, choose “log(inhibitor) vs. response”. This will calculate the IC50 values, which can be found in the “Results” section.

### Example 22: Protocol for In Vitro Inhibition Assay for Dipeptidyl Peptidase IV, Dipeptidyl Peptidase 8, Dipeptidyl Peptidase 9, Dipeptidyl Peptidase II, Fibroblast Activation Protein or Prolyl Oligopeptidase

This assay may be used to determine the IC50 of various inhibitors against recombinant human dipeptidyl peptidase IV (DPPIV), dipeptidyl peptidase 8 (DPP8), dipeptidyl peptidase 9 (DPP9), dipeptidyl peptidase II, fibroblast activation protein (FAP) or prolyl oligopeptidase (PREP).

### Materials

### Enzymes

Recombinant human DPPIV (R&D Systems, Cat. No. 1180-SE)
Recombinant human DPP8 (Enzo Life Sciences, Cat. No. BML-SE527)
Recombinant human DPP9 (R&D Systems, Cat. No. 5419-SE)
Recombinant human DPPII (R&D Systems, Cat. No. 3438-SE)
Recombinant human FAP (R&D Systems, Cat. No. 3715-SE)
Recombinant human PREP (R&D Systems, Cat. No. 4308-SE)

### Assay Buffers

mM Tris, pH 8.0 (DPPIV and DPP9)
50 mM Tris, pH 7.5 (DPP8)
mM MES, pH 6.0 (DPPII)
50 mM Tris, 140 mM NaCl, pH 7.5 (FAP)
mM Tris, 0.25 M NaCl, pH 7.5 (PREP)

### Substrates

4000× substrate solution (100 mM Gly-Pro-AMC (VWR, Cat. No. 100042-646) in DMSO, DPPIV, DPP8 and DPP9)
4000× substrate solution (100 mM Lys-Pro-AMC (Bachem, Cat. No. I-1745) in DMSO, DPPII)
100× substrate solution (2.5 mM Z-Gly-Pro-AMC (VWR, Cat. No. I-1145.0050BA) in DMSO, FAP and PREP)

### General Materials

Compound
96-well black clear-bottom plates (Costar, Cat. No. 3603)

### Instrumentation

Plate shaker
Molecular Devices SpectraMax® M2e microplate reader

### Protocol

1. To prepare the compound for the assay, dissolve it in either DMSO or, if cyclization is suspected, in pH 2.0 water (0.01 N HCl) to a final concentration of 100 mM. For pH 2.0 stocks, incubate at room temperature for a minimum of four hours and up to overnight. From this, prepare a 1 mM stock at pH 7.4 in 50 mM Tris. If the inhibitor is insoluble at this concentration, dilute the 100 mM stock 1:10 to 10 mM. Using this stock, prepare a 0.1 mM stock as described above.

2. Prepare a dilution plate for the compound stocks to be tested. Add the 0.1 and/or 1 mM stocks prepared previously to row A of a 96-well plate. From this, perform 1:10 serial dilutions into the appropriate assay buffer down the columns as shown below:

3. Prepare 20× substrate solution by diluting the DMSO stocks into the appropriate assay buffer.

4. Dilute the enzymes into their appropriate assay buffers. The dilution factor is lot dependent and must be determined prior to performing the assay. The final enzyme concentrations should be 0.1, 0.8, 0.4, 0.2, 1.2, and 0.6 nM for DPPIV, 8, 9, II, FAP and PREP respectively. Add 180 μL to each well needed in columns 2-10. Column 1 should be prepared as shown below:

5. Add 20 μL of the compound of interest from the dilution plate prepared in step 2 to columns 2-10 of the assay plate where appropriate. Each sample should be tested in triplicate. Allow this to incubate for 10 minutes at room temperature, shaking the plate for the first two minutes.

6. Add 10 μL of 20× substrate prepared in step 3 to each well and allow this to incubate for 15 minutes at room temperature, shaking the plate for the first two minutes.

7. Read the fluorescence at λex: 380, λem: 460.

### Data Analysis

1. Average the values for the blanks in wells A1, B1 and C1 and subtract this from the remaining wells. Import the resulting fluorescence values into Prism as the y values. For the compound concentrations, which are the x values, be sure to divide the concentrations in the dilution plate by 10.5 to account for their dilution in the assay plate. These must be converted into log values prior to their importation into Prism

2. Once the values have been entered, under “Analyze” and choose “Nonlinear regression (curve fit)”. At the subsequent prompt, choose “log(inhibitor) vs. response”. This will calculate the IC50 values, which can be found in the “Results” section.

### CITED REFERENCES

1. Okondo M C, Rao S D, Taabazuing C Y, Chui A J, Poplawski S E, Johnson D C, Bachovchin D A. Inhibition of Dpp8/9 Activates the Nlrp1b Inflammasome. Cell Chem Biol. 2018; 25(3):262-7 e5. Epub 2018/02/06. doi: 10.1016/j.chembiol.2017.12.013. PubMed PMID: 29396289; PMCID: PMC5856610.
2. Johnson D C, Taabazuing C Y, Okondo M C, Chui A J, Rao S D, Brown F C, Reed C, Peguero E, de Stanchina E, Kentsis A, Bachovchin D A. DPP8/DPP9 inhibitor-induced pyroptosis for treatment of acute myeloid leukemia. Nat Med. 2018; 24(8):1151-6. Epub 2018/07/04. doi: 10.1038/s41591-018-0082-y. PubMed PMID: 29967349; PMCID: PMC6082709.
3. Zhong F L, Robinson K, Teo D E T, Tan K Y, Lim C, Harapas C R, Yu C H, Xie W H, Sobota R M, Au V B, Hopkins R, D'Osualdo A, Reed J C, Connolly J E, Masters S L, Reversade B. Human DPP9 represses NLRP1 inflammasome and protects against autoinflammatory diseases via both peptidase activity and FUND domain binding. J Biol Chem. 2018; 293(49):18864-78. Epub 2018/10/07. doi: 10.1074/jbc.RA118.004350. PubMed PMID: 30291141; PMCID: PMC6295727.
4. Rayamajhi M, Zhang Y, Miao E A. Detection of pyroptosis by measuring released lactate dehydrogenase activity. Methods Mol Biol. 2013; 1040:85-90. doi: 10.1007/978-1-62703-523-1_7. PubMed PMID: 23852598; PMCID: PMC3756820.
5. Okondo M C, Johnson D C, Sridharan R, Go E B, Chui A J, Wang M S, Poplawski S E, Wu W, Liu Y, Lai J H, Sanford D G, Arciprete M O, Golub T R, Bachovchin W W, Bachovchin D A. DPP8 and DPP9 inhibition induces pro-caspase-1-dependent monocyte and macrophage pyroptosis. Nature chemical biology. 2017; 13(1):46-53. doi: 10.1038/nchembio.2229. PubMed PMID: 27820798.
6. de Vasconcelos N M, Vliegen G, Goncalves A, De Hert E, Martin-Perez R, Van Opdenbosch N, Jallapally A, Geiss-Friedlander R, Lambeir A M, Augustyns K, Van Der Veken P, De Meester I, Lamkanfi M. DPP8/DPP9 inhibition elicits canonical Nlrplb inflammasome hallmarks in murine macrophages. Life Sci Alliance. 2019; 2(1). Epub 2019/02/06. doi: 10.26508/lsa.201900313. PubMed PMID: 30718379; PMCID: PMC6362307.
7. Dinarello C A. Biologic basis for interleukin-1 in disease. Blood. 1996; 87(6):2095-147. Epub 1996/03/15. PubMed PMID: 8630372.
8. Adams S, Miller G T, Jesson M I, Watanabe T, Jones B, Wallner B P. PT-100, a small molecule dipeptidyl peptidase inhibitor, has potent antitumor effects and augments antibody-mediated cytotoxicity via a novel immune mechanism. Cancer Res. 2004; 64(15):5471-80. Epub 2004/08/04. doi: 10.1158/0008-5472.CAN-04-044764/15/5471 [pii]. PubMed PMID: 15289357.
9. Jones B, Adams S, Miller G T, Jesson M I, Watanabe T, Wallner B P. Hematopoietic stimulation by a dipeptidyl peptidase inhibitor reveals a novel regulatory mechanism and therapeutic treatment for blood cell deficiencies. Blood. 2003; 102(5):1641-8. Epub 2003/05/10. doi: 10.1182/blood-2003-01-02082003-01-0208 [pii]. PubMed PMID: 12738665.
10. Keane F M, Yao T W, Seelk S, Gall M G, Chowdhury S, Poplawski S E, Lai J H, Li Y, Wu W, Farrell P, Vieira de Ribeiro A J, Osborne B, Yu D M, Seth D, Rahman K, Haber P, Topaloglu A K, Wang C, Thomson S, Hennessy A, Prins J, Twigg S M, McLennan S V, McCaughan G W, Bachovchin W W, Gorrell M D. Quantitation of fibroblast activation protein (FAP)-specific protease activity in mouse, baboon and human fluids and organs. FEBS open bio. 2013; 4:43-54. doi: 10.1016/j.fob.2013.12.001. PubMed PMID: 24371721; PMCID: 3871272.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

1. A binder-drug conjugate comprising: (i) a cell binding moiety that binds to a cell surface feature on a target cell in a disease state of a tissue, which cell surface feature undergoes slow internalization when bound by the binder-drug conjugate; (ii) a drug moiety that has a pharmacological effect on bystander cells proximate to the target cell, which drug moiety has an EC50 for the pharmacological effect which is attenuated by at least 10 fold when part of the binder-drug conjugate relative to a free drug moiety released from the binder-drug conjugate; and (iii) a linker moiety covalently linking the polypeptide binder moiety to the drug moiety, which linker moiety includes a substrate recognition sequence that is cleavable by an enzyme present extracellularly in the disease tissue, wherein in the presence of the enzyme the linker moiety can be cleaved and releases the free drug moiety.

2. The binder-drug conjugate of claim 1, wherein the disease tissue is a tumor; and the target cell is a tumor cell.

3-6. (canceled)

7. The binder-drug conjugate of claim 2, wherein the cell surface feature is a checkpoint protein or a co-stimulatory receptor.

8. The binder-drug conjugate of claim 7, wherein the surface feature is a checkpoint protein selected from the group consisting of CTLA-4, PD-1, LAG-3, BTLA, KIR, TIM-3, PD-L1, PD-L2, B7-H3, B7-H4, HVEM, GAL9, CD160, VISTA, BTNL2, TIGIT, PVR, BTN1A1, BTN2A2, BTN3A2 and CSF-1R; and the binder moiety is a checkpoint antagonist.

9. (canceled)

10. The binder-drug conjugate of claim 1, wherein the cell binding moiety is an antibody.

11. The binder-drug conjugate of claim 1, wherein the binder moiety is a non-antibody scaffold.

12. The binder-drug conjugate of claim 1, represented by any one of the formula 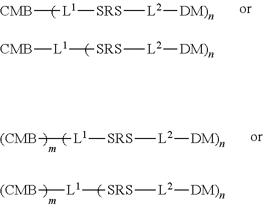
wherein
CBM represents a cell binding moiety-which may be the same or different for each occurrence;
L¹ represents a spacer or a bond;
SRS represents a substrate recognition sequence;
L² represents a self immolative linker or a bond;
DM represents a drug moiety;
m represents an integer from 1 to 6; and
n represents an integer from 1 to 500.

13. The binder-drug conjugate of claim 12, wherein L¹ is a hydrocarbon, N-Succinimidyl 4-(2-pyridylthio) pentanoate, N-Succinimidyl 4-(N-maleimidomethyl) cyclohexane-1 carboxylate, N-Succinimidyl (4-iodo-acetyl) aminobenzoate, or a polyether.

14. The binder-drug conjugate of claim 12, wherein CBM includes a thiol, and L¹ is a poly(ethylene glycol) coupled to the thiol group through a maleimide moiety, L¹ being represented in the formula 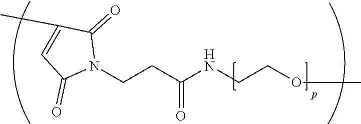
wherein, p represents an integer from 1 to 100.

15. The binder-drug conjugate of claim 12, wherein CBM comprises a thiol, and L¹ is a hydrocarbon moiety coupled to the thiol group through a maleimide moiety, L¹ being represented in the formula 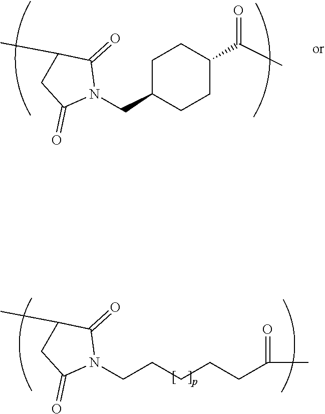
wherein p represents an integer from 1 to 20.

16. The binder-drug conjugate of claim 1, wherein the substrate recognition sequence is cleaved by a protease, preferably a serine protease, metal protease or cysteine protease.

17-22. (canceled)

23. The binder-drug conjugate of claim 12, wherein the substrate recognition sequence is cleaved by fibroblast activating protein alpha (FAPα) and represented by 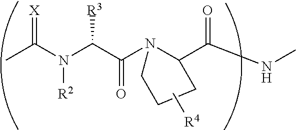
wherein
R² represents H or a (C₁-C₆) alkyl;
R³ represents H or a (C₁-C₆) alkyl;
R⁴ is absent or represents a (C₁-C₆) alkyl, —OH, —NH₂, or halogen;
X represents O or S; and
—NH— represents an amine that is pan of L² if L² is a self immolative linker or part of DM if L² is a bond.

24. The binder-drug conjugate of claim 12, wherein L² is a self immolative linker selected from the group consisting of —NH—(CH2)4-C(═O)—, —NH—(CH2)3-C(═O)—, p-aminobenzyloxycarbonyl (PABC) and 2,4-bis(hydroxymethyl)aniline.

25. (canceled)

26. (canceled)

27. The binder-drug conjugate of claim 1, wherein the drug moiety is an immunomodulator.

28. The binder-drug conjugate of claim 27, wherein the drug moiety is an immune activating agent.

29. (canceled)

30. (canceled)

31. The binder-drug conjugate of claim 27, wherein the immune activating agent is a STING agonist.

32. The binder-drug conjugate of claim 27, wherein the immune activating agent is a RIG-1 agonist.

33. The binder-drug conjugate of claim 27, wherein the immune activating agent is a Toll-like receptor (TLR) agonist.

34-38. (canceled)

39. The binder-drug conjugate of claim 27, wherein the immunomodulator is the low-molecular inhibitor having a molecular weight less than 5000 amu.

40. A binder-drug conjugate comprising a polypeptide including one or more affimer sequences that bind to a cell surface protein on cells in a tumor, and having one or more drug-conjugate moieties appended thereto, which drug-conjugate moieties are represented in the formulas 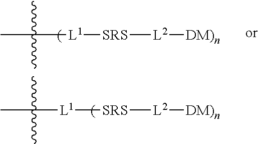
wherein
L¹ represents a spacer or a bond;
SRS represents a substrate recognition sequence for an extracellular protease which is expressed in the extracellular space of a tumor;
L² represents a self immolative linker or a bond;
DM represents a drug moiety;
m represents an integer from 1 to 6; and
n represents an integer from 1 to 500.

41-76. (canceled)

77. An combination PD-L1 inhibitor/innate immunity stimulator comprising a PD-L1 binding polypeptide and a drug moiety conjugated thereto which is a sterile inducer of an innate immune response, wherein the PD-L1 binding polypeptide causes accumulation of the PD-L1 inhibitor/innate stimulator in tumors relative to other tissue of a patient, and wherein the drug moiety is selectively released from the PD-L1 binding polypeptide in the tumor microenvironment relative to other tissue of a patient.

78. A pharmaceutical preparation suitable for therapeutic use in a human patient, comprising (i) a binder-drug conjugate of claim 1, and (ii) one or more pharmaceutically acceptable excipients, buffers, or salts.

79. A method of treating cancer in a subject in need thereof, comprising administering a binder-drug conjugate of claim 1 to the subject.

80. (canceled)

81. A binder-drug conjugate for killing AML cells comprising: i) a cell binding moiety that binds to a cell surface feature selectively expressed on AML cells, which cell surface feature is internalized by AML cells when bound by the binder-drug conjugate; (ii) an immuno-DASH inhibitor moiety, which when released from the conjugate as a free immuno-DASH inhibitor, is toxic to the AML cells; and (iii) a linker moiety covalently linking the cell binding moiety to the I-DASH Inhibitor moiety, which linker moiety includes a substrate recognition sequence that is cleavable by an enzyme present intracellular in the AML cells, wherein internalization of the binder-drug conjugate by AML cells upon binding the cell surface feature results in exposure of the linker moiety to the of the intracellular enzyme and cleavage of the linker moiety and intracellular release the free immuno-DASHG moiety in the AML cells.

82-84. (canceled)


SEQUENCE LISTING (225 sequences)

SEQ ID NO: 1 - protein, 49 aa, Artificial Sequence
  Description of Artificial Sequence: Synthetic polypeptide
MIPGGLSEAKPATPEIQEIVDKVKPQLEEKTNETYGKLEAVQYKTQVLA

SEQ ID NO: 2 - protein, 24 aa, Artificial Sequence
  Description of Artificial Sequence: Synthetic peptide
GTNYYIKVRAGDNKYMHLKVFKSL

SEQ ID NO: 3 - protein, 21 aa, Artificial Sequence
  Description of Artificial Sequence: Synthetic peptide
EDLVLTGYQVDKNKDDELTGF

SEQ ID NO: 4 - protein, 134 aa, Artificial Sequence
  Description of Artificial Sequence: Synthetic polypeptide
  MOD_RES (4)..(4): Gly, Ala, Val, Arg, Lys, Asp or Glu
  MOD_RES (50)..(69): Any amino acid
  MISC_FEATURE (50)..(69): This region may encompass 3-20 residues
  MOD_RES (70)..(70): Gly, Ala, Val, Ser or Thr
  MOD_RES (91)..(91): Arg, Lys, Asn, Gln, Ser or Thr
  MOD_RES (92)..(92): Gly, Ala, Val, Ser or Thr
  MOD_RES (93)..(93): Ala, Val, Ile, Leu, Gly or Pro
  MOD_RES (94)..(113): Any amino acid
  MISC_FEATURE (94)..(113): This region may encompass 3-20 residues
  MOD_RES (114)..(114): Gly, Ala, Val, Asp or Glu
  MOD_RES (116)..(116): Ala, Val, Ile, Leu, Arg or Lys
  See specification as filed for detailed description of substitutions and preferred embodiments
MIPXGLSEAKPATPEIQEIVDKVKPQLEEKTNETYGKLEAVQYKTQVLAXXXXXXXXXXXXXXXXXXXXXTNYYIKVRAGDNKYMHLKVFXXXXXXXXXXXXXXXXXXXXXXXXDXVLTGYQVDKNKDDELTGF

SEQ ID NO: 5 - protein, 134 aa, Artificial Sequence
  Description of Artificial Sequence: Synthetic polypeptide
  MOD_RES (50)..(69): Any amino acid
  MISC_FEATURE (50)..(69): This region may encompass 3-20 residues
  MOD_RES (94)..(113): Any amino acid
  MISC_FEATURE (94)..(113): This region may encompass 3-20 residues
  See specification as filed for detailed description of substitutions and preferred embodiments
MIPRGLSEAKPATPEIQEIVDKVKPQLEEKTNETYGKLEAVQYKTQVLAXXXXXXXXXXXXXXXXXXXXSTNYYIKVRAGDNKYMHLKVFNGPXXXXXXXXXXXXXXXXXXXXADRVLTGYQVDKNKDDELTGF

SEQ ID NO: 6 - protein, 9 aa, Artificial Sequence
  Description of Artificial Sequence: Synthetic peptide
KAWGPKQWW

SEQ ID NO: 7 - protein, 9 aa, Artificial Sequence
  Description of Artificial Sequence: Synthetic peptide
KPYGPRDWD

SEQ ID NO: 8 - protein, 9 aa, Artificial Sequence
  Description of Artificial Sequence: Synthetic peptide
KEYGPEEWW

SEQ ID NO: 9 - protein, 9 aa, Artificial Sequence
  Description of Artificial Sequence: Synthetic peptide
HAYGPRDWD

SEQ ID NO: 10 - protein, 9 aa, Artificial Sequence
  Description of Artificial Sequence: Synthetic peptide
KDHGPIAWW

SEQ ID NO: 11 - protein, 9 aa, Artificial Sequence
  Description of Artificial Sequence: Synthetic peptide
NKHFHQRFW

SEQ ID NO: 12 - protein, 9 aa, Artificial Sequence
  Description of Artificial Sequence: Synthetic peptide
NKHFPIHFW

SEQ ID NO: 13 - protein, 9 aa, Artificial Sequence
  Description of Artificial Sequence: Synthetic peptide
HEFGPAEWD

SEQ ID NO: 14 - protein, 9 aa, Artificial Sequence
  Description of Artificial Sequence: Synthetic peptide
NAHFPQSFW

SEQ ID NO: 15 - protein, 9 aa, Artificial Sequence
  Description of Artificial Sequence: Synthetic peptide
KEHGPDSWW

SEQ ID NO: 16 - protein, 9 aa, Artificial Sequence
  Description of Artificial Sequence: Synthetic peptide
NQHFPHSFW

SEQ ID NO: 17 - protein, 9 aa, Artificial Sequence
  Description of Artificial Sequence: Synthetic peptide
NAHFGPRFW

SEQ ID NO: 18 - protein, 9 aa, Artificial Sequence
  Description of Artificial Sequence: Synthetic peptide
NTWFPESFW

SEQ ID NO: 19 - protein, 9 aa, Artificial Sequence
  Description of Artificial Sequence: Synthetic peptide
NQHFPQSFW

SEQ ID NO: 20 - protein, 9 aa, Artificial Sequence
  Description of Artificial Sequence: Synthetic peptide
KQYGPDDWW

SEQ ID NO: 21 - protein, 9 aa, Artificial Sequence
  Description of Artificial Sequence: Synthetic peptide
KDWGPSNWW

SEQ ID NO: 22 - protein, 9 aa, Artificial Sequence
  Description of Artificial Sequence: Synthetic peptide
KQFGPKDWW

SEQ ID NO: 23 - protein, 9 aa, Artificial Sequence
  Description of Artificial Sequence: Synthetic peptide
NHHFPKRFW

SEQ ID NO: 24 - protein, 8 aa, Artificial Sequence
  Description of Artificial Sequence: Synthetic peptide
YRHFPQWH

SEQ ID NO: 25 - protein, 9 aa, Artificial Sequence
  Description of Artificial Sequence: Synthetic peptide
NIHFPPNFW

SEQ ID NO: 26 - protein, 8 aa, Artificial Sequence
  Description of Artificial Sequence: Synthetic peptide
YTHFPQWT

SEQ ID NO: 27 - protein, 9 aa, Artificial Sequence
  Description of Artificial Sequence: Synthetic peptide
NDHFPHTFW

SEQ ID NO: 28 - protein, 9 aa, Artificial Sequence
  Description of Artificial Sequence: Synthetic peptide
NQHFPSYFW

SEQ ID NO: 29 - protein, 9 aa, Artificial Sequence
  Description of Artificial Sequence: Synthetic peptide
NQYFPPHFW

SEQ ID NO: 30 - protein, 9 aa, Artificial Sequence
  Description of Artificial Sequence: Synthetic peptide
KKHFPASFW

SEQ ID NO: 31 - protein, 9 aa, Artificial Sequence
  Description of Artificial Sequence: Synthetic peptide
KKFFPKHFW

SEQ ID NO: 32 - protein, 9 aa, Artificial Sequence
  Description of Artificial Sequence: Synthetic peptide
KLHFPRSFW

SEQ ID NO: 33 - protein, 9 aa, Artificial Sequence
  Description of Artificial Sequence: Synthetic peptide
YKHFPPNFW

SEQ ID NO: 34 - protein, 9 aa, Artificial Sequence
  Description of Artificial Sequence: Synthetic peptide
EEHFPFQFW

SEQ ID NO: 35 - protein, 9 aa, Artificial Sequence
  Description of Artificial Sequence: Synthetic peptide
KPHFPDNFW

SEQ ID NO: 36 - protein, 9 aa, Artificial Sequence
  Description of Artificial Sequence: Synthetic peptide
YQYFPDQFN

SEQ ID NO: 37 - protein, 9 aa, Artificial Sequence
  Description of Artificial Sequence: Synthetic peptide
VQWFPRSFW

SEQ ID NO: 38 - protein, 9 aa, Artificial Sequence
  Description of Artificial Sequence: Synthetic peptide
AAHFPEHFW

SEQ ID NO: 39 - protein, 9 aa, Artificial Sequence
  Description of Artificial Sequence: Synthetic peptide
REGRQDWVL

SEQ ID NO: 40 - protein, 9 aa, Artificial Sequence
  Description of Artificial Sequence: Synthetic peptide
WVPFPHQQL

SEQ ID NO: 41 - protein, 5 aa, Artificial Sequence
  Description of Artificial Sequence: Synthetic peptide
GRTIQ

SEQ ID NO: 42 - protein, 9 aa, Artificial Sequence
  Description of Artificial Sequence: Synthetic peptide
EPQLDTSPI

SEQ ID NO: 43 - protein, 9 aa, Artificial Sequence
  Description of Artificial Sequence: Synthetic peptide
GDYEQVLIH

SEQ ID NO: 44 - protein, 9 aa, Artificial Sequence
  Description of Artificial Sequence: Synthetic peptide
PADHVLEEA

SEQ ID NO: 45 - protein, 9 aa, Artificial Sequence
  Description of Artificial Sequence: Synthetic peptide
EDTNTDGAL

SEQ ID NO: 46 - protein, 9 aa, Artificial Sequence
  Description of Artificial Sequence: Synthetic peptide
GQSWDQRRQ

SEQ ID NO: 47 - protein, 9 aa, Artificial Sequence
  Description of Artificial Sequence: Synthetic peptide
SKSPIDLPF

SEQ ID NO: 48 - protein, 9 aa, Artificial Sequence
  Description of Artificial Sequence: Synthetic peptide
DPQDVYLNQ

SEQ ID NO: 49 - protein, 9 aa, Artificial Sequence
  Description of Artificial Sequence: Synthetic peptide
GSLHSFGST

SEQ ID NO: 50 - protein, 9 aa, Artificial Sequence
  Description of Artificial Sequence: Synthetic peptide
QEKNQWVEE

SEQ ID NO: 51 - protein, 9 aa, Artificial Sequence
  Description of Artificial Sequence: Synthetic peptide
QKNYEEDPH

SEQ ID NO: 52 - protein, 9 aa, Artificial Sequence
  Description of Artificial Sequence: Synthetic peptide
WDGHKRFAD

SEQ ID NO: 53 - protein, 9 aa, Artificial Sequence
  Description of Artificial Sequence: Synthetic peptide
DDNQERQEH

SEQ ID NO: 54 - protein, 9 aa, Artificial Sequence
  Description of Artificial Sequence: Synthetic peptide
AVTQEDQAV

SEQ ID NO: 55 - protein, 9 aa, Artificial Sequence
  Description of Artificial Sequence: Synthetic peptide
EVDWKYQDH

SEQ ID NO: 56 - protein, 9 aa, Artificial Sequence
  Description of Artificial Sequence: Synthetic peptide
VDDKTLSKD

SEQ ID NO: 57 - protein, 9 aa, Artificial Sequence
  Description of Artificial Sequence: Synthetic peptide
QGQGKDPSQ

SEQ ID NO: 58 - protein, 9 aa, Artificial Sequence
  Description of Artificial Sequence: Synthetic peptide
GHQSEVQHS

SEQ ID NO: 59 - protein, 9 aa, Artificial Sequence
  Description of Artificial Sequence: Synthetic peptide
TGTSIWNQD

SEQ ID NO: 60 - protein, 11 aa, Artificial Sequence
  Description of Artificial Sequence: Synthetic peptide
GVHDSLQGYDA

SEQ ID NO: 61 - protein, 9 aa, Artificial Sequence
  Description of Artificial Sequence: Synthetic peptide
QKGQKIDKF

SEQ ID NO: 62 - protein, 9 aa, Artificial Sequence
  Description of Artificial Sequence: Synthetic peptide
DDELHDTRH

SEQ ID NO: 63 - protein, 9 aa, Artificial Sequence
  Description of Artificial Sequence: Synthetic peptide
ATTGDEWDR

SEQ ID NO: 64 - protein, 9 aa, Artificial Sequence
  Description of Artificial Sequence: Synthetic peptide
SHPHSNHTS

SEQ ID NO: 65 - protein, 9 aa, Artificial Sequence
  Description of Artificial Sequence: Synthetic peptide
WRTDYKYEE

SEQ ID NO: 66 - protein, 9 aa, Artificial Sequence
  Description of Artificial Sequence: Synthetic peptide
NDPHDSVPH

SEQ ID NO: 67 - protein, 9 aa, Artificial Sequence
  Description of Artificial Sequence: Synthetic peptide
GQQRENEQE

SEQ ID NO: 68 - protein, 9 aa, Artificial Sequence
  Description of Artificial Sequence: Synthetic peptide
GERQQDDAN

SEQ ID NO: 69 - protein, 9 aa, Artificial Sequence
  Description of Artificial Sequence: Synthetic peptide
AYREGSQWT

SEQ ID NO: 70 - protein, 9 aa, Artificial Sequence
  Description of Artificial Sequence: Synthetic peptide
EFYDHGIIQ

SEQ ID NO: 71 - protein, 9 aa, Artificial Sequence
  Description of Artificial Sequence: Synthetic peptide
ENEATRDQH

SEQ ID NO: 72 - protein, 9 aa, Artificial Sequence
  Description of Artificial Sequence: Synthetic peptide
GYDHEDNRG

SEQ ID NO: 73 - protein, 9 aa, Artificial Sequence
  Description of Artificial Sequence: Synthetic peptide
QPADMSAEF

SEQ ID NO: 74 - protein, 9 aa, Artificial Sequence
  Description of Artificial Sequence: Synthetic peptide
WVPFPHQQL

SEQ ID NO: 75 - protein, 9 aa, Artificial Sequence
  Description of Artificial Sequence: Synthetic peptide
REGRQDWVL

SEQ ID NO: 76 - protein, 112 aa, Artificial Sequence
  Description of Artificial Sequence: Synthetic polypeptide
MIPRGLSEAKPATPEIQEIVDKVKPQLEEKTNETYGKLEAVQYKTQVLAKEHGPDSWWSTNYYIKVRAGDNKYMHLKVFNGPQEKNQWVEEADRVLTGYQVDKNKDDELTGF

SEQ ID NO: 77 - protein, 112 aa, Artificial Sequence
  Description of Artificial Sequence: Synthetic polypeptide
MIPRGLSEAKPATPEIQEIVDKVKPQLEEKTNETYGKLEAVQYKTQVLAKEYGPEEWWSTNYYIKVRAGDNKYMHLKVFNGPGDYEQVLIHADRVLTGYQVDKNKDDELTGF

SEQ ID NO: 78 - protein, 112 aa, Artificial Sequence
  Description of Artificial Sequence: Synthetic polypeptide
MIPRGLSEAKPATPEIQEIVDKVKPQLEEKTNETYGKLEAVQYKTQVLAKDHGPIAWWSTNYYIKVRAGDNKYMHLKVFNGPEDTNTDGALADRVLTGYQVDKNKDDELTGF

SEQ ID NO: 79 - protein, 112 aa, Artificial Sequence
  Description of Artificial Sequence: Synthetic polypeptide
MIPRGLSEAKPATPEIQEIVDKVKPQLEEKTNETYGKLEAVQYKTQVLAKDWGPSNWWSTNYYIKVRAGDNKYMHLKVFNGPVDDKTLSKDADRVLTGYQVDKNKDDELTGF

SEQ ID NO: 80 - protein, 112 aa, Artificial Sequence
  Description of Artificial Sequence: Synthetic polypeptide
MIPRGLSEAKPATPEIQEIVDKVKPQLEEKTNETYGKLEAVQYKTQVLANTWFPESFWSTNYYIKVRAGDNKYMHLKVFNGPDDNQERQEHADRVLTGYQVDKNKDDELTGF

SEQ ID NO: 81 - protein, 112 aa, Artificial Sequence
  Description of Artificial Sequence: Synthetic polypeptide
MIPRGLSEAKPATPEIQEIVDKVKPQLEEKTNETYGKLEAVQYKTQVLAKPYGPRDWDSTNYYIKVRAGDNKYMHLKVFNGPEPQLDTSPIADRVLTGYQVDKNKDDELTGF

SEQ ID NO: 82 - protein, 112 aa, Artificial Sequence
  Description of Artificial Sequence: Synthetic polypeptide
MIPRGLSEAKPATPEIQEIVDKVKPQLEEKTNETYGKLEAVQYKTQVLAHAYGPRDWDSTNYYIKVRAGDNKYMHLKVFNGPPADHVLEEAADRVLTGYQVDKNKDDELTGF

SEQ ID NO: 83 - protein, 112 aa, Artificial Sequence
  Description of Artificial Sequence: Synthetic polypeptide
MIPRGLSEAKPATPEIQEIVDKVKPQLEEKTNETYGKLEAVQYKTQVLAAAHFPEHFWSTNYYIKVRAGDNKYMHLKVFNGPQPADMSAEFADRVLTGYQVDKNKDDELTGF

SEQ ID NO: 84 - protein, 112 aa, Artificial Sequence
  Description of Artificial Sequence: Synthetic polypeptide
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGF

SEQ ID NO: 85 - DNA, 369 bp, Artificial Sequence
  Description of Artificial Sequence: Synthetic polynucleotide
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaaaagattggggtccatctaactggtggtccaccaactattacattaaggttcgtgccggtgacaataagtatatgcacctgaaagtgttcaacggcccggttgatgataaaaccctgtctaaagatgcggaccgtgttctgaccggttaccaggttgacaagaacaaagatgacgagctgacgggtttcgcggccgcgggtcatcaccaccaccaccattag

SEQ ID NO: 86 - DNA, 369 bp, Artificial Sequence
  Description of Artificial Sequence: Synthetic polynucleotide
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaaaagatcatggtccaatcgcatggtggtccaccaactattacattaaggttcgtgccggtgacaataagtatatgcacctgaaagtgttcaacggcccggaagataccaacaccgatggtgcactggcggaccgtgttctgaccggttaccaggttgacaagaacaaagatgacgagctgacgggtttcgcggccgcgggtcatcaccaccaccaccattag

SEQ ID NO: 87 - DNA, 369 bp, Artificial Sequence
  Description of Artificial Sequence: Synthetic polynucleotide
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaaaaccatacggtccacgtgattgggattccaccaactattacattaaggttcgtgccggtgacaataagtatatgcacctgaaagtgttcaacggcccggaaccacagctggatacctctccaatcgcggaccgtgttctgaccggttaccaggttgacaagaacaaagatgacgagctgacgggtttcgcggccgcgggtcatcaccaccaccaccattag

SEQ ID NO: 88 - DNA, 369 bp, Artificial Sequence
  Description of Artificial Sequence: Synthetic polynucleotide
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaaacacctggtttccagaatctttttggtccaccaactattacattaaggttcgtgccggtgacaataagtatatgcacctgaaagtgttcaacggcccggatgataaccaggaacgtcaggaacatgcggaccgtgttctgaccggttaccaggttgacaagaacaaagatgacgagctgacgggtttcgcggccgcgggtcatcaccaccaccaccattag

SEQ ID NO: 89 - DNA, 369 bp, Artificial Sequence
  Description of Artificial Sequence: Synthetic polynucleotide
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcacgtgaaggtcgtcaggattgggttctgtccaccaactattacattaaggttcgtgccggtgacaataagtatatgcacctgaaagtgttcaacggcccgtgggttccatttccacatcagcagctggcggaccgtgttctgaccggttaccaggttgacaagaacaaagatgacgagctgacgggtttcgcggccgcgggtcatcaccaccaccaccattag

SEQ ID NO: 90 - DNA, 369 bp, Artificial Sequence
  Description of Artificial Sequence: Synthetic polynucleotide
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcacatgcatacggtccacgtgattgggattccaccaactattacattaaggttcgtgccggtgacaataagtatatgcacctgaaagtgttcaacggcccgccagcagatcatgttctggaagaagcagcggaccgtgttctgaccggttaccaggttgacaagaacaaagatgacgagctgacgggtttcgcggccgcgggtcatcaccaccaccaccattag

SEQ ID NO: 91 - DNA, 369 bp, Artificial Sequence
  Description of Artificial Sequence: Synthetic polynucleotide
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaaaagaatacggtccagaagaatggtggtccaccaactattacattaaggttcgtgccggtgacaataagtatatgcacctgaaagtgttcaacggcccgggtgattacgaacaggttctgatccatgcggaccgtgttctgaccggttaccaggttgacaagaacaaagatgacgagctgacgggtttcgcggccgcgggtcatcaccaccaccaccattag

SEQ ID NO: 92 - DNA, 371 bp, Artificial Sequence
  Description of Artificial Sequence: Synthetic polynucleotide
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcagctgctcatttcccggaacatttctggtccaccaactattacattaaggttcgtgccggtgacaataagtatatgcacctgaaagtgttcaacggcccgcagccggctgatatgtctgctgaattcgcggaccgtgttctgaccggttaccaggttgacaagaacaaagatgacgagctgacgggtttcctgcaggcggccgcgcaccaccaccaccaccactg

SEQ ID NO: 93 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 227 aa, Artificial Sequence; identical to SEQ ID NO: 93
  Description of Artificial Sequence: Synthetic polypeptide

SEQ ID NO: 95 - protein, 225 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 96 - protein, 227 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 227 aa, Artificial Sequence
  Description of Artificial Sequence: Synthetic polypeptide
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 232 aa, Artificial Sequence
  Description of Artificial Sequence: Synthetic polypeptide
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 227 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 226 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 101 - protein, 227 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 227 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 227 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 227 aa, Artificial Sequence
  Description of Artificial Sequence: Synthetic polypeptide
DKTHTCPPCPAPELLGGPSVFLFPPKPKDTLMISRTPEVTCVVVDVSHEDPEVKFNWYVDGVEVHNAKTKPREEQYHSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 233 aa, Homo sapiens
DKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 106 - protein, 233 aa, Artificial Sequence
  Description of Artificial Sequence: Synthetic polypeptide
DKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 107 - skipped

SEQ ID NO: 108 - protein, 382 aa, Artificial Sequence
  Description of Artificial Sequence: Synthetic polypeptide
MPLLLLLPLLWAGALAIPRGLSEAKPATPEIQEIVDKVKPQLEEKTGETYGKLEAVQYKTQVLAREGRQDWVLSTNYYIKVRAGDNKYMHLKVFNGPWVPFPHQQLADRVLTGYQVDKNKDDELTGFAAAGGGGSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - skipped

SEQ ID NO: 110 - protein, 382 aa, Homo sapiens
MPLLLLLPLLWAGALAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSIPRGLSEAKPATPEIQEIVDKVKPQLEEKTGETYGKLEAVQYKTQVLAREGRQDWVLSTNYYIKVRAGDNKYMHLKVFNGPWVPFPHQQLADRVLTGYQVDKNKDDELTGFAAA

SEQ ID NO: 111 - protein, 585 aa, Homo sapiens [sequence omitted]

SEQ ID NO: 112 - protein, 590 aa, Artificial Sequence
  Description of Artificial Sequence: Synthetic polypeptide
MPLLLLLPLLWAGALAQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIPRGLSEAKPATPEIQEIVDKVKPQLEEKTNETYGKLEAVQYKTQVLAAAHFPEHFWSTNYYIKVRAGDNKYMHLKVFNGPQPADMSAEFADRVLTGYQVDKNKDDELTGF

SEQ ID NO: 113 - protein, 231 aa, Artificial Sequence
  Description of Artificial Sequence: Synthetic polypeptide
MPLLLLLPLLWAGALAEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 594 aa, Artificial Sequence
  Description of Artificial Sequence: Synthetic polypeptide
MPLLLLLPLLWAGALAEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSIPRGLSEAKPATPEIQEIVDKVKPQLEEKTGETYGKLEAVQYKTQVLAREGRQDWVLSTNYYIKVRAGDNKYMHLKVFNGPWVPFPHQQLADRVLTGYQVDKNKDDELTGF

SEQ ID NO: 115 - protein, 230 aa, Artificial Sequence
  Description of Artificial Sequence: Synthetic polypeptide
MPLLLLLPLLWAGALADIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 596 aa, Artificial Sequence
  Description of Artificial Sequence: Synthetic polypeptide
MPLLLLLPLLWAGALAEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAEAAAKEAAAKEAAAKIPRGLSEAKPATPEIQEIVDKVKPQLEEKTGETYGKLEAVQYKTQVLAREGRQDWVLSTNYYIKVRAGDNKYMHLKVFNGPWVPFPHQQLADRVLTGYQVDKNKDDELTGF

SEQ ID NO: 117 - protein, 366 aa, Artificial Sequence
  Description of Artificial Sequence: Synthetic polypeptide
IPRGLSEAKPATPEIQEIVDKVKPQLEEKTGETYGKLEAVQYKTQVLAREGRQDWVLSTNYYIKVRAGDNKYMHLKVFNGPWVPFPHQQLADRVLTGYQVDKNKDDELTGFAAAGGGGSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 368 aa, Artificial Sequence
  Description of Artificial Sequence: Synthetic polypeptide
IPRGLSEAKPATPEIQEIVDKVKPQLEEKTGETYGKLEAVQYKTQVLAFALPEFEYMSTNYYIKVRAGDNKYMHLKVFNGPPMIRRKNEVADRVLTGYQVDKNKDDELTGFLHAAAGGGGSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 267 aa, Artificial Sequence
  Description of Artificial Sequence: Synthetic polypeptide
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFLQAAAHHHHHHC

SEQ ID NO: 120 - protein, 368 aa, Artificial Sequence
  Description of Artificial Sequence: Synthetic polypeptide
IPRGLSEAKPATPEIQEIVDKVKPQLEEKTGETYGKLEAVQYKTQVLAGGGGGGGGGSTNYYIKVRAGDNKYMHLKVFNGPGGGGGGGGGADRVLTGYQVDKNKDDELTGFLQAAAGGGGSGGGGSGGGGSGGGGSEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 134 aa, Artificial Sequence
  Description of Artificial Sequence: Synthetic polypeptide
  MOD_RES (50)..(69): Any amino acid
  MISC_FEATURE (50)..(69): This region may encompass 3-20 residues
  MOD_RES (94)..(113): Any amino acid
  MISC_FEATURE (94)..(113): This region may encompass 3-20 residues
  See specification as filed for detailed description of substitutions and preferred embodiments
MIPGGLSEAKPATPEIQEIVDKVKPQLEEKTNETYGKLEAVQYKTQVLAXXXXXXXXXXXXXXXXXXXXGTNYYIKVRAGDNKYMHLKVFKSLXXXXXXXXXXXXXXXXXXXXEDLVLTGYQVDKNKDDELTGF

SEQ ID NO: 122 - protein, 9 aa, Artificial Sequence
  Description of Artificial Sequence: Synthetic peptide
  MOD_RES (1)..(1): Any amino acid with a basic sidechain or aromatic sidechain
  MOD_RES (2)..(2): Any amino acid
  MOD_RES (3)..(3): Any amino acid with an aromatic or basic sidechain
  MOD_RES (6)..(6): Any amino acid with a neutral polar or non-polar sidechain or a charged (acidic or basic) sidechain
  MOD_RES (7)..(7): Any amino acid with a neutral polar or a charged (acidic or basic) or a small aliphatic or an aromatic sidechain
  See specification as filed for detailed description of substitutions and preferred embodiments
XXXFPXXFW

SEQ ID NO: 123 - protein, 8 aa, Artificial Sequence
  Description of Artificial Sequence: Synthetic peptide
TGRGPSWV

SEQ ID NO: 124 - protein, 8 aa, Artificial Sequence
  Description of Artificial Sequence: Synthetic peptide
SARGPSRW

SEQ ID NO: 125 - protein, 8 aa, Artificial Sequence
  Description of Artificial Sequence: Synthetic peptide
TARGPSFK

SEQ ID NO: 126 - protein, 8 aa, Artificial Sequence
  Description of Artificial Sequence: Synthetic peptide
LSGRSDNH

SEQ ID NO: 127 - protein, 8 aa, Artificial Sequence
  Description of Artificial Sequence: Synthetic peptide
GGWHTGRN

SEQ ID NO: 128 - protein, 8 aa, Artificial Sequence
  Description of Artificial Sequence: Synthetic peptide
HTGRSGAL

SEQ ID NO: 129 - protein, 8 aa, Artificial Sequence
  Description of Artificial Sequence: Synthetic peptide
PLTGRSGG

SEQ ID NO: 130 - protein, 8 aa, Artificial Sequence
  Description of Artificial Sequence: Synthetic peptide
AARGPAIH

SEQ ID NO: 131 - protein, 8 aa, Artificial Sequence
  Description of Artificial Sequence: Synthetic peptide
RGPAFNPM

SEQ ID NO: 132 - protein, 8 aa, Artificial Sequence
  Description of Artificial Sequence: Synthetic peptide
SSRGPAYL

SEQ ID NO: 133 - protein, 8 aa, Artificial Sequence
  Description of Artificial Sequence: Synthetic peptide
RGPATPIM

SEQ ID NO: 134 - protein, 4 aa, Artificial Sequence
  Description of Artificial Sequence: Synthetic peptide
RGPA

SEQ ID NO: 135 - protein, 10 aa, Artificial Sequence
  Description of Artificial Sequence: Synthetic peptide
GGQPSGMWGW

SEQ ID NO: 136 - protein, 10 aa, Artificial Sequence
  Description of Artificial Sequence: Synthetic peptide
FPRPLGITGL

SEQ ID NO: 137 - protein, 10 aa, Artificial Sequence
  Description of Artificial Sequence: Synthetic peptide
VHMPLGFLGP

SEQ ID NO: 138 - protein, 8 aa, Artificial Sequence
  Description of Artificial Sequence: Synthetic peptide
SPLTGRSG

SEQ ID NO: 139 - protein, 8 aa, Artificial Sequence
  Description of Artificial Sequence: Synthetic peptide
SAGFSLPA

SEQ ID NO: 140 - protein, 9 aa, Artificial Sequence
  Description of Artificial Sequence: Synthetic peptide
LAPLGLQRR

SEQ ID NO: 141 - protein, 8 aa, Artificial Sequence
  Description of Artificial Sequence: Synthetic peptide
SGGPLGVR

SEQ ID NO: 142 - protein, 4 aa, Artificial Sequence
  Description of Artificial Sequence: Synthetic peptide
PLGL

SEQ ID NO: 143 - protein, 7 aa, Artificial Sequence
  Description of Artificial Sequence: Synthetic peptide
GPRSFGL

SEQ ID NO: 144 - protein, 6 aa, Artificial Sequence
  Description of Artificial Sequence: Synthetic peptide
GPRSFG

SEQ ID NO: 145 - protein, 8 aa, Artificial Sequence
  Description of Artificial Sequence: Synthetic peptide
ISSGLLSS

SEQ ID NO: 146 - protein, 8 aa, Artificial Sequence
  Description of Artificial Sequence: Synthetic peptide
QNQALRMA

SEQ ID NO: 147 - protein, 8 aa, Artificial Sequence
  Description of Artificial Sequence: Synthetic peptide
AQNLLGMV

SEQ ID NO: 148 - protein, 8 aa, Artificial Sequence
  Description of Artificial Sequence: Synthetic peptide
STFPFGMF

SEQ ID NO: 149 - protein, 8 aa, Artificial Sequence
  Description of Artificial Sequence: Synthetic peptide
PVGYTSSL

SEQ ID NO: 150 - protein, 8 aa, Artificial Sequence
  Description of Artificial Sequence: Synthetic peptide
DWLYWPGI

SEQ ID NO: 151 - protein, 8 aa, Artificial Sequence
  Description of Artificial Sequence: Synthetic peptide
MIAPVAYR

SEQ ID NO: 152 - protein, 8 aa, Artificial Sequence
  Description of Artificial Sequence: Synthetic peptide
RPSPMWAY

SEQ ID NO: 153 - protein, 8 aa, Artificial Sequence
  Description of Artificial Sequence: Synthetic peptide
WATPRPMR

SEQ ID NO: 154 - protein, 8 aa, Artificial Sequence
  Description of Artificial Sequence: Synthetic peptide
FRLLDWQW

SEQ ID NO: 155 - protein, 8 aa, Artificial Sequence
  Description of Artificial Sequence: Synthetic peptide
LKAAPRWA

SEQ ID NO: 156 - protein, 8 aa, Artificial Sequence
  Description of Artificial Sequence: Synthetic peptide
GPSHLVLT

SEQ ID NO: 157 - protein, 8 aa, Artificial Sequence
  Description of Artificial Sequence: Synthetic peptide
LPGGLSPW

SEQ ID NO: 158 - protein, 8 aa, Artificial Sequence
  Description of Artificial Sequence: Synthetic peptide
MGLFSEAG

SEQ ID NO: 159 - protein, 8 aa, Artificial Sequence
  Description of Artificial Sequence: Synthetic peptide
SPLPLRVP

SEQ ID NO: 160 - protein, 8 aa, Artificial Sequence
  Description of Artificial Sequence: Synthetic peptide
RMHLRSLG

SEQ ID NO: 161 - protein, 8 aa, Artificial Sequence
  Description of Artificial Sequence: Synthetic peptide
LAAPLGLL

SEQ ID NO: 162 - protein, 8 aa, Artificial Sequence
  Description of Artificial Sequence: Synthetic peptide
AVGLLAPP

SEQ ID NO: 163 - protein, 8 aa, Artificial Sequence
  Description of Artificial Sequence: Synthetic peptide
LLAPSHRA

SEQ ID NO: 164 - protein, 8 aa, Artificial Sequence
  Description of Artificial Sequence: Synthetic peptide
PAGLWLDP

SEQ ID NO: 165 - protein, 7 aa, Artificial Sequence
  Description of Artificial Sequence: Synthetic peptide
ISSGLSS

SEQ ID NO: 166 - protein, 6 aa, Artificial Sequence
  Description of Artificial Sequence: Synthetic 6xHis tag
HHHHHH

SEQ ID NO: 167 - protein, 18 aa, Homo sapiens
MKWVTFISLLFLFSSAYS

SEQ ID NO: 168 - protein, 22 aa, Unknown
  Description of Unknown: Ig kappa light chain signal sequence
MDMRAPAGIFGFLLVLFPGYRS

SEQ ID NO: 169 - protein, 19 aa, Homo sapiens
MTRLTVLALLAGLLASSRA

SEQ ID NO: 170 - protein, 19 aa, Unknown
  Description of Unknown: IgG heavy chain signal sequence
MELGLSWIFLLAILKGVQC

SEQ ID NO: 171 - protein, 19 aa, Unknown
  Description of Unknown: IgG heavy chain signal sequence
MELGLRWVFLVAILEGVQC

SEQ ID NO: 172 - protein, 19 aa, Unknown
  Description of Unknown: IgG heavy chain signal sequence
MKHLWFFLLLVAAPRWVLS

SEQ ID NO: 173 - protein, 19 aa, Unknown
  Description of Unknown: IgG heavy chain signal sequence
MDWTWRILFLVAAATGAHS

SEQ ID NO: 174 - protein, 19 aa, Unknown
  Description of Unknown: IgG heavy chain signal sequence
MDWTWRFLFVVAAATGVQS

SEQ ID NO: 175 - protein, 19 aa, Unknown
  Description of Unknown: IgG heavy chain signal sequence
MEFGLSWLFLVAILKGVQC

SEQ ID NO: 176 - protein, 19 aa, Unknown
  Description of Unknown: IgG heavy chain signal sequence
MEFGLSWVFLVALFRGVQC

SEQ ID NO: 177 - protein, 26 aa, Unknown
  Description of Unknown: IgG heavy chain signal sequence
MDLLHKNMKHLWFFLLLVAAPRWVLS

SEQ ID NO: 178 - protein, 22 aa, Unknown
  Description of Unknown: IgG Kappa light signal sequence
MDMRVPAQLLGLLLLWLSGARC

SEQ ID NO: 179 - protein, 22 aa, Unknown
  Description of Unknown: IgG Kappa light signal sequence
MKYLLPTAAAGLLLLAAQPAMA

SEQ ID NO: 180 - protein, 17 aa, Gaussia princeps
MGVKVLFALICIAVAEA

SEQ ID NO: 181 - protein, 18 aa, Homo sapiens
MAFLWLLSCWALLGTTFG

SEQ ID NO: 182 - protein, 14 aa, Homo sapiens
MQLLSCIALILALV

SEQ ID NO: 183 - protein, 15 aa, Homo sapiens
MNLLLILTFVAAAVA

SEQ ID NO: 184 - protein, 16 aa, Homo sapiens
MPLLLLLPLLWAGALA

SEQ ID NO: 185 - protein, 30 aa, Unknown
  Description of Unknown: Prolactin signal sequence
MDSKGSSQKGSRLLLLLVVSNLLLCQGVVS

SEQ ID NO: 186 - protein, 23 aa, Homo sapiens
MDAMKRGLCCVLLLCGAVFVSPS

SEQ ID NO: 187 - protein, 16 aa, Artificial Sequence
  Description of Artificial Sequence: Synthetic peptide
MLLLLLLLLLLALALA

SEQ ID NO: 188 - protein, 21 aa, Artificial Sequence
  Description of Artificial Sequence: Synthetic peptide
MWWRLWWLLLLLLLLWPMVWA

SEQ ID NO: 189 - protein, 5 aa, Artificial Sequence
  Description of Artificial Sequence: Synthetic peptide
GGGGS

SEQ ID NO: 190 - protein, 5 aa, Artificial Sequence
  Description of Artificial Sequence: Synthetic peptide
EAAAK

SEQ ID NO: 191 - protein, 30 aa, Artificial Sequence
  Description of Artificial Sequence: Synthetic polypeptide
  MISC_FEATURE (1)..(30): This sequence may encompass 1-6 “Gly Gly Gly Gly Ser” repeating units
GGGGSGGGGSGGGGSGGGGSGGGGSGGGGS

SEQ ID NO: 192 - protein, 8 aa, Artificial Sequence
  Description of Artificial Sequence: Synthetic peptide
GGGGGGGG

SEQ ID NO: 193 - protein, 6 aa, Artificial Sequence
  Description of Artificial Sequence: Synthetic peptide
GGGGGG

SEQ ID NO: 194 - protein, 18 aa, Artificial Sequence
  Description of Artificial Sequence: Synthetic peptide
KESGSVSSEQLAQFRSLD

SEQ ID NO: 195 - protein, 14 aa, Artificial Sequence
  Description of Artificial Sequence: Synthetic peptide
EGKSSGSGSESKST

SEQ ID NO: 196 - protein, 12 aa, Artificial Sequence
  Description of Artificial Sequence: Synthetic peptide
GSAGSAAGSGEF

SEQ ID NO: 197 - protein, 30 aa, Artificial Sequence
  Description of Artificial Sequence: Synthetic polypeptide
  MISC_FEATURE (1)..(30): This sequence may encompass 1-6 “Glu Ala Ala Ala Lys” repeating units
EAAAKEAAAKEAAAKEAAAKEAAAKEAAAK

SEQ ID NO: 198 - protein, 46 aa, Artificial Sequence
  Description of Artificial Sequence: Synthetic polypeptide
AEAAAKEAAAKEAAAKEAAAKALEAEAAAKEAAAKEAAAKEAAAKA

SEQ ID NO: 199 - protein, 5 aa, Artificial Sequence
  Description of Artificial Sequence: Synthetic peptide
PAPAP

SEQ ID NO: 200 - protein, 12 aa, Artificial Sequence
  Description of Artificial Sequence: Synthetic peptide
AEAAAKEAAAKA

SEQ ID NO: 201 - protein, 4 aa, Artificial Sequence
  Description of Artificial Sequence: Synthetic peptide
GGSG

SEQ ID NO: 202 - protein, 4 aa, Artificial Sequence
  Description of Artificial Sequence: Synthetic peptide
GSGS

SEQ ID NO: 203 - protein, 4 aa, Artificial Sequence
  Description of Artificial Sequence: Synthetic peptide
GGGS

SEQ ID NO: 204 - protein, 22 aa, Artificial Sequence
  Description of Artificial Sequence: Synthetic peptide
  MISC_FEATURE (2)..(22): This region may encompass 1-7 “Gly Gly Ser” repeating units
SGGSGGSGGSGGSGGSGGSGGS

SEQ ID NO: 205 - protein, 7 aa, Artificial Sequence
  Description of Artificial Sequence: Synthetic peptide
GGGSGGG

SEQ ID NO: 206 - protein, 8 aa, Artificial Sequence
  Description of Artificial Sequence: Synthetic peptide
ESGGGGVT

SEQ ID NO: 207 - protein, 9 aa, Artificial Sequence
  Description of Artificial Sequence: Synthetic peptide
LESGGGGVT

SEQ ID NO: 208 - protein, 6 aa, Artificial Sequence
  Description of Artificial Sequence: Synthetic peptide
GRAQVT

SEQ ID NO: 209 - protein, 6 aa, Artificial Sequence
  Description of Artificial Sequence: Synthetic peptide
WRAQVT

SEQ ID NO: 210 - protein, 8 aa, Artificial Sequence
  Description of Artificial Sequence: Synthetic peptide
ARGRAQVT

SEQ ID NO: 211 - protein, 16 aa, Homo sapiens
DKTHTCPPCPAPELLG

SEQ ID NO: 212 - protein, 16 aa, Artificial Sequence
  Description of Artificial Sequence: Synthetic peptide
GSTHTCPPCPAPELLG

SEQ ID NO: 213 - protein, 21 aa, Artificial Sequence
  Description of Artificial Sequence: Synthetic peptide
EPKSCDKTHTCPPCPAPELLG

SEQ ID NO: 214 - protein, 24 aa, Artificial Sequence
  Description of Artificial Sequence: Synthetic peptide
EPKSCDKTHTCPPCPAPELLGGPS

SEQ ID NO: 215 - protein, 24 aa, Artificial Sequence
  Description of Artificial Sequence: Synthetic peptide
EPKSSDKTHTCPPCPAPELLGGPS

SEQ ID NO: 216 - protein, 24 aa, Artificial Sequence
  Description of Artificial Sequence: Synthetic peptide
EPKSSDKTHTCPPCPAPELLGGSS

SEQ ID NO: 217 - protein, 24 aa, Artificial Sequence
  Description of Artificial Sequence: Synthetic peptide
EPKSSGSTHTCPPCPAPELLGGSS

SEQ ID NO: 218 - protein, 19 aa, Artificial Sequence
  Description of Artificial Sequence: Synthetic peptide
DKTHTCPPCPAPELLGGPS

SEQ ID NO: 219 - protein, 19 aa, Artificial Sequence
  Description of Artificial Sequence: Synthetic peptide
DKTHTCPPCPAPELLGGSS

SEQ ID NO: 220 - protein, 6 aa, Homo sapiens
RGVFRR

SEQ ID NO: 221 - protein, 16 aa, Artificial Sequence
  Description of Artificial Sequence: Synthetic peptide
AEAAAKEAAAKEAAAK

SEQ ID NO: 222 - protein, 118 aa, Artificial Sequence [sequence omitted]

SEQ ID NO: 223 - protein, 122 aa, Artificial Sequence
  Description of Artificial Sequence: Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTK

SEQ ID NO: 224 - protein, 108 aa, Artificial Sequence
  Description of Artificial Sequence: Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 225 - protein, 15 aa, Artificial Sequence
  Description of Artificial Sequence: Synthetic peptide
GGGGSGGGGSGGGGS